SEQUENCE LISTING (25 sequences)

SEQ ID NO: 1 - RNA, 100 nt, Artificial Sequence
  Description of Artificial Sequence Synthetic polynucleotide
cucucagcugguacacggcaguuuuagagcuagaaauagcaaguuaaaauaaggcuaguccguuaucaacuugaaaaaguggcaccgagucggugcuuuu

SEQ ID NO: 2 - RNA, 100 nt, Artificial Sequence
  Description of Artificial Sequence Synthetic polynucleotide
ggccucggcgcugacgaucuguuuuagagcuagaaauagcaaguuaaaauaaggcuaguccguuaucaacuugaaaaaguggcaccgagucggugcuuuu

SEQ ID NO: 3 - RNA, 100 nt, Artificial Sequence
  Description of Artificial Sequence Synthetic polynucleotide
ggccacggagcgagacaucuguuuuagagcuagaaauagcaaguuaaaauaaggcuaguccguuaucaacuugaaaaaguggcaccgagucggugcuuuu

SEQ ID NO: 4 - RNA, 100 nt, Artificial Sequence
  Description of Artificial Sequence Synthetic polynucleotide
uggucagggcaagagcuauuguuuuagagcuagaaauagcaaguuaaaauaaggcuaguccguuaucaacuugaaaaaguggcaccgagucggugcuuuu

SEQ ID NO: 5 - RNA, 100 nt, Artificial Sequence
  Description of Artificial Sequence Synthetic polynucleotide
acagcgacgccgcgagccagguuuuagagcuagaaauagcaaguuaaaauaaggcuaguccguuaucaacuugaaaaaguggcaccgagucggugcuuuu

SEQ ID NO: 6 - RNA, 100 nt, Artificial Sequence
  Description of Artificial Sequence Synthetic polynucleotide
ccaauaucaggagacuaggaguuuuagagcuagaaauagcaaguuaaaauaaggcuaguccguuaucaacuugaaaaaguggcaccgagucggugcuuuu

SEQ ID NO: 7 - RNA, 74 nt, Artificial Sequence
  Description of Artificial Sequence Synthetic oligonucleotide
aacagcauagcaaguuaaaauaaggcuaguccguuaucaacuugaaaaaguggcaccgagucggugcuuuuuuu

SEQ ID NO: 8 - protein, 1379 aa, Artificial Sequence
  Description of Artificial Sequence Synthetic polypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 9 - DNA, 4140 bp, Artificial Sequence
  Description of Artificial Sequence Synthetic polynucleotide
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 10 - DNA, 4140 bp, Artificial Sequence
  Description of Artificial Sequence Synthetic polynucleotide
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 11 - RNA, 4197 nt, Artificial Sequence
  Description of Artificial Sequence Synthetic polynucleotide
auggacaagaaguacuccaucggccuggacaucggcaccaacuccgugggcugggccgugaucaccgacgaguacaaggugcccuccaagaaguucaaggugcugggcaacaccgaccggcacuccaucaagaagaaccugaucggcgcccugcuguucgacuccggcgagaccgccgaggccacccggcugaagcggaccgcccggcggcgguacacccggcggaagaaccggaucugcuaccugcaggagaucuucuccaacgagauggccaagguggacgacuccuucuuccaccggcuggaggaguccuuccugguggaggaggacaagaagcacgagcggcaccccaucuucggcaacaucguggacgagguggccuaccacgagaaguaccccaccaucuaccaccugcggaagaagcugguggacuccaccgacaaggccgaccugcggcugaucuaccuggcccuggcccacaugaucaaguuccggggccacuuccugaucgagggcgaccugaaccccgacaacuccgacguggacaagcuguucauccagcuggugcagaccuacaaccagcuguucgaggagaaccccaucaacgccuccggcguggacgccaaggccauccuguccgcccggcuguccaagucccggcggcuggagaaccugaucgcccagcugcccggcgagaagaagaacggccuguucggcaaccugaucgcccugucccugggccugacccccaacuucaaguccaacuucgaccuggccgaggacgccaagcugcagcuguccaaggacaccuacgacgacgaccuggacaaccugcuggcccagaucggcgaccaguacgccgaccuguuccuggccgccaagaaccuguccgacgccauccugcuguccgacauccugcgggugaacaccgagaucaccaaggccccccuguccgccuccaugaucaagcgguacgacgagcaccaccaggaccugacccugcugaaggcccuggugcggcagcagcugcccgagaaguacaaggagaucuucuucgaccaguccaagaacggcuacgccggcuacaucgacggcggcgccucccaggaggaguucuacaaguucaucaagcccauccuggagaagauggacggcaccgaggagcugcuggugaagcugaaccgggaggaccugcugcggaagcagcggaccuucgacaacggcuccaucccccaccagauccaccugggcgagcugcacgccauccugcggcggcaggaggacuucuaccccuuccugaaggacaaccgggagaagaucgagaagauccugaccuuccggauccccuacuacgugggcccccuggcccggggcaacucccgguucgccuggaugacccggaaguccgaggagaccaucacccccuggaacuucgaggaggugguggacaagggcgccuccgcccaguccuucaucgagcggaugaccaacuucgacaagaaccugcccaacgagaaggugcugcccaagcacucccugcuguacgaguacuucaccguguacaacgagcugaccaaggugaaguacgugaccgagggcaugcggaagcccgccuuccuguccggcgagcagaagaaggccaucguggaccugcuguucaagaccaaccggaaggugaccgugaagcagcugaaggaggacuacuucaagaagaucgagugcuucgacuccguggagaucuccggcguggaggaccgguucaacgccucccugggcaccuaccacgaccugcugaagaucaucaaggacaaggacuuccuggacaacgaggagaacgaggacauccuggaggacaucgugcugacccugacccuguucgaggaccgggagaugaucgaggagcggcugaagaccuacgcccaccuguucgacgacaaggugaugaagcagcugaagcggcggcgguacaccggcuggggccggcugucccggaagcugaucaacggcauccgggacaagcaguccggcaagaccauccuggacuuccugaaguccgacggcuucgccaaccggaacuucaugcagcugauccacgacgacucccugaccuucaaggaggacauccagaaggcccagguguccggccagggcgacucccugcacgagcacaucgccaaccuggccggcucccccgccaucaagaagggcauccugcagaccgugaaggugguggacgagcuggugaaggugaugggccggcacaagcccgagaacaucgugaucgagauggcccgggagaaccagaccacccagaagggccagaagaacucccgggagcggaugaagcggaucgaggagggcaucaaggagcugggcucccagauccugaaggagcaccccguggagaacacccagcugcagaacgagaagcuguaccuguacuaccugcagaacggccgggacauguacguggaccaggagcuggacaucaaccggcuguccgacuacgacguggaccacaucgugccccaguccuuccugaaggacgacuccaucgacaacaaggugcugacccgguccgacaagaaccggggcaaguccgacaacgugcccuccgaggagguggugaagaagaugaagaacuacuggcggcagcugcugaacgccaagcugaucacccagcggaaguucgacaaccugaccaaggccgagcggggcggccuguccgagcuggacaaggccggcuucaucaagcggcagcugguggagacccggcagaucaccaagcacguggcccagauccuggacucccggaugaacaccaaguacgacgagaacgacaagcugauccgggaggugaaggugaucacccugaaguccaagcugguguccgacuuccggaaggacuuccaguucuacaaggugcgggagaucaacaacuaccaccacgcccacgacgccuaccugaacgccguggugggcaccgcccugaucaagaaguaccccaagcuggaguccgaguucguguacggcgacuacaagguguacgacgugcggaagaugaucgccaaguccgagcaggagaucggcaaggccaccgccaaguacuucuucuacuccaacaucaugaacuucuucaagaccgagaucacccuggccaacggcgagauccggaagcggccccugaucgagaccaacggcgagaccggcgagaucgugugggacaagggccgggacuucgccaccgugcggaaggugcuguccaugccccaggugaacaucgugaagaagaccgaggugcagaccggcggcuucuccaaggaguccauccugcccaagcggaacuccgacaagcugaucgcccggaagaaggacugggaccccaagaaguacggcggcuucgacucccccaccguggccuacuccgugcuggugguggccaagguggagaagggcaaguccaagaagcugaaguccgugaaggagcugcugggcaucaccaucauggagcgguccuccuucgagaagaaccccaucgacuuccuggaggccaagggcuacaaggaggugaagaaggaccugaucaucaagcugcccaaguacucccuguucgagcuggagaacggccggaagcggaugcuggccuccgccggcgagcugcagaagggcaacgagcuggcccugcccuccaaguacgugaacuuccuguaccuggccucccacuacgagaagcugaagggcucccccgaggacaacgagcagaagcagcuguucguggagcagcacaagcacuaccuggacgagaucaucgagcagaucuccgaguucuccaagcgggugauccuggccgacgccaaccuggacaaggugcuguccgccuacaacaagcaccgggacaagcccauccgggagcaggccgagaacaucauccaccuguucacccugaccaaccugggcgcccccgccgccuucaaguacuucgacaccaccaucgaccggaagcgguacaccuccaccaaggaggugcuggacgccacccugauccaccaguccaucaccggccuguacgagacccggaucgaccugucccagcugggcggcgacggcggcggcucccccaagaagaagcggaagguguccgaguccgccacccccgaguccguguccggcuggcggcuguucaagaagaucuccuga

SEQ ID NO: 12 - DNA, 4607 bp, Artificial Sequence
  Description of Artificial Sequence Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgcggccgccaccatgggatcttggacactgtgttgcgtgtccctgtgcatcctggtggccaagcacacagatgccggcgtgatccagtctcctagacacgaagtgaccgagatgggccaagaagtgaccctgcgctgcaagcctatcagcggccacgattacctgttctggtacagacagaccatgatgagaggcctggaactgctgatctacttcaacaacaacgtgcccatcgacgacagcggcatgcccgaggatagattcagcgccaagatgcccaacgccagcttcagcaccctgaagatccagcctagcgagcccagagatagcgccgtgtacttctgcgccagcagaaagacaggcggctacagcaatcagccccagcactttggagatggcacccggctgagcatcctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcaggggcggctccggtgccacaaacttctccctgctcaagcaggccggagatgtggaagagaaccctggccctatggaaaccctgctgaaggtgctgagcggcacactgctgtggcagctgacatgggtccgatctcagcagcctgtgcagtctcctcaggccgtgattctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgtggcaccgcctggatcaacgactacaagctgtctttcggagccggcaccacagtgacagtgcgggccaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagactgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttcagaatcctgctgctcaaggtggccggcttcaacctgctgatgaccctgagactgtggtccagctaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 13 - DNA, 4869 bp, Artificial Sequence
  Description of Artificial Sequence Synthetic polynucleotide
taatcagaattggttaattggttgtaacattattcagattgggcttgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacaccacgcgtttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgagggccgcggcagcctgctgacctgcggcgacgtggaggagaatcccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaagaattctctggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttc

SEQ ID NO: 14 - DNA, 4607 bp, Artificial Sequence; identical to SEQ ID NO: 12
  Description of Artificial Sequence Synthetic polynucleotide

SEQ ID NO: 15 - DNA, 3294 bp, Artificial Sequence
  Description of Artificial Sequence Synthetic polynucleotide
tgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagccccatccttaggcctcctccttccgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaacatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgctagcgctaccggactcaatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgttagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgt

SEQ ID NO: 16 - DNA, 4250 bp, Artificial Sequence
  Description of Artificial Sequence Synthetic polynucleotide
gaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagccccatccttaggcctcctccttagttattaatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaacatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgctagcgctaccggactcaatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtgtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgttcccctgcatcccccttcccctgcatcccccagaggccccaggccacctacttggcctggaccccacgagaggccaccccagccctgtctaccaggctgccttttgggtggattctcctccaa

SEQ ID NO: 17 - DNA, 1556 bp, Artificial Sequence
  Description of Artificial Sequence Synthetic polynucleotide
gagggccgcggcagcctgctgacctgcggcgacgtggaggagaatcccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctct

SEQ ID NO: 18 - DNA, 4619 bp, Artificial Sequence
  Description of Artificial Sequence Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgcggccgccaccatgggatgtagacttctgtgttgcgccgtgctgtgtctgcttggagctggcgaactggtgcctatggaaaccggcgtgacccagacacctagacacctggtcatgggcatgacaaacaagaaaagcctgaagtgcgagcagcacctgggccacaatgccatgtactggtacaagcagagcgccaagaaacccctggaactgatgttcgtgtacagcctggaagagagggtcgagaacaacagcgtgcccagcagattcagccctgagtgccctaatagcagccacctgtttctgcatctgcacaccctgcagcctgaggactctgccctgtatctgtgtgccagcagccaggactacctggtgtccaacgagaagctgttcttcggcagcggcacacagctgagcgtgctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggaaaagccactctgtacgctgtgctggtgtccgctctggtgctgatggccatggtcaagcggaaggatagcaggggcggctccggtgccacaaacttctccctgctcaagcaggccggagatgtggaagagaaccctggccctatgatcagcctgagagtgctgctggtcatcctgtggctgcagctgtcttgggtctggtcccagcggaaagaggtggaacaggaccccggacctttcaatgtgcctgaaggcgccaccgtggccttcaactgcacctacagcaatagcgccagccagagcttcttctggtacagacaggactgccggaaagaacccaagctgctgatgagcgtgtacagcagcggcaacgaggacggcagattcacagcccagctgaacagagccagccagtacatcagcctgctgatccgggatagcaagctgagcgatagcgccacctacctgtgcgtggtcaacctgctgtctaatcaaggcggcaagctgatcttcggccagggcacagagctgagcgtgaagcccaacattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccggatcctgctgctgaaagtggccggcttcaacctcctgatgaccctgagactgtggtccagctaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 19 - RNA, 20 nt, Artificial Sequence
  Description of Artificial Sequence Synthetic oligonucleotide
cucucagcugguacacggca

SEQ ID NO: 20 - RNA, 20 nt, Artificial Sequence
  Description of Artificial Sequence Synthetic oligonucleotide
ggccucggcgcugacgaucu

SEQ ID NO: 21 - protein, 6 aa, Artificial Sequence
  Description of Artificial Sequence Synthetic 6xHis tag
HHHHHH

SEQ ID NO: 22 - protein, 8 aa, Artificial Sequence
  Description of Artificial Sequence Synthetic 8xHis tag
HHHHHHHH

SEQ ID NO: 23 - RNA, 13 nt, Artificial Sequence
  Description of Artificial Sequence Synthetic oligonucleotide
gccgccrccaugg

SEQ ID NO: 24 - RNA, 10 nt, Artificial Sequence
  Description of Artificial Sequence Synthetic oligonucleotide
gccrccaugg

SEQ ID NO: 25 - RNA, 100 nt, Artificial Sequence
  Description of Artificial Sequence Synthetic polynucleotide
  misc_feature (1)..(100): This sequence may encompass 95-100 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2024/0300977 A1
(43) Pub. Date: Sep. 12, 2024

(54) INHIBITORS OF DNA-DEPENDENT PROTEIN KINASE AND COMPOSITIONS AND USES THEREOF

(71) Applicant: Intellia Therapeutics, Inc., Cambridge, MA (US)

(72) Inventors: Micah Maetani, Cambridge, MA (US); Xin Jenny Xie, Sudbury, MA (US); Anthony Forget, Charlton, MA (US); Aaron Prodeus, Marlborough, MA (US); Stephanie Yazinski, Lynnfield, MA (US); Rubina Giare Parmar, Acton, MA (US)

(21) Appl. No.: 18/287,227

(22) PCT Filed: Apr. 15, 2022
(86) PCT No.: PCT/US22/25075

§ 371 Date: Oct. 17, 2023

Related U.S. Application Data
(60) Provisional application No. 63/176,225, filed on Apr. 17, 2021.

(51) Int. Cl. C07D 519/00; C12N 5/071; C12N 5/0783; C12N 9/22; C12N 15/113; C12N 15/90

(52) U.S. Cl. CPC C07D 519/00; C12N 5/0636; C12N 5/067; C12N 9/22; C12N 15/113; C12N 15/907; C12N 2310/20; C12N 2510/00

(57) ABSTRACT

The present disclosure relates to inhibitors of DNA protein kinase, and compositions and methods of use thereof. In some embodiments, the inhibitors have the structure of Formula I:

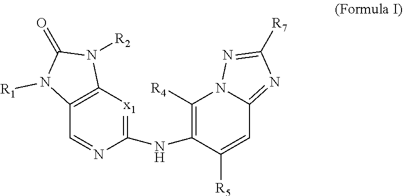

or a salt thereof,
wherein:
   x₁ is C—R₃ or N;
   R₁ is C₁-C₃ alkyl;
   R₂ is cycloalkyl or heterocyclyl, and cycloalkyl and heterocyclyl are optionally substituted with one or more R₆;
   R₃ is H or C₁-C₃ alkyl;
   R₄ is H or C₁-C₃ alkyl;
   R₅ is C₁-C₃ alkyl;
   each R₆ is independently selected from hydroxy, halo, alkyl, alkoxy, cycloalkyl, amino, and cyano, or two R₆, taken together with the atom or atoms to which they are bonded, form a spirocyclic or fused ring; and
   R₇ is H or C₁-C₃ alkyl.

### CROSS-REFERENCE TO RELATED APPLICATIONS

This application is the U.S. national phase of International Application PCT/US22/25705, filed Apr. 15, 2022, which claims the benefit of priority to U.S. Provisional Patent Application No. 63/176,225, filed Apr. 17, 2021, the entire contents of each of which are incorporated herein by reference.

### SEQUENCE LISTING

This application contains a Sequence Listing in ASCII computer readable form. The Sequence Listing is incorporated herein by reference. Said ASCII file, created on Jun. 20, 2022, is named ILH-00801_SL.txt and is 73,728 bytes in size.

### BACKGROUND

The ability to modify the genome of any cell at a precise location has improved with the recent discovery and implementation of CRISPR/Cas9 editing technology. However, the capacity to introduce specific directed changes at given loci is hindered by the fact that the major cellular repair pathway that occurs following Cas9-mediated DNA cleavage is the erroneous non-homologous end joining (NHEJ) pathway. Homology-directed recombination (HDR) is less efficient than NHEJ, reducing editing efficiencies in eukaryotic cells.

DNA-dependent protein kinase (DNA-PK) is a nuclear serine/threonine kinase that has been shown to be essential in DNA double stranded break repair machinery. In mammals, the predominant pathway for repair of double stranded DNA breaks is the non-homologous end joining (NHEJ) pathway which is functional regardless of the phase of the cell cycle and acts by removing non-ligatable ends and ligating ends of double strand breaks. DNA-PK inhibitors (DNA-PKI) are a structurally diverse class of inhibitors of DNA-PK, and the NHEJ pathway.

There exists a substantial need for efficient systems and techniques for modifying genomes. There is also a need for efficient methods for editing of nucleic acid molecules with template nucleic acids.

### BRIEF SUMMARY

The present disclosure relates to DNA-PKI, and compositions and methods of use thereof.

In certain embodiments, the DNA-PKI is a compound having the structure of Formula I:

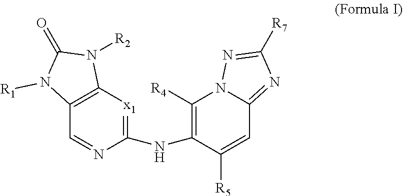

or a salt thereof,
wherein:
   x₁ is C—R₃ or N;
   R₁ is C₁-C₃ alkyl;
   R₂ is cycloalkyl or heterocyclyl, and cycloalkyl and heterocyclyl are optionally substituted with one or more R₆;
   R₃ is H or C₁-C₃ alkyl;
   R₄ is H or C₁-C₃ alkyl;
   R₅ is C₁-C₃ alkyl;
   each R₆ is independently selected from hydroxy, halo, alkyl, alkoxy, cycloalkyl, amino, and cyano, or two R₆, taken together with the atom or atoms to which they are bonded, form a spirocyclic or fused ring; and
   R₇ is H or C₁-C₃ alkyl,
provided that at least one of the following applies:
   (a) x₁ is C—R₃;
   (b) R₁ is C₂-C₃ alkyl;
   (c) R₄ is C₁-C₃ alkyl;
   (d) R₂ is substituted with one R₆, and R₆ is halo;
   (e) R₂ is substituted with two R₆ that, taken together with the atom or atoms to which they are bonded, form a spirocyclic or fused ring; and
   (f) R₂ is C₃-C₅ cycloalkyl optionally substituted with one or more R₆.

In preferred embodiments, the disclosure relates to a compound selected from:

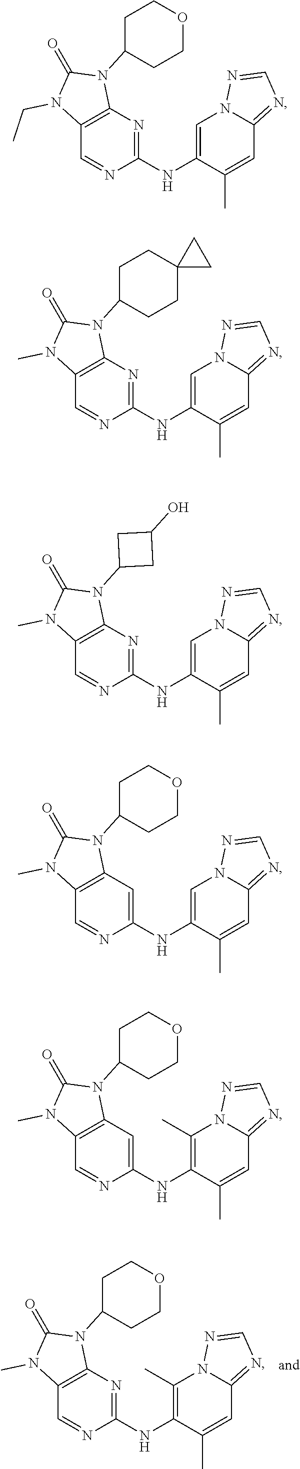

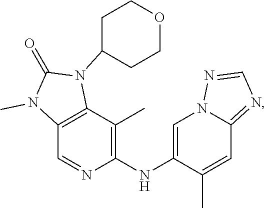 [SMILES: Cc1cc2ncnn2cc1Nc1ncc2c(c1C)n(C1CCOCC1)c(=O)n2C]

or a salt thereof.

In certain embodiments, the disclosure relates to DNA-PKI compositions comprising
a) a DNA protein kinase inhibitor (DNA-PKI);
b) a DNA cutting agent;
c) optionally, a cell; and
d) optionally, a donor DNA;
wherein the DNA-PKI is a compound of Formula I

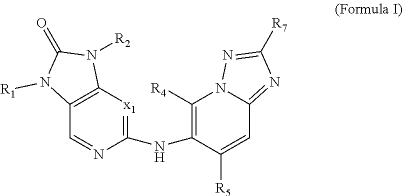

or a salt thereof,
wherein:
   x₁ is C—R₃ or N;
   R₁ is C₁-C₃ alkyl;
   R₂ is cycloalkyl or heterocyclyl, and cycloalkyl and heterocyclyl are optionally substituted with one or more R₆;
   R₃ is H or C₁-C₃ alkyl;
   R₄ is H or C₁-C₃ alkyl;
   R₅ is C₁-C₃ alkyl;
   each R₆ is independently selected from hydroxy, halo, alkyl, alkoxy, cycloalkyl, amino, and cyano, or two R₆, taken together with the atom or atoms to which they are bonded, form a spirocyclic or fused ring; and
   R₇ is H or C₁-C₃ alkyl.

In certain embodiments, the disclosure relates to a method for targeted genome editing in a cell or a method of repairing a double stranded DNA break in the genome of a cell or a method of inhibiting or suppressing repair of a DNA break in a cell via a non-homologous end joining (NHEJ) pathway, comprising contacting the cell with a DNA cutting agent and a DNA-PKI, wherein the DNA-PKI is a compound of Formula I

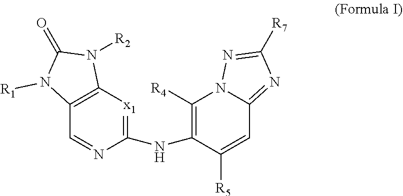

or a salt thereof,
wherein:
   x₁ is C—R₃ or N;
   R₁ is C₁-C₃ alkyl;
   R₂ is cycloalkyl or heterocyclyl, and cycloalkyl and heterocyclyl are optionally substituted with one or more R₆;
   R₃ is H or C₁-C₃ alkyl;
   R₄ is H or C₁-C₃ alkyl;
   R₅ is C₁-C₃ alkyl;
   each R₆ is independently selected from hydroxy, halo, alkyl, alkoxy, cycloalkyl, amino, and cyano, or two R₆, taken together with the atom or atoms to which they are bonded, form a spirocyclic or fused ring; and
   R₇ is H or C₁-C₃ alkyl.

### BRIEF DESCRIPTION OF DRAWINGS

FIGS. 1A-1B show the effect of DNA-PKI compounds on GFP insertion into the TRAC locus. FIG. 1A shows the percent of CD3⁻ cells following GFP insertion into the TRAC locus with compounds (Compound 1, Compound 2, Compound 3, Compound 4, Compound 5, Compound 6, Compound 7, Compound 8, and Compound 9), and FIG. 1B shows the insertion efficiency as percent of CD3⁻ cells which were GFP+.

FIGS. 2A-2C show editing at the TRAC locus with compounds Compound 1, Compound 3, and Compound 4. FIG. 2A shows the percent CD8⁺ cells. FIG. 2B shows the residual TCR⁺ cells after editing, and FIG. 2C shows the percent WT1-TCR⁺ cells after editing.

FIGS. 3A-3D shows the cytotoxicity of WT1-T cells engineered with compounds Compound 1 or Compound 3. FIGS. 3A and 3B show specific lysis of luciferase-expressing 697 ALL cells incubated WT1-T cells engineered from Donors 007HD and 008HD, respectively. FIGS. 3C and 3D show specific lysis of K562-luc2 cells transduced to express HLA-A*02:01 after incubation with WT1-T cells engineered from Donors 007HD and 008HD, respectively.

FIGS. 4A-4H show release of cytokines by T cells engineered with Compound 1 or Compound 3 after incubation with target cells. FIGS. 4A and 4B show the release of Granzyme B after incubation with 697 ALL cells and K562-luc2 cells transduced to express HLA-A*02:01, respectively. FIGS. 4C and 4D show the release of interferon-gamma (IFNg) after incubation with 697 ALL cells and K562-luc2 cells transduced to express HLA-A*02:01, respectively. FIGS. 4E and 4F show the release of interleukin-2 (IL-2) after incubation with 697 ALL cells and K562-luc2 cells transduced to express HLA-A*02:01, respectively. FIGS. 4G and 4H show the release of TNF-alpha after incubation with 697 ALL cells and K562-luc2 cells transduced to express HLA-A*02:01, respectively.

FIG. 5 shows the percent of B2M negative cells representing the population of B cells with effective gene disruption following editing with Compound 1 or Compound 4.

FIG. 6A shows the mean percent editing at AAVS1 assessed by NGS following treatment with LNP composition and varying doses of Compound 1 or Compound 4.

FIG. 6B shows the percent of NK cells with high GFP expression (GFP++) following editing to insert GFP at the AAVS1 locus with Compound 1 or Compound 4.

FIG. 7A shows the percent of CD3eta+, Vb8− cells, representing the population of T cells without gene disruption at the TRAC or TRBC1/2 loci.

FIG. 7B shows the percent of CD3eta+, Vb8+ cells, representing the population of T cells with WT1 TCR insertion at the TRAC.

FIG. 7C shows the percent of HLA-A2− cells, representing the population of T cells with effective gene disruption at the HLA locus.

FIG. 7D shows the percent of HLA-DRDPDQ− cells, representing the population of T cells with effective gene disruption at the CIITA locus.

FIG. 7E shows the percent of GFP+ cells, representing the population of T cells with GFP insertion at the AAVS1 locus.

FIG. 7F shows the percent of Vb8+ GFP+ HLA-A-HLA-DRDPDQ− cells, representing the population of T cells harboring 5 genome edits.

FIGS. 8A-8B show the percent of GFP+ cells, representing the population of T cells following editing in alternative media conditions for two LNP compositions. FIG. 8A shows cells treated with LNP compositions with lipid molar ratio of 50% ionizable lipid/38.5% cholesterol/10% DSPC/1.5% PEG Lipid. FIG. 8B shows cells treated with LNP compositions with lipid molar ratio of 35% ionizable lipid/47.5% cholesterol/15% DSPC/2.5% PEG lipid.

FIG. 9A shows the unintended percent structural variance following editing with Compound 3 and Compound 4.

FIG. 9B shows the percent GFP positive cells following editing with Compound 3 and Compound 4.

FIGS. 10A-B show the percent indels and percent HD3 TCR insertion in the presence and absence of DNApki Compound 4 at varied doses of sgRNA. FIG. 10A shows TRAC editing percent. FIG. 10B shows the percent of CD3⁺Vβ7.2⁺ T cells.

### DETAILED DESCRIPTION

Described herein are small molecule inhibitors of DNA-dependent protein kinase (DNA-PKI), which are useful for reducing NHEJ-mediated mutagenesis events or increasing the rate or probability of HDR following generation of a double-strand break (DSB) resulting from Cas9 cleavage. Exemplary DNA-PKI are provided, for example, in WO 2018/114999; WO 2014/183850; WO 03/024949; Fok, J. H. L., et al., Nat, Commun, 10, 5065 (2019); Griffin, R. J. et al., J. Med. Chem. 2005, 48, 569-585; Goldberg, F. W., et al., J. Med. Chem. 2020, 63, 3461-3471; and U.S. Pat. No. 10,786,512.

In some embodiments, the DNAPK inhibitors (DNA-PKI) are used in compositions and methods for delivering biologically active agents, including nucleic acids, such as CRISPR/Cas component RNAs and/or gRNA (the “cargo”), to a cell.

Methods of gene editing and methods of making engineered cells using the DNA-PKI described herein and compositions comprising them are also provided.

In some embodiments, the compositions and methods provided herein result in an editing efficiency of greater than about 80%, greater than about 90%, or greater than about 95%. In some embodiments, the compositions and methods result in an editing efficiency of about 80-95%, about 90-95%, about 80-99%, about 90-99%, or about 95-99%.

### DNA PK Inhibitors

The present disclosure relates to DNA-PKI, and compositions and methods of use thereof.

In certain embodiments, the disclosure relates to a compound having the structure of Formula I:

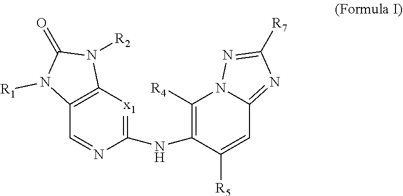

or a salt thereof,
wherein:
   x₁ is C—R₃ or N;
   R₁ is C₁-C₃ alkyl;
   R₂ is cycloalkyl or heterocyclyl, and cycloalkyl and heterocyclyl are optionally substituted with one or more R₆;
   R₃ is H or C₁-C₃ alkyl;
   R₄ is H or C₁-C₃ alkyl;
   R₅ is C₁-C₃ alkyl;
   each R₆ is independently selected from hydroxy, halo, alkyl, alkoxy, cycloalkyl, amino, and cyano, or two R₆, taken together with the atom or atoms to which they are bonded, form a spirocyclic or fused ring; and
   R₇ is H or C₁-C₃ alkyl,
provided that at least one of the following applies:
   (a) x₁ is C—R₃;
   (b) R₁ is C₂-C₃ alkyl;
   (c) R₄ is C₁-C₃ alkyl;
   (d) R₂ is substituted with one R₆, and R₆ is halo;
   (e) R₂ is substituted with two R₆ that, taken together with the atom or atoms to which they are bonded, form a spirocyclic or fused ring; and
   (f) R₂ is C₃-C₅ cycloalkyl optionally substituted with one or more R₆.

In certain embodiments, the disclosure relates to any of the compounds described herein, wherein x₁ is C—R₃. For example, R₃ may be H or methyl. In other embodiments, the compound relates to any of the compounds described herein, wherein x₁ is N.

In certain embodiments, the disclosure relates to any of the compounds described herein, wherein R₁ is C₂-C₃ alkyl, for example, R₁ is selected from methyl and ethyl, preferably, R₁ is methyl.

In some embodiments, the disclosure relates to any of the compounds described herein, wherein R₄ is C₁-C₃ alkyl, for example, R₄ is H or methyl, preferably, R₄ is H.

In certain embodiments, the disclosure relates to any of the compounds described herein, wherein R₂ is cycloalkyl, for example, R₂ is C₃-C₇ cycloalkyl, preferably R₂ is cyclohexyl or C₃-C₅ cycloalkyl.

In certain embodiments, the disclosure relates to any of the compounds described herein, wherein R₂ is heterocyclyl, for example, R₂ is 5- to 7-membered heterocyclyl, preferably R₂ is tetrahydropyranyl or tetrahydrofuranyl. In certain embodiments, the disclosure relates to any of the compounds described herein, wherein R₂ is optionally substituted with one or more R₆ independently selected from hydroxy, halo, and cycloalkyl, or two R₆, taken together with the atom or atoms to which they are bonded, form a spirocyclic or fused ring, for example, wherein R₂ is substituted with one or more R₆; and each R₆ is halo or hydroxyl, such as R₂ is substituted with one R₆, and R₆ is halo. In some embodiments, each R₆ is fluoro. In some embodiments, the disclosure relates to any of the compounds described herein, wherein R₂ is substituted with two R₆ that, taken together with the atom or atoms to which they are bonded, form a spirocyclic or fused ring. In particular embodiments, R₂ is optionally substituted with one or more R₆ independently selected from hydroxy, methoxy, and methyl.

In certain embodiments, the disclosure relates to any of the compounds described herein, wherein R₅ is methyl.

In some embodiments, the disclosure relates to any of the compounds described herein, wherein R₇ is H or methyl.

In preferred embodiments, the disclosure relates to a compound selected from:

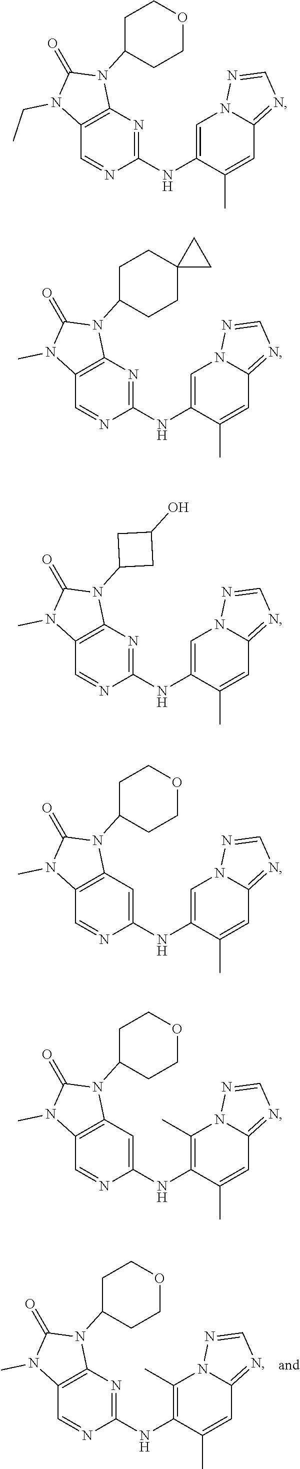

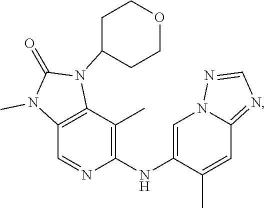 [SMILES: Cc1cc2ncnn2cc1Nc1ncc2c(c1C)n(C1CCOCC1)c(=O)n2C]

or a salt thereof.

In specific embodiments, the compound is

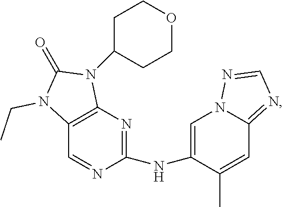 [SMILES: CCn1c(=O)n(C2CCOCC2)c2nc(Nc3cn4ncnc4cc3C)ncc21]

or a salt thereof.

In specific embodiments, the compound is

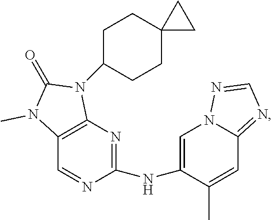 [SMILES: Cc1cc2ncnn2cc1Nc1ncc2c(n1)n(C1CCC3(CC1)CC3)c(=O)n2C]

or a salt thereof.

In specific embodiments, the compound is

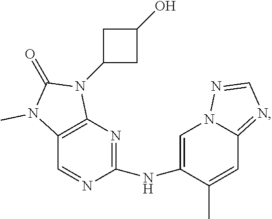 [SMILES: Cc1cc2ncnn2cc1Nc1ncc2c(n1)n(C1CC(O)C1)c(=O)n2C]

or a salt thereof.

In specific embodiments, the compound is

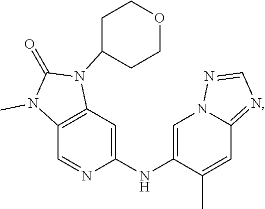 [SMILES: Cc1cc2ncnn2cc1Nc1cc2c(cn1)n(C)c(=O)n2C1CCOCC1]

or a salt thereof.

In specific embodiments, the compound is

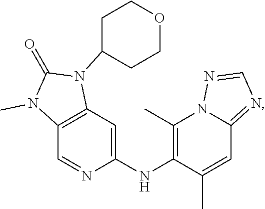 [SMILES: Cc1cc2ncnn2c(C)c1Nc1cc2c(cn1)n(C)c(=O)n2C1CCOCC1]

or a salt thereof.

In specific embodiments, the compound is

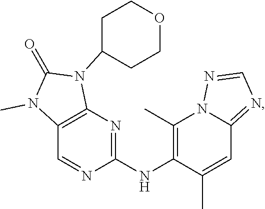 [SMILES: Cc1cc2ncnn2c(C)c1Nc1ncc2c(n1)n(C1CCOCC1)c(=O)n2C]

or a salt thereof.

In specific embodiments, the compound is

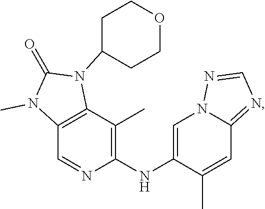 [SMILES: Cc1cc2ncnn2cc1Nc1ncc2c(c1C)n(C1CCOCC1)c(=O)n2C]

or a salt thereof.

In certain embodiments, the disclosure relates to any of the compounds described herein, wherein the compound is a free base.

In certain embodiments, the disclosure relates to any of the compounds described herein, wherein the compound is a salt, for example, a triflate salt.

### DNA-PKI Compositions

Described herein are DNA-PKI compositions comprising
a) a DNA protein kinase inhibitor (DNA-PKI);
b) a DNA cutting agent;
c) optionally, a cell; and
d) optionally, a donor DNA;
wherein the DNA-PKI is a compound of Formula I

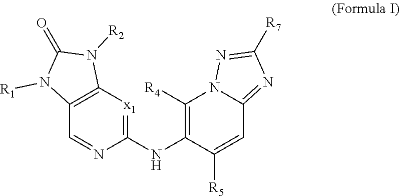

or a salt thereof,
wherein:
   x₁ is C—R₃ or N;
   R₁ is C₁-C₃ alkyl;
   R₂ is cycloalkyl or heterocyclyl, and cycloalkyl and heterocyclyl are optionally substituted with one or more R₆;
   R₃ is H or C₁-C₃ alkyl;
   R₄ is H or C₁-C₃ alkyl;
   R₅ is C₁-C₃ alkyl;
   each R₆ is independently selected from hydroxy, halo, alkyl, alkoxy, cycloalkyl, amino, and cyano, or two R₆, taken together with the atom or atoms to which they are bonded, form a spirocyclic or fused ring; and
   R₇ is H or C₁-C₃ alkyl.

In certain embodiments, the disclosure relates to any of the compositions described herein, wherein x₁ is N.

In certain embodiments, the disclosure relates to any of the compositions described herein, wherein R₁ is methyl.

In certain embodiments, the disclosure relates to any of the compositions described herein, wherein R₄ is H.

In certain embodiments, the disclosure relates to any of the compositions described herein, wherein R₂ is cyclohexyl. In other embodiments, R₂ is tetrahydropyranyl. In still other embodiments, R₂ is tetrahydrofuranyl. In certain embodiments, the disclosure relates to any of the compositions described herein, wherein R₂ is optionally substituted with one or more R₆ independently selected from hydroxy, methoxy, and methyl.

In certain embodiments, the disclosure relates to any of the compositions described herein, wherein R₅ is methyl.

In certain embodiments, the disclosure relates to any of the compositions described herein, wherein R₇ is H or methyl.

In certain embodiments, the disclosure relates to any of the compositions described herein, wherein the DNA-PKI is any of the compounds described herein.

In certain embodiments, the disclosure relates a composition comprising
a) a DNA protein kinase inhibitor (DNA-PKI);
b) a DNA cutting agent;
c) optionally, a cell; and
d) optionally, a donor DNA;
wherein the DNA-PKI is selected from:

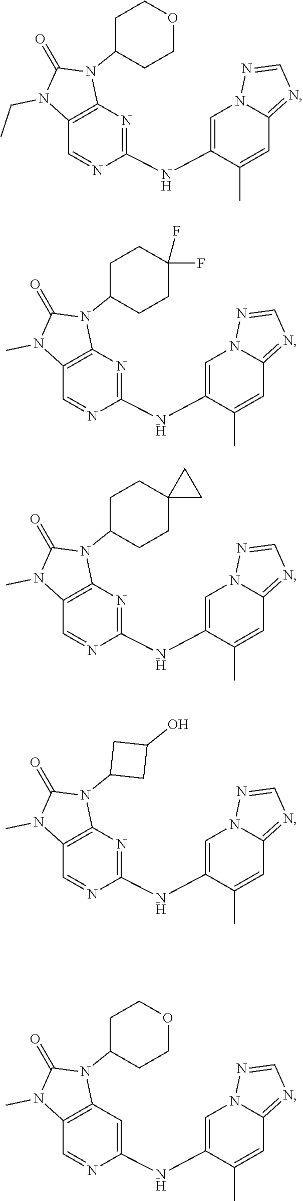 [SMILES: CCn1c(=O)n(C2CCOCC2)c2nc(Nc3cn4ncnc4cc3C)ncc21 ; Cc1cc2ncnn2cc1Nc1ncc2c(n1)n(C1CCC(F)(F)CC1)c(=O)n2C ; Cc1cc2ncnn2cc1Nc1ncc2c(n1)n(C1CCC3(CC1)CC3)c(=O)n2C ; Cc1cc2ncnn2cc1Nc1ncc2c(n1)n(C1CC(O)C1)c(=O)n2C ; Cc1cc2ncnn2cc1Nc1cc2c(cn1)n(C)c(=O)n2C1CCOCC1]

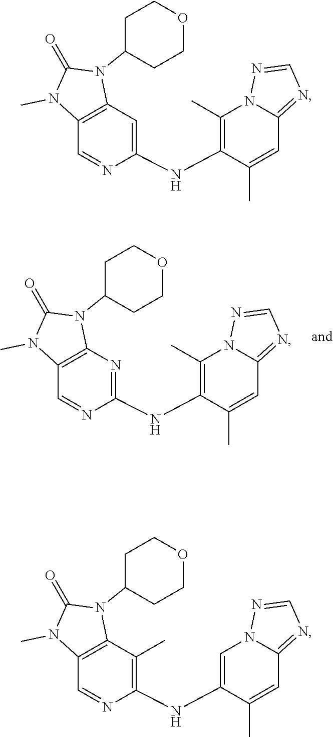 [SMILES: Cc1cc2ncnn2c(C)c1Nc1cc2c(cn1)n(C)c(=O)n2C1CCOCC1 ; Cc1cc2ncnn2c(C)c1Nc1ncc2c(n1)n(C1CCOCC1)c(=O)n2C ; Cc1cc2ncnn2cc1Nc1ncc2c(c1C)n(C1CCOCC1)c(=O)n2C]

or a salt thereof.

In specific embodiments, the DNA-PKI in the composition is

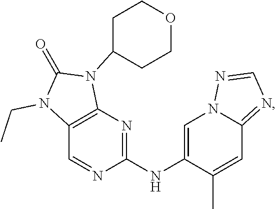 [SMILES: CCn1c(=O)n(C2CCOCC2)c2nc(Nc3cn4ncnc4cc3C)ncc21]

or a salt thereof.

In specific embodiments, the DNA-PKI in the composition is

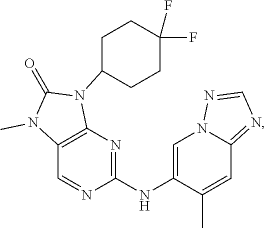 [SMILES: Cc1cc2ncnn2cc1Nc1ncc2c(n1)n(C1CCC(F)(F)CC1)c(=O)n2C]

or a salt thereof.

In specific embodiments, the DNA-PKI in the composition is

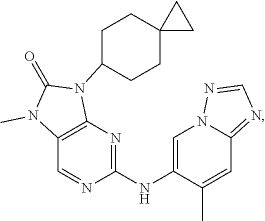 [SMILES: Cc1cc2ncnn2cc1Nc1ncc2c(n1)n(C1CCC3(CC1)CC3)c(=O)n2C]

or a salt thereof.

In specific embodiments, the DNA-PKI in the composition is

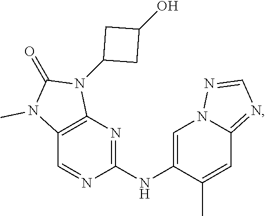 [SMILES: Cc1cc2ncnn2cc1Nc1ncc2c(n1)n(C1CC(O)C1)c(=O)n2C]

or a salt thereof.

In specific embodiments, the DNA-PKI in the composition is

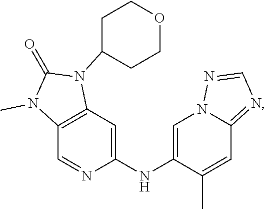 [SMILES: Cc1cc2ncnn2cc1Nc1cc2c(cn1)n(C)c(=O)n2C1CCOCC1]

or a salt thereof.

In specific embodiments, the DNA-PKI in the composition is

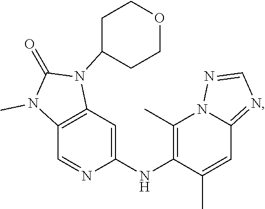 [SMILES: Cc1cc2ncnn2c(C)c1Nc1cc2c(cn1)n(C)c(=O)n2C1CCOCC1]

or a salt thereof.

In specific embodiments, the DNA-PKI in the composition is

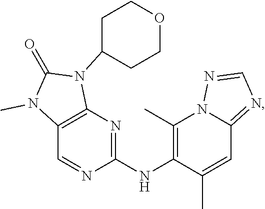 [SMILES: Cc1cc2ncnn2c(C)c1Nc1ncc2c(n1)n(C1CCOCC1)c(=O)n2C]

or a salt thereof.

In specific embodiments, the DNA-PKI in the composition is

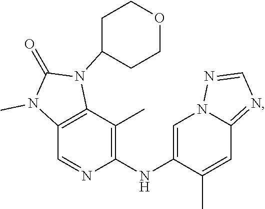 [SMILES: Cc1cc2ncnn2cc1Nc1ncc2c(c1C)n(C1CCOCC1)c(=O)n2C]

or a salt thereof.

In certain embodiments, the disclosure relates to any of the compositions described herein, wherein the concentration of the DNA-PKI in the composition is about 1 μM or less, for example, about 0.25 μM or less, such as about 0.1-1 μM, preferably about 0.1-0.5 μM.

In some embodiments, the disclosure relates to any of the compositions described herein, wherein the composition comprises a cell, for example, a eukaryotic cell, such as a liver cell or an immune cell. In some embodiments, the disclosure relates to any of the compositions described herein, wherein the cell is useful in adoptive cell therapy (ACT). Examples of ACT include autologous and allogeneic cell therapies. In some embodiments, the disclosure relates to any of the compositions described herein, wherein the cell is a stem cell. In some embodiments, the disclosure relates to any of the compositions described herein, wherein the cell is a stem cell. In some embodiments, the disclosure relates to any of the compositions described herein, wherein the cell is a hematopoietic stem cell (HSC) or an induced pluripotent stem cell (iPSC). In certain embodiments, the disclosure relates to any of the compositions described herein, wherein the immune cell is a leukocyte or a lymphocyte, for example, the immune cell is a lymphocyte, such as a T cell, a B cell, or an NK cell, preferably the lymphocyte is a T cell. In some embodiments, the disclosure relates to any of the compositions described herein, wherein the T cell is a primary T cell. In certain embodiments, the disclosure relates to any of the compositions described herein, wherein the T cell is a regulatory T cell. In certain embodiments, the disclosure relates to any of the compositions described herein, wherein the lymphocyte is an activated T cell or a non-activated T cell.

In certain embodiments, the disclosure relates to any of the compositions described herein, wherein the cell is a human cell.

In some embodiments, the disclosure relates to any of the compositions described herein, wherein the DNA cutting agent comprises a CRISPR/Cas nuclease component and optionally a guide RNA component. In some embodiments, the disclosure relates to any of the compositions described herein, comprising a DNA cutting agent or a nucleic acid encoding the DNA cutting agent, for example, an mRNA encoding a DNA cutting agent, wherein the DNA cutting agent is selected from a zinc finger nuclease, a TALE effector domain nuclease (TALEN), a CRISPR/Cas nuclease component, and combinations thereof, preferably wherein the DNA cutting agent is a CRISPR/Cas nuclease component. In some embodiments, the DNA cutting agent is a CRISPR/Cas nuclease component and a guide RNA component. In some embodiments, the disclosure relates to any of the compositions described herein, wherein the CRISPR/Cas nuclease component comprises a Cas nuclease or an mRNA encoding the Cas nuclease, for example, the CRISPR/Cas nuclease component comprises an mRNA encoding the Cas nuclease, such as a Class 2 Cas nuclease. In certain embodiments, the disclosure relates to any of the compositions described herein, wherein the Cas nuclease is a Cas9 nuclease, such as a S. pyogenes Cas9 nuclease or a N. meningitidis Cas9 nuclease. In certain embodiments, the disclosure relates to any of the compositions described herein, wherein the Cas nuclease is Nme2Cas9. In certain embodiments, the disclosure relates to any of the compositions described herein, wherein the Cas nuclease is a Cas12a nuclease.

In some embodiments, the disclosure relates to any of the compositions described herein, comprising a modified RNA.

In certain embodiments, the disclosure relates to any of the compositions described herein, comprising a guide RNA nucleic acid, such as a gRNA. In certain embodiments, the disclosure relates to any of the compositions described herein, wherein the guide RNA nucleic acid is or encodes a dual-guide RNA (dgRNA). In certain embodiments, the disclosure relates to any of the compositions described herein, wherein the guide RNA nucleic acid is or encodes a single-guide (sgRNA). In some embodiments, the disclosure relates to any of the compositions described herein, wherein the gRNA is a modified gRNA, for example wherein the modified gRNA comprises a modification at one or more of the first five nucleotides at the 5′ end or the modified gRNA comprises a modification at one or more of the last five nucleotides at the 3′ end. In some embodiments, the gRNA is complexed with a Cas nuclease, such as a Cas9 nuclease.

In certain embodiments, the disclosure relates to any of the compositions described herein, wherein the composition comprises a guide RNA nucleic acid and a Class 2 Cas nuclease mRNA; and the ratio of the mRNA to the guide RNA nucleic acid is from about 2:1 to 1:4 by weight.

In some embodiments, the disclosure relates to any of the compositions described herein, comprising the DNA cutting agent, wherein the DNA cutting agent is present in a lipid nucleic acid assembly composition.

In some embodiments, the disclosure relates to any of the compositions described herein, comprising the donor DNA.

In certain embodiments, the disclosure relates to any of the compositions described herein, wherein the donor DNA (also referred to herein as a “template nucleic acid” or an “exogenous nucleic acid”) comprises a sequence encoding a protein, a regulatory sequence, or a sequence encoding structural RNA.

In certain embodiments, the disclosure relates to any of the compositions described herein, wherein the lipid nucleic acid assembly composition is a lipid nanoparticle (LNP) composition. In some embodiments, the LNP composition is any of the LNP compositions described herein.

In certain embodiments, the disclosure relates to any of the compositions described herein, wherein the LNP has a diameter of about 10-200 nm, about 20-150 nm, about 50-150 nm, about 50-100 nm, about 50-120 nm, about 60-100 nm, about 75-150 nm, about 75-120 nm, or about 75-100 nm. In certain embodiments, the disclosure relates to any of the compositions described herein, wherein the composition comprises a population of the LNPs with an average diameter of about 10-200 nm, about 20-150 nm, about 50-150 nm, about 50-100 nm, about 50-120 nm, about 60-100 nm, about 75-150 nm, about 75-120 nm, or about 75-100 nm. For example, the average diameter may be a Z-average diameter.

In certain embodiments, the disclosure relates to any of the compositions described herein, wherein the lipid nucleic acid assembly composition is a lipoplex.

In some embodiments, the disclosure relates to any of the compositions described herein, wherein the lipid nucleic acid assembly composition comprises an ionizable lipid, for example, any of the ionizable lipids described herein. In certain embodiments, the disclosure relates to any of the compositions described herein, wherein the ionizable lipid has a pKa of from about 5.1 to about 8.0, for example from about 5.5 to about 7.6 or from about 5.1 to 7.4, such as from about 5.5 to 6.6, from about 5.6 to 6.4, from about 5.8 to 6.2, or from about 5.8 to 6.5.

In certain embodiments, the disclosure relates to any of the compositions described herein, the lipid nucleic acid assembly composition comprises a helper lipid.

In certain embodiments, the disclosure relates to any of the compositions described herein, wherein the lipid nucleic acid assembly composition comprises a neutral lipid.

In some embodiments, the disclosure relates to any of the compositions described herein, wherein the lipid nucleic acid assembly composition comprises a PEG lipid.

In certain embodiments, the disclosure relates to any of the compositions described herein, wherein the N/P ratio of the lipid nucleic acid assembly composition is about 3-10, for example from about 5-7, preferably about 6.

In some embodiments, the disclosure relates to any of the compositions described herein, further comprising a vector, for example, wherein the vector encodes the DNA cutting agent or the donor DNA. In certain embodiments, the disclosure relates to any of the compositions described herein, wherein the vector is a viral vector. In other embodiments, the disclosure relates to any of the compositions described herein, wherein the vector is a non-viral vector. In certain embodiments, the disclosure relates to any of the compositions described herein, wherein the vector is a lentiviral vector. In some embodiments, the disclosure relates to any of the compositions described herein, wherein the vector is a retroviral vector. In certain embodiments, the disclosure relates to any of the compositions described herein, wherein the vector is an AAV.

In certain embodiments, the disclosure relates to any of the compositions described herein, comprising a cell, for example, wherein the cell is not a cancer cell.

### DNA-PKI Methods

In certain embodiments, the disclosure relates to a method for targeted genome editing in a cell, comprising contacting the cell with a DNA cutting agent and a DNA-PKI, wherein the DNA-PKI is a compound of Formula I

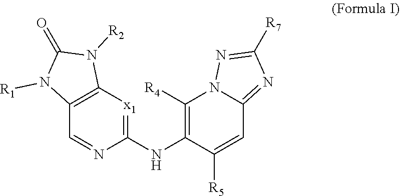

or a salt thereof,
wherein:
   x₁ is C—R₃ or N;
   R₁ is C₁-C₃ alkyl;
   R₂ is cycloalkyl or heterocyclyl, and cycloalkyl and heterocyclyl are optionally
   substituted with one or more R₆;
   R₃ is H or C₁-C₃ alkyl;
   R₄ is H or C₁-C₃ alkyl;
   R₅ is C₁-C₃ alkyl;
   each R₆ is independently selected from hydroxy, halo, alkyl, alkoxy, cycloalkyl, amino, and cyano, or two R₆, taken together with the atom or atoms to which they are bonded, form a spirocyclic or fused ring; and
   R₇ is H or C₁-C₃ alkyl.

In some embodiments, the disclosure relates to a method of repairing a double stranded DNA break in the genome of a cell, comprising contacting the cell with a DNA cutting agent and a DNA-PKI, wherein the DNA-PKI is a compound of Formula I

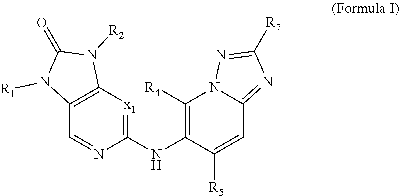

or a salt thereof,
wherein:
   x₁ is C—R₃ or N;
   R₁ is C₁-C₃ alkyl;
   R₂ is cycloalkyl or heterocyclyl, and cycloalkyl and heterocyclyl are optionally substituted with one or more R₆;
   R₃ is H or C₁-C₃ alkyl;
   R₄ is H or C₁-C₃ alkyl;
   R₅ is C₁-C₃ alkyl;
   each R₆ is independently selected from hydroxy, halo, alkyl, alkoxy, cycloalkyl, amino, and cyano, or two R₆, taken together with the atom or atoms to which they are bonded, form a spirocyclic or fused ring; and
   R₇ is H or C₁-C₃ alkyl.

In some embodiments, the disclosure relates to a method of inhibiting or suppressing repair of a DNA break in a cell via a non-homologous end joining (NHEJ) pathway, comprising contacting the cell with a DNA cutting agent and a DNA-PKI, wherein the DNA-PKI is a compound of Formula I

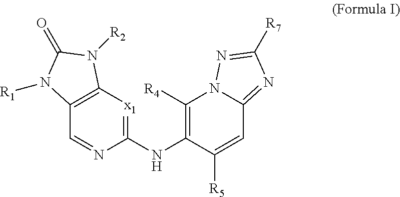

or a salt thereof,
wherein:
   x₁ is C—R₃ or N;
   R₁ is C₁-C₃ alkyl;
   R₂ is cycloalkyl or heterocyclyl, and cycloalkyl and heterocyclyl are optionally substituted with one or more R₆;
   R₃ is H or C₁-C₃ alkyl;
   R₄ is H or C₁-C₃ alkyl;
   R₅ is C₁-C₃ alkyl;
   each R₆ is independently selected from hydroxy, halo, alkyl, alkoxy, cycloalkyl, amino, and cyano, or two R₆, taken together with the atom or atoms to which they are bonded, form a spirocyclic or fused ring; and
   R₇ is H or C₁-C₃ alkyl.

In some embodiments, the disclosure relates to a method of targeted insertion of a donor DNA into the genome of a cell, comprising contacting the cell with a DNA cutting agent, the donor DNA, and a DNA-PKI, wherein the DNA-PKI is a compound of Formula I

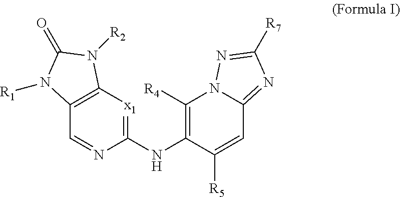

or a salt thereof,
wherein:
   x₁ is C—R₃ or N;
   R₁ is C₁-C₃ alkyl;
   R₂ is cycloalkyl or heterocyclyl, and cycloalkyl and heterocyclyl are optionally substituted with one or more R₆;
   R₃ is H or C₁-C₃ alkyl;
   R₄ is H or C₁-C₃ alkyl;
   R₅ is C₁-C₃ alkyl;
   each R₆ is independently selected from hydroxy, halo, alkyl, alkoxy, cycloalkyl, amino, and cyano, or two R₆, taken together with the atom or atoms to which they are bonded, form a spirocyclic or fused ring; and
   R₇ is H or C₁-C₃ alkyl.

In some embodiments, the description relates to methods for adoptive cell transfer (ACT) therapies, such as for immunooncology. For example, in certain embodiments, the methods described herein result in cells modified at one or more specific target sequences in their genome, including as modified by introduction of CRISPR systems that include gRNA molecules which target said target sequences. Certain embodiments provide for gRNA molecules, CRISPR systems, cells, and methods useful for genome editing of immune cells, e.g., T cells engineered to lack endogenous TCR expression, e.g., T cells suitable for further engineering to insert a nucleic acid of interest, e.g., T cells further engineered to express a TCR, such as a transgenic TCR (tgTCR), and useful for ACT therapies; and for genome editing of B cells, e.g., B cells engineered to lack endogenous B cell receptor (BCR) expression, e.g., B cells suitable for further engineering to insert a nucleic acid of interest, e.g., B cells further engineered to express a BCR, such as a transgenic BCR (tgBCR), or for expression of an antibody, and useful for ACT therapies.

In certain embodiments, the disclosure relates to any method of gene editing described herein, comprising administering the LNP composition to an animal, for example a human. In certain embodiments, the method comprises administering the LNP composition to a cell, such as a eukaryotic cell, and in particular a human cell. In some embodiments, the cell is a type of cell useful in a therapy, for example, adoptive cell therapy (ACT). Examples of ACT include autologous and allogeneic cell therapies. In some embodiments, the cell is a stem cell, such as a hematopoietic stem cell, an induced pluripotent stem cell, or another multipotent or pluripotent cell. In some embodiments, the cell is a stem cell, for example, a mesenchymal stem cell that can develop into a bone, cartilage, muscle, or fat cell. In some embodiments, the stem cells comprise ocular stem cells. In certain embodiments, the cell is selected from mesenchymal stem cells, hematopoietic stem cells (HSCs), mononuclear cells, endothelial progenitor cells (EPCs), neural stem cells (NSCs), limbal stem cells (LSCs), tissue-specific primary cells or cells derived therefrom (TSCs), induced pluripotent stem cells (iPSCs), ocular stem cells, pluripotent stem cells (PSCs), embryonic stem cells (ESCs), and cells for organ or tissue transplantations.

In certain embodiments, the disclosure relates to any of the methods described herein, comprising growing the cell in a cell medium free of the DNA-PKI and adding the DNA-PKI to the cell medium.

In certain embodiments, the disclosure relates to any of the methods described herein, comprising contacting the cell with the DNA cutting agent before contacting the cell with the DNA-PKI, for example, within about six hours of contacting the cell with the DNA cutting agent, preferably, within about three hours of contacting the cell with the DNA cutting agent.

In other embodiments, the disclosure relates to any of the methods described herein, comprising contacting the cell with the DNA cutting agent simultaneously with the DNA-PKI.

In still other embodiments, the disclosure relates to any of the methods described herein, comprising contacting the cell with the DNA cutting agent after contacting the cell with the DNA-PKI, for example, within about three hours of contacting the cell with the DNA-PKI.

In certain embodiments, the disclosure relates to any of the methods described herein, comprising growing the cell in a cell medium comprising the DNA-PKI.

In some embodiments, the disclosure relates to any of the methods described herein, wherein the cell is contacted with the DNA cutting agent and the DNA-PKI for at least about one day, for example, for about one day to one week, preferably for about five days.

In certain embodiments, the disclosure relates to any of the methods described herein, wherein x₁ is N.

In certain embodiments, the disclosure relates to any of the methods described herein, wherein R₁ is methyl.

In certain embodiments, the disclosure relates to any of the methods described herein, wherein R₄ is H.

In some embodiments, the disclosure relates to any of the methods described herein, wherein R₂ is cyclohexyl, tetrahydropyranyl, or tetrahydrofuranyl. In certain embodiments, the disclosure relates to any of the methods described herein, wherein R₂ is optionally substituted with one or more R₆ independently selected from hydroxy, methoxy, and methyl.

In certain embodiments, the disclosure relates to any of the methods described herein, wherein R₅ is methyl.

In certain embodiments, the disclosure relates to any of the methods described herein, wherein R₇ is H or methyl.

In preferred embodiments, the disclosure relates to any of the methods described herein, wherein the DNA-PKI is any of the compounds described herein.

In certain embodiments, the disclosure relates to a method for targeted genome editing in a cell, comprising contacting the cell with a DNA cutting agent and a DNA-PKI, wherein the DNA-PKI is selected from:

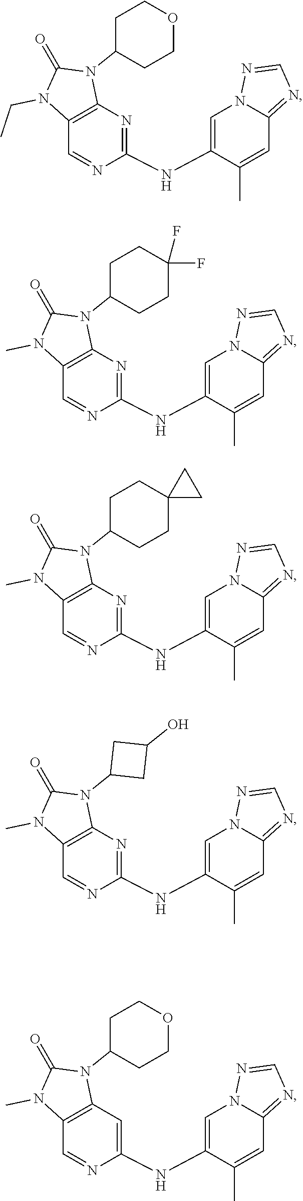 [SMILES: CCn1c(=O)n(C2CCOCC2)c2nc(Nc3cn4ncnc4cc3C)ncc21 ; Cc1cc2ncnn2cc1Nc1ncc2c(n1)n(C1CCC(F)(F)CC1)c(=O)n2C ; Cc1cc2ncnn2cc1Nc1ncc2c(n1)n(C1CCC3(CC1)CC3)c(=O)n2C ; Cc1cc2ncnn2cc1Nc1ncc2c(n1)n(C1CC(O)C1)c(=O)n2C ; Cc1cc2ncnn2cc1Nc1cc2c(cn1)n(C)c(=O)n2C1CCOCC1]

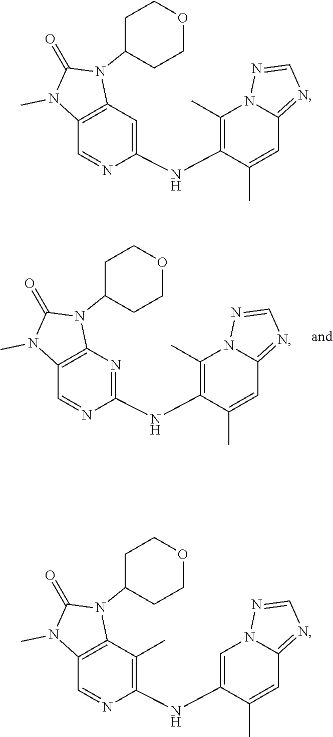 [SMILES: Cc1cc2ncnn2c(C)c1Nc1cc2c(cn1)n(C)c(=O)n2C1CCOCC1 ; Cc1cc2ncnn2c(C)c1Nc1ncc2c(n1)n(C1CCOCC1)c(=O)n2C ; Cc1cc2ncnn2cc1Nc1ncc2c(c1C)n(C1CCOCC1)c(=O)n2C]

or a salt thereof.

In certain embodiments, the disclosure relates to a method of repairing a double stranded DNA break in the genome of a cell, comprising contacting the cell with a DNA cutting agent and a DNA-PKI, wherein the DNA-PKI is selected from:

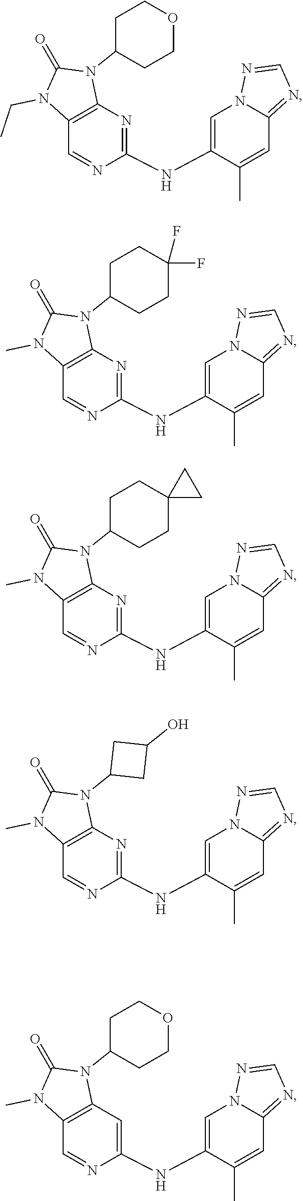 [SMILES: CCn1c(=O)n(C2CCOCC2)c2nc(Nc3cn4ncnc4cc3C)ncc21 ; Cc1cc2ncnn2cc1Nc1ncc2c(n1)n(C1CCC(F)(F)CC1)c(=O)n2C ; Cc1cc2ncnn2cc1Nc1ncc2c(n1)n(C1CCC3(CC1)CC3)c(=O)n2C ; Cc1cc2ncnn2cc1Nc1ncc2c(n1)n(C1CC(O)C1)c(=O)n2C ; Cc1cc2ncnn2cc1Nc1cc2c(cn1)n(C)c(=O)n2C1CCOCC1]

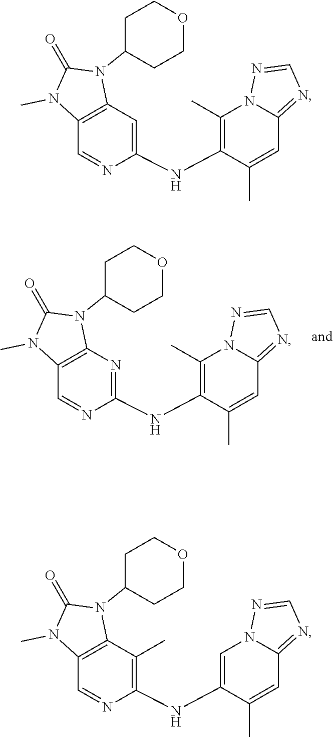 [SMILES: Cc1cc2ncnn2c(C)c1Nc1cc2c(cn1)n(C)c(=O)n2C1CCOCC1 ; Cc1cc2ncnn2c(C)c1Nc1ncc2c(n1)n(C1CCOCC1)c(=O)n2C ; Cc1cc2ncnn2cc1Nc1ncc2c(c1C)n(C1CCOCC1)c(=O)n2C]

or a salt thereof.

In certain embodiments, the disclosure relates to a method of inhibiting or suppressing repair of a DNA break in a cell via a non-homologous end joining (NHEJ) pathway, comprising contacting the cell with a DNA cutting agent and a DNA-PKI, wherein the DNA-PKI is selected from:

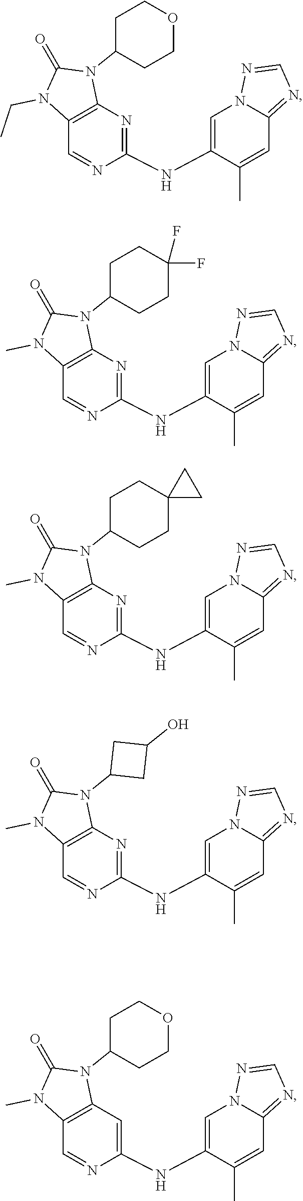 [SMILES: CCn1c(=O)n(C2CCOCC2)c2nc(Nc3cn4ncnc4cc3C)ncc21 ; Cc1cc2ncnn2cc1Nc1ncc2c(n1)n(C1CCC(F)(F)CC1)c(=O)n2C ; Cc1cc2ncnn2cc1Nc1ncc2c(n1)n(C1CCC3(CC1)CC3)c(=O)n2C ; Cc1cc2ncnn2cc1Nc1ncc2c(n1)n(C1CC(O)C1)c(=O)n2C ; Cc1cc2ncnn2cc1Nc1cc2c(cn1)n(C)c(=O)n2C1CCOCC1]

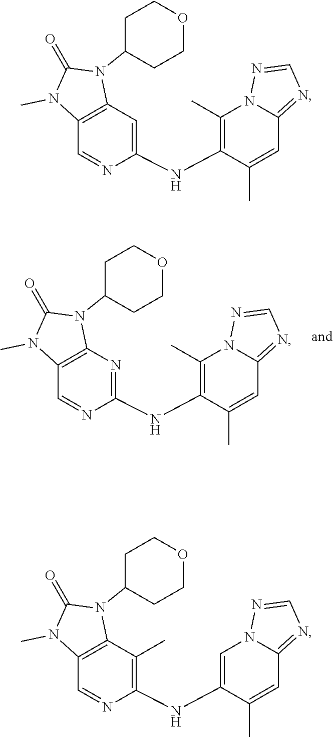 [SMILES: Cc1cc2ncnn2c(C)c1Nc1cc2c(cn1)n(C)c(=O)n2C1CCOCC1 ; Cc1cc2ncnn2c(C)c1Nc1ncc2c(n1)n(C1CCOCC1)c(=O)n2C ; Cc1cc2ncnn2cc1Nc1ncc2c(c1C)n(C1CCOCC1)c(=O)n2C]

or a salt thereof.

In certain embodiments, the disclosure relates to a method of targeted insertion of a donor DNA into the genome of a cell, comprising contacting the cell with a DNA cutting agent, the donor DNA, and a DNA-PKI, wherein the DNA-PKI is selected from:

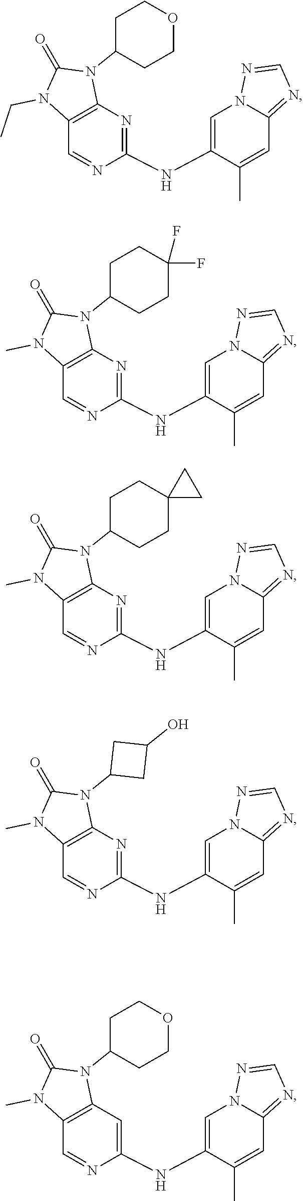 [SMILES: CCn1c(=O)n(C2CCOCC2)c2nc(Nc3cn4ncnc4cc3C)ncc21 ; Cc1cc2ncnn2cc1Nc1ncc2c(n1)n(C1CCC(F)(F)CC1)c(=O)n2C ; Cc1cc2ncnn2cc1Nc1ncc2c(n1)n(C1CCC3(CC1)CC3)c(=O)n2C ; Cc1cc2ncnn2cc1Nc1ncc2c(n1)n(C1CC(O)C1)c(=O)n2C ; Cc1cc2ncnn2cc1Nc1cc2c(cn1)n(C)c(=O)n2C1CCOCC1]

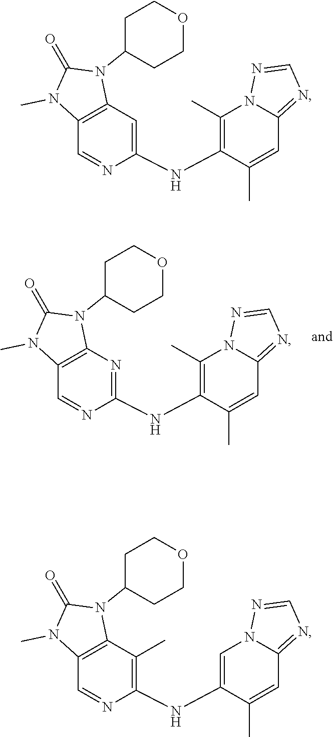 [SMILES: Cc1cc2ncnn2c(C)c1Nc1cc2c(cn1)n(C)c(=O)n2C1CCOCC1 ; Cc1cc2ncnn2c(C)c1Nc1ncc2c(n1)n(C1CCOCC1)c(=O)n2C ; Cc1cc2ncnn2cc1Nc1ncc2c(c1C)n(C1CCOCC1)c(=O)n2C]

or a salt thereof.

In specific embodiments, the DNA-PKI used in the method is

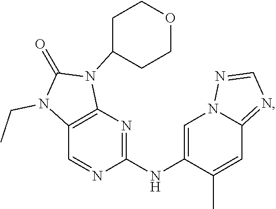 [SMILES: CCn1c(=O)n(C2CCOCC2)c2nc(Nc3cn4ncnc4cc3C)ncc21]

or a salt thereof.

In specific embodiments, the DNA-PKI used in the method is

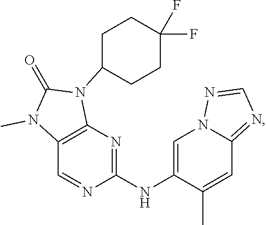 [SMILES: Cc1cc2ncnn2cc1Nc1ncc2c(n1)n(C1CCC(F)(F)CC1)c(=O)n2C]

or a salt thereof.

In specific embodiments, the DNA-PKI used in the method is

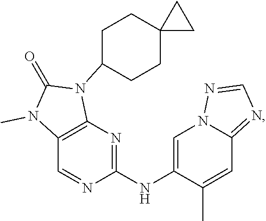 [SMILES: Cc1cc2ncnn2cc1Nc1ncc2c(n1)n(C1CCC3(CC1)CC3)c(=O)n2C]

or a salt thereof.

In specific embodiments, the DNA-PKI used in the method is

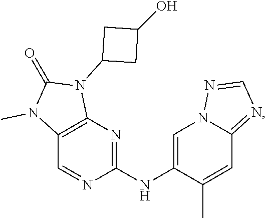 [SMILES: Cc1cc2ncnn2cc1Nc1ncc2c(n1)n(C1CC(O)C1)c(=O)n2C]

or a salt thereof.

In specific embodiments, the DNA-PKI used in the method is

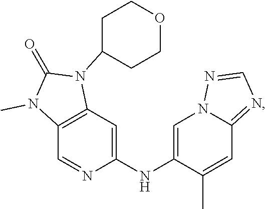 [SMILES: Cc1cc2ncnn2cc1Nc1cc2c(cn1)n(C)c(=O)n2C1CCOCC1]

or a salt thereof.

In specific embodiments, the DNA-PKI used in the method is

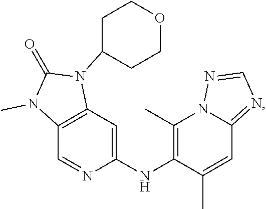 [SMILES: Cc1cc2ncnn2c(C)c1Nc1cc2c(cn1)n(C)c(=O)n2C1CCOCC1]

or a salt thereof.

In specific embodiments, the DNA-PKI used in the method is

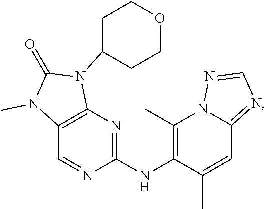 [SMILES: Cc1cc2ncnn2c(C)c1Nc1ncc2c(n1)n(C1CCOCC1)c(=O)n2C]

or a salt thereof.

In specific embodiments, the DNA-PKI used in the method is

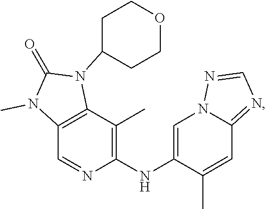 [SMILES: Cc1cc2ncnn2cc1Nc1ncc2c(c1C)n(C1CCOCC1)c(=O)n2C]

or a salt thereof.

In certain embodiments, the disclosure relates to any of the methods described herein, wherein the cell is contacted with the DNA-PKI in a cell medium, wherein the concentration of the DNA-PKI in the cell medium is about 1 μM or less, for example, about 0.25 μM or less, such as, from about 0.1-1 μM, preferably from about 0.1-0.5 μM.

In certain embodiments, the disclosure relates to any of the methods described herein, wherein the cell is a eukaryotic cell.

In some embodiments, the disclosure relates to any of the methods described herein, wherein the composition comprises a cell, for example, a eukaryotic cell, such as a liver cell or an immune cell. In certain embodiments, the cell is useful in adoptive cell therapy (ACT). Examples of ACT include autologous and allogeneic cell therapies. In certain embodiments, the cell is a stem cell. In certain embodiments, the stem cell is a hematopoietic stem cell (HSC). In certain embodiments, the cell is an induced pluripotent stem cell (iPSC). In certain embodiments, the disclosure relates to any of the methods described herein, wherein the immune cell is a leukocyte or a lymphocyte, for example, the immune cell is a lymphocyte, such as a T cell, a B cell, or an NK cell, preferably the lymphocyte is a T cell. In some embodiments, the disclosure relates to any of the methods described herein, wherein the T cell is a primary T cell. In certain embodiments, the disclosure relates to any of the methods described herein, wherein the T cell is a regulatory T cell. In certain embodiments, the disclosure relates to any of the methods described herein, wherein the lymphocyte is an activated T cell or a non-activated T cell.

In certain embodiments, the disclosure relates to any of the methods described herein, wherein the cell is a human cell.

In some embodiments, the disclosure relates to any of the methods described herein, comprising a DNA cutting agent, for example, wherein the DNA cutting agent is selected from a zinc finger nuclease, a TALE effector domain nuclease (TALEN), a CRISPR/Cas nuclease component, and combinations thereof, preferably wherein the DNA cutting agent is a CRISPR/Cas nuclease component.

In some embodiments, the disclosure relates to any of the methods described herein, wherein the CRISPR/Cas nuclease component comprises a Cas nuclease or an mRNA encoding the Cas nuclease, for example, the CRISPR/Cas nuclease component comprises an mRNA encoding the Cas nuclease, such as a Class 2 Cas nuclease. In certain embodiments, the disclosure relates to any of the methods described herein, wherein the Cas nuclease is a Cas9 nuclease, such as a S. pyogenes Cas9 nuclease or a N. meningitidis Cas9 nuclease. In certain embodiments, the disclosure relates to any of the methods described herein, wherein the Cas nuclease is Nme2Cas9. In certain embodiments, the disclosure relates to any of the methods described herein, wherein the Cas nuclease is a Cas12a nuclease.

In some embodiments, the disclosure relates to any of the methods described herein, comprising a modified RNA.

In certain embodiments, the disclosure relates to any of the methods described herein, comprising a guide RNA nucleic acid, such as a gRNA. In certain embodiments, the disclosure relates to any of the methods described herein, wherein the guide RNA nucleic acid is or encodes a dual-guide RNA (dgRNA). In certain embodiments, the disclosure relates to any of the methods described herein, wherein the guide RNA nucleic acid is or encodes a single-guide (sgRNA). In some embodiments, the disclosure relates to any of the methods described herein, wherein the gRNA is a modified gRNA, for example wherein the modified gRNA comprises a modification at one or more of the first five nucleotides at the 5′ end or the modified gRNA comprises a modification at one or more of the last five nucleotides at the 3′ end.

In certain embodiments, the disclosure relates to any of the methods described herein, wherein the composition comprises a guide RNA nucleic acid and a Class 2 Cas nuclease mRNA; and the ratio of the mRNA to the guide RNA nucleic acid is from about 2:1 to 1:4 by weight. In some embodiments, the composition comprises a Class 2 Cas nuclease and guide RNA complex.

In some embodiments, the disclosure relates to any of the methods described herein, comprising the DNA cutting agent, wherein the DNA cutting agent is present in a lipid nucleic acid assembly composition.

In some embodiments, the disclosure relates to any of the methods described herein, comprising the donor DNA.

In certain embodiments, the disclosure relates to any of the methods described herein, wherein the donor DNA comprises a sequence encoding a protein, a regulatory sequence, or a sequence encoding structural RNA.

In certain embodiments, the disclosure relates to any of the methods described herein, wherein the template sequence is integrated into the genome of the cell via homology directed repair (HDR).

In certain embodiments, the disclosure relates to any of the methods described herein, comprising contacting the cell with a lipid nucleic acid assembly composition comprising the DNA cutting agent.

In certain embodiments, the disclosure relates to any of the methods described herein, wherein the lipid nucleic acid assembly composition is a lipid nanoparticle (LNP) composition. In some embodiments, the LNP composition is any of the LNP compositions described herein.

In certain embodiments, the disclosure relates to any of the compositions and methods described herein, wherein the LNP has a diameter of about 10-200 nm, about 20-150 nm, about 50-150 nm, about 50-100 nm, about 50-120 nm, about 60-100 nm, about 75-150 nm, about 75-120 nm, or about 75-100 nm. In certain embodiments, the disclosure relates to any of the methods described herein, wherein the composition comprises a population of the LNPs with an average diameter of about 10-200 nm, about 20-150 nm, about 50-150 nm, about 50-100 nm, about 50-120 nm, about 60-100 nm, about 75-150 nm, about 75-120 nm, or about 75-100 nm. For example, the average diameter may be a Z-average diameter.

In certain embodiments, the disclosure relates to any of the methods described herein, wherein the lipid nucleic acid assembly composition is a lipoplex.

In some embodiments, the disclosure relates to any of the methods described herein, wherein the lipid nucleic acid assembly composition comprises an ionizable lipid, for example, any of the ionizable lipids described herein. In certain embodiments, the disclosure relates to any of the methods described herein, wherein the ionizable lipid has a pKa from about 5.1 to 7.4, such as from about 5.5 to 6.6, from about 5.6 to 6.4, from about 5.8 to 6.2, or from about 5.8 to 6.5.

In certain embodiments, the disclosure relates to any of the methods described herein, the lipid nucleic acid assembly composition comprises a helper lipid.

In certain embodiments, the disclosure relates to any of the methods described herein, wherein the lipid nucleic acid assembly composition comprises a neutral lipid.

In some embodiments, the disclosure relates to any of the methods described herein, wherein the lipid nucleic acid assembly composition comprises a PEG lipid.

In certain embodiments, the disclosure relates to any of the methods described herein, wherein the N/P ratio of the lipid nucleic acid assembly composition is about 3-10, for example from about 5-7, preferably about 6.

In some embodiments, the disclosure relates to any of the methods described herein, further comprising a vector, for example, wherein the vector encodes the DNA cutting agent or the donor DNA. In certain embodiments, the disclosure relates to any of the methods described herein, wherein the vector is a viral vector. In other embodiments, the disclosure relates to any of the methods described herein, wherein the vector is a non-viral vector. In certain embodiments, the disclosure relates to any of the methods described herein, wherein the vector is a lentiviral vector. In some embodiments, the disclosure relates to any of the methods described herein, wherein the vector is a retroviral vector. In certain embodiments, the disclosure relates to any of the methods described herein, wherein the vector is an AAV.

In certain embodiments, the disclosure relates to any of the methods described herein, wherein the cell is not a cancer cell.

In certain embodiments, the disclosure relates to any of the methods described herein, wherein the DNA cutting agent interacts with a target sequence within the genome of the cell, resulting in a double stranded DNA break (DSB).

In some preferred embodiments, the disclosure relates to any of the methods described herein, wherein the method results in a gene knockout.

In some preferred embodiments, the disclosure relates to any of the methods described herein, wherein the method results in a gene correction.

In certain embodiments, the disclosure relates to any method of gene editing described herein, wherein the gene editing results in an insertion. In some embodiments, the insertion is a gene insertion.

In certain embodiments, the disclosure relates to any of the methods described herein, wherein the donor DNA comprises a template comprising an exogenous nucleic acid encoding a protein. In certain embodiments, the protein is selected from a cytokine, an immunosuppressor, an antibody, a receptor, and an enzyme. In certain embodiments, the protein is a receptor. In certain embodiments, the receptor is selected from an immunological receptor, a T-cell receptor (TCR), and a chimeric antigen receptor. In certain embodiments, the receptor is an immunological receptor. In certain embodiments, the receptor is a TCR. In certain embodiments, the exogenous nucleic acid encodes a TCR α chain and/or a TCR β chain of a TCR. In certain embodiments, the receptor a chimeric antigen receptor.

In certain embodiments, the disclosure relates to any method of gene editing described herein, wherein the DNA cutting agent interacts with a target sequence within the genome of the cell, resulting in a double stranded DNA break (DSB). In certain embodiments, the DNA cutting agent interacts with a target sequence within the TRAC gene of the T-cell. In certain embodiments, the template is integrated into the TRAC gene of the T-cell. In certain embodiments, the template comprises a first homology arm and a second homology arm that are complementary to sequences located upstream and downstream of the cleavage site, respectively.

The DNA cutting agent, such as a protein, RNA, or nucleic acid encoding the same, may be delivered to the cell by electroporation, lipid-based delivery, e.g. via lipid nucleic acid assemblies such as lipid nanoparticles, or other delivery technology known in the art.

### Ionizable Lipids

In some embodiments, methods and compositions are provided wherein nucleic acid assemblies comprise the DNA cutting agent and serve to deliver the DNA cutting agent to cells. Ionizable lipids and other “biodegradable lipids” suitable for use in the lipid nucleic acid assemblies described herein are biodegradable in vivo or ex vivo. The ionizable lipids have low toxicity (e.g., are tolerated in animal models without adverse effect in amounts of greater than or equal to 10 mg/kg). Biodegradable lipids suitable for use in the lipid nucleic acid assemblies described herein include, for example the biodegradable lipids of WO/2020/219876, WO/2020/118041, WO/2020/072605, WO/2019/067992, WO/2017/173054, WO2015/095340, and WO2014/136086, each of which is hereby incorporated by reference in its entirety, and specifically the ionizable lipids and compositions of each are hereby incorporated by reference.

In some embodiments, lipid nucleic acid assembly compositions comprise an ionizable lipid such as Lipid A or its equivalents, including acetal analogs of Lipid A.

In some embodiments, the ionizable lipid is Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

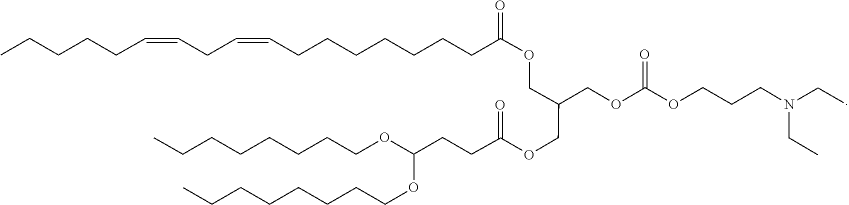

Lipid A may be synthesized according to WO2015/095340 (e.g., pp. 84-86).

In some embodiments, the ionizable lipid is Lipid D, which is nonyl 8-((7,7-bis(octyloxy)heptyl)(2-hydroxyethyl)amino)octanoate. Lipid D can be depicted as:

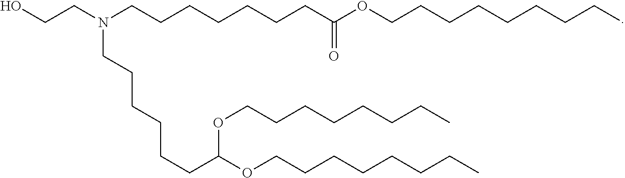

Lipid D may be synthesized according to WO2020/072605.

The ionizable lipids of the present disclosure may form salts depending upon the pH of the medium they are in. For example, in a slightly acidic medium, the ionizable lipids may be protonated and thus bear a positive charge. Conversely, in a slightly basic medium, such as, for example, blood where pH is approximately 7.35, the ionizable lipids may not be protonated and thus bear no charge. In some embodiments, the ionizable lipids of the present disclosure may be predominantly protonated at a pH of at least about 9. In some embodiments, the ionizable lipids of the present disclosure may be predominantly protonated at a pH of at least about 10.

The pH at which an ionizable lipid is predominantly protonated is related to its intrinsic pKa. In some embodiments, a salt of an ionizable lipid of the present disclosure has a pKa in the range of from about 5.1 to about 8.0, even more preferably from about 5.5 to about 7.6. In some embodiments, a salt of an ionizable lipid of the present disclosure has a pKa in the range of from about 5.7 to about 8, from about 5.7 to about 7.6, from about 6 to about 8, from about 6 to about 7.5, from about 6 to about 7, or from about 6 to about 6.5. In some embodiments, a salt of an ionizable lipid of the present disclosure has a pKa of about 6.0, about 6.1, about 6.1, about 6.2, about 6.3, about 6.4, about 6.6, or about 6.6. Alternatively, a salt of an ionizable lipid of the present disclosure has a pKa in the range of from about 6 to about 8. The pKa can be an important consideration in formulating LNPs, as it has been found that LNPs formulated with certain lipids having a pKa ranging from about 5.5 to about 7.0 are effective for delivery of cargo in vivo, e.g. to the liver. Further, it has been found that LNPs formulated with certain lipids having a pKa ranging from about 5.3 to about 6.4 are effective for delivery in vivo, e.g. to tumors. See, e.g., WO 2014/136086. In some embodiments, the ionizable lipids are positively charged at an acidic pH but neutral in the blood.

### Additional Lipids

“Neutral lipids” suitable for use in a lipid composition of the disclosure include, for example, a variety of neutral, uncharged or zwitterionic lipids. Examples of neutral phospholipids suitable for use in the present disclosure include, but are not limited to, dipalmitoylphosphatidylcholine (DPPC), distearoylphosphatidylcholine (DSPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In certain embodiments, the neutral phospholipid may be selected from distearoylphosphatidylcholine (DSPC) and dimyristoyl phosphatidyl ethanolamine (DMPE), preferably distearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In certain embodiments, the helper lipid may be cholesterol or a derivative thereof, such as cholesterol hemisuccinate.

In some embodiments, the LNP compositions include polymeric lipids, such as PEG lipids, which can affect the length of time the nanoparticles can exist in vivo or ex vivo (e.g., in the blood or medium). PEG lipids may assist in the formulation process by, for example, reducing particle aggregation and controlling particle size. PEG lipids used herein may modulate pharmacokinetic properties of the LNP compositions. Typically, the PEG lipid comprises a lipid moiety and a polymer moiety based on PEG (sometimes referred to as poly(ethylene oxide)) (a PEG moiety). PEG lipids suitable for use in a lipid composition of the present disclosure and information about the biochemistry of such lipids can be found in Romberg et al., Pharmaceutical Research 25(1), 2008, pp. 55-71 and Hoekstra et al., Biochimica et Biophysica Acta 1660 (2004) 41-52. Additional suitable PEG lipids are disclosed, e.g., in WO 2015/095340 (p. 31, line 14 to p. 37, line 6), WO 2006/007712, and WO 2011/076807 (“stealth lipids”), which are incorporated by reference.

In some embodiments, the lipid moiety may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. In some embodiments, the alkyl chain length comprises about C10 to C20. The dialkylglycerol or dialkylglycamide group can further comprise one or more substituted alkyl groups. The chain lengths may be symmetrical or asymmetric.

Unless otherwise indicated, the term “PEG” as used herein means any polyethylene glycol or other polyalkylene ether polymer, such as an optionally substituted linear or branched polymer of ethylene glycol or ethylene oxide. In certain embodiments, the PEG moiety is unsubstituted. Alternatively, the PEG moiety may be substituted, e.g., by one or more alkyl, alkoxy, acyl, hydroxy, or aryl groups. For example, the PEG moiety may comprise a PEG copolymer such as PEG-polyurethane or PEG-polypropylene (see, e.g., J. Milton Harris, Poly(ethylene glycol) chemistry: biotechnical and biomedical applications (1992)); alternatively, the PEG moiety may be a PEG homopolymer. In certain embodiments, the PEG moiety has a molecular weight of from about 130 to about 50,000, such as from about 150 to about 30,000, or even from about 150 to about 20,000. Similarly, the PEG moiety may have a molecular weight of from about 150 to about 15,000, from about 150 to about 10,000, from about 150 to about 6,000, or even from about 150 to about 5,000. In certain preferred embodiments, the PEG moiety has a molecular weight of from about 150 to about 4,000, from about 150 to about 3,000, from about 300 to about 3,000, from about 1,000 to about 3,000, or from about 1,500 to about 2,500.

In certain preferred embodiments, the PEG moiety is a “PEG-2K,” also termed “PEG 2000,” which has an average molecular weight of about 2,000 daltons. PEG-2K is represented herein by the following formula (III)

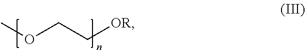

wherein n is about 45, meaning that the number averaged degree of polymerization comprises about 45 subunits. However, other PEG embodiments known in the art may be used, including, e.g., those where the number-averaged degree of polymerization comprises about 23 subunits (n=23), and/or 68 subunits (n=68). In some embodiments, n may range from about 30 to about 60. In some embodiments, n may range from about 35 to about 55. In some embodiments, n may range from about 40 to about 50. In some embodiments, n may range from about 42 to about 48. In some embodiments, n may be 45. In some embodiments, R may be selected from H, substituted alkyl, and unsubstituted alkyl. In some embodiments, R may be unsubstituted alkyl, such as methyl.

In any of the embodiments described herein, the PEG lipid may be selected from PEG-dilauroylglycerol, PEG-dimyristoylglycerol (PEG-DMG) (catalog #GM-020 from NOF, Tokyo, Japan), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000](PEG2k-DMPE), or 1,2-dimyristoyl-rac-glycero-3-methoxypolyethylene glycol-2000 (PEG2k-DMG), 1,2-distearoyl-sn-glycero-3-phosphoethanolamine-N-[methoxy(polyethylene glycol)-2000] (PEG2k-DSPE) (cat. #880120C from Avanti Polar Lipids, Alabaster, Alabama, USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy(polyethylene glycol)-2000] (PEG2k-DSA). In certain such embodiments, the PEG lipid may be PEG2k-DMG. In some embodiments, the PEG lipid may be PEG2k-DSG. In other embodiments, the PEG lipid may be PEG2k-DSPE. In some embodiments, the PEG lipid may be PEG2k-DMA. In yet other embodiments, the PEG lipid may be PEG2k-C-DMA. In certain embodiments, the PEG lipid may be compound S027, disclosed in WO2016/010840 (paragraphs [00240] to [00244]). In some embodiments, the PEG lipid may be PEG2k-DSA. In other embodiments, the PEG lipid may be PEG2k-C11. In some embodiments, the PEG lipid may be PEG2k-C14. In some embodiments, the PEG lipid may be PEG2k-C16. In some embodiments, the PEG lipid may be PEG2k-C18.

In preferred embodiments, the PEG lipid includes a glycerol group. In preferred embodiments, the PEG lipid includes a dimyristoylglycerol (DMG) group. In preferred embodiments, the PEG lipid comprises PEG-2k. In preferred embodiments, the PEG lipid is a PEG-DMG. In preferred embodiments, the PEG lipid is a PEG-2k-DMG. In preferred embodiments, the PEG lipid is 1,2-dimyristoyl-rac-glycero-3-methoxypolyethylene glycol-2000. In preferred embodiments, the PEG-2k-DMG is 1,2-dimyristoyl-rac-glycero-3-methoxypolyethylene glycol-2000.

In some embodiments, methods and compositions are provided wherein nucleic acid assemblies comprise a cationic lipid composition and the DNA cutting agent and serve to deliver the DNA cutting agent to cells. Cationic lipids suitable for use in a lipid compositions described herein include, but are not limited to, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), 1,2-Dioleoyl-3-Dimethylammonium-propane (DODAP), N-(1-(2,3-dioleyloxy)propyl)-N,N,N-trimethylammonium chloride (DOTMA), 1,2-Dioleoylcarbamyl-3-Dimethylammonium-propane (DOCDAP), 1,2-Dilineoyl-3-Dimethylammonium-propane (DLINDAP), dilauryl(C12:0) trimethyl ammonium propane (DLTAP), Dioctadecylamidoglycyl spermine (DOGS), DC-Choi, Dioleoyloxy-N-[2-(sperminecarboxamido)ethyl]-N,N-dimethyl-1-propanaminiumtrifluoroacetate (DOSPA), 1,2-Dimyristyloxypropyl-3-dimethyl-hydroxyethyl ammonium bromide (DMRIE), 3-Dimethylamino-2-(Cholest-5-en-3-beta-oxybutan-4-oxy)-1-(cis,cis-9,12-octadecadienoxy)propane (CLinDMA), N,N-dimethyl-2,3-dioleyloxy)propylamine (DODMA), 2-[5′-(cholest-5-en-3[beta]-oxy)-3′-oxapentoxy)-3-dimethyl-1-(cis,cis-9′,1-2′-octadecadienoxy) propane (CpLinDMA), N,N-Dimethyl-3,4-dioleyloxybenzylamine (DMOBA), and 1,2-N,N′-Dioleylcarbamyl-3-dimethylaminopropane (DOcarbDAP). In some embodiments, the cationic lipid is DOTAP or DLTAP.

In further embodiments, methods and compositions are provided wherein nucleic acid assemblies comprise an anionic lipid composition and the DNA cutting agent and serve to deliver the DNA cutting agent to cells. Anionic lipids suitable for use in the compositions described herein include, but are not limited to, phosphatidylglycerol, cardiolipin, diacylphosphatidylserine, diacylphosphatidic acid, N-dodecanoyl phosphatidyl ethanolamine, N-succinyl phosphatidylethanolamine, N-glutaryl phosphatidylethanolamine cholesterol hemisuccinate (CHEMS), and lysylphosphatidylglycerol.

### Lipid Compositions

Described herein are lipid compositions comprising at least one ionizable, cationic, or anionic lipid, such as an ionizable lipid, or a salt thereof (e.g., a pharmaceutically acceptable salt thereof), optionally at least one helper lipid, at least one neutral lipid, and at least one polymeric lipid. In some embodiments, the lipid composition comprises an ionizable lipid, or a salt thereof, a neutral lipid, a helper lipid, and a PEG lipid. In some embodiments, the neutral lipid is DSPC or DPME. In some embodiments, the helper lipid is cholesterol, 5-heptadecylresorcinol, or cholesterol hemisuccinate.

In preferred embodiments, the ionizable lipid is

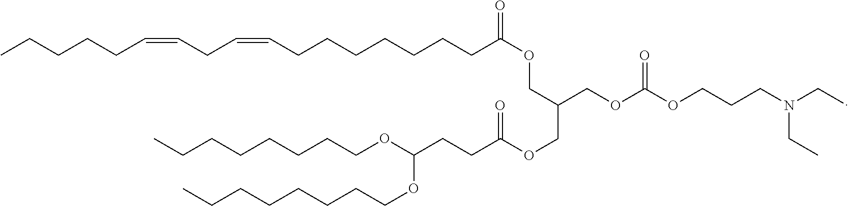

In preferred embodiments, the neutral lipid is DSPC. In preferred embodiments, the helper lipid is cholesterol. In preferred embodiments, the PEG lipid is 1,2-dimyristoyl-rac-glycero-3-methoxypolyethylene glycol-2000. In particularly preferred embodiments, the ionizable lipid is

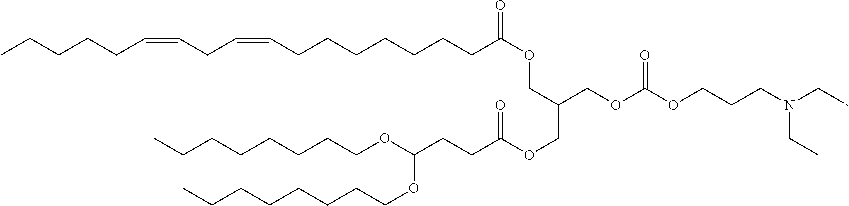

the neutral lipid is DSPC, the helper lipid is cholesterol, and the PEG lipid is 1,2-dimyristoyl-rac-glycero-3-methoxypolyethylene glycol-2000.

In preferred embodiments, the ionizable lipid is

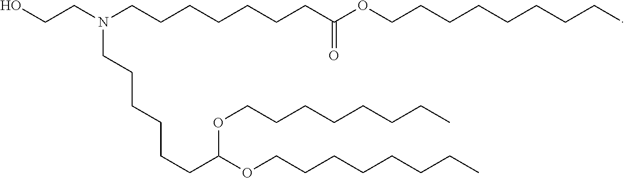

In preferred embodiments, the neutral lipid is DSPC. In preferred embodiments, the helper lipid is cholesterol. In preferred embodiments, the PEG lipid is 1,2-dimyristoyl-rac-glycero-3-methoxypolyethylene glycol-2000.

In particularly preferred embodiments, the ionizable lipid is

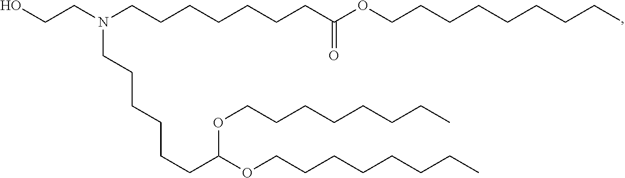

the neutral lipid is DSPC, the helper lipid is cholesterol, and the PEG lipid is 1,2-dimyristoyl-rac-glycero-3-methoxypolyethylene glycol-2000.

In some embodiments, the lipid composition further comprises one or more additional lipid components. In some embodiments, the lipid composition further comprises at least one cationic lipid and/or at least one anionic lipid. In further embodiments, the lipid composition further comprises a cationic lipid, optionally with one or more additional lipid components. In further embodiments, the lipid composition further comprises an anionic lipid, optionally with one or more additional lipid components.

In some embodiments, the lipid composition is in the form of a liposome. In preferred embodiments, the lipid composition is in the form of a lipid nanoparticle (LNP) composition. “Lipid nanoparticle” or “LNP” refers to, without limiting the meaning, a particle that comprises a plurality of (i.e., more than one) LNP components physically associated with each other by intermolecular forces. In certain embodiments the lipid composition is suitable for delivery in vivo. In certain embodiments the lipid composition is suitable for delivery to an organ, such as the liver. In certain embodiments the lipid composition is suitable for delivery to a tissue ex vivo. In certain embodiments the lipid composition is suitable for delivery to a cell in vitro.

Lipid compositions may be in various forms, including, but not limited to, particle forming delivery agents including microparticles, nanoparticles and transfection agents that are useful for delivering various molecules to cells. Specific compositions are effective at transfecting or delivering biologically active agents. Preferred biologically active agents are RNAs and DNAs. In further embodiments, the biologically active agent is chosen from mRNA, gRNA, and DNA. The gRNA may be a dgRNA or an sgRNA. In certain embodiments, the cargo includes an mRNA encoding an RNA-guided DNA-cutting agent (e.g. a Cas nuclease, a Class 2 Cas nuclease, or Cas9), a gRNA or a nucleic acid encoding a gRNA, or a combination of mRNA and gRNA.

The compounds or compositions will generally, but not necessarily, include one or more pharmaceutically acceptable excipients. The term “excipient” includes any ingredient other than the compound(s) of the disclosure, the other lipid component(s) and the biologically active agent. An excipient may impart either a functional (e.g. drug release rate controlling) and/or a non-functional (e.g. processing aid or diluent) characteristic to the compositions. The choice of excipient will to a large extent depend on factors such as the particular mode of administration, the effect of the excipient on solubility and stability, and the nature of the dosage form.

Parenteral formulations are typically aqueous or oily solutions or suspensions. Where the formulation is aqueous, excipients such as sugars (including but not restricted to glucose, mannitol, sorbitol, etc.) salts, carbohydrates and buffering agents (preferably to a pH of from 3 to 9), but, for some applications, they may be more suitably formulated with a sterile non-aqueous solution or as a dried form to be used in conjunction with a suitable vehicle such as sterile, pyrogen-free water (WFI).

### LNP Compositions

The lipid compositions may be provided as LNP compositions, and LNP compositions described herein may be provided as lipid compositions. Lipid nanoparticles may be, e.g., microspheres (including unilamellar and multilamellar vesicles, e.g. “liposomes”-lamellar phase lipid bilayers that, in some embodiments are substantially spherical, and, in more particular embodiments can comprise an aqueous core, e.g., comprising a substantial portion of RNA molecules), a dispersed phase in an emulsion, micelles or an internal phase in a suspension.

Described herein are LNP compositions comprising at least one ionizable lipid, or a salt thereof (e.g., a pharmaceutically acceptable salt thereof), at least one helper lipid, at least one neutral lipid, and at least one polymeric lipid. In some embodiments, the LNP composition comprises at least one ionizable lipid, or a pharmaceutically acceptable salt thereof, at least one neutral lipid, at least one helper lipid, and at least one PEG lipid. In some embodiments, the neutral lipid is DSPC or DPME. In some embodiments, the helper lipid is cholesterol, 5-heptadecylresorcinol, or cholesterol hemisuccinate.

In preferred embodiments, the ionizable lipid is

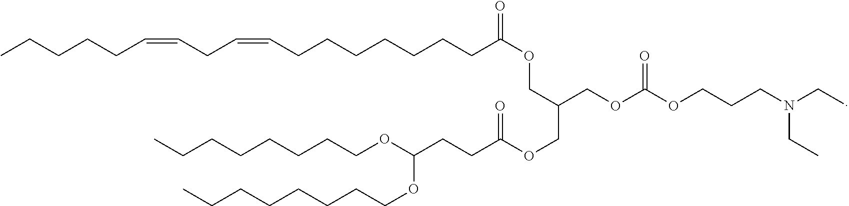

In preferred embodiments, the neutral lipid is DSPC. In preferred embodiments, the helper lipid is cholesterol. In preferred embodiments, the PEG lipid is 1,2-dimyristoyl-rac-glycero-3-methoxypolyethylene glycol-2000. In particularly preferred embodiments, the ionizable lipid is

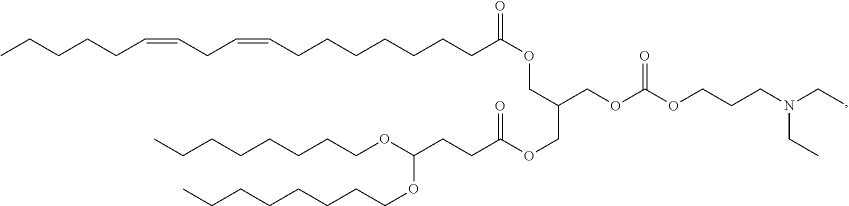

the neutral lipid is DSPC, the helper lipid is cholesterol, and the PEG lipid is 1,2-dimyristoyl-rac-glycero-3-methoxypolyethylene glycol-2000.

In preferred embodiments, the ionizable lipid is

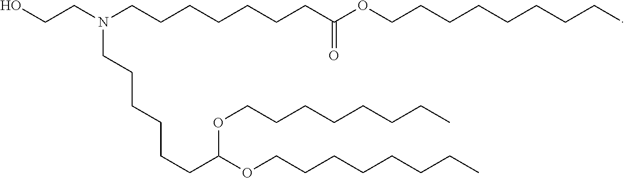

In preferred embodiments, the neutral lipid is DSPC. In preferred embodiments, the helper lipid is cholesterol. In preferred embodiments, the PEG lipid is 1,2-dimyristoyl-rac-glycero-3-methoxypolyethylene glycol-2000.

In particularly preferred embodiments, the ionizable lipid is

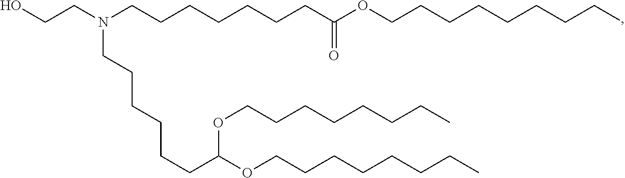

the neutral lipid is DSPC, the helper lipid is cholesterol, and the PEG lipid is 1,2-dimyristoyl-rac-glycero-3-methoxypolyethylene glycol-2000.

Embodiments of the present disclosure provide lipid compositions described according to the respective molar ratios of the component lipids in the composition. In certain embodiments, the amount of the ionizable lipid is from about 25 mol % to about 60 mol %; the amount of the neutral lipid is from about 5 mol % to about 30 mol %; the amount of the helper lipid is from about 20 mol % to about 65 mol %; and the amount of the PEG lipid is from about 0.5 mol % to about 10 mol %. All mol % numbers are given as a fraction of the lipid component of the lipid composition or, more specifically, the LNP compositions. In some embodiments, the lipid mol % of a lipid relative to the lipid component will be ±30%, ±25%, ±20%, ±15%, ±10%, ±5%, or ±2.5% of the specified, nominal, or actual mol %. In some embodiments, the lipid mol % of a lipid relative to the lipid component will be ±4 mol %, +3 mol %, +2 mol %, +1.5 mol %, +1 mol %, +0.5 mol %, +0.25 mol %, or +0.05 mol % of the specified, nominal, or actual mol % of the lipid component. In certain embodiments, the lipid mol % will vary by less than 15%, less than 10%, less than 5%, less than 1%, or less than 0.5% from the specified, nominal, or actual mol % of the lipid. In some embodiments, the mol % numbers are based on nominal concentration. As used herein, “nominal concentration” refers to concentration based on the input amounts of substances combined to form a resulting composition. For example, if 100 mg of solute is added to 1 L water, the nominal concentration is 100 mg/L. In some embodiments, the mol % numbers are based on actual concentration, e.g., concentration determined by an analytic method. In some embodiments, actual concentration of the lipids of the lipid component may be determined, for example, from chromatography, such as liquid chromatography, followed by a detection method, such as charged aerosol detection. In some embodiments, actual concentration of the lipids of the lipid component may be characterized by lipid analysis, AF4-MALS, NTA, and/or cryo-EM. All mol % numbers are given as a percentage of the lipids of the lipid component of an LNP composition.

In some embodiments, the aqueous component comprises a DNA cutting agent. In some embodiments, the aqueous component comprises a polypeptide DNA cutting agent, optionally in combination with a nucleic acid. In some embodiments, the aqueous component comprises a nucleic acid DNA cutting agent, such as an RNA that encodes a nuclease or nickase. In some embodiments, the aqueous component is a nucleic acid component. In some embodiments, the nucleic acid component comprises DNA and it can be called a DNA component. In some embodiments, the nucleic acid component comprises RNA. In some embodiments, the aqueous component, such as an RNA component may comprise an mRNA, such as an mRNA encoding an RNA-guided DNA-cutting agent. In some embodiments, the RNA-guided DNA-cutting agent is a Cas nuclease. In certain embodiments, aqueous component may comprise an mRNA that encodes a Cas nuclease, such as Cas9. In certain embodiments, the DNA cutting agent is a Cas nuclease mRNA. In certain embodiments, the DNA cutting agent is a Class 2 Cas nuclease mRNA. In certain embodiments, the DNA cutting agent is a Cas9 nuclease mRNA. In certain embodiments, the aqueous component may comprise a modified RNA. In some embodiments, the aqueous component may comprise a guide RNA nucleic acid. In certain embodiments, the aqueous component may comprise a gRNA. In certain embodiments, the aqueous component may comprise a dgRNA. In certain embodiments, the aqueous component may comprise a modified gRNA. In some compositions comprising an mRNA encoding an RNA-guided DNA-cutting agent, the composition further comprises a gRNA nucleic acid, such as a gRNA. In some embodiments, the aqueous component comprises an RNA-guided DNA-cutting agent and a gRNA. In some embodiments, the aqueous component comprises a Cas nuclease mRNA and a gRNA. In some embodiments, the aqueous component comprises a Class 2 Cas nuclease mRNA and a gRNA.

In certain embodiments, a lipid composition, such as an LNP composition, may comprise an mRNA encoding a Cas nuclease such as a Class 2 Cas nuclease, an ionizable lipid or a pharmaceutically acceptable salt thereof, a helper lipid, optionally a neutral lipid, and a PEG lipid. In certain compositions comprising an mRNA encoding a Cas nuclease such as a Class 2 Cas nuclease, the helper lipid is cholesterol. In other compositions comprising an mRNA encoding a Cas nuclease such as a Class 2 Cas nuclease, the neutral lipid is DSPC. In additional embodiments comprising an mRNA encoding a Cas nuclease such as a Class 2 Cas nuclease, e.g. Cas9, the PEG lipid is PEG2k-DMG. In specific compositions comprising an mRNA encoding a Cas nuclease such as a Class 2 Cas nuclease, and an ionizable lipid or a pharmaceutically acceptable salt thereof. In certain compositions, the composition further comprises a gRNA, such as a dgRNA or an sgRNA.

In some embodiments, a lipid composition, such as an LNP composition, may comprise a gRNA. In certain embodiments, a composition may comprise an ionizable lipid or a pharmaceutically acceptable salt thereof, a gRNA, a helper lipid, optionally a neutral lipid, and a PEG lipid. In certain LNP compositions comprising a gRNA, the helper lipid is cholesterol. In some compositions comprising a gRNA, the neutral lipid is DSPC. In additional embodiments comprising a gRNA, the PEG lipid is PEG2k-DMG. In certain compositions, the gRNA is selected from dgRNA and sgRNA.

In certain embodiments, a lipid composition, such as an LNP composition, comprises an mRNA encoding an RNA-guided DNA-cutting agent and a gRNA, which may be an sgRNA, in an aqueous component and an ionizable lipid in a lipid component. For example, an LNP composition may comprise an ionizable lipid or a pharmaceutically acceptable salt thereof, an mRNA encoding a Cas nuclease, a gRNA, a helper lipid, a neutral lipid, and a PEG lipid. In certain compositions comprising an mRNA encoding a Cas nuclease and a gRNA, the helper lipid is cholesterol. In some compositions comprising an mRNA encoding a Cas nuclease and a gRNA, the neutral lipid is DSPC. In additional embodiments comprising an mRNA encoding a Cas nuclease and a gRNA, the PEG lipid is PEG2k-DMG.

In certain embodiments, the lipid compositions, such as LNP compositions include an RNA-guided DNA-cutting agent, such as a Class 2 Cas mRNA and at least one gRNA. In some embodiments, the gRNA is a sgRNA. In some embodiments, the RNA-guided DNA-cutting agent is a Cas9 mRNA In certain embodiments, the LNP composition includes a ratio of gRNA to RNA-guided DNA-cutting agent mRNA, such as Class 2 Cas nuclease mRNA of about 1:1 or about 1:2. In some embodiments, the ratio of by weight is from about 25:1 to about 1:25, about 10:1 to about 1:10, about 8:1 to about 1:8, about 4:1 to about 1:4, about 2:1 to about 1:2, about 2:1 to 1:4 by weight, or about 1:1 to about 1:2.

The compositions and methods disclosed herein may include a template nucleic acid, e.g., a DNA template. The template nucleic acid may be delivered at the same time as, or separately from, the lipid compositions comprising an ionizable lipid or a pharmaceutically acceptable salt thereof, including as LNP compositions. In some embodiments, the template nucleic acid may be single- or double-stranded, depending on the desired repair mechanism. The template may have regions of homology to the target DNA, e.g. within the target DNA sequence, and/or to sequences adjacent to the target DNA.

In some embodiments, LNP compositions are formed by mixing an aqueous RNA solution with an organic solvent-based lipid solution. Suitable solutions or solvents include or may contain: water, PBS, Tris buffer, NaCl, citrate buffer, acetate buffer, ethanol, chloroform, diethylether, cyclohexane, tetrahydrofuran, methanol, isopropanol. For example, the organic solvent may be 100% ethanol. A pharmaceutically acceptable buffer, e.g., for in vivo administration of LNP compositions, may be used. In certain embodiments, a buffer is used to maintain the pH of the composition comprising LNPs at or above pH 6.5. In certain embodiments, a buffer is used to maintain the pH of the composition comprising LNPs at or above pH 7.0. In certain embodiments, the composition has a pH ranging from about 7.2 to about 7.7. In additional embodiments, the composition has a pH ranging from about 7.3 to about 7.7 or ranging from about 7.4 to about 7.6. The pH of a composition may be measured with a micro pH probe. In certain embodiments, a cryoprotectant is included in the composition. Non-limiting examples of cryoprotectants include sucrose, trehalose, glycerol, DMSO, and ethylene glycol. Exemplary compositions may include up to 10% cryoprotectant, such as, for example, sucrose. In certain embodiments, the composition may comprise tris saline sucrose (TSS). In certain embodiments, the composition is an LNP composition, which may include about 1, 2, 3, 4, 5, 6, 7, 8, 9, or 10% cryoprotectant. In certain embodiments, the composition is an LNP composition, which may include about 1, 2, 3, 4, 5, 6, 7, 8, 9, or 10% sucrose. In some embodiments, the composition includes a buffer. In some embodiments, the buffer may comprise a phosphate buffer (PBS), a Tris buffer, a citrate buffer, and mixtures thereof. In certain exemplary embodiments, the buffer comprises NaCl. In certain embodiments, the buffer lacks NaCl. Exemplary amounts of NaCl may range from about 20 mM to about 45 mM. Exemplary amounts of NaCl may range from about 40 mM to about 50 mM. In some embodiments, the amount of NaCl is about 45 mM. In some embodiments, the buffer is a Tris buffer. Exemplary amounts of Tris may range from about 20 mM to about 60 mM. Exemplary amounts of Tris may range from about 40 mM to about 60 mM. In some embodiments, the amount of Tris is about 50 mM. In some embodiments, the buffer comprises NaCl and Tris. Certain exemplary embodiments of the compositions contain 5% sucrose and 45 mM NaCl in Tris buffer. In other exemplary embodiments, compositions contain sucrose in an amount of about 5% w/v, about 45 mM NaCl, and about 50 mM Tris at pH 7.5. The salt, buffer, and cryoprotectant amounts may be varied such that the osmolality of the overall composition is maintained. For example, the final osmolality may be maintained at less than 450 mOsm/L. In further embodiments, the osmolality is between 350 and 250 mOsm/L. Certain embodiments have a final osmolality of 300+/−20 mOsm/L or 310+/−40 mOsm/L.

In some embodiments, microfluidic mixing, T-mixing, or cross-mixing of the aqueous RNA solution and the lipid solution in an organic solvent is used to make LNP compositions. In certain aspects, flow rates, junction size, junction geometry, junction shape, tube diameter, solutions, and/or RNA and lipid concentrations may be varied. LNPs or LNP compositions may be concentrated or purified, e.g., via dialysis, centrifugal filter, tangential flow filtration, or chromatography. The LNP compositions may be stored as a suspension, an emulsion, or a lyophilized powder, for example. In some embodiments, an LNP composition is stored at 2-8° C., in certain aspects, the LNP compositions are stored at room temperature. In additional embodiments, an LNP composition is stored frozen, for example at −20° C. or −80° C. In other embodiments, an LNP composition is stored at a temperature ranging from about 0° C. to about −80° C. Frozen LNP compositions may be thawed before use, for example on ice, at room temperature, or at 25° C.

Preferred lipid compositions, such as LNP compositions, are biodegradable, in that they do not accumulate to cytotoxic levels in vivo at a therapeutically effective dose. In some embodiments, the compositions do not cause an innate immune response that leads to substantial adverse effects at a therapeutic dose level. In some embodiments, the compositions provided herein do not cause toxicity at a therapeutic dose level.

In some embodiments, the concentration of the LNPs in the LNP composition is about 1-10 μg/mL, about 2-10 μg/mL, about 2.5-10 μg/mL, about 1-5 μg/mL, about 2-5 μg/mL, about 2.5-5 μg/mL, about 0.04 μg/mL, about 0.08 μg/mL, about 0.16 μg/mL, about 0.25 μg/mL, about 0.63 μg/mL, about 1.25 μg/mL, about 2.5 μg/mL, or about 5 μg/mL.

In some embodiments, Dynamic Light Scattering (“DLS”) may be used to characterize the polydispersity index (PDI) and size of the LNPs of the present disclosure. DLS measures the scattering of light that results from subjecting a sample to a light source. PDI, as determined from DLS measurements, represents the distribution of particle size (around the mean particle size) in a population, with a perfectly uniform population having a PDI of zero.

In some embodiments, the LNPs disclosed herein have a PDI from about 0.005 to about 0.75. In some embodiments, the LNPs disclosed herein have a PDI from about 0.005 to about 0.1. In some embodiments, the LNPs disclosed herein have a PDI from about 0.005 to about 0.09, about 0.005 to about 0.08, about 0.005 to about 0.07, or about 0.006 to about 0.05. In some embodiments, the LNP have a PDI from about 0.01 to about 0.5. In some embodiments, the LNP have a PDI from about zero to about 0.4. In some embodiments, the LNP have a PDI from about zero to about 0.35. In some embodiments, the LNP PDI may range from about zero to about 0.3. In some embodiments, the LNP have a PDI that may range from about zero to about 0.25. In some embodiments, the LNP PDI may range from about zero to about 0.2. In some embodiments, the LNP have a PDI from about zero to about 0.05. In some embodiments, the LNP have a PDI from about zero to about 0.01. In some embodiments, the LNP have a PDI less than about 0.01, about 0.02, about 0.05, about 0.08, about 0.1, about 0.15, about 0.2, or about 0.4.

LNP size may be measured by various analytical methods known in the art. In some embodiments, LNP size may be measured using Asymetric-Flow Field Flow Fractionation-Multi-Angle Light Scattering (AF4-MALS). In certain embodiments, LNP size may be measured by separating particles in the composition by hydrodynamic radius, followed by measuring the molecular weights, hydrodynamic radii and root mean square radii of the fractionated particles. In some embodiments, LNP size and particle concentration may be measured by nanoparticle tracking analysis (NTA, Malvern Nanosight). In certain embodiments, LNP samples are diluted appropriately and injected onto a microscope slide. A camera records the scattered light as the particles are slowly infused through field of view. After the movie is captured, the Nanoparticle Tracking Analysis processes the movie by tracking pixels and calculating a diffusion coefficient. This diffusion coefficient can be translated into the hydrodynamic radius of the particle. Such methods may also count the number of individual particles to give particle concentration. In some embodiments, LNP size, morphology, and structural characteristics may be determined by cryo-electron microscopy (“cryo-EM”).

The LNPs of the LNP compositions disclosed herein, for example, have a size (e.g. Z-average diameter) of about 1 to about 250 nm. In some embodiments, the LNPs have a size of about 10 to about 200 nm. In further embodiments, the LNPs have a size of about 20 to about 150 nm. In some embodiments, the LNPs have a size of about 50 to about 150 nm or about 70 to 130 nm. In some embodiments, the LNPs have a size of about 50 to about 100 nm. In some embodiments, the LNPs have a size of about 50 to about 120 nm. In some embodiments, the LNPs have a size of about 60 to about 100 nm. In some embodiments, the LNPs have a size of about 75 to about 150 nm. In some embodiments, the LNPs have a size of about 75 to about 120 nm. In some embodiments, the LNPs have a size of about 75 to about 100 nm. In some embodiments, the LNPs have a size of about 50 to about 145 nm, about 50 to about 120 nm, about 50 to about 120 nm, about 50 to about 115 nm, about 50 to about 100 nm, about 60 to about 145 nm, about 60 to about 120 nm, about 60 to about 115 nm, or about 60 to about 100 nm. In some embodiments, the LNPs have a size of less than about 145 nm, less than about 120 nm, less than about 115 nm, or less than about 100 nm. In some embodiments, the LNPs have a size of greater than about 50 nm or greater than about 60 nm. In some embodiments, the particle size is a Z-average particle size. In some embodiments, the particle size is a number-average particle size. In some embodiments, the particle size is the size of an individual LNP. Unless indicated otherwise, all sizes referred to herein are the average sizes (diameters) of the fully formed nanoparticles, as measured by dynamic light scattering on a Malvern Zetasizer or Wyatt NanoStar. The nanoparticle sample is diluted in phosphate buffered saline (PBS) so that the count rate is approximately 200-400 kcps.

The LNPs may have a size (e.g. Z-average diameter) of about 1 to about 250 nm. In some embodiments, the LNPs have a size of about 10 to about 200 nm. In further embodiments, the LNPs have a size of about 20 to about 150 nm. In some embodiments, the LNPs have a size of about 50 to about 150 nm or about 70 to 130 nm. In some embodiments, the LNPs have a size of about 50 to about 100 nm. In some embodiments, the LNPs have a size of about 50 to about 120 nm. In some embodiments, the LNPs have a size of about 60 to about 100 nm. In some embodiments, the LNPs have a size of about 75 to about 150 nm. In some embodiments, the LNPs have a size of about 75 to about 120 nm. In some embodiments, the LNPs have a size of about 75 to about 100 nm. In some embodiments, the LNPs have a size of about 40 to about 125 nm, about 40 to about 110 nm, about 40 to about 100 nm, about 40 to about 90 nm. In some embodiments, the particle size is a Z-average particle size. In some embodiments, the particle size is a number-average particle size. In some embodiments, the particle size is the size of an individual LNP. Unless indicated otherwise, all sizes referred to herein are the average sizes (diameters) of the fully formed nanoparticles, as measured by dynamic light scattering on a Malvern Zetasizer or Wyatt NanoStar. The nanoparticle sample is diluted in phosphate buffered saline (PBS) so that the count rate is approximately 200-400 kcps.

In some embodiments, the LNP compositions are formed with an average encapsulation efficiency ranging from about 50% to about 100%. In some embodiments, the LNP compositions are formed with an average encapsulation efficiency ranging from about 50% to about 95%. In some embodiments, the LNP compositions are formed with an average encapsulation efficiency ranging from about 70% to about 90%. In some embodiments, the LNP compositions are formed with an average encapsulation efficiency ranging from about 90% to about 100%. In some embodiments, the LNP compositions are formed with an average encapsulation efficiency ranging from about 75% to about 95%. In some embodiments, the LNP compositions are formed with an average encapsulation efficiency ranging from about 90% to about 100%. In some embodiments, the LNP compositions are formed with an average encapsulation efficiency ranging from about 95% to about 100%. In some embodiments, the LNP compositions are formed with an average encapsulation efficiency ranging from about 98% to about 100%. In some embodiments, the LNP compositions are formed with an average encapsulation efficiency ranging from about 99% to about 100%.

### Cargo

The cargo delivered via LNP composition may be a DNA cutting agent, such as an RNA-guided DNA cutting agent. In certain embodiments, the cargo is or comprises one or more DNA cutting agent, such as mRNA, gRNA, expression vector, RNA-guided DNA-cutting agent, e.g. a CRISPR Cas nuclease or mRNA encoding the nuclease, optionally in combination with a guide RNA. The above list of DNA cutting agents is exemplary only, and is not intended to be limiting. Such compounds may be purified or partially purified, and may be naturally occurring or synthetic, and may be chemically modified.

The cargo delivered via LNP composition may be an RNA, such as an mRNA molecule encoding a DNA cutting agent. For example, an mRNA for expressing a protein such as an RNA-guided DNA-cutting agent, or a Cas nuclease is included. LNP compositions that include a Cas nuclease mRNA, for example a Class 2 Cas nuclease mRNA that allows for expression in a cell of a Class 2 Cas nuclease such as a Cas9 or Cpf1 (also referred to as Cas12a) protein are provided. Further, the cargo may contain one or more gRNAs or nucleic acids encoding gRNAs. A template nucleic acid, e.g., for repair or recombination, may also be used in the methods described herein. In a sub-embodiment, the cargo comprises an mRNA that encodes a Streptococcus pyogenes Cas9, optionally and an S. pyogenes gRNA. In a further sub-embodiment, the cargo comprises an mRNA that encodes a Neisseria meningitidis Cas9, optionally and an Nme (Neisseria meningitidis) gRNA.

“mRNA” refers to a polynucleotide and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions.

### DNA Cutting Agents

In some embodiments, the compositions or methods comprise a DNA cutting agent, such as a protein or RNA component or a nucleic acid encoding the same. As used herein, the term DNA cutting agent is any component of a genome editing system (or gene editing system) necessary or helpful for producing an edit in the genome of a cell. In some embodiments, the present disclosure provides for methods of delivering a DNA cutting agent of a genome editing system (for example a zinc finger nuclease system, a TALEN system, a meganuclease system or a CRISPR/Cas system) to a cell (or population of cells). DNA cutting agents include, for example, nucleases capable of making single or double strand break in the DNA or RNA of a cell, e.g., in the genome of a cell and nucleic acids encoding the same, such as RNAs. The DNA cutting agents, e.g. nucleases, may optionally modify the genome of a cell without cleaving the nucleic acid, or nickases. A DNA cutting nuclease or nickase agent may be encoded by an mRNA. Such nucleases and nickases include, for example, RNA-guided DNA cutting agents, and CRISPR/Cas components. DNA cutting agents include fusion proteins, including e.g., a nickase fused to an effector domain such as an editor domain. DNA cutting agents include any component necessary or helpful for accomplishing a genome edit that introduces a DNA break, such as, for example, guide RNA, sgRNA, dgRNA, and the like.

Various suitable gene editing systems comprising DNA cutting agents for use with the DNA-PKI compounds described herein, including but not limited to the CRISPR/Cas system; zinc finger nuclease (ZFN) system; and the transcription activator-like effector nuclease (TALEN) system. Generally, the DNA cutting agents involve the use of engineered cleavage systems to induce a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA to guide specific cleavage or nicking of a target DNA sequence. Further, targeted nucleases are being developed based on the Argonaute system (e.g., from T. thermophilus, known as ‘TtAgo’, see Swarts et al (2014) Nature 507(7491): 258-261), which also may have the potential for uses in genome editing and gene therapy.

In certain embodiments, the disclosed compositions comprise one or more DNA modifying agents, such as a DNA cutting agent. A variety of DNA modifying agents may be included in the LNP compositions described herein. For example, DNA modifying agents include nucleases (both sequence-specific and non-specific), topoisomerases, methylases, acetylases, chemicals, pharmaceuticals, and other agents. In some embodiments, proteins that bind to a given DNA sequence or set of sequences may be employed to induce DNA modification such as strand breakage. Proteins can either be modified by many means, such as incorporation of ¹²⁵I, the radioactive decay of which would cause strand breakage, or modifying cross-linking reagents such as 4-azidophenacylbromide which form a cross-link with DNA on exposure to UV-light. Such protein-DNA cross-links can subsequently be converted to a double-stranded DNA break by treatment with piperidine. Yet another approach to DNA modification involves antibodies raised against specific proteins bound at one or more DNA sites, such as transcription factors or architectural chromatin proteins, and used to isolate the DNA from nucleoprotein complexes.

In certain embodiments, the disclosed compositions comprise one or more DNA cutting agents. DNA cutting agents include technologies such as Zinc-Finger Nucleases (ZFN), Transcription Activator-Like Effector Nucleases (TALEN), mito-TALEN, and meganuclease systems. TALEN and ZFN technologies use a strategy of tethering endonuclease catalytic domains to modular DNA binding proteins for inducing targeted DNA double-stranded breaks (DSB) at specific genomic loci. Additional DNA cutting agents include small interfering RNA, micro RNA, anti-microRNA, antagonist, small hairpin RNA, and aptamers (RNA, DNA or peptide based (including affimers)).

In some embodiments, the gene editing system is a TALEN system. Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to a desired DNA sequence, to promote DNA cleavage at specific locations (see, e.g., Boch, 2011, Nature Biotech). The restriction enzymes can be introduced into cells, for use in gene editing or for genome editing in situ, a technique known as genome editing with engineered nucleases. Such methods and compositions for use therein are known in the art. See, e.g., WO2019147805, WO2014040370, WO2018073393, the contents of which are hereby incorporated in their entireties.

In some embodiments, the gene editing system is a zinc-finger system. Zinc-finger nucleases (ZFNs) are artificial restriction enzymes generated by fusing a zinc finger DNA-binding domain to a DNA-cleavage domain. Zinc finger domains can be engineered to target specific desired DNA sequences to enables zinc-finger nucleases to target unique sequences within complex genomes. The non-specific cleavage domain from the type IIs restriction endonuclease FokI is typically used as the cleavage domain in ZFNs. Cleavage is repaired by endogenous DNA repair machinery, allowing ZFN to precisely alter the genomes of higher organisms. Such methods and compositions for use therein are known in the art. See, e.g., WO2011091324, the contents of which are hereby incorporated in their entireties.

In preferred embodiments, the disclosed compositions comprise an mRNA encoding a DNA cutting agent, such as a Cas nuclease. In particular embodiments, the disclosed compositions comprise an mRNA encoding a Class 2 Cas nuclease, such as S. pyogenes Cas9.

As used herein, an “RNA-guided DNA-cutting agent” means a polypeptide or complex of polypeptides having DNA-binding and cutting activity, or a DNA-binding subunit of such a complex, wherein the DNA-binding activity is sequence-specific and depends on the sequence of the RNA that is capable of introducing an ssDNA or dsDNA break. Exemplary RNA-guided DNA-cutting agents include Cas cleavases/nickases and inactivated forms thereof (“dCas DNA-cutting agents”). “Cas nuclease”, as used herein, encompasses Cas cleavases, Cas nickases, and dCas DNA-cutting agents. Cas cleavases/nickases and dCas DNA-cutting agents include a Csm or Cmr complex of a type III CRISPR system, the Cas10, Csml, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA-cutting activity. Class 2 Cas nucleases include Class 2 Cas cleavases/nickases (e.g., H840A, D10A, or N863A variants), which further have RNA-guided DNA cleavases or nickase activity, and Class 2 dCas DNA-cutting agents, in which cleavase/nickase activity is inactivated. Class 2 Cas nucleases that may be used with the LNP compositions described herein include, for example, Cas9, Cpf1, C2cl, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables 2 and 4. See, e.g., Makarova et al., Nat Rev Microbiol, 13(11): 722-36 (2015); Shmakov et al., Molecular Cell, 60:385-397 (2015).

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonfex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In other embodiments, the Cas nuclease is the Cas9 nuclease from Streptococcus thermophilus. In still other embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In other embodiments, the Cas nuclease is the Cpf1 nuclease from Acidaminococcus sp. In still other embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In some embodiments, the Cas nuclease is a Cpf1 nuclease from an Acidaminococcus or Lachnospiraceae.

Wild type Cas9 has two nuclease domains: RuvC and HNH. The RuvC domain cleaves the non-target DNA strand, and the HNH domain cleaves the target strand of DNA. In some embodiments, the Cas9 nuclease comprises more than one RuvC domain and/or more than one HNH domain. In some embodiments, the Cas9 nuclease is a wild type Cas9. In some embodiments, the Cas9 is capable of inducing a double strand break in target DNA. In other embodiments, the Cas nuclease may cleave dsDNA, it may cleave one strand of dsDNA, or it may not have DNA cleavase or nickase activity. In some embodiments, two nickases are combined to create a dsDNA break.

In some embodiments, a Cas nuclease such as a chimeric Cas nuclease is used, where one domain or region of the protein is fused to a portion of a different protein, e.g. a heterologous polypeptide, and optionally comprising a linker polypeptide between the Cas nuclease portion and heterologous functional domain portion of the chimeric Cas9. In some embodiments, a Cas nuclease domain may be fused to, e.g. via a linker, a domain from a different nuclease such as Fok1. In some embodiments, a Cas nuclease may be a modified nuclease, such as a nickase or dCas9.

In other embodiments, the Cas nuclease or Cas nickase may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-cutting agent has single-strand nickase activity, i.e., can cut one DNA strand to produce a single-strand break, also known as a “nick.” In some embodiments, the RNA-guided DNA-cutting agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-cutting agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB-A0Q7Q2 (CPF1_FRATN)).

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA. In some embodiments, a nickase, such as a Cas9 nickase is fused to a heterologous functional domain such as a deaminase polypeptide.

In some embodiments, the RNA-guided DNA-cutting agent lacks cleavase and nickase activity. In some embodiments, the RNA-guided DNA-cutting agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-cutting agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the RNA-guided DNA-cutting agent comprises one or more heterologous functional domains (e.g., is or comprises a fusion polypeptide).

In some embodiments, the heterologous functional domain may facilitate transport of the RNA-guided DNA-cutting agent into the nucleus of a cell. For example, the heterologous functional domain may be a nuclear localization signal (NLS).

In some embodiments, the heterologous functional domain may be capable of modifying the intracellular half-life of the RNA-guided DNA cutting agent. In some embodiments, the half-life of the RNA-guided DNA cutting agent may be increased. In some embodiments, the half-life of the RNA-guided DNA-cutting agent may be reduced. In some embodiments, the heterologous functional domain may be capable of increasing the stability of the RNA-guided DNA-cutting agent. In some embodiments, the heterologous functional domain may be capable of reducing the stability of the RNA-guided DNA-cutting agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-cutting agent may be modified by addition of ubiquitin or a polyubiquitin chain. In some embodiments, the ubiquitin may be a ubiquitinlike protein (UBL). Non-limiting examples of ubiquitin-like proteins include small ubiquitinlike modifier (SUMO), ubiquitin cross-reactive protein (UCRP, also known as interferonstimulated gene-15 (ISG15)), ubiquitin-related modifier-1 (URM1), neuronal-precursor-cellexpressed developmentally downregulated protein-8 (NEDD8, also called Rub1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellowl), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AUl, AU5, E, ECS, E2, FLAG, HA, nus, Softag 1, Softag 3, Strep, SBP, Glu-Glu, HSV, KT3, S, S1, T7, V5, VSV-G, 6×His (SEQ ID NO: 21), 8×His (SEQ ID NO: 22), biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-guided DNA-cutting agent to a specific organelle, cell type, tissue, or organ. In some embodiments, the heterologous functional domain may target the RNA-guided DNA-cutting agent to mitochondria.

In further embodiments, the heterologous functional domain may be an effector domain such as an editor domain. When the RNA-guided DNA-cutting agent is directed to its target sequence, e.g., when a Cas nuclease is directed to a target sequence by a gRNA, the effector domain such as an editor domain may modify or affect the target sequence. In some embodiments, the effector domain such as an editor domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes,” Cell 154:442-51 (2013). As such, the RNA-guided DNA-cutting agent essentially becomes a transcription factor that can be directed to bind a desired target sequence using a guide RNA. In some embodiments, the DNA modification domain is a methylation domain, such as a demethylation or methyltransferase domain. In some embodiments, the effector domain is a DNA modification domain, such as a base-editing domain. In particular embodiments, the DNA modification domain is a nucleic acid editing domain that introduces a specific modification into the DNA, such as a deaminase domain. See, e.g., WO 2015/089406; US 2016/0304846. The nucleic acid editing domains, deaminase domains, and Cas9 variants described in WO 2015/089406 and U.S. 2016/0304846 are hereby incorporated by reference.

In some embodiments, the RNA-guided DNA cutting agent or Cas nickase, such as Cas9 nickase, comprises a APOBEC deaminase. In some embodiments, an APOBEC deaminase is a APOBEC3 deaminase, such as a APOBEC3A (A3A). In some embodiments, the A3A is a human A3A. In some embodiments, the A3A is a wild-type A3A.

In some embodiments, the RNA-guided DNA cutting agent comprises an editor. An exemplary editor is BC22n which comprises a H. sapiens APOBEC3A fused to S. pyogenes-D10A Cas9 nickase by an XTEN linker. In some embodiments, the editor is provided with a uracil glycosylase inhibitor (“UGI”). In some embodiments, the editor is fused to the UGI. In some embodiments, the mRNA encoding the editor and an mRNA encoding the UGI are formulated together in an LNP composition. In other embodiments, the editor and UGI are provided in separate LNP compositions.

The RNA-guided DNA cutting agent may comprise at least one domain that interacts with a guide RNA (“gRNA”). Additionally, it may be directed to a target sequence by a gRNA. In Class 2 Cas nuclease systems, the gRNA interacts with the nuclease as well as the target sequence, such that it directs binding to the target sequence. In some embodiments, the gRNA provides the specificity for the targeted cleavage, and the nuclease may be universal and paired with different gRNAs to cleave different target sequences. Class 2 Cas nuclease may pair with a gRNA scaffold structure of the types, orthologs, and exemplary species listed above.

As used herein, “ribonucleoprotein” (RNP) or “RNP complex” refers to a gRNA together with an RNA-guided DNA-cutting agent, such as a Cas nuclease, e.g., a Cas cleavase, Cas nickase, or dCas DNA-cutting agent such as a dCas9 fusion protein (e.g., Cas9). In some embodiments, the gRNA guides the RNA-guided DNA-cutting agent such as Cas9 to a target sequence, and the gRNA hybridizes with and the agent binds to the target sequence; in cases where the agent is a cleavase or nickase, binding can be followed by cleaving or nicking.

In some embodiments of the present disclosure, the cargo for the LNP composition includes at least one gRNA comprising guide sequences that direct an RNA-guided DNA-cutting agent, which can be a nuclease (e.g., a Cas nuclease such as Cas9), to a target DNA. The gRNA may guide the Cas nuclease or Class 2 Cas nuclease to a target sequence on a target nucleic acid molecule. In some embodiments, a gRNA binds with and provides specificity of cleavage by a Class 2 Cas nuclease. In some embodiments, the gRNA and the Cas nuclease may form a ribonucleoprotein (RNP), e.g., a CRISPR/Cas complex such as a CRISPR/Cas9 complex. In some embodiments, the CRISPR/Cas complex may be a Type-II CRISPR/Cas9 complex. In some embodiments, the CRISPR/Cas complex may be a Type-V CRISPR/Cas complex, such as a Cpf1/gRNA complex. Cas nucleases and cognate gRNAs may be paired. The gRNA scaffold structures that pair with each Class 2 Cas nuclease vary with the specific CRISPR/Cas system. “Guide RNA”, “gRNA”, and simply “guide” are used herein interchangeably to refer to a cognate guide nucleic acid for an RNA-guided DNA-cutting agent. Guide RNAs can include modified RNAs as described herein. A gRNA may be either a crRNA (also known as CRISPR RNA), or the combination of a crRNA and a trRNA (also known as tracrRNA). The crRNA and trRNA may be associated as a single RNA molecule (single guide RNA, sgRNA) or in two or more separate RNA molecules (dual guide RNA, dgRNA), optionally covalently linked. “Guide RNA” or “gRNA” refers to each type. The trRNA may be a naturally-occurring sequence, or a trRNA sequence with modifications or variations compared to naturally-occurring sequences.

In some embodiments, an mRNA encoding a RNA-guided DNA cutting agent is formulated in a first LNP composition and a gRNA nucleic acid is formulated in a second LNP composition. In some embodiments, the first and second lipid nucleic acid assembly compositions are administered simultaneously. In other embodiments, the first and second lipid nucleic acid assembly compositions are administered sequentially. In some embodiments, the first and second lipid nucleic acid assembly compositions are combined prior to the preincubation step. In other embodiments, the first and second lipid nucleic acid assembly compositions are preincubated separately.

In certain embodiments, the compositions and methods described herein involve a modified RNA. In some embodiments, the compositions and methods described herein involve guide RNA nucleic acid. In certain embodiments, the compositions and methods described herein involve a gRNA, such as a dgRNA or a modified gRNA. In some compositions comprising an mRNA encoding an RNA-guided DNA-cutting agent, the compositions further comprise a gRNA nucleic acid, such as a gRNA. In some embodiments, the compositions and methods described herein involve an RNA-guided DNA-cutting agent and a gRNA. In some embodiments, the compositions and methods described herein involve a Cas nuclease mRNA and a gRNA, such as a Class 2 Cas nuclease mRNA and a gRNA.

In some embodiments, the cargo may comprise a DNA molecule. In some embodiments, the nucleic acid may comprise a nucleotide sequence encoding a crRNA. In some embodiments, the nucleotide sequence encoding the crRNA comprises a targeting sequence flanked by all or a portion of a repeat sequence from a naturally-occurring CRISPR/Cas system. In some embodiments, the nucleic acid may comprise a nucleotide sequence encoding a tracr RNA. In certain embodiments, the crRNA and the tracr RNA may be encoded by two separate nucleic acids. In other embodiments, the crRNA and the tracr RNA may be encoded by a single nucleic acid. In some embodiments, the crRNA and the tracr RNA may be encoded by opposite strands of a single nucleic acid. In other embodiments, the crRNA and the tracr RNA may be encoded by the same strand of a single nucleic acid. In some embodiments, the gRNA nucleic acid encodes an sgRNA. In some embodiments, the gRNA nucleic acid encodes a Cas9 nuclease sgRNA. In come embodiments, the gRNA nucleic acid encodes a Cpf1 nuclease sgRNA.

The nucleotide sequence encoding the guide RNA may be operably linked to at least one transcriptional or regulatory control sequence, such as a promoter, a 3 UTR, or a 5 UTR. In one example, the promoter may be a tRNA promoter, e.g., tRNALys3, or a tRNA chimera. See Mefferd et al., RNA. 2015 21:1683-9; Scherer et al., Nucleic Acids Res. 2007 35: 2620-2628. In some embodiments, the promoter may be recognized by RNA polymerase III (Pol III). Non-limiting examples of Pol III promoters also include U6 and H1 promoters. In some embodiments, the nucleotide sequence encoding the guide RNA may be operably linked to a mouse or human U6 promoter. In some embodiments, the gRNA nucleic acid is a modified nucleic acid. In some embodiments, the gRNA nucleic acid includes a modified nucleoside or nucleotide. In some embodiments, the gRNA nucleic acid includes a 5 end modification, for example a modified nucleoside or nucleotide to stabilize and prevent integration of the nucleic acid. In other embodiments, the gRNA nucleic acid comprises a double-stranded DNA having a 5□end modification on each strand. In some embodiments, the gRNA nucleic acid includes an inverted dideoxy-T or an inverted abasic nucleoside or nucleotide as the 5□end modification. In some embodiments, the gRNA nucleic acid includes a label such as biotin, desthiobiotin-TEG, digoxigenin, and fluorescent markers, including, for example, FAM, ROX, TAMRA, and AlexaFluor.

As used herein, a “guide sequence” refers to a sequence within a gRNA that is complementary to a target sequence and functions to direct a gRNA to a target sequence for binding or modification (e.g., cleavage) by an RNA-guided DNA-cutting agent. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. In some embodiments, the target sequence is in a gene or on a chromosome, for example, and is complementary to the guide sequence. In some embodiments, the degree of complementarity or identity between a guide sequence and its corresponding target sequence may be about or at least 75%, 80%, 85%, 90%, 95%, 96%, 97%, 98%, 99%, or 100%. In some embodiments, the guide sequence and the target region may be 100% complementary or identical over a region of at least 15, 16, 17, 18, 19, or 20 contiguous nucleotides. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

In certain embodiments, multiple LNP compositions may be used collaboratively and/or for separate purposes. In some embodiments, a cell may be contacted with first and second LNP compositions described herein. In some embodiments, the first and second LNP compositions each independently comprise one or more of an mRNA, a gRNA, and a gRNA nucleic acid, for example. In some embodiments, the first and second LNP compositions are administered simultaneously. In some embodiments, the first and second LNP compositions are administered sequentially.

In some embodiments, a method of producing multiple genome edits in a cell is provided (sometimes referred to herein and elsewhere as “multiplexing” or “multiplex gene editing” or “multiplex genome editing”). The ability to engineer multiple attributes into a single cell depends on the ability to perform edits in multiple targeted genes efficiently, including knockouts and in locus insertions, while retaining viability and the desired cell phenotype. In some embodiments, the method comprises culturing a cell in vitro, contacting the cell with two or more lipid nucleic acid assembly compositions, wherein each lipid nucleic acid assembly composition comprises a nucleic acid genome editing tool capable of editing a target site, and expanding the cell in vitro. The method results in a cell having more than one genome edit, wherein the genome edits differ. In certain embodiments, the first LNP composition comprises a first gRNA and the second LNP composition comprises a second gRNA, wherein the first and second gRNAs comprise different guide sequences that are complementary to different targets. In such embodiments, the LNP compositions may allow for multiplex gene editing.

Target sequences for RNA-guided DNA-cutting proteins such as Cas proteins include both the positive and negative strands of genomic DNA (i.e., the sequence given and the sequence's reverse compliment), as a nucleic acid substrate for a Cas protein is a double stranded nucleic acid. Accordingly, where a guide sequence is said to be “complementary to a target sequence”, it is to be understood that the guide sequence may direct a gRNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

The length of the targeting sequence may depend on the CRISPR/Cas system and components used. For example, different Class 2 Cas nucleases from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the targeting sequence length is 0, 1, 2, 3, 4, or 5 nucleotides longer or shorter than the guide sequence of a naturally-occurring CRISPR/Cas system. In certain embodiments, the Cas nuclease and gRNA scaffold will be derived from the same CRISPR/Cas system. In some embodiments, the targeting sequence may comprise or consist of 18-24 nucleotides. In some embodiments, the targeting sequence may comprise or consist of 19-21 nucleotides. In some embodiments, the targeting sequence may comprise or consist of 20 nucleotides.

In some embodiments, the sgRNA is a “Cas9 sgRNA” capable of mediating RNA-guided DNA cleavage by a Cas9 protein. In some embodiments, the sgRNA is a “Cpf1 sgRNA” capable of mediating RNA-guided DNA cleavage by a Cpf1 protein. In certain embodiments, the gRNA comprises a crRNA and tracr RNA sufficient for forming an active complex with a Cas9 protein and mediating RNA-guided DNA cleavage. In certain embodiments, the gRNA comprises a crRNA sufficient for forming an active complex with a Cpf1 protein and mediating RNA-guided DNA cleavage. See Zetsche 2015.

Certain embodiments also provide nucleic acids, e.g., expression cassettes, encoding the gRNA described herein. A “guide RNA nucleic acid” is used herein to refer to a gRNA (e.g. an sgRNA or a dgRNA) and a gRNA expression cassette, which is a nucleic acid that encodes one or more gRNAs.

### Modified RNAs

In certain embodiments, the lipid compositions, such as LNP compositions comprise modified nucleic acids, including modified RNAs.

Modified nucleosides or nucleotides can be present in an RNA, for example a gRNA or mRNA. A gRNA or mRNA comprising one or more modified nucleosides or nucleotides, for example, is called a “modified” RNA to describe the presence of one or more non-naturally and/or naturally occurring components or configurations that are used instead of or in addition to the canonical A, G, C, and U residues. In some embodiments, a modified RNA is synthesized with a non-canonical nucleoside or nucleotide, here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the polynucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification). Certain embodiments comprise a 5′ end modification to an mRNA, gRNA, or nucleic acid. Certain embodiments comprise a modification to an mRNA, gRNA, or nucleic acid. Certain embodiments comprise a 3′ end modification to an mRNA, gRNA, or nucleic acid. A modified RNA can contain 5′ end and 3′ end modifications. A modified RNA can contain one or more modified residues at non-terminal locations. In certain embodiments, a gRNA includes at least one modified residue. In certain embodiments, an mRNA includes at least one modified residue. In certain embodiments, the modified gRNA comprises a modification at one or more of the first five nucleotides at a 5′ end. In certain embodiments, the modified gRNA comprises a modification at one or more of the first five nucleotides at a 5′ end. The LNP composition of claim 52 or 53, wherein the modified gRNA comprises a modification at one or more of the last five nucleotides at a 3′ end.

Unmodified nucleic acids can be prone to degradation by, e.g., intracellular nucleases or those found in serum. For example, nucleases can hydrolyze nucleic acid phosphodiester bonds. Accordingly, in one aspect the RNAs (e.g. mRNAs, gRNAs) described herein can contain one or more modified nucleosides or nucleotides, e.g., to introduce stability toward intracellular or serum-based nucleases. In some embodiments, the modified RNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

Accordingly, in some embodiments, an RNA or nucleic acid comprises at least one modification which confers increased or enhanced stability to the nucleic acid, including, for example, improved resistance to nuclease digestion in vivo. As used herein, the terms “modification” and “modified” as such terms relate to the nucleic acids provided herein, include at least one alteration which preferably enhances stability and renders the RNA or nucleic acid more stable (e.g., resistant to nuclease digestion) than the wild-type or naturally occurring version of the RNA or nucleic acid. As used herein, the terms “stable” and “stability” and such terms relate to the nucleic acids described herein, and particularly with respect to the RNA, refer to increased or enhanced resistance to degradation by, for example nucleases (i.e., endonucleases or exonucleases) which are normally capable of degrading such RNA. Increased stability can include, for example, less sensitivity to hydrolysis or other destruction by endogenous enzymes (e.g., endonucleases or exonucleases) or conditions within the target cell or tissue, thereby increasing or enhancing the residence of such RNA or nucleic acid in the target cell, tissue, subject and/or cytoplasm. The stabilized RNA or nucleic acid molecules provided herein demonstrate longer half-lives relative to their naturally occurring, unmodified counterparts (e.g. the wild-type version of the molecule). Also contemplated by the terms “modification” and “modified” as such terms related to the mRNA of the LNP compositions disclosed herein are alterations which improve or enhance translation of mRNA nucleic acids, including for example, the inclusion of sequences which function in the initiation of protein translation (e.g., the Kozak consensus sequence). (Kozak, M., Nucleic Acids Res 15 (20): 8125-48 (1987)).

In some embodiments, the RNA or nucleic acid has undergone a chemical or biological modification to render it more stable. Exemplary modifications to an RNA or nucleic acid include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring RNA or nucleic acids, for example, covalent modifications such as the introduction of modified nucleotides, (e.g., nucleotide analogs, or the inclusion of pendant groups which are not naturally found in such RNA, such as a deoxynucleoside, or nucleic acid molecules).

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens. The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

In some embodiments, a composition or formulation disclosed herein comprises an mRNA comprising an open reading frame (ORF), such as, e.g. an ORF encoding an RNA-guided DNA binding agent, such as a Cas nuclease, or Class 2 Cas nuclease as described herein. In some embodiments, an mRNA comprising an ORF encoding an RNA-guided DNA binding agent, such as a Cas nuclease or Class 2 Cas nuclease, is provided, used, or administered. In some embodiments, the ORF is codon optimized. In some embodiments, the ORF encoding an RNA-guided DNA binding agent is a “modified RNA-guided DNA binding agent ORF” or simply a “modified ORF,” which is used as shorthand to indicate that the ORF is modified in one or more of the following ways: (1) the modified ORF has a uridine content ranging from its minimum uridine content to 150% of the minimum uridine content; (2) the modified ORF has a uridine dinucleotide content ranging from its minimum uridine dinucleotide content to 150% of the minimum uridine dinucleotide content; (3) the modified ORF consists of a set of codons of which at least 75% of the codons are minimal uridine codon(s) for a given amino acid, e.g. the codon(s) with the fewest uridines (usually 0 or 1 except for a codon for phenylalanine, where the minimal uridine codon has 2 uridines); or (4) the modified ORF comprises at least one modified uridine. In some embodiments, the modified ORF is modified in at least two, three, or four of the foregoing ways. In some embodiments, the modified ORF comprises at least one modified uridine and is modified in at least one, two, three, or all of (1)-(3) above.

“Modified uridine” is used herein to refer to a nucleoside other than thymidine with the same hydrogen bond acceptors as uridine and one or more structural differences from uridine. In some embodiments, a modified uridine is a substituted uridine, i.e., a uridine in which one or more non-proton substituents (e.g., alkoxy, such as methoxy) takes the place of a proton. In some embodiments, a modified uridine is pseudouridine. In some embodiments, a modified uridine is a substituted pseudouridine, i.e., a pseudouridine in which one or more non-proton substituents (e.g., alkyl, such as methyl) takes the place of a proton. In some embodiments, a modified uridine is any of a substituted uridine, pseudouridine, or a substituted pseudouridine.

Uridine position” as used herein refers to a position in a polynucleotide occupied by a uridine or a modified uridine. Thus, for example, a polynucleotide in which “100% of the uridine positions are modified uridines” contains a modified uridine at every position that would be a uridine in a conventional RNA (where all bases are standard A, U, C, or G bases) of the same sequence. Unless otherwise indicated, a U in a polynucleotide sequence of a sequence table or sequence listing in, or accompanying, this disclosure can be a uridine or a modified uridine.

### Minimal Uridine Codons:

| | Amino Acid | Minimal uridine codon |
|---|---|---|
| A | Alanine | GCA or GCC or GCG |
| G | Glycine | GGA or GGC or GGG |
| V | Valine | GUC or GUA or GUG |
| D | Aspartic acid | GAC |
| E | Glutamic acid | GAA or GAG |
| I | Isoleucine | AUC or AUA or AUG |
| T | Threonine | ACA or ACC or ACG |
| N | Asparagine | AAC |
| K | Lysine | AAG or AAA |
| S | Serine | AGC |
| R | Arginine | AGA or AGG |
| L | Leucine | CUG or CUA or CUC |
| P | Proline | CCG or CCA or CCC |
| H | Histidine | CAC or CAA or CAG |
| Q | Glutamine | CAG or CAA |
| F | Phenylalanine | UUC |
| Y | Tyrosine | UAC |
| C | Cysteine | UGC |
| W | Tryptophan | UGG |
| M | Methionine | AUG |

In any of the foregoing embodiments, the modified ORF may consist of a set of codons of which at least 75%, 80%, 85%, 90%, 95%, 98%, 99%, or 100% of the codons are codons listed in the Table above of minimal uridine codons.

In any of the foregoing embodiments, the modified ORF may have a uridine content ranging from its minimum uridine content to 150%, 145%, 140%, 135%, 130%, 125%, 120%, 115%, 110%, 105%, 104%, 103%, 102%, or 101% of the minimum uridine content.

In any of the foregoing embodiments, the modified ORF may have a uridine dinucleotide content ranging from its minimum uridine dinucleotide content to 150%, 145%, 140%, 135%, 130%, 125%, 120%, 115%, 110%, 105%, 104%, 103%, 102%, or 101% of the minimum uridine dinucleotide content.

In any of the foregoing embodiments, the modified ORF may comprise a modified uridine at least at one, a plurality of, or all uridine positions. In some embodiments, the modified uridine is a uridine modified at the 5 position, e.g., with a halogen, methyl, or ethyl. In some embodiments, the modified uridine is a pseudouridine modified at the 1 position, e.g., with a halogen, methyl, or ethyl. The modified uridine can be, for example, pseudouridine, N1-methyl-pseudouridine, 5-methoxyuridine, 5-iodouridine, or a combination thereof. In some embodiments, the modified uridine is 5-methoxyuridine. In some embodiments, the modified uridine is 5-iodouridine. In some embodiments, the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, at least 10%, 15%, 20%, 25%, 30%, 35%, 40%, 45%, 50%, 55%, 60%, 65%, 70%, 75%, 80%, 85%, 90%, 95%, 98%, 99%, or 100% of the uridine positions in an mRNA according to the disclosure are modified uridines. In some embodiments, 10%-25%, 15-25%, 25-35%, 35-45%, 45-55%, 55-65%, 65-75%, 75-85%, 85-95%, or 90-100% of the uridine positions in an mRNA according to the disclosure are modified uridines, e.g., 5-methoxyuridine, 5-iodouridine, N1-methyl pseudouridine, pseudouridine, or a combination thereof. In some embodiments, 10%-25%, 15-25%, 25-35%, 35-45%, 45-55%, 55-65%, 65-75%, 75-85%, 85-95%, or 90-100% of the uridine positions in an mRNA according to the disclosure are 5-methoxyuridine. In some embodiments, 10%-25%, 15-25%, 25-35%, 35-45%, 45-55%, 55-65%, 65-75%, 75-85%, 85-95%, or 90-100% of the uridine positions in an mRNA according to the disclosure are pseudouridine. In some embodiments, 10%-25%, 15-25%, 25-35%, 35-45%, 45-55%, 55-65%, 65-75%, 75-85%, 85-95%, or 90-100% of the uridine positions in an mRNA according to the disclosure are N1-methyl pseudouridine. In some embodiments, 10%-25%, 15-25%, 25-35%, 35-45%, 45-55%, 55-65%, 65-75%, 75-85%, 85-95%, or 90-100% of the uridine positions in an mRNA according to the disclosure are 5-iodouridine. In some embodiments, 10%-25%, 15-25%, 25-35%, 35-45%, 45-55%, 55-65%, 65-75%, 75-85%, 85-95%, or 90-100% of the uridine positions in an mRNA according to the disclosure are 5-methoxyuridine, and the remainder are N1-methyl pseudouridine. In some embodiments, 10%-25%, 15-25%, 25-35%, 35-45%, 45-55%, 55-65%, 65-75%, 75-85%, 85-95%, or 90-100% of the uridine positions in an mRNA according to the disclosure are 5-iodouridine, and the remainder are N1-methyl pseudouridine.

In any of the foregoing embodiments, the modified ORF may comprise a reduced uridine dinucleotide content, such as the lowest possible uridine dinucleotide (UU) content, e.g. an ORF that (a) uses a minimal uridine codon (as discussed above) at every position and (b) encodes the same amino acid sequence as the given ORF. The uridine dinucleotide (UU) content can be expressed in absolute terms as the enumeration of UU dinucleotides in an ORF or on a rate basis as the percentage of positions occupied by the uridines of uridine dinucleotides (for example, AUUAU would have a uridine dinucleotide content of 40% because 2 of 5 positions are occupied by the uridines of a uridine dinucleotide). Modified uridine residues are considered equivalent to uridines for the purpose of evaluating minimum uridine dinucleotide content.

In some embodiments, the mRNA comprises at least one UTR from an expressed mammalian mRNA, such as a constitutively expressed mRNA. An mRNA is considered constitutively expressed in a mammal if it is continually transcribed in at least one tissue of a healthy adult mammal. In some embodiments, the mRNA comprises a 5′ UTR, 3′ UTR, or 5′ and 3′ UTRs from an expressed mammalian RNA, such as a constitutively expressed mammalian mRNA. Actin mRNA is an example of a constitutively expressed mRNA.

In some embodiments, the mRNA comprises at least one UTR from Hydroxysteroid 17-Beta Dehydrogenase 4 (HSD17B4 or HSD), e.g., a 5′ UTR from HSD. In some embodiments, the mRNA comprises at least one UTR from a globin mRNA, for example, human alpha globin (HBA) mRNA, human beta globin (HBB) mRNA, or Xenopus laevis beta globin (XBG) mRNA. In some embodiments, the mRNA comprises a 5′ UTR, 3′ UTR, or 5′ and 3′ UTRs from a globin mRNA, such as HBA, HBB, or XBG. In some embodiments, the mRNA comprises a 5′ UTR from bovine growth hormone, cytomegalovirus (CMV), mouse Hba-a1, HSD, an albumin gene, HBA, HBB, or XBG. In some embodiments, the mRNA comprises a 3′ UTR from bovine growth hormone, cytomegalovirus, mouse Hba-a1, HSD, an albumin gene, HBA, HBB, or XBG. In some embodiments, the mRNA comprises 5′ and 3′ UTRs from bovine growth hormone, cytomegalovirus, mouse Hba-a1, HSD, an albumin gene, HBA, HBB, XBG, heat shock protein 90 (Hsp90), glyceraldehyde 3-phosphate dehydrogenase (GAPDH), beta-actin, alpha-tubulin, tumor protein (p53), or epidermal growth factor receptor (EGFR).

In some embodiments, the mRNA comprises 5′ and 3′ UTRs that are from the same source, e.g., a constitutively expressed mRNA such as actin, albumin, or a globin such as HBA, HBB, or XBG.

In some embodiments, the mRNA does not comprise a 5′ UTR, e.g., there are no additional nucleotides between the 5′ cap and the start codon. In some embodiments, the mRNA comprises a Kozak sequence (described below) between the 5′ cap and the start codon, but does not have any additional 5′ UTR. In some embodiments, the mRNA does not comprise a 3′ UTR, e.g., there are no additional nucleotides between the stop codon and the poly-A tail.

In some embodiments, the mRNA comprises a Kozak sequence. The Kozak sequence can affect translation initiation and the overall yield of a polypeptide translated from an mRNA. A Kozak sequence includes a methionine codon that can function as the start codon. A minimal Kozak sequence is NNNRUGN wherein at least one of the following is true: the first N is A or G and the second N is G. In the context of a nucleotide sequence, R means a purine (A or G). In some embodiments, the Kozak sequence is RNNRUGN, NNNRUGG, RNNRUGG, RNNAUGN, NNNAUGG, or RNNAUGG. In some embodiments, the Kozak sequence is rccRUGg with zero mismatches or with up to one or two mismatches to positions in lowercase.

In some embodiments, the Kozak sequence is rccAUGg with zero mismatches or with up to one or two mismatches to positions in lowercase. In some embodiments, the Kozak sequence is gccRccAUGG (SEQ ID NO: 24) with zero mismatches or with up to one, two, or three mismatches to positions in lowercase. In some embodiments, the Kozak sequence is gccAccAUG with zero mismatches or with up to one, two, three, or four mismatches to positions in lowercase. In some embodiments, the Kozak sequence is GCCACCAUG. In some embodiments, the Kozak sequence is gccgccRccAUGG (SEQ ID NO: 23) with zero mismatches or with up to one, two, three, or four mismatches to positions in lowercase.

In some embodiments, an mRNA disclosed herein comprises a 5′ cap, such as a Cap0, Cap1, or Cap2. A 5′ cap is generally a 7-methylguanine ribonucleotide (which may be further modified, as discussed below e.g. with respect to ARCA) linked through a 5′-triphosphate to the 5′ position of the first nucleotide of the 5′-to-3′ chain of the mRNA, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl(3 CO-methyl)GpppG and 7-methyl(3′deoxy)GpppG,” RNA 7: 1486-1495. The ARCA structure is shown below.

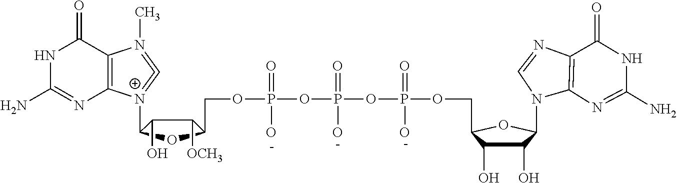

CleanCap™ AG (m7G(5)ppp(5)(2OMeA)pG; TriLink Biotechnologies Cat. No. N-7113) or CleanCap™ GG (m7G(5)ppp(5)(2OMeG)pG; TriLink Biotechnologies Cat. No. N-7133) can be used to provide a CapI structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below.

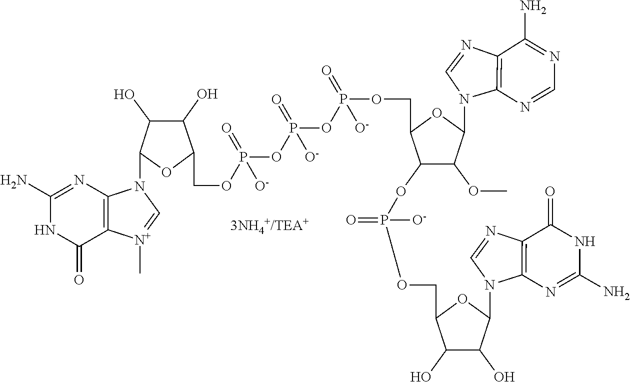 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479.

In some embodiments, the mRNA further comprises a poly-adenylated (poly-A) tail. In some embodiments, the poly-A tail comprises at least 20, 30, 40, 50, 60, 70, 80, 90, or 100 adenines, optionally up to 300 adenines. In some embodiments, the poly-A tail comprises 95, 96, 97, 98, 99, or 100 adenine nucleotides (SEQ ID NO: 25). In some instances, the poly-A tail is “interrupted” with one or more non-adenine nucleotide “anchors” at one or more locations within the poly-A tail. The poly-A tails may comprise at least 8 consecutive adenine nucleotides, but also comprise one or more non-adenine nucleotide. As used herein, “non-adenine nucleotides” refer to any natural or non-natural nucleotides that do not comprise adenine. Guanine, thymine, and cytosine nucleotides are exemplary non-adenine nucleotides. Thus, the poly-A tails on the mRNA described herein may comprise consecutive adenine nucleotides located 3′ to nucleotides encoding an RNA-guided DNA-binding agent or a sequence of interest. In some instances, the poly-A tails on mRNA comprise non-consecutive adenine nucleotides located 3′ to nucleotides encoding an RNA-guided DNA-binding agent or a sequence of interest, wherein non-adenine nucleotides interrupt the adenine nucleotides at regular or irregularly spaced intervals.

As used herein, “non-adenine nucleotides” refer to any natural or non-natural nucleotides that do not comprise adenine. Guanine, thymine, and cytosine nucleotides are exemplary non-adenine nucleotides. Thus, the poly-A tails on the mRNA described herein may comprise consecutive adenine nucleotides located 3′ to nucleotides encoding an RNA-guided DNA-binding agent or a sequence of interest. In some instances, the poly-A tails on mRNA comprise non-consecutive adenine nucleotides located 3′ to nucleotides encoding an RNA-guided DNA-binding agent or a sequence of interest, wherein non-adenine nucleotides interrupt the adenine nucleotides at regular or irregularly spaced intervals.

In some embodiments, the mRNA is purified. In some embodiments, the mRNA is purified using a precipitation method (e.g., LiCl precipitation, alcohol precipitation, or an equivalent method, e.g., as described herein). In some embodiments, the mRNA is purified using a chromatography-based method, such as an HPLC-based method or an equivalent method (e.g., as described herein). In some embodiments, the mRNA is purified using both a precipitation method (e.g., LiCl precipitation) and an HPLC-based method.

In some embodiments, at least one gRNA is provided in combination with an mRNA disclosed herein. In some embodiments, a gRNA is provided as a separate molecule from the mRNA. In some embodiments, a gRNA is provided as a part, such as a part of a UTR, of an mRNA disclosed herein.

mRNAs

In some embodiments, a composition or formulation disclosed herein comprises an mRNA comprising an open reading frame (ORF) encoding DNA cutting agent, such as an RNA-guided DNA-cutting agent, such as a Cas nuclease, or Class 2 Cas nuclease as described herein. In some embodiments, an mRNA comprising an ORF encoding an RNA-guided DNA-cutting agent, such as a Cas nuclease or Class 2 Cas nuclease, is provided, used, or administered. An mRNA may comprise one or more of a 5′ cap, a 5′ untranslated region (UTR), a 3′ UTRs, and a polyadenine tail. The mRNA may comprise a modified open reading frame, for example to encode a nuclear localization sequence or to use alternate codons to encode the protein.

The mRNA in the disclosed LNP compositions may encode, for example, a secreted hormone, enzyme, receptor, polypeptide, peptide or other protein of interest that is normally secreted. In some embodiments, the mRNA may optionally have chemical or biological modifications which, for example, improve the stability and/or half-life of such mRNA or which improve or otherwise facilitate protein production.

In addition, suitable modifications include alterations in one or more nucleotides of a codon such that the codon encodes the same amino acid but is more stable than the codon found in the wild-type version of the mRNA. For example, an inverse relationship between the stability of RNA and a higher number cytidines (C's) and/or uridines (U's) residues has been demonstrated, and RNA devoid of C and U residues have been found to be stable to most RNases (Heidenreich, et al. J Biol Chem 269, 2131-8 (1994)). In some embodiments, the number of C and/or U residues in an mRNA sequence is reduced. In another embodiment, the number of C and/or U residues is reduced by substitution of one codon encoding a particular amino acid for another codon encoding the same or a related amino acid. Contemplated modifications to the mRNA nucleic acids also include the incorporation of pseudouridines. The incorporation of pseudouridines into the mRNA nucleic acids may enhance stability and translational capacity, as well as diminishing immunogenicity in vivo. See, e.g., Karikó, K., et al., Molecular Therapy 16 (11): 1833-1840 (2008). Substitutions and modifications to the mRNA may be performed by methods readily known to one or ordinary skill in the art.

The constraints on reducing the number of C and U residues in a sequence will likely be greater within the coding region of an mRNA, compared to an untranslated region, (i.e., it will likely not be possible to eliminate all of the C and U residues present in the message while still retaining the ability of the message to encode the desired amino acid sequence). The degeneracy of the genetic code, however presents an opportunity to allow the number of C and/or U residues that are present in the sequence to be reduced, while maintaining the same coding capacity (i.e., depending on which amino acid is encoded by a codon, several different possibilities for modification of RNA sequences may be possible).

The term modification also includes, for example, the incorporation of non-nucleotide linkages or modified nucleotides into the mRNA sequences (e.g., modifications to one or both the 3′ and 5′ ends of an mRNA molecule encoding a functional secreted protein or enzyme). Such modifications include the addition of bases to an mRNA sequence (e.g., the inclusion of a poly A tail or a longer poly A tail), the alteration of the 3′ UTR or the 5′ UTR, complexing the mRNA with an agent (e.g., a protein or a complementary nucleic acid molecule), and inclusion of elements which change the structure of an mRNA molecule (e.g., which form secondary structures).

The poly A tail is thought to stabilize natural messengers. Therefore, a long poly A tail may be added to an mRNA molecule thus rendering the mRNA more stable. Poly A tails can be added using a variety of art-recognized techniques. For example, long poly A tails can be added to synthetic or in vitro transcribed mRNA using poly A polymerase (Yokoe, et al. Nature Biotechnology. 1996; 14: 1252-1256). A transcription vector can also encode long poly A tails. In addition, poly A tails can be added by transcription directly from PCR products. In some embodiments, the length of the poly A tail is at least about 90, 200, 300, 400 at least 500 nucleotides. In certain embodiments, the length of the poly A tail is adjusted to control the stability of a modified mRNA molecule and, thus, the transcription of protein. For example, since the length of the poly A tail can influence the half-life of an mRNA molecule, the length of the poly A tail can be adjusted to modify the level of resistance of the mRNA to nucleases and thereby control the time course of protein expression in a cell. In some embodiments, the stabilized mRNA molecules are sufficiently resistant to in vivo degradation (e.g., by nucleases), such that they may be delivered to the target cell without a transfer vehicle.

In certain embodiments, an mRNA can be modified by the incorporation 3′ and/or 5′ untranslated (UTR) sequences which are not naturally found in the wild-type mRNA. In some embodiments, 3′ and/or 5′ flanking sequence which naturally flanks an mRNA and encodes a second, unrelated protein can be incorporated into the nucleotide sequence of an mRNA molecule encoding a therapeutic or functional protein in order to modify it. For example, 3′ or 5′ sequences from mRNA molecules which are stable (e.g., globin, actin, GAPDH, tubulin, histone, or citric acid cycle enzymes) can be incorporated into the 3′ and/or 5′ region of a sense mRNA nucleic acid molecule to increase the stability of the sense mRNA molecule. See, e.g., US2003/0083272.

More detailed descriptions of the mRNA modifications can be found in US2017/0210698A1, at pages 57-68, the contents of which are incorporated herein.

### Template Nucleic Acid

The methods disclosed herein may include using a template nucleic acid. The template may be used to alter or insert a nucleic acid sequence at or near a target site for an RNA-guided DNA-cutting protein such as a Cas nuclease, e.g., a Class 2 Cas nuclease. In some embodiments, the methods comprise introducing a template to the cell. In some embodiments, a single template may be provided. In other embodiments, two or more templates may be provided such that editing may occur at two or more target sites. For example, different templates may be provided to edit a single gene in a cell, or two different genes in a cell.

In some embodiments, the template may be used in homologous recombination. In some embodiments, the homologous recombination may result in the integration of the template sequence or a portion of the template sequence into the target nucleic acid molecule. In other embodiments, the template may be used in homology-directed repair, which involves DNA strand invasion at the site of the cleavage in the nucleic acid. In some embodiments, the homology-directed repair may result in including the template sequence in the edited target nucleic acid molecule. In yet other embodiments, the template may be used in gene editing mediated by non-homologous end joining. In some embodiments, the template sequence has no similarity to the nucleic acid sequence near the cleavage site. In some embodiments, the template or a portion of the template sequence is incorporated. In some embodiments, the template includes flanking inverted terminal repeat (ITR) sequences.

In some embodiments, the template may comprise a first homology arm and a second homology arm (also called a first and second nucleotide sequence) that are complementary to sequences located upstream and downstream of the cleavage site, respectively. Where a template contains two homology arms, each arm can be the same length or different lengths, and the sequence between the homology arms can be substantially similar or identical to the target sequence between the homology arms, or it can be entirely unrelated. In some embodiments, the degree of complementarity or percent identity between the first nucleotide sequence on the template and the sequence upstream of the cleavage site, and between the second nucleotide sequence on the template and the sequence downstream of the cleavage site, may permit homologous recombination, such as, e.g., high-fidelity homologous recombination, between the template and the target nucleic acid molecule. In some embodiments, the degree of complementarity may be about 50%, 55%, 60%, 65%, 70%, 75%, 80%, 85%, 90%, 95%, 97%, 98%, 99%, or 100%. In some embodiments, the degree of complementarity may be about 95%, 97%, 98%, 99%, or 100%. In some embodiments, the degree of complementarity may be at least 98%, 99%, or 100%. In some embodiments, the degree of complementarity may be 100%. In some embodiments, the percent identity may be about 50%, 55%, 60%, 65%, 70%, 75%, 80%, 85%, 90%, 95%, 97%, 98%, 99%, or 100%. In some embodiments, the percent identity may be about 95%, 97%, 98%, 99%, or 100%. In some embodiments, the percent identity may be at least 98%, 99%, or 100%. In some embodiments, the percent identity may be 100%.

In some embodiments, the template sequence may correspond to, comprise, or consist of an endogenous sequence of a target cell. It may also or alternatively correspond to, comprise, or consist of an exogenous sequence of a target cell. As used herein, the term “endogenous sequence” refers to a sequence that is native to the cell. The term “exogenous sequence” refers to a sequence that is not native to a cell, or a sequence whose native location in the genome of the cell is in a different location. In some embodiments, the endogenous sequence may be a genomic sequence of the cell. In some embodiments, the endogenous sequence may be a chromosomal or extrachromosomal sequence. In some embodiments, the endogenous sequence may be a plasmid sequence of the cell. In some embodiments, the template sequence may be substantially identical to a portion of the endogenous sequence in a cell at or near the cleavage site, but comprise at least one nucleotide change. In some embodiments, editing the cleaved target nucleic acid molecule with the template may result in a mutation comprising an insertion, deletion, or substitution of one or more nucleotides of the target nucleic acid molecule. In some embodiments, the mutation may result in one or more amino acid changes in a protein expressed from a gene comprising the target sequence.

In some embodiments, the mutation may result in one or more nucleotide changes in an RNA expressed from the target insertion site. In some embodiments, the mutation may alter the expression level of a target gene. In some embodiments, the mutation may result in increased or decreased expression of the target gene. In some embodiments, the mutation may result in gene knock-down. In some embodiments, the mutation may result in gene knock-out. In some embodiments, the mutation may result in restored gene function. In some embodiments, editing of the cleaved target nucleic acid molecule with the template may result in a change in an exon sequence, an intron sequence, a regulatory sequence, a transcriptional control sequence, a translational control sequence, a splicing site, or a non-coding sequence of the target nucleic acid molecule, such as DNA.

In other embodiments, the template sequence may comprise an exogenous sequence. In some embodiments, the exogenous sequence may comprise a coding sequence. In some embodiments, the exogenous sequence may comprise a protein or RNA coding sequence (e.g., an ORF) operably linked to an exogenous promoter sequence such that, upon integration of the exogenous sequence into the target nucleic acid molecule, the cell is capable of expressing the protein or RNA encoded by the integrated sequence. In other embodiments, upon integration of the exogenous sequence into the target nucleic acid molecule, the expression of the integrated sequence may be regulated by an endogenous promoter sequence. In some embodiments, the exogenous sequence may provide a cDNA sequence encoding a protein or a portion of the protein. In yet other embodiments, the exogenous sequence may comprise or consist of an exon sequence, an intron sequence, a regulatory sequence, a transcriptional control sequence, a translational control sequence, a splicing site, or a non-coding sequence. In some embodiments, the integration of the exogenous sequence may result in restored gene function. In some embodiments, the integration of the exogenous sequence may result in a gene knock-in. In some embodiments, the integration of the exogenous sequence may result in a gene knock-out.

The template may be of any suitable length. In some embodiments, the template may comprise 10, 15, 20, 25, 50, 75, 100, 150, 200, 500, 1000, 1500, 2000, 2500, 3000, 3500, 4000, 4500, 5000, 5500, 6000, or more nucleotides in length. The template may be a single-stranded nucleic acid. The template can be double-stranded or partially double-stranded nucleic acid. In some embodiments, the single stranded template is 20, 30, 40, 50, 75, 100, 125, 150, 175, or 200 nucleotides in length. In some embodiments, the template may comprise a nucleotide sequence that is complementary to a portion of the target nucleic acid molecule comprising the target sequence (i.e., a “homology arm”). In some embodiments, the template may comprise a homology arm that is complementary to the sequence located upstream or downstream of the cleavage site on the target nucleic acid molecule.

In some embodiments, the template contains ssDNA or dsDNA containing flanking invert-terminal repeat (ITR) sequences. In some embodiments, the template is provided as a vector, plasmid, minicircle, nanocircle, or PCR product.

In some embodiments, the nucleic acid is purified. In some embodiments, the nucleic acid is purified using a precipitation method (e.g., LiCl precipitation, alcohol precipitation, or an equivalent method, e.g., as described herein). In some embodiments, the nucleic acid is purified using a chromatography-based method, such as an HPLC-based method or an equivalent method (e.g., as described herein). In some embodiments, the nucleic acid is purified using both a precipitation method (e.g., LiCl precipitation) and an HPLC-based method. In some embodiments, the nucleic acid is purified by tangential flow filtration (TFF).

### Cell Types

In some embodiments, the cell is a eukaryotic cell, such as a human cell in a subject. In some embodiments the cell is a cell in vivo, e.g. in a tissue, organ, or organism. In some embodiments the cell is a cell in vitro. In some embodiments, the cell is an immune cell. As used herein, “immune cell” refers to a cell of the immune system, including e.g., a lymphocyte (e.g., T cell, B cell, natural killer cell (“NK cell”, and NKT cell, or iNKT cell)), monocyte, macrophage, mast cell, dendritic cell, or granulocyte (e.g., neutrophil, eosinophil, and basophil). In some embodiments, the cell is a primary immune cell. In some embodiments, the immune system cell may be selected from CD3+, CD4+ and CD8+ T cells, regulatory T cells (Tregs), B cells, NK cells, and dendritic cells (DC). In some embodiments, the immune cell is allogeneic. In some embodiments, the cell is a lymphocyte. In some embodiments, the cell is an adaptive immune cell. In some embodiments, the cell is a T cell. In some embodiments, the cell is a B cell. In some embodiments, the cell is a NK cell.

As used herein, a T cell can be defined as a cell that expresses a T cell receptor (“TCR” or “αβ TCR” or “γδ TCR”), however in some embodiments, the TCR of a T cell may be genetically modified to reduce its expression (e.g., by genetic modification to the TRAC or TRBC genes), therefore expression of the protein CD3 may be used as a marker to identify a T cell by standard flow cytometry methods. CD3 is a multi-subunit signaling complex that associates with the TCR. Thus, a T cell may be referred to as CD3+. In some embodiments, a T cell is a cell that expresses a CD3+ marker and either a CD4+ or CD8+ marker.

In some embodiments, the T cell expresses the glycoprotein CD8 and therefore is CD8+ by standard flow cytometry methods and may be referred to as a “cytotoxic” T cell. In some embodiments, the T cell expresses the glycoprotein CD4 and therefore is CD4+ by standard flow cytometry methods and may be referred to as a “helper” T cell. CD4+ T cells can differentiate into subsets and may be referred to as a Th1 cell, Th2 cell, Th9 cell, Thl7 cell, Th22 cell, T regulatory (“Treg”) cell, or T follicular helper cells (“Tfh”). Each CD4+ subset releases specific cytokines that can have either proinflammatory or anti-inflammatory functions, survival or protective functions. A T cell may be isolated from a subject by CD4+ or CD8+ selection methods.

In some embodiments, the T cell is a memory T cell. In the body, a memory T cell has encountered antigen. A memory T cell can be located in the secondary lymphoid organs (central memory T cells) or in recently infected tissue (effector memory T cells). A memory T cell may be a CD8+ T cell. A memory T cell may be a CD4+ T cell. As used herein, a “central memory T cell” can be defined as an antigen-experienced T cell, and for example, may expresses CD62L and CD45RO. A central memory T cell may be detected as CD62L+ and CD45RO+ by Central memory T cells also express CCR7, therefore may be detected as CCR7+ by standard flow cytometry methods.

As used herein, an “early stem-cell memory T cell” (or “Tscm”) can be defined as a T cell that expresses CD27 and CD45RA, and therefore is CD27+ and CD45RA+ by standard flow cytometry methods. A Tscm does not express the CD45 isoform CD45RO, therefore a Tscm will further be CD45RO− if stained for this isoform by standard flow cytometry methods. A CD45RO− CD27+ cell is therefore also an early stem-cell memory T cell. Tscm cells further express CD62L and CCR7, therefore may be detected as CD62L+ and CCR7+ by standard flow cytometry methods. Early stem-cell memory T cells have been shown to correlate with increased persistence and therapeutic efficacy of cell therapy products.

In some embodiments, the cell is a B cell. As used herein, a “B cell” can be defined as a cell that expresses CD19 and/or CD20, and/or B cell mature antigen (“BCMA”), and therefore a B cell is CD19+, and/or CD20+, and/or BCMA+ by standard flow cytometry methods. A B cell is further negative for CD3 and CD56 by standard flow cytometry methods. The B cell may be a plasma cell. The B cell may be a memory B cell. The B cell may be a naïve B cell. The B cell may be IgM+ or has a class-switched B cell receptor (e.g., IgG+, or IgA+).

Cells used in ACT therapy are included, such as mesenchymal stem cells (e.g., isolated from bone marrow (BM), peripheral blood (PB), placenta, umbilical cord (UC) or adipose); hematopoietic stem cells (HSCs; e.g. isolated from BM); mononuclear cells (e.g., isolated from BM or PB); endothelial progenitor cells (EPCs; isolated from BM, PB, and UC); neural stem cells (NSCs); limbal stem cells (LSCs); or tissue-specific primary cells or cells derived therefrom (TSCs). Cells used in ACT therapy further include induced pluripotent stem cells (iPSCs) that may be induced to differentiate into other cell types including e.g., islet cells, neurons, and blood cells; ocular stem cells; pluripotent stem cells (PSCs); embryonic stem cells (ESCs); cells for organ or tissue transplantations such as islet cells, cardiomyocytes, thyroid cells, thymocytes, neuronal cells, skin cells, retinal cells, chondrocytes, myocytes, and keratinocytes.

In some embodiments, the cell is a human cell, such as a cell from a subject. In some embodiments, the cell is isolated from a human subject, such as a human donor. In some embodiments, the cell is isolated from human donor PBMCs or leukopaks. In some embodiments, the cell is from a subject with a condition, disorder, or disease. In some embodiments, the cell is from a human donor with Epstein Barr Virus (“EBV”).

In some embodiments, the cell is a mononuclear cell, such as from bone marrow or peripheral blood. In some embodiments, the cell is a peripheral blood mononuclear cell (“PBMC”). In some embodiments, the cell is a PBMC, e.g. a lymphocyte or monocyte. In some embodiments, the cell is a peripheral blood lymphocyte (“PBL”).

In some embodiments, the methods are carried out ex vivo. As used herein, “ex vivo” refers to an in vitro method wherein the cell is capable of being transferred into a subject, e.g., as an ACT therapy. In some embodiments, an ex vivo method is an in vitro method involving an ACT therapy cell or cell population.

In some embodiments, the cell is maintained in culture. In some embodiments, the cell is transplanted into a patient. In some embodiments, the cell is removed from a subject, genetically modified ex vivo, and then administered back to the same patient. In some embodiments, the cell is removed from a subject, genetically modified ex vivo, and then administered to a subject other than the subject from which it was removed.

In some embodiments, the cell is from a cell line. In some embodiments, the cell line is derived from a human subject. In some embodiments, the cell line is a lymphoblastoid cell line (“LCL”). The cell may be cryopreserved and thawed. The cell may not have been previously cryopreserved.

In some embodiments, the cell is from a cell bank. In some embodiments, the cell is genetically modified and then transferred into a cell bank. In some embodiments the cell is removed from a subject, genetically modified ex vivo, and transferred into a cell bank. In some embodiments, a genetically modified population of cells is transferred into a cell bank. In some embodiments, a genetically modified population of immune cells is transferred into a cell bank.

In some embodiments, a genetically modified population of immune cells comprising a first and second subpopulations, wherein the first and second sub-populations have at least one common genetic modification and at least one different genetic modification are transferred into a cell bank.

In some embodiments, the T cell is activated by polyclonal activation (or “polyclonal stimulation”) (not antigen-specific stimulation). In some embodiments, the T cell is activated by CD3 stimulation (e.g., providing an anti-CD3 antibody). In some embodiments, the T cell is activated by CD3 and CD28 stimulation (e.g., providing an anti-CD3 antibody and an anti-CD28 antibody). In some embodiments, the T cell is activated using a ready-to-use reagent to activate the T cell (e.g., via CD3/CD28 stimulation). In some embodiments, the T cell is activated by via CD3/CD28 stimulation provided by beads. In some embodiments, the T cell is activated by via CD3/CD28 stimulation wherein one or more components is soluble and/or one or more components is bound to a solid surface (e.g., plate or bead). In some embodiments, the T cell is activated by an antigen-independent mitogen (e.g., a lectin, including e.g., concanavalin A (“ConA”), or PHA).

In some embodiments, one or more cytokines are used for activation of T cells. IL-2 is provided for T cell activation and/or to promote T cell survival. In some embodiments, the cytokine(s) for activation of T cells is a cytokine that binds to the common gamma chain (γc) receptor. In some embodiments, IL-2 is provided for T cell activation. In some embodiments, IL-7 is provided for T cell activation. In some embodiments, IL-15 is provided for T cell activation. In some embodiments, IL-21 is provided for T cell activation. In some embodiments, a combination of cytokines is provided for T cell activation, including, e.g., IL-2, IL-7, IL-15, and/or IL-21.

In some embodiments, the T cell is activated by exposing the cell to an antigen (antigen stimulation). A T cell is activated by antigen when the antigen is presented as a peptide in a major histocompatibility complex (“MHC”) molecule (peptide-MHC complex). A cognate antigen may be presented to the T cell by co-culturing the T cell with an antigen-presenting cell (feeder cell) and antigen. In some embodiments, the T cell is activated by co-culture with an antigen-presenting cell that has been pulsed with antigen. In some embodiments, the antigen-presenting cell has been pulsed with a peptide of the antigen.

In some embodiments, the T cell may be activated for 12 to 72 hours. In some embodiments, the T cell may be activated for 12 to 48 hours. In some embodiments, the T cell may be activated for 12 to 24 hours. In some embodiments, the T cell may be activated for 24 to 48 hours. In some embodiments, the T cell may be activated for 24 to 72 hours. In some embodiments, the T cell may be activated for 12 hours. In some embodiments, the T cell may be activated for 48 hours. In some embodiments, the T cell may be activated for 72 hours.

### Definitions

It should be noted that, as used in this application, the singular form “a”, “an” and “the” include plural references unless the context clearly dictates otherwise. Thus, for example, reference to “a composition” includes a plurality of compositions and reference to “a cell” includes a plurality of cells and the like. The use of “or” is inclusive and means “and/or” unless stated otherwise.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; embodiments in the specification that recite “about” various components are also contemplated as “at” the recited components; and embodiments in the specification that recite “consisting essentially of” various components are also contemplated as “consisting of” or “comprising” the recited components (this interchangeability does not apply to the use of these terms in the claims).

Numeric ranges are inclusive of the numbers defining the range. Measured and measurable values are understood to be approximate, taking into account significant digits and the error associated with the measurement. As used in this application, the terms “about” and “approximately” have their art-understood meanings; use of one vs the other does not necessarily imply different scope. Unless otherwise indicated, numerals used in this application, with or without a modifying term such as “about” or “approximately”, should be understood to encompass normal divergence and/or fluctuations as would be appreciated by one of ordinary skill in the relevant art. In certain embodiments, the term “approximately” or “about” can referf to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.1% or less in either direction (greater than or less than) of a stated reference value unless otherwise stated or otherwise evident from the context (except where such number would exceed 100% of a possible value).

As used herein, the term “contacting” means establishing a physical connection between two or more entities. For example, contacting a mammalian cell with a nanoparticle composition means that the mammalian cell and a nanoparticle are made to share a physical connection. Methods of contacting cells with external entities both in vivo and ex vivo are well known in the biological arts. For example, contacting a nanoparticle composition and a mammalian cell disposed within a mammal may be performed by varied routes of administration (e.g., intravenous, intramuscular, intradermal, and subcutaneous) and may involve varied amounts of nanoparticle compositions. Moreover, more than one mammalian cell may be contacted by a nanoparticle composition.

As used herein, the term “delivering” means providing an entity to a destination. For example, delivering a therapeutic and/or prophylactic to a subject may involve administering a nanoparticle composition including the therapeutic and/or prophylactic to the subject (e.g., by an intravenous, intramuscular, intradermal, or subcutaneous route). Administration of a nanoparticle composition to a mammal or mammalian cell may involve contacting one or more cells with the nanoparticle composition.

As used herein, “encapsulation efficiency” refers to the amount of a therapeutic and/or prophylactic that becomes part of a nanoparticle composition, relative to the initial total amount of therapeutic and/or prophylactic used in the preparation of a nanoparticle composition. For example, if 97 mg of therapeutic and/or prophylactic are encapsulated in a nanoparticle composition out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

As used herein, the terms “editing efficiency”, “editing percentage”, “indel efficiency”, and “percent indels” refer to the total number of sequence reads with insertions or deletions relative to the total number of sequence reads. For example, editing efficiency at a target location in a genome may be measured by isolating and sequencing genomic DNA to identify the presence of insertions and deletions introduced by gene editing. In some embodiments, editing efficiency is measured as a percentage of cells that no longer contain a gene (e.g., CD3) after treatment, relative to the number of the cells that initially contained that gene (e.g., CD3+ cells).

As used herein, “knockdown” refers to a decrease in expression of a particular gene product (e.g., protein, mRNA, or both). Knockdown of a protein can be measured by detecting total cellular amount of the protein from a sample, such as a tissue, fluid, or cell population of interest. It can also be measured by measuring a surrogate, marker, or activity for the protein. Methods for measuring knockdown of mRNA are known and include sequencing of mRNA isolated from a sample of interest. In some embodiments, “knockdown” may refer to some loss of expression of a particular gene product, for example a decrease in the amount of mRNA transcribed or a decrease in the amount of protein expressed by a population of cells (including in vivo populations such as those found in tissues).

As used herein, “knockout” refers to a loss of expression from a particular gene or of a particular protein in a cell. Knockout can be measured by detecting total cellular amount of a protein in a cell, a tissue or a population of cells, for example. Knockout can also be detected at the genome or mRNA level, for example.

As used herein, the term “biodegradable” is used to refer to materials that, when introduced into cells, are broken down by cellular machinery (e.g., enzymatic degradation) or by hydrolysis into components that cells can either reuse or dispose of without significant toxic effect(s) on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis.

As used herein, the “N/P ratio” is the molar ratio of ionizable nitrogen atom-containing lipid (e.g. Compound of Formula I) to phosphate groups in RNA, e.g., in a nanoparticle composition including a lipid component and an RNA.

Compositions may also include salts of one or more compounds. Salts may be pharmaceutically acceptable salts. As used herein, “pharmaceutically acceptable salts” refers to derivatives of the disclosed compounds wherein the parent compound is altered by converting an existing acid or base moiety to its salt form (e.g., by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

As used herein, the “polydispersity index” is a ratio that describes the homogeneity of the particle size distribution of a system. A small value, e.g., less than 0.3, indicates a narrow particle size distribution. In some embodiments, the polydispersity index may be less than 0.1.

As used herein, “transfection” refers to the introduction of a species (e.g., an RNA) into a cell. Transfection may occur, for example, in vitro, ex vivo, or in vivo.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched (i.e., linear). The alkyl group can also be substituted or unsubstituted. For example, the alkyl group can be substituted with one or more groups including, but not limited to, alkyl, aryl, heteroaryl, cycloalkyl, alkoxy, amino, ether, halide, hydroxy, nitro, silyl, sulfoxo, sulfonate, carboxylate, or thiol, as described herein. A “lower alkyl” group is an alkyl group containing from one to six (e.g., from one to four) carbon atoms.

The term “alkenyl”, as used herein, refers to an aliphatic group containing at least one carbon-carbon double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, an alkenyl group may be substituted by one or more alkyl, carbocyclyl, aryl, heterocyclyl, or heteroaryl groups is contemplated. Exemplary alkenyl groups include, but are not limited to, vinyl (—CH═CH₂), allyl (—CH₂CH═CH₂), cyclopentenyl (—C₅H7), and 5-hexenyl (—CH₂CH₂CH₂CH₂CH═CH₂).

An “alkylene” group refers to a divalent alkyl radical, which may be branched or unbranched (i.e., linear). Any of the above mentioned monovalent alkyl groups may be converted to an alkylene by abstraction of a second hydrogen atom from the alkyl. Representative alkylenes include C₂₋₄ alkylene and C₂₋₃ alkylene. Typical alkylene groups include, but are not limited to —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like. The alkylene group can also be substituted or unsubstituted. For example, the alkylene group can be substituted with one or more groups including, but not limited to, alkyl, aryl, heteroaryl, cycloalkyl, alkoxy, amino, ether, halide, hydroxy, nitro, silyl, sulfoxo, sulfonate, carboxylate, or thiol, as described herein.

The term “alkenylene” includes divalent, straight or branched, unsaturated, acyclic hydrocarbyl groups having at least one carbon-carbon double bond and, in one embodiment, no carbon-carbon triple bonds. Any of the above-mentioned monovalent alkenyl groups may be converted to an alkenylene by abstraction of a second hydrogen atom from the alkenyl. Representative alkenylenes include C₂₋₆alkenylenes.

The term “C_{x-y}” when used in conjunction with a chemical moiety, such as alkyl or alkylene, is meant to include groups that contain from x to y carbons in the chain. For example, the term “C_{x-y} alkyl” refers to substituted or unsubstituted saturated hydrocarbon groups, including straight-chain and branched-chain alkyl and alkylene groups that contain from x to y carbons in the chain.

The term “alkoxy” refers to an alkyl group, preferably a lower alkyl group, having an oxygen attached thereto. Representative alkoxy groups include methoxy, ethoxy, propoxy, tert-butoxy and the like.

While the inventions are described in conjunction with the illustrated embodiments, it is understood that they are not intended to limit the invention to those embodiments. On the contrary, the disclosure is intended to cover all alternatives, modifications, and equivalents, including equivalents of specific features, which may be included within the inventions as defined by the appended claims.

Both the foregoing general description and detailed description, as well as the following examples, are exemplary and explanatory only and are not restrictive of the teachings. The section headings used herein are for organizational purposes only and are not to be construed as limiting the desired subject matter in any way. In the event that any literature incorporated by reference contradicts any term defined in this specification, this specification controls. All ranges given in the application encompass the endpoints unless stated otherwise.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicant reserves the right to physically incorporate into this application any and all materials and information from any such articles, patents, patent applications, or other physical and electronic documents.

### EXAMPLES

### Example 1—Materials and Methods

### 1.1. T Cell Culture Media Preparation.

T cell culture media compositions used below are described here. “X-VIVO Base Media” consists of X-VIVO™ 15 Media, 1% Penstrep, 50 μM Beta-Mercaptoethanol, 10 mM NAC. In addition to above mentioned components, other variable media components used were: 1. Serum (Fetal Bovine Serum (FBS)); and 2. Cytokines (IL-2, IL-7, IL-15).

### 1.2. T Cell Preparation

Healthy human donor leukapheresis was obtained commercially (Hemacare). T cells were isolated by negative selection using the EasySep Human T cell Isolation Kit (Stem Cell Technology, Cat. 17951) or by CD4/CD8 positive selection using the StraightFrom® Leukopak® CD4/CD8 MicroBeads (Milteny, Catalog #130-122-352) on the MultiMACS™ Cell24 Separator Plus instrument following manufacturers instruction. T cells were cryopreserved in Cryostor CS10 freezing media (Cat. #07930) for future use.

Upon thaw, T cells were cultured in complete T cell growth media composed of CTS OpTmizer Base Media (CTS OpTmizer Media (Gibco, A3705001) supplemented with 1× GlutaMAX, 10 mM HEPES buffer (10 mM), and 1% pen-strep (Gibco, 15140-122) further supplemented with 200 IU/mL IL-2 (Peprotech, 200-02), 5 ng/mL IL-7 (Peprotech, 200-07), 5 ng/mL IL-15 (Peprotech, 200-15), and 2.5% human serum (Gemini, 100-512). After overnight rest, T cells at a density of 10⁶/mL were activated with T cell TransAct Reagent (1:100 dilution, Miltenyi) and incubated at 37° C. for 24 or 48 hours. Post incubation, cells at a density of 0.5×10⁶/mL were used for editing applications.

Unless otherwise indicated, the same process was used for non-activated T cells with the following exceptions. Upon thaw, non-Activated T cells were cultured in the CTS complete growth media composed of CTS OpTmizer Base Media (Thermofisher, A10485-01), 1% pen-strep (Corning, 30-002-CI) 1× GlutaMAX (Thermofisher, 35050061), 10 mM HEPES (Thermofisher, 15630080)) which was further supplemented with 200 U/mL IL-2 (Peprotech, 200-02), 5 ng/mL IL-7 (Peprotech, 200-07), 5 ng/mL IL-15 (Peprotech, 200-15) with 5% human AB serum (Gemini, 100-512) were incubated for 24 hrs with no activation. T cells were plated at a cell density of 10⁶/mL in 100 uL of CTS OpTmizer base media, described above, containing 2.5% human serum and cytokines for editing applications.

### 1.3. Preparation of Lipid Nanoparticles.

Unless otherwise specified, the lipid components were dissolved in 100% ethanol at various molar ratios. The RNA cargos (e.g., Cas9 mRNA and sgRNA) were dissolved in 25 mM citrate, 100 mM NaCl, pH 5.0, resulting in a concentration of RNA cargo of approximately 0.45 mg/mL.

Unless otherwise specified, the LNPs contained ionizable Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG in a 50:38:9:3 molar ratio, respectively. The lipid nucleic acid assemblies were formulated with a lipid amine to RNA phosphate (N:P) molar ratio of about 6, and a ratio of gRNA to mRNA of 1:1 by weight, unless otherwise specified. In Examples 13-16, a ratio of gRNA to mRNA of 1:2 by weight was used, unless otherwise specified.

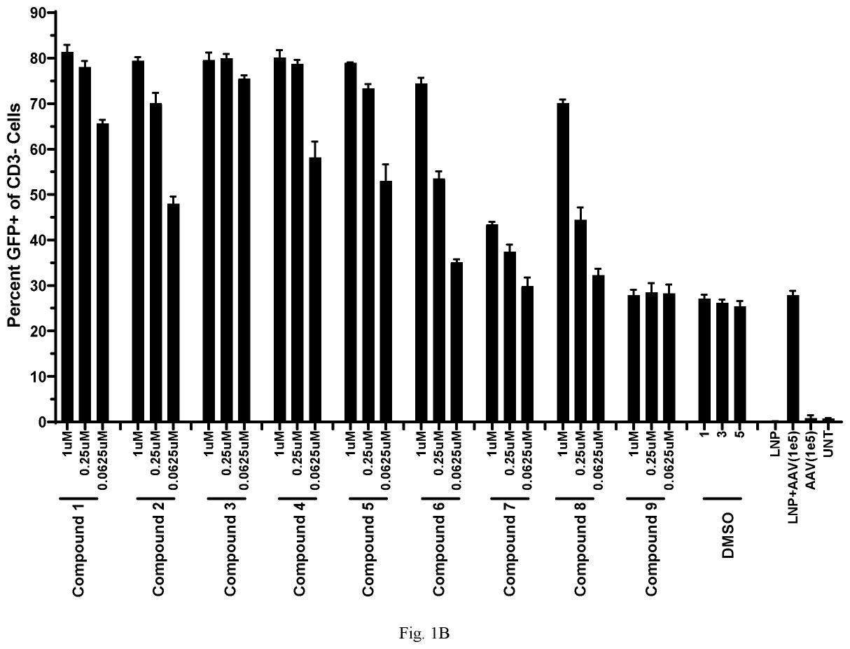

Lipid nanoparticles (LNPs) were prepared using a cross-flow technique utilizing impinging jet mixing of the lipid in ethanol with two volumes of RNA solutions and one volume of water. The lipids in ethanol were mixed through a mixing cross with the two volumes of RNA solution. A fourth stream of water was mixed with the outlet stream of the cross through an inline tee (See WO2016010840 FIG. 2.). The LNPs were held for 1 hour at room temperature (RT), and further diluted with water (approximately 1:1 v/v). LNPs were concentrated using tangential flow filtration, e.g., on a flat sheet cartridge (Sartorius, 100kD 0) and buffer exchanged using PD-10 desalting columns (GE) into 50 mM Tris, 45 mM NaCl, 5% (w/v) sucrose, pH 7.5 (TSS). Alternatively, the LNP's were optionally concentrated using 100 kDa Amicon spin filter and buffer exchanged using PD-10 desalting columns (GE) into TSS. The resulting mixture was then filtered using a 0.2 m sterile filter. The final LNP was stored at 4° C. or −80° C. until further use. 1.4. Next-generation sequencing (“NGS”) and analysis for on-target cleavage efficiency

Genomic DNA was extracted using QuickExtract™ DNA Extraction Solution (Lucigen, Cat. QE09050) according to manufacturer's protocol.

To quantitatively determine the efficiency of editing at the target location in the genome, next generation sequencing was utilized to identify the presence of insertions and deletions introduced by gene editing. PCR primers were designed around the target site within the gene of interest (e.g. TRAC), and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the human (e.g., hg38) reference genome after eliminating those having low quality scores. The resulting files containing the reads were mapped to the reference genome (BAM files), where reads that overlapped the target region of interest were selected and the number of wild-type reads versus the number of reads which contain an insertion or deletion (“indel”) was calculated.

The editing percentage (e.g., the “editing efficiency” or “percent editing”) is defined as the total number of sequence reads with insertions or deletions (“indels”) over the total number of sequence reads, including wild type.

1.5. In Vitro Transcription (“IVT”) of Nuclease mRNA

Capped and polyadenylated mRNA containing N1-methyl pseudo-U was generated by in vitro transcription using a linearized plasmid DNA template and T7 RNA polymerase. Plasmid DNA containing a T7 promoter, a sequence for transcription, and a polyadenylation region was linearized by incubating at 37° C. for 2 hours with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts. The IVT reaction to generate modified mRNA was performed by incubating at 37° C. for 1.5-4 hours in the following conditions: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. TURBO DNase (ThermoFisher) was added to a final concentration of 0.01 U/μL, and the reaction was incubated for an additional 30 minutes to remove the DNA template. The mRNA was purified using a MegaClear Transcription Clean-up kit (ThermoFisher) or a RNeasy Maxi kit (Qiagen) per the manufacturers' protocols.

Alternatively, the mRNA was purified through a precipitation protocol, which in some cases was followed by HPLC-based purification. Briefly, after the DNase digestion, mRNA is purified using LiCl precipitation, ammonium acetate precipitation and sodium acetate precipitation. For IPLC purified mRNA, after the LiCl precipitation and reconstitution, the mRNA was purified by RP-TP IPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

Streptococcus pyogenes (“Spy”) Cas9 mRNA was generated from plasmid DNA encoding an open reading frame according to SEQ ID Nos: 9-10 (see sequences in Additional Sequence Table). When the sequences cited in this paragraph are referred to below with respect to RNAs, it is understood that Ts should be replaced with Us (which can be modified nucleosides as described above). Messenger RNAs used in the Examples include a 5′ cap and a 3′ polyadenylation sequence e.g., up to 100 nts and are identified by in Additional Sequence Table.

Guide RNAs are chemically synthesized by methods known in the art.

### Compound Synthesis

### General Information

All reagents and solvents were purchased and used as received from commercial vendors or synthesized according to cited procedures. All intermediates and final compounds were purified using flash column chromatography on silica gel. NMR spectra were recorded on a Bruker or Varian 400 MHz spectrometer, and NMR data were collected in CDCl3 at ambient temperature. Chemical shifts are reported in parts per million (ppm) relative to CDCl3 (7.26). Data for 1H NMR are reported as follows: chemical shift, multiplicity (br=broad, s=singlet, d=doublet, t=triplet, q=quartet, dd=doublet of doublets, dt=doublet of triplets m=multiplet), coupling constant, and integration. MS data were recorded on a Waters SQD2 mass spectrometer with an electrospray ionization (ESI) source. Purity of the final compounds was determined by UPLC-MS-ELS using a Waters Acquity H-Class liquid chromatography instrument equipped with SQD2 mass spectrometer with photodiode array (PDA) and evaporative light scattering (ELS) detectors.

TABLE 1
DNA PK inhibitor Compounds

| Compound | Structure |
|---|---|
| 1 | 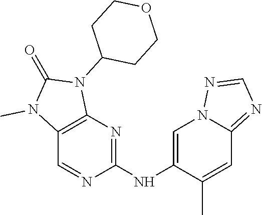 [SMILES: Cc1cc2ncnn2cc1Nc1ncc2c(n1)n(C1CCOCC1)c(=O)n2C] |
| 2 | 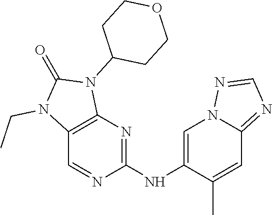 [SMILES: CCn1c(=O)n(C2CCOCC2)c2nc(Nc3cn4ncnc4cc3C)ncc21] |
| 3 | 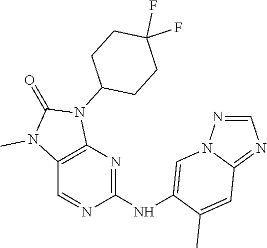 [SMILES: Cc1cc2ncnn2cc1Nc1ncc2c(n1)n(C1CCC(F)(F)CC1)c(=O)n2C] |
| 4 | 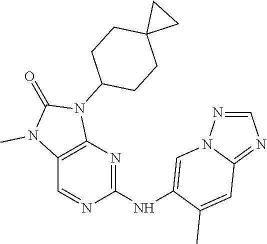 [SMILES: Cc1cc2ncnn2cc1Nc1ncc2c(n1)n(C1CCC3(CC1)CC3)c(=O)n2C] |
| 5 | 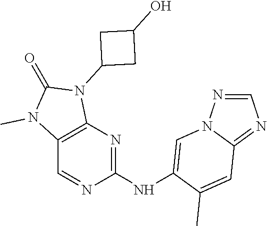 [SMILES: Cc1cc2ncnn2cc1Nc1ncc2c(n1)n(C1CC(O)C1)c(=O)n2C] |
| 6 | 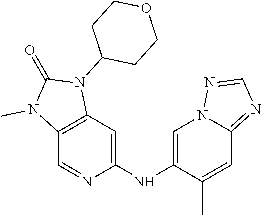 [SMILES: Cc1cc2ncnn2cc1Nc1cc2c(cn1)n(C)c(=O)n2C1CCOCC1] |
| 7 | 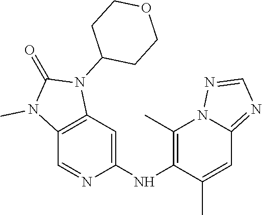 [SMILES: Cc1cc2ncnn2c(C)c1Nc1cc2c(cn1)n(C)c(=O)n2C1CCOCC1] |
| 8 | 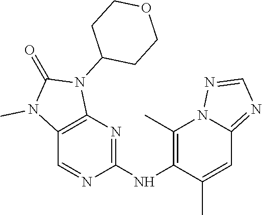 [SMILES: Cc1cc2ncnn2c(C)c1Nc1ncc2c(n1)n(C1CCOCC1)c(=O)n2C] |
| 9 | 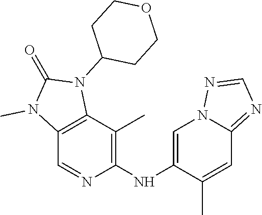 [SMILES: Cc1cc2ncnn2cc1Nc1ncc2c(c1C)n(C1CCOCC1)c(=O)n2C] |

### Example 2—Compound 1

### Intermediate 1a: (E)-N,N-dimethyl-N-(4-methyl-5-nitropyridin-2-yl)formimidamide

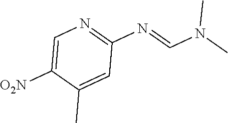

To a solution of 4-methyl-5-nitro-pyridin-2-amine (5 g, 1.0 equiv.) in toluene (0.3 M) was added DMF-DMA (3.0 equiv.). The mixture was stirred at 110° C. for 2 h. The reaction mixture was concentrated under reduced pressure to give a residue and purified by column chromatography to afford product as a yellow solid (59%). ¹H NMR (400 MHz, (CD₃)₂SO) δ 8.82 (s, 1H), 8.63 (s, 1H), 6.74 (s, 1H), 3.21 (m, 6H).

### Intermediate 1b: (E)-N-hydroxy-N-(4-methyl-5-nitropyridin-2-yl)formimidamide

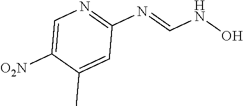

To a solution of Intermediate 1a (4 g, 1.0 equiv.) in MeOH (0.2 M) was added NH₂OH HCl (2.0 equiv.). The reaction mixture was stirred at 80° C. for 1 h. The reaction mixture was filtered, and the filtrate was concentrated under reduced pressure to give a residue. The residue was partitioned between H₂O and EtOAc, followed by 2× extraction with EtOAc. The organic phases were concentrated under reduced pressure to give a residue and purified by column chromatography to afford product as a white solid (66%). 1H NMR (400 MHz, (CD₃)₂SO) δ 10.52 (d, J=3.8 Hz, 1H), 10.08 (dd, J=9.9, 3.7 Hz, 1H), 8.84 (d, J=3.8 Hz, 1H), 7.85 (dd, J=9.7, 3.8 Hz, 1H), 7.01 (d, J=3.9 Hz, 1H), 3.36 (s, 3H).

### Intermediate 1c: 7-methyl-6-nitro-[1,2,4]triazolo[1,5-a]pyridine

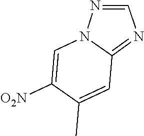

To a solution of Intermediate 1b (2.5 g, 1.0 equiv.) in THE (0.4 M) was added trifluoroacetic anhydride (1.0 equiv.) at 0° C. The mixture was stirred at 25° C. for 18 h. The reaction mixture was filtered, and the filtrate was concentrated under reduced pressure to give a residue. The residue was purified by column chromatography to afford product as a white solid (44%). ¹H NMR (400 MHz, CDCl₃) δ 9.53 (s, 1H), 8.49 (s, 1H), 7.69 (s, 1H), 2.78 (d, J=1.0 Hz, 3H).

### Intermediate 1d: 7-methyl-[1,2,4]triazolo[1,5-a]pyridin-6-amine

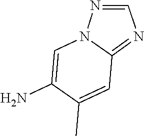

To a mixture of Pd/C (10% w/w, 0.2 equiv.) in EtOH (0.1 M) was added Intermediate 1c (1.0 equiv. and ammonium formate (5.0 equiv.). The mixture was heated at 105° C. for 2 h. The reaction mixture was filtered, and the filtrate was concentrated under reduced pressure to give a residue. The residue was purified by column chromatography to afford product as a pale brown solid. ¹H NMR (400 MHz, (CD₃)₂SO) δ 8.41 (s, 2H), 8.07 (d, J=9.0 Hz, 2H), 7.43 (s, 1H), 2.22 (s, 3H).

### Intermediate 1e: ethyl 2-chloro-4-((tetrahydro-2H-pyran-4-yl)amino)pyrimidine-5-carboxylate

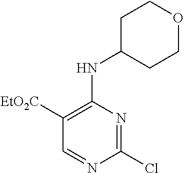 [SMILES: CCOC(=O)c1cnc(Cl)nc1NC1CCOCC1]

To a solution of tetrahydropyran-4-amine (5 g, 1.0 equiv.) and ethyl 2,4-dichloropyrimidine-5-carboxylate (1.0 equiv.) in MeCN (0.25-2.0 M) was added K₂CO₃ (1.0-3.0 equiv.). The mixture was stirred at 20-25° C. for at least 12 h. The reaction mixture was filtered, and the filtrate was concentrated under reduced pressure to give a residue. The residue was purified by column chromatography to afford product as a pale yellow solid (21%). 1H NMR (400 MHz, (CD₃)₂SO) δ 8.60 (s, 1H), 8.29 (d, J=7.7 Hz, 1H), 4.28 (q, J=7.1 Hz, 2H), 4.14 (dtt, J=11.3, 8.3, 4.0 Hz, 1H), 3.82 (dt, J=12.1, 3.6 Hz, 2H), 3.57 (s, 1H), 1.87-1.78 (m, 2H), 1.76-1.67 (m, 1H), 1.54 (qd, J=10.9, 4.3 Hz, 2H), 1.28 (t, J=7.1 Hz, 3H).

Intermediate 1f: 2-chloro-4-((tetrahydro-2H-pyran-4-yl)amino)pyrimidine-5-carboxylic acid

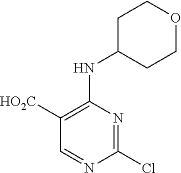 [SMILES: O=C(O)c1cnc(Cl)nc1NC1CCOCC1]

To a solution of LiOH (2.5 equiv.) in 1:1 THF/H₂O (0.25-1.0 M) was added Intermediate 1e (3.0 g, 1.0 equiv.). The mixture was stirred at 25° C. for 12 h. The mixture was concentrated under reduced pressure to remove THF. The residue was adjusted pH to 2 by 2 M HCl, and the resulting precipitate was collected by filtration, washed with water, and dried under vacuum to get a residue. The residue was purified by column chromatography to afford product as a white solid (74%) or used directly as a crude product.

### Intermediate 1g: 2-chloro-9-(tetrahydro-2H-pyran-4-yl)-7,9-dihydro-8H-purin-8-one

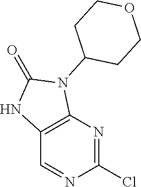 [SMILES: O=c1[nH]c2cnc(Cl)nc2n1C1CCOCC1]

To a solution of Intermediate if (2 g, 1.0 equiv.) in MeCN (0.2-0.5 M) was added Et₃N (1.0 equiv.). The mixture was stirred at 25° C. for 30 min. Then DPPA (1.0 equiv.) was added to the mixture. The mixture was stirred at 100° C. for at least 7 h. The reaction mixture was poured into water, and the resulting precipitate was collected by filtration, washed with water, and dried under vacuum to get a residue. The residue was purified by column chromatography to afford product as a white solid (56%). ¹H NMR (400 MHz, CDCl₃) δ 9.50 (s, 1H), 8.09 (s, 1H), 4.53 (tt, J=12.4, 4.2 Hz, 1H), 4.07 (dt, J=9.5, 4.8 Hz, 2H), 3.48 (td, J=12.1, 1.9 Hz, 2H), 2.69 (qd, J=12.5, 4.7 Hz, 2H), 1.67 (dd, J=12.1, 3.9 Hz, 2H).

### Intermediate 1h: 2-chloro-7-methyl-9-(tetrahydro-2H-pyran-4-yl)-7,9-dihydro-8H-purin-8-one

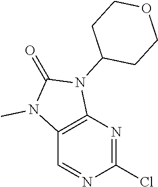 [SMILES: Cn1c(=O)n(C2CCOCC2)c2nc(Cl)ncc21]

To a mixture of Intermediate 1g (300 mg, 1.0 equiv.) and NaOH (5.0 equiv.) in 1:1 THF/H₂O (0.25-1.0 M) was added iodomethane (2.0 equiv.). The reaction mixture was stirred at 25° C. for 12 h. The reaction mixture was concentrated under reduced pressure to give a residue and purified by column chromatography to afford product as a white solid (47%). ¹H NMR (400 MHz, (CD₃)₂SO) δ 8.34 (s, 1H), 4.43 (ddt, J=12.2, 8.5, 4.2 Hz, 1H), 3.95 (dd, J=11.5, 4.6 Hz, 2H), 3.43 (td, J=12.1, 1.9 Hz, 2H), 2.45 (s, 3H), 2.40 (td, J=12.5, 4.7 Hz, 2H), 1.66 (ddd, J=12.2, 4.4, 1.9 Hz, 2H).

Compound 1: 7-methyl-2-((7-methyl-[1,2,4]triazolo[1,5-a]pyridin-6-yl)amino)-9-(tetrahydro-2H-pyran-4-yl)-7,9-dihydro-8H-purin-8-one

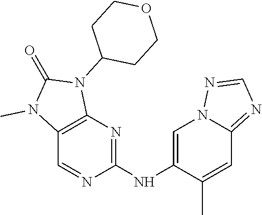 [SMILES: Cc1cc2ncnn2cc1Nc1ncc2c(n1)n(C1CCOCC1)c(=O)n2C]

A mixture of Intermediate 1h (1.3 g, 1.0 equiv.), Intermediate 1d (1.0 equiv.), Pd(dppf)Cl₂ (0.1-0.2 equiv.), XantPhos (0.1-0.2 equiv.) and Cs₂CO₃ (2.0 equiv.) in DMF (0.05-0.3 M) was degassed and purged 3× with N₂ and the mixture was stirred at 100-130° C. for at least 12 h under N₂ atmosphere. The reaction mixture was then poured into water and extracted 3× with DCM. The combined organic phase was washed with brine, dried with anhydrous Na₂SO₄, filtered, and the filtrate was concentrated in vacuum. The residue was purified by column chromatography to afford product as a pale yellow solid. ¹H NMR (400 MHz, (CD₃)₂SO) δ 9.13 (s, 1H), 8.69 (s, 1H), 8.39 (s, 1H), 8.10 (s, 1H), 7.72 (s, 1H), 4.50-4.36 (m, 1H), 3.98 (dd, J=11.6, 4.4 Hz, 2H), 3.44 (d, J=11.9 Hz, 2H), 3.32 (s, 3H), 2.44-2.38 (m, 3H), 1.69 (d, J=11.6 Hz, 2H). MS: 381.3 m/z [M+H].

### Example 3—Compound 2

### Intermediate 2a: 2-chloro-7-ethyl-9-(tetrahydro-2H-pyran-4-yl)-7,9-dihydro-8H-purin-8-one

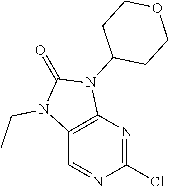 [SMILES: CCn1c(=O)n(C2CCOCC2)c2nc(Cl)ncc21]

To a mixture of Intermediate 1h (800 mg, 1.0 equiv.) and NaOH (5.0 equiv.) in THE (0.4 M) and H₂O (0.8 M) was added EtI (3.0 equiv.). The reaction mixture was stirred at 20° C. for 12 h. The reaction mixture concentrated under reduced pressure to give a residue and purified by column chromatography to afford product as a white solid (45%). ¹H NMR (400 MHz, (CD₃)₂SO) δ 8.50 (s, 1H), 4.52 (tt, J=12.2, 4.2 Hz, 1H), 4.03 (dd, J=11.5, 4.6 Hz, 2H), 3.95 (q, J=7.2 Hz, 2H), 3.51 (td, J=12.1, 1.9 Hz, 2H), 2.48 (td, J=12.5, 4.7 Hz, 2H), 1.79-1.71 (m, 2H), 1.31 (t, J=7.2 Hz, 3H).

### Compound 2: 7-ethyl-2-((7-methyl-[1,2,4]triazolo[1,5-a]pyridin-6-yl)amino)-9-(tetrahydro-2H-pyran-4-yl)-7,9-dihydro-8H-purin-8-one

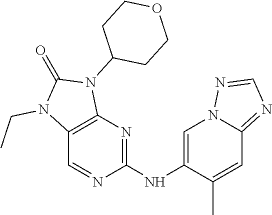 [SMILES: CCn1c(=O)n(C2CCOCC2)c2nc(Nc3cn4ncnc4cc3C)ncc21]

Compound 2 was synthesized as a TFA salt from Intermediate 1d and Intermediate 2a using the method employed for Compound 1, followed by a purification by reverse-phase HPLC. ¹H NMR (400 MHz, (CD₃)₂SO) δ 9.11 (s, 1H), 8.69 (s, 1H), 8.38 (s, 1H), 8.15 (s, 1H), 7.71 (t, J=1.0 Hz, 1H), 4.42 (ddd, J=12.1, 7.9, 4.1 Hz, 1H), 3.96 (dd, J=11.7, 4.4 Hz, 2H), 3.83 (q, J=7.2 Hz, 2H), 3.41 (t, J=11.9 Hz, 2H), 2.40 (d, J=1.0 Hz, 3H), 1.68 (d, J=11.0 Hz, 2H), 1.23 (t, J=7.2 Hz, 3H). MS: 395.3 m/z [M+H].

### Example 4—Compound 3

### Intermediate 3a: ethyl 2-chloro-4-((4,4-difluorocyclohexyl)amino)pyrimidine-5-carboxylate

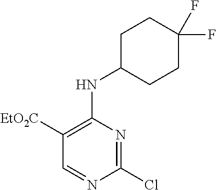 [SMILES: CCOC(=O)c1cnc(Cl)nc1NC1CCC(F)(F)CC1]

Intermediate 3a was synthesized from ethyl 2,4-dichloropyrimidine-5-carboxylate and 4,4-difluorocyclohexanamine hydrochloride using the method employed in Intermediate 1e. ¹H NMR (400 MHz, (CD₃)₂SO) δ 8.61 (s, 1H), 8.30 (d, J=7.7 Hz, 1H), 4.29 (q, J=7.1 Hz, 2H), 4.19-4.09 (m, 1H), 2.09-1.90 (m, 6H), 1.69-1.58 (m, 2H), 1.29 (t, J=7.1 Hz, 3H).

### Intermediate 3b: 2-chloro-4-((4,4-difluorocyclohexyl)amino)pyrimidine-5-carboxylic acid

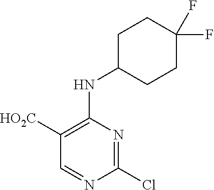 [SMILES: O=C(O)c1cnc(Cl)nc1NC1CCC(F)(F)CC1]

Intermediate 3b was synthesized (78%) from Intermediate 3a using the method employed in Intermediate 1f. ¹H NMR (400 MHz, (CD₃)₂SO) δ 13.77 (s, 1H), 8.57 (s, 1H), 8.53 (d, J=7.8 Hz, 1H), 4.12 (d, J=10.2 Hz, 1H), 2.14-1.89 (m, 6H), 1.62 (ddt, J=17.0, 10.3, 6.0 Hz, 2H).

### Intermediate 3c: 2-chloro-9-(4,4-difluorocyclohexyl)-7,9-dihydro-8H-purin-8-one

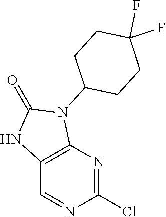 [SMILES: O=c1[nH]c2cnc(Cl)nc2n1C1CCC(F)(F)CC1]

Intermediate 3c was synthesized (56%) from Intermediate 3b using the method employed in Intermediate 1g. ¹H NMR (400 MHz, (CD₃)₂SO) δ 11.76-11.65 (m, 1H), 8.20 (s, 1H), 4.47 (dq, J=12.6, 6.2, 4.3 Hz, 1H), 2.34-1.97 (m, 6H), 1.90 (d, J=12.9 Hz, 2H).

### Intermediate 3d: 2-chloro-9-(4,4-difluorocyclohexyl)-7-methyl-7,9-dihydro-8H-purin-8-one

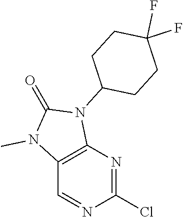 [SMILES: Cn1c(=O)n(C2CCC(F)(F)CC2)c2nc(Cl)ncc21]

To a mixture of Intermediate 3c (1.4 g, 1.0 equiv.), NaOH (5.0 equiv.) in 5:1 THF/H₂O (0.3 M) was added Mel (2.0 equiv.). The mixture was stirred at 20° C. for 12 h under N₂ atmosphere. The reaction mixture was concentrated under reduced pressure to give a residue and purified by column chromatography to afford product as a yellow solid (47%). ¹H NMR (400 MHz, CDCl₃) δ 8.01 (s, 1H), 4.53-4.39 (m, 1H), 3.43 (s, 3H), 2.73 (qd, J=12.7, 12.1, 3.8 Hz, 2H), 2.32-2.20 (m, 2H), 2.03-1.82 (m, 4H).

### Compound 3: 9-(4,4-difluorocyclohexyl)-7-methyl-2-((7-methyl-[1,2,4]triazolo[1,5-a]pyridin-6-yl)amino)-7,9-dihydro-8H-purin-8-one

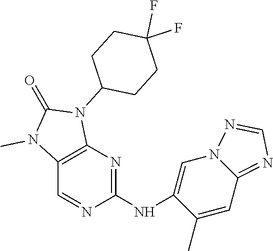 [SMILES: Cc1cc2ncnn2cc1Nc1ncc2c(n1)n(C1CCC(F)(F)CC1)c(=O)n2C]

Compound 3 was synthesized from Intermediate 1d and Intermediate 3d using the method employed for Compound 1, followed by a purification by reverse-phase HPLC. 1H NMR (400 MHz, (CD₃)₂SO) δ 9.03 (s, 1H), 8.66 (s, 1H), 8.38 (s, 1H), 8.10 (s, 1H), 7.71 (d, J=1.4 Hz, 1H), 4.36 (d, J=12.3 Hz, 1H), 3.31 (s, 3H), 2.38 (d, J=1.0 Hz, 3H), 2.11-1.96 (m, 4H), 1.81 (d, J=12.6 Hz, 2H). MS: 415.5 m/z [M+H].

### Example 5—Compound 4

### Intermediate 4a: 8-methylene-1,4-dioxaspiro[4.5]decane

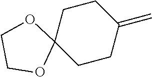

To a solution of methyl(triphenyl)phosphonium bromide (1.15 equiv.) in THE (0.6 M) was added n-BuLi (1.1 equiv.) at −78° C. dropwise, and the mixture was stirred at 0° C. for 1 h. Then, 1,4-dioxaspiro[4.5]decan-8-one (50 g, 1.0 equiv.) was added to the reaction mixture. The mixture was stirred at 25° C. for 12 h. The reaction mixture was poured into aq. NH₄Cl at 0° C., diluted with H₂O, and extracted 3× with EtOAc. The combined organic layers were concentrated under reduced pressure to give a residue and purified by column chromatography to afford product as a colorless oil (51%). ¹H NMR (400 MHz, CDCl₃) δ 4.67 (s, 1H), 3.96 (s, 4H), 2.82 (t, J=6.4 Hz, 4H), 1.70 (t, J=6.4 Hz, 4H).

### Intermediate 4b: 7,10-dioxadispiro[2.2.4⁶.2³]dodecane

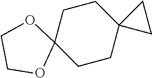 [SMILES: C1COC2(CCC3(CC3)CC2)O1]

To a solution of Intermediate 4a (5 g, 1.0 equiv.) in toluene (3 M) was added ZnEt₂ (2.57 equiv.) dropwise at −40° C. and the mixture was stirred at −40° C. for 1 h. Then diiodomethane (6.0 equiv.) was added dropwise to the mixture at −40° C. under N₂. The mixture was then stirred at 20° C. for 17 h under N₂ atmosphere. The reaction mixture was poured into aq. NH₄Cl at 0° C. and extracted 2× with EtOAc. The combined organic phases were washed with brine (20 mL), dried with anhydrous Na₂SO₄, filtered, and the filtrate was concentrated in vacuum. The residue was purified by column chromatography to afford product as a pale-yellow oil (73%).

### Intermediate 4c: spiro[2.5]octan-6-one

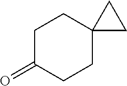

To a solution of Intermediate 4b (4 g, 1.0 equiv.) in 1:1 THF/H₂O (1.0 M) was added TFA (3.0 equiv.). The mixture was stirred at 20° C. for 2 h under N₂ atmosphere. The reaction mixture was concentrated under reduced pressure to remove THF, and the residue adjusted pH to 7 with 2 M NaOH (aq.). The mixture was poured into water and 3× extracted with EtOAc. The combined organic phase was washed with brine, dried with anhydrous Na₂SO₄, filtered, and the filtrate was concentrated in vacuum. The residue was purified by column chromatography to afford product as a pale-yellow oil (68%). ¹H NMR (400 MHz, CDCl₃) δ 2.35 (t, J=6.6 Hz, 4H), 1.62 (t, J=6.6 Hz, 4H), 0.42 (s, 4H).

### Intermediate 4d: N-(4-methoxybenzyl)spiro[2.5]octan-6-amine

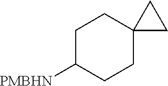

To a mixture of Intermediate 4c (2 g, 1.0 equiv.) and (4-methoxyphenyl)methanamine (1.1 equiv.) in DCM (0.3 M) was added AcOH (1.3 equiv.). The mixture was stirred at 20° C. for 1 h under N₂ atmosphere. Then, NaBH(OAc)₃ (3.3 equiv.) was added to the mixture at 0° C., and the mixture was stirred at 20° C. for 17 h under N₂ atmosphere. The reaction mixture was concentrated under reduced pressure to remove DCM, and the resulting residue was diluted with H₂O and extracted 3× with EtOAc. The combined organic layers were washed with brine, dried over Na₂SO₄, filtered, and the filtrate was concentrated under reduced pressure to give a residue. The residue was purified by column chromatography to afford product as a gray solid (51%). ¹H NMR (400 MHz, (CD₃)₂SO) δ 7.15-7.07 (m, 2H), 6.77-6.68 (m, 2H), 3.58 (s, 3H), 3.54 (s, 2H), 2.30 (ddt, J=10.1, 7.3, 3.7 Hz, 1H), 1.69-1.62 (m, 2H), 1.37 (td, J=12.6, 3.5 Hz, 2H), 1.12-1.02 (m, 2H), 0.87-0.78 (m, 2H), 0.13-0.04 (m, 2H).

### Intermediate 4e: spiro[2.5]octan-6-amine

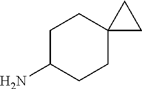

To a suspension of Pd/C (10% w/w, 1.0 equiv.) in MeOH (0.25 M) was added Intermediate 4d (2 g, 1.0 equiv.) and the mixture was stirred at 80° C. at 50 Psi for 24 h under H₂ atmosphere. The reaction mixture was filtered, and the filtrate was concentrated under reduced pressure to give a residue that was purified by column chromatography to afford product as a white solid. ¹H NMR (400 MHz, (CD₃)₂SO) δ 2.61 (tt, J=10.8, 3.9 Hz, 1H), 1.63 (ddd, J=9.6, 5.1, 2.2 Hz, 2H), 1.47 (td, J=12.8, 3.5 Hz, 2H), 1.21-1.06 (m, 2H), 0.82-0.72 (m, 2H), 0.14-0.05 (m, 2H).

### Intermediate 4f: ethyl 2-chloro-4-(spiro[2.5]octan-6-ylamino)pyrimidine-5-carboxylate

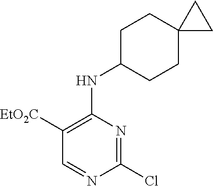 [SMILES: CCOC(=O)c1cnc(Cl)nc1NC1CCC2(CC1)CC2]

Intermediate 4f was synthesized (54%) from Intermediate 4e using the method employed in Intermediate 1e. ¹H NMR (400 MHz, (CD₃)₂SO) δ 8.64 (s, 1H), 8.41 (d, J=7.9 Hz, 1H), 4.33 (q, J=7.1 Hz, 2H), 4.08 (d, J=9.8 Hz, 1H), 1.90 (dd, J=12.7, 4.8 Hz, 2H), 1.64 (t, J=12.3 Hz, 2H), 1.52 (q, J=10.7, 9.1 Hz, 2H), 1.33 (t, J=7.1 Hz, 3H), 1.12 (d, J=13.0 Hz, 2H), 0.40-0.21 (m, 4H).

### Intermediate 4g: 2-chloro-4-(spiro[2.5]octan-6-ylamino)pyrimidine-5-carboxylic acid

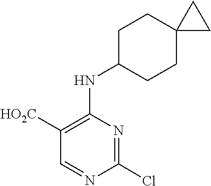 [SMILES: O=C(O)c1cnc(Cl)nc1NC1CCC2(CC1)CC2]

Intermediate 4g was synthesized (82%) from Intermediate 4f using the method employed in Intermediate if ¹H NMR (400 MHz, (CD₃)₂SO) δ 13.54 (s, 1H), 8.38 (d, J=8.0 Hz, 1H), 8.35 (s, 1H), 3.82 (qt, J=8.2, 3.7 Hz, 1H), 1.66 (dq, J=12.8, 4.1 Hz, 2H), 1.47-1.34 (m, 2H), 1.33-1.20 (m, 2H), 0.86 (dt, J=13.6, 4.2 Hz, 2H), 0.08 (dd, J=8.3, 4.8 Hz, 4H).

### Intermediate 4h: 2-chloro-9-(spiro[2.5]octan-6-yl)-7,9-dihydro-8H-purin-8-one

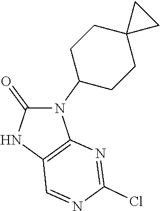 [SMILES: O=c1[nH]c2cnc(Cl)nc2n1C1CCC2(CC1)CC2]

Intermediate 4h was synthesized (67%) from Intermediate 4g using the method employed in Intermediate 1g. ¹H NMR (400 MHz, (CD₃)₂SO) δ 11.68 (s, 1H), 8.18 (s, 1H), 4.26 (ddt, J=12.3, 7.5, 3.7 Hz, 1H), 2.42 (qd, J=12.6, 3.7 Hz, 2H), 1.95 (td, J=13.3, 3.5 Hz, 2H), 1.82-1.69 (m, 2H), 1.08-0.95 (m, 2H), 0.39 (tdq, J=11.6, 8.7, 4.2, 3.5 Hz, 4H).

### Intermediate 4i: 2-chloro-7-methyl-9-(spiro[2.5]octan-6-yl)-7,9-dihydro-8H-purin-8-one

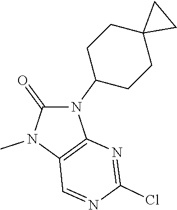 [SMILES: Cn1c(=O)n(C2CCC3(CC2)CC3)c2nc(Cl)ncc21]

Intermediate 4i was synthesized (67%) from Intermediate 4h using the method employed in Intermediate 1h. ¹H NMR (400 MHz, CDCl₃) δ 7.57 (s, 1H), 4.03 (tt, J=12.5, 3.9 Hz, 1H), 3.03 (s, 3H), 2.17 (qd, J=12.6, 3.8 Hz, 2H), 1.60 (td, J=13.4, 3.6 Hz, 2H), 1.47-1.34 (m, 2H), 1.07 (s, 1H), 0.63 (dp, J=14.0, 2.5 Hz, 2H), −0.05 (s, 4H).

Compound 4: 7-methyl-2-((7-methyl-[1,2,4]triazolo[1,5-a]pyridin-6-yl)amino)-9-(spiro[2.5]octan-6-yl)-7,9-dihydro-8H-purin-8-one

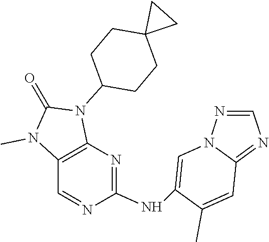 [SMILES: Cc1cc2ncnn2cc1Nc1ncc2c(n1)n(C1CCC3(CC1)CC3)c(=O)n2C]

Compound 4 was synthesized from Intermediate 4i and Intermediate 1d using the method employed in Compound 1. ¹H NMR (400 MHz, (CD₃)₂SO) δ 9.09 (s, 1H), 8.73 (s, 1H), 8.44 (s, 1H), 8.16 (s, 1H), 7.78 (s, 1H), 4.21 (t, J=12.5 Hz, 1H), 3.36 (s, 3H), 2.43 (s, 3H), 2.34 (dt, J=13.0, 6.5 Hz, 2H), 1.93-1.77 (m, 2H), 1.77-1.62 (m, 2H), 0.91 (d, J=13.2 Hz, 2H), 0.31 (t, J=7.1 Hz, 2H). MS: 405.5 m/z [M+H].

### Example 6—Compound 5

### Intermediate 5a: ethyl 2-chloro-4-((3-hydroxycyclobutyl)amino)pyrimidine-5-carboxylate

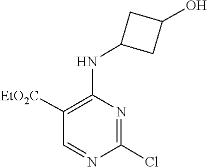 [SMILES: CCOC(=O)c1cnc(Cl)nc1NC1CC(O)C1]

Intermediate 5a was synthesized (49%) from 3-aminocyclobutanol using the method employed in Intermediate 1e. ¹H NMR (400 MHz, (CD₃)₂SO, mixture of rotamers) δ 8.62 (s, 1H), 8.45 (dd, J=25.7, 7.1 Hz, 1H), 5.17 (dd, J=6.0, 2.7 Hz, 1H), 4.32 (q, J=7.1 Hz, 2H), 3.96 (dp, J=50.4, 7.2 Hz, 2H), 2.67 (ddd, J=11.6, 5.8, 2.8 Hz, 1H), 2.25 (td, J=8.1, 7.0, 4.0 Hz, 1H), 1.85 (qd, J=8.7, 2.8 Hz, 1H), 1.32 (t, J=7.1 Hz, 3H).

### Intermediate 5b: 2-chloro-4-((3-hydroxycyclobutyl)amino)pyrimidine-5-carboxylic acid

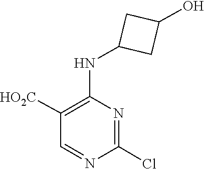 [SMILES: O=C(O)c1cnc(Cl)nc1NC1CC(O)C1]

Intermediate 5b was synthesized (67%) from Intermediate 5a using the method employed in Intermediate 1f. ¹H NMR (400 MHz, (CD₃)₂SO, mixture of rotamers) δ 13.82 (s, 1H), 8.70 (dd, J=25.0, 7.1 Hz, 1H), 8.63 (s, 1H), 4.65-4.29 (m, 1H), 4.17-4.02 (m, 1H), 3.95 (p, J=7.2 Hz, 1H), 2.74 (dh, J=11.8, 3.1 Hz, 2H), 2.30 (t, J=6.2 Hz, 1H), 1.88 (qd, J=8.5, 2.8 Hz, 1H).

### Intermediate 5c: 2-chloro-9-(3-hydroxycyclobutyl)-7,9-dihydro-8H-purin-8-one

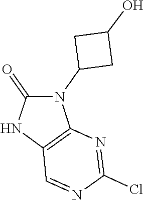 [SMILES: O=c1[nH]c2cnc(Cl)nc2n1C1CC(O)C1]

Intermediate 5c was synthesized from Intermediate 5b using the method employed in Intermediate 1g. ¹H NMR (400 MHz, (CD₃)₂SO, mixture of rotamers) δ 8.12 (d, J=1.7 Hz, 1H), 7.29-7.13 (m, 1H), 4.26 (tt, J=9.8, 7.5 Hz, 1H), 4.00-3.87 (m, 1H), 2.78 (dtd, J=9.9, 8.1, 2.8 Hz, 2H), 2.59-2.52 (m, 2H).

### Intermediate 5d: 2-chloro-9-(3-hydroxycyclobutyl)-7-methyl-7,9-dihydro-8H-purin-8-one

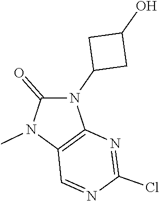 [SMILES: Cn1c(=O)n(C2CC(O)C2)c2nc(Cl)ncc21]

Intermediate 5d was synthesized (61%) from Intermediate 5c using the method employed in Intermediate 1h. ¹H NMR (400 MHz, (CD₃)₂SO) δ 8.32 (d, J=2.4 Hz, 1H), 4.26 (tt, J=9.8, 7.5 Hz, 1H), 3.98-3.85 (m, 1H), 3.31 (d, J=2.4 Hz, 3H), 2.81-2.65 (m, 2H), 2.53 (ddt, J=7.5, 4.1, 2.0 Hz, 2H).

### Compound 5: 9-(3-hydroxycyclobutyl)-7-methyl-2-((7-methyl-[1,2,4]triazolo[1,5-a]pyridin-6-yl)amino)-7,9-dihydro-8H-purin-8-one

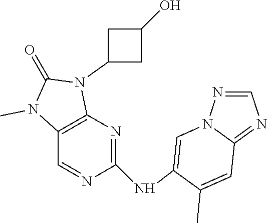 [SMILES: Cc1cc2ncnn2cc1Nc1ncc2c(n1)n(C1CC(O)C1)c(=O)n2C]

Compound 5 was synthesized from Intermediate 5d and Intermediate 1d using the method employed in Compound 1. ¹H NMR (400 MHz, (CD₃)₂SO) δ 9.15 (s, 1H), 8.61 (s, 1H), 8.38 (s, 1H), 8.10 (s, 1H), 7.72 (s, 1H), 5.15 (d, J=6.1 Hz, 1H), 4.26-4.17 (m, 1H), 3.94 (hept, J=6.8 Hz, 1H), 3.30 (s, 3H), 2.78 (qd, J=8.3, 2.6 Hz, 2H), 2.61-2.54 (m, 2H), 2.41-2.39 (m, 3H). MS: 367.4 m/z [M+H].

### Example 7—Compound 6

### Intermediate 6a: ethyl 6-chloro-4-((tetrahydro-2H-pyran-4-yl)amino)nicotinate

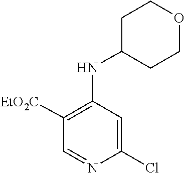 [SMILES: CCOC(=O)c1cnc(Cl)cc1NC1CCOCC1]

Intermediate 6a was synthesized (46%) from 4,6-dichloropyridine-3-carboxylate and tetrahydropyran-4-amine using the method employed in Intermediate 1e. ¹H NMR (400 MHz, (CD₃)₂SO) δ 8.61 (s, 1H), 8.13 (d, J=7.9 Hz, 1H), 7.05 (s, 1H), 4.36 (q, J=7.1 Hz, 2H), 3.90 (dt, J=11.7, 3.8 Hz, 3H), 3.54 (td, J=11.4, 2.2 Hz, 2H), 1.96 (dd, J=12.6, 3.6 Hz, 2H), 1.52 (dtd,J=12.7, 10.6, 4.3 Hz, 2H), 1.38 (t, J=7.1 Hz, 3H).

### Intermediate 6b: 6-chloro-4-((tetrahydro-2H-pyran-4-yl)amino)nicotinic acid

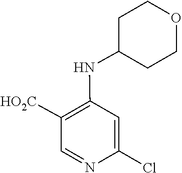 [SMILES: O=C(O)c1cnc(Cl)cc1NC1CCOCC1]

Intermediate 6b was synthesized (74%) from Intermediate 6a using the method employed in Intermediate 1f. ¹H NMR (400 MHz, (CD₃)₂SO) δ 8.57 (s, 1H), 8.36 (d, J=8.0 Hz, 1H), 7.00 (s, 1H), 3.92-3.81 (m, 3H), 3.54 (td, J=11.4, 2.2 Hz, 3H), 2.04-1.90 (m, 2H), 1.56-1.42 (m, 2H).

### Intermediate 6c: 6-chloro-1-(tetrahydro-2H-pyran-4-yl)-1,3-dihydro-2H-imidazo[4,5-c]pyridin-2-one

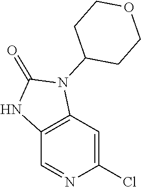 [SMILES: O=c1[nH]c2cnc(Cl)cc2n1C1CCOCC1]

Intermediate 6c was synthesized (76%) from Intermediate 6b using the method employed in Intermediate 1g. ¹H NMR (400 MHz, (CD₃)₂SO) δ 11.32 (s, 1H), 7.94 (s, 1H), 7.44 (s, 1H), 4.38 (tt, J=12.2, 4.2 Hz, 1H), 3.94 (dd, J=11.5, 4.5 Hz, 2H), 3.42 (td, J=11.9, 1.9 Hz, 2H), 2.31 (qd, J=12.4, 4.6 Hz, 2H), 1.69-1.56 (m, 2H).

### Intermediate 6d: 6-chloro-3-methyl-1-(tetrahydro-2H-pyran-4-yl)-1,3-dihydro-2H-imidazo[4,5-c]pyridin-2-one

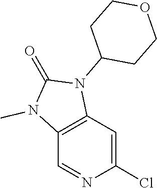 [SMILES: Cn1c(=O)n(C2CCOCC2)c2cc(Cl)ncc21]

Intermediate 6d was synthesized (63%) from Intermediate 6c in 2:1 THF/H-20 using the method employed in Intermediate 1h. ¹H NMR (400 MHz, (CD3)₂SO) δ 8.15 (s, 1H), 7.50 (s, 1H), 4.43 (tt, J=12.1, 4.2 Hz, 1H), 3.94 (dd, J=11.5, 4.5 Hz, 2H), 3.43 (td, J=11.9, 1.9 Hz, 2H), 3.32 (s, 3H), 2.32 (qd, J=12.4, 4.6 Hz, 2H), 1.63 (ddd, J=12.2, 4.3, 1.9 Hz, 2H).

### Compound 6: 3-methyl-6-((7-methyl-[1,2,4]triazolo[1,5-a]pyridin-6-yl)amino)-1-(tetrahydro-2H-pyran-4-yl)-1,3-dihydro-2H-imidazo[4,5-c]pyridin-2-one

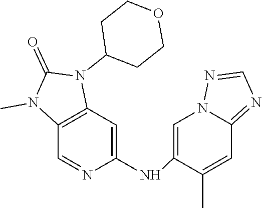 [SMILES: Cc1cc2ncnn2cc1Nc1cc2c(cn1)n(C)c(=O)n2C1CCOCC1]

Compound 6 was synthesized from Intermediate 6d and Intermediate if using the method employed in Compound 1. ¹H NMR (400 MHz, (CD₃)₂SO) δ 9.78 (s, 1H), 8.31 (s, 1H), 8.03 (s, 1H), 8.00 (s, 1H), 7.69 (s, 1H), 7.24 (s, 1H), 4.44 (d, J=12.5 Hz, 1H), 4.04 (dd, J=11.6, 4.4 Hz, 2H), 3.52 (t, J=11.7 Hz, 2H), 2.50-2.46 (m, 3H), 2.32 (tt, J=12.3, 7.0 Hz, 2H), 1.75-1.67 (m, 2H). MS: 380.4 m/z [M+H].

### Example 8—Compound 7

### Intermediate 7a: 4,6-dimethyl-5-nitropyridin-2-amine

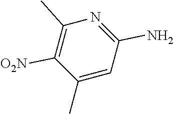

To a solution of 4,6-dimethylpyridin-2-amine (50 g, 1.0 equiv.) in H₂SO₄ was added a mixture of HNO₃ (3.25 equiv.) and H₂SO₄ (2.3 equiv.) at −10° C. dropwise. After addition, the mixture was stirred at this temperature for 1 h. The reaction mixture was quenched by addition NH₃·H₂O at 0° C. dropwise, diluted with H₂O, and extracted 3× with EtOAc. The combined organic layers were concentrated under reduced pressure to give a residue and purified by column chromatography to afford product as a yellow solid (19%). ¹H NMR (400 MHz, CDCl₃) δ 7.70 (s, 1H), 2.53 (s, 3H), 2.45 (s, 3H).

### Intermediate 7b: (E)-NC(4,6-dimethyl-5-nitropyridin-2-yl)-N,N-dimethylformimidamide

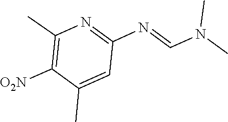 [SMILES: Cc1cc(/N=C/N(C)C)nc(C)c1[N+](=O)[O-]]

To a solution of Intermediate 7a (11.8 g, 1.0 equiv.) and DMF-DMA (1.1 equiv.) in toluene (0.7 M) was degassed and purged 3× with N₂ and the mixture was stirred at 110° C. for 2 h under N atmosphere. The reaction mixture was concentrated under reduced pressure to give a residue and used directly in the next reaction without additional purification.

### Intermediate 7c: (E)-NC(4,6-dimethyl-5-nitropyridin-2-yl)-N-hydroxyformimidamide

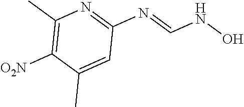

A mixture of Intermediate 7b (10 g, 1.0 equiv.), hydroxylamine hydrochloride (2.0 equiv.) in MeOH (0.4-0.5 M) was degassed and purged 3× with N₂, and the mixture was stirred at 80° C. for 1 h under N₂ atmosphere. The reaction mixture was concentrated under reduced pressure, and the resulting residue was diluted with aq. NaHCO₃ and extracted 3× with EtOAc. The combined organic layers were concentrated under reduced pressure and purified by column chromatography to afford product (19%) as a yellow solid. ¹H NMR (400 MHz, CDCl₃) δ 9.97 (d, J=9.6 Hz, 1H), 8.27 (d, J=9.5 Hz, 1H), 6.68 (s, 1H), 2.54 (s, 3H), 2.46 (s, 3H).

### Intermediate 7d: 5,7-dimethyl-6-nitro-[1,2,4]triazolo[1,5-a]pyridine

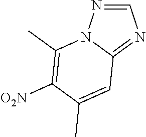

To a mixture of Intermediate 7c (2.2 g, 1.0 equiv.) in THE (0.5 M) was added TFAA (1.5 equiv.). The mixture was stirred at 25° C. for 18 h under N₂ atmosphere. The reaction mixture was concentrated under reduced pressure to remove solvent. The residue was diluted with aq. NaHCO₃ and extracted 3× with EtOAc. The combined organic layers were concentrated under reduced pressure and purified by column chromatography to afford product as a pale yellow solid (55%).

### Intermediate 7e: 5,7-dimethyl-[1,2,4]triazolo[1,5-a]pyridin-6-amine

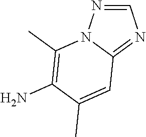

To a solution of Intermediate 7d (1.1 g, 1.0 equiv.) in EtOH (0.5-0.6 M) was added NH₄CO₂H (1.0 equiv.) and Pd/C (10% w/w, 1.0 eqiuv.). The mixture was stirred at 105° C. for 2 h. The reaction mixture was filtered, concentrated under reduced pressure, and purified by column chromatography to afford product as a white solid (64%). ¹H NMR (400 MHz, (CD₃)₂SO) δ 8.15 (s, 1H), 7.38 (s, 1H), 4.77 (s, 2H), 2.58 (s, 3H), 2.28 (s, 3H).

### Compound 7: 6-((5,7-dimethyl-[1,2,4]triazolo[1,5-a]pyridin-6-yl)amino)-3-methyl-1-(tetrahydro-2H-pyran-4-yl)-1,3-dihydro-2H-imidazo[4,5-c]pyridin-2-one

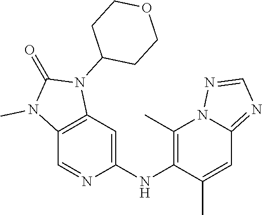 [SMILES: Cc1cc2ncnn2c(C)c1Nc1cc2c(cn1)n(C)c(=O)n2C1CCOCC1]

A mixture of Intermediate 7e (1.0 equiv.), Intermediate 6d (1.0 equiv.), BrettPhos Pd G3 (0.1 equiv.), Cs₂CO₃ (2.0 equiv.) in DMF (0.15 M) was degassed and purged 3× with N₂, and the mixture was stirred at 100° C. for 18 h under N₂ atmosphere. The reaction mixture was poured into water and extracted 3× with DCM. The combined organic phase was washed with brine, dried with anhydrous Na₂SO₄, filtered, and the filtrate was concentrated in vacuum. The residue was purified by column chromatography to afford product as a pale yellow solid. ¹H NMR (400 MHz, (CD₃)₂SO) δ 8.44 (s, 1H), 8.05 (s, 1H), 7.71 (s, 1H), 7.63 (s, 1H), 6.68 (s, 1H), 4.06-3.96 (m, 2H), 3.50 (d, J=11.9 Hz, 2H), 3.26 (s, 3H), 2.60 (s, 3H), 2.29 (s, 5H), 1.70 (d, J=11.5 Hz, 2H). MS: 394.4 m/z [M+H].

### Example 9—Compound 8

### Compound 8: 2-((5,7-dimethyl-[1,2,4]triazolo[1,5-a]pyridin-6-yl)amino)-7-methyl-9-(tetrahydro-2H-pyran-4-yl)-7,9-dihydro-8H-purin-8-one

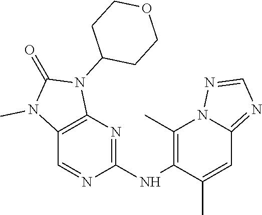 [SMILES: Cc1cc2ncnn2c(C)c1Nc1ncc2c(n1)n(C1CCOCC1)c(=O)n2C]

A mixture of Intermediate 7e (1.0 equiv.), Intermediate 1h (1.0 equiv.), Cs₂CO₃ (2.0 equiv.), Pd(dppf)Cl₂ (0.2 equiv.), XantPhos (0.4 equiv.) in DMF (0.1-0.2 M) was degassed and purged 3× with N₂ and then the mixture was stirred at 130° C. for 24 h under N₂ atmosphere. The mixture was poured into water and extracted 3× with DCM. The combined organic phase was washed with brine, dried with anhydrous Na₂SO₄, filtered, and the filtrate was concentrated in vacuum. The residue was purified by column chromatography to afford product as a brown solid. ¹H NMR (400 MHz, (CD₃)₂SO) δ 8.76 (s, 1H), 8.49 (s, 1H), 7.98 (s, 1H), 7.68 (s, 1H), 4.44 (s, 1H), 4.03-3.93 (m, 2H), 3.47 (d, J=12.5 Hz, 2H), 3.32 (s, 3H), 2.64 (s, 3H), 2.34 (s, 3H), 1.71 (d, J=12.3 Hz, 2H). MS: 395.4 m/z [M+H].

### Example 10—Compound 9

### Intermediate 9a: ethyl 4,6-dichloro-5-methylnicotinate

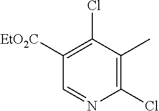

To a mixture of 4,6-dichloro-5-methyl-pyridine-3-carboxylic acid (1.8 g, 1.0 equiv.) in EtOH (0.4-0.5 M) was added H₂SO₄ (1.0 equiv.) dropwise. The mixture was stirred at 80° C. and stirred for 12 h. The reaction mixture was poured into aq. NaHCO₃ and extracted 2× with EtOAc. The combined organic phase was washed with brine, dried with anhydrous Na₂SO₄, filtered, and the filtrate was concentrated in vacuum. The residue was purified by column chromatography to afford product as a colorless oil (59%). ¹H NMR (400 MHz, CDCl₃) δ 8.55 (d, J=4.2 Hz, 1H), 4.36 (pd, J=6.9, 3.9 Hz, 2H), 2.49 (d, J=4.3 Hz, 3H), 1.36 (td, J=7.3, 4.0 Hz, 3H).

### Intermediate 9b: ethyl 6-chloro-5-methyl-4-((tetrahydro-2H-pyran-4-yl)amino)nicotinate

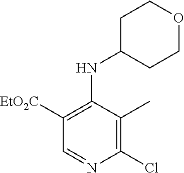 [SMILES: CCOC(=O)c1cnc(Cl)c(C)c1NC1CCOCC1]

Intermediate 9b was synthesized (50%) from Intermediate 9a using the method employed in Intermediate 1e. ¹H NMR (400 MHz, (CD₃)₂SO) δ 8.44 (s, 1H), 7.43 (d, J=9.3 Hz, 1H), 4.31 (q, J=7.1 Hz, 2H), 3.79 (dt, J=11.7, 3.8 Hz, 2H), 3.67 (tq, J=9.7, 4.9, 4.1 Hz, 1H), 3.36 (dd, J=11.5, 2.2 Hz, 2H), 2.30 (s, 3H), 1.84-1.75 (m, 2H), 1.41 (dtd, J=17.5, 10.6, 9.6, 4.4 Hz, 2H), 1.31 (t, J=7.1 Hz, 3H).

### Intermediate 9c: 6-chloro-5-methyl-4-((tetrahydro-2H-pyran-4-yl)amino)nicotinic acid

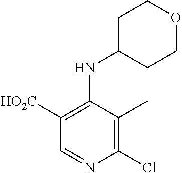 [SMILES: Cc1c(Cl)ncc(C(=O)O)c1NC1CCOCC1]

Intermediate 9c was synthesized (83%) from Intermediate 9b using the method employed in Intermediate if.

### Intermediate 9d: 6-chloro-7-methyl-1-(tetrahydro-2H-pyran-4-yl)-1,3-dihydro-2H-imidazo[4,5-c]pyridin-2-one

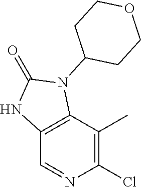 [SMILES: Cc1c(Cl)ncc2[nH]c(=O)n(C3CCOCC3)c12]

To a mixture of Intermediate 9c (0.45 g, 1.0 equiv.), Et₃N (1.0 equiv.) in DMA (0.16 M) was added DPPA (1.0 equiv.). The mixture was stirred at 120° C. for 8 h under N₂ atmosphere. The reaction mixture was poured into water, and the precipitate was collected by filtration, washed with water, and dried under vacuum to give a residue that was used directly in the next reaction without further purification (67%).

### Intermediate 9e: 6-chloro-3,7-dimethyl-1-(tetrahydro-2H-pyran-4-yl)-1,3-dihydro-2H-imidazo[4,5-c]pyridin-2-one

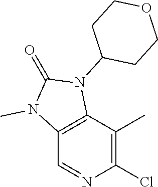 [SMILES: Cc1c(Cl)ncc2c1n(C1CCOCC1)c(=O)n2C]

Intermediate 9e was synthesized (79%) from Intermediate 9d using the method employed in Intermediate 1h. ¹H NMR (400 MHz, CDCl₃) δ 7.79 (s, 1H), 4.55 (tt, J=12.0, 4.2 Hz, 1H), 4.08 (dd, J=11.8, 4.7 Hz, 2H), 3.40 (td, J=12.2, 2.0 Hz, 2H), 2.81 (qd, J=12.5, 4.6 Hz, 2H), 1.66 (ddd, J=12.5, 4.2, 1.9 Hz, 2H).

### Compound 9: 3,7-dimethyl-6-((7-methyl-[1,2,4]triazolo[1,5-a]pyridin-6-yl)amino)-1-(tetrahydro-2H-pyran-4-yl)-1,3-dihydro-2H-imidazo[4,5-c]pyridin-2-one

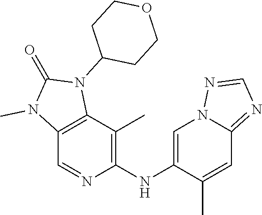 [SMILES: Cc1cc2ncnn2cc1Nc1ncc2c(c1C)n(C1CCOCC1)c(=O)n2C]

Compound 9 was synthesized from Intermediate 1d and Intermediate 9e using the method employed in Compound. 1H NMR (400 MHz, DMSO) δ 8.63 (s, 1H), 8.30 (s, 1H), 7.72 (s, 1H), 7.64 (s, 1H), 7.54 (s, 1H), 4.65-4.56 (m, 1H), 3.95 (dd, J=11.4, 4.5 Hz, 2H), 3.43 (t, J=11.8 Hz, 2H), 3.22 (s, 3H), 2.69-2.52 (m, 2H), 2.50 (s, 3H), 2.21 (d, J=1.1 Hz, 3H), 1.71 (d, J=11.4 Hz, 2H). MS: 394.5 m/z [M+H].

### Example 11—TRAC LNP Treatment of T Cells with or without DNA-PK Inhibitors

T cells were thawed from liquid N2 storage and rested overnight in 2.5% human serum T growth activation media (TCGM: CTS OpTmizer (Thermofisher #A3705001) with 2.5% heat inactivated human AB serum (Gemini #100-512), 1× GlutaMAX (Thermofisher #35050061), 1% Penicillin/Streptomycin (Thermofisher #15140-122), 10 mM HEPES pH 7.4 (Thermofisher #15630080), IL-2 (200 U/mL, Peptrotech #200-02), IL-7 (5 ng/mL, Peptrotech #200-07), and IL-15 (5 ng/mL, Peptrotech #200-15).

After overnight rest T cells were activated with TransAct (1:100 dilution, Miltenyi) for 48 hours prior to insertion. T cells were harvested, washed, and resuspended in TCGM without serum to a concentration of 1.25×10⁶ cells/mL. LNP-ApoE solution was prepared at an LNP concentration of 5 μg/mL in 5% human serum TCGM with 1 μg/mL ApoE3 and incubated in 37 degree for 10 min. LNP compositions were formulated with mRNA encoding Cas9 (SEQ ID: NO 8) and sgRNA targeting human TRAC (G013006 SEQ ID: 1) as described in Example 1 at a lipid molar ratio of 50/38.5/10/1.5 or 35/47.5/15/2.5 of component lipids Lipid A, cholesterol, DSPC, and PEG2k-DMG, respectively. The cargo ratio of sgRNA to Cas9 mRNA was 1:2 by weight. The LNP-ApoE mix and T cells (50,000 cells/well) were mixed 1:1 by volume. AAV encoding a homology directed repair template for insertion of a GFP open reading frame (OFR) into the TRAC locus (SEQ ID NO: 13) was added at a MOI of 3×10⁵ viral genomes/cell. DNA-PK inhibitors (Compound 1, Compound 2, Compound 3, Compound 4, Compound 5, Compound 6, Compound 7, Compound 8, or Compound 9) were diluted in 2.5% serum TCGM and added to cells to achieve a final concentration of 1, 0.25, or 0.0625 μM. The next day T cells were spun down in 96-well plate at 500 g for 5 min to remove the media, washed once, and resuspended in 2.5% serum TCGM, and expanded for 5 days. During the 5-day expansion, cells were split once at day 2 into new 2.5% serum TCGM to prevent overgrowth.

### 11.1. Flow Cytometry

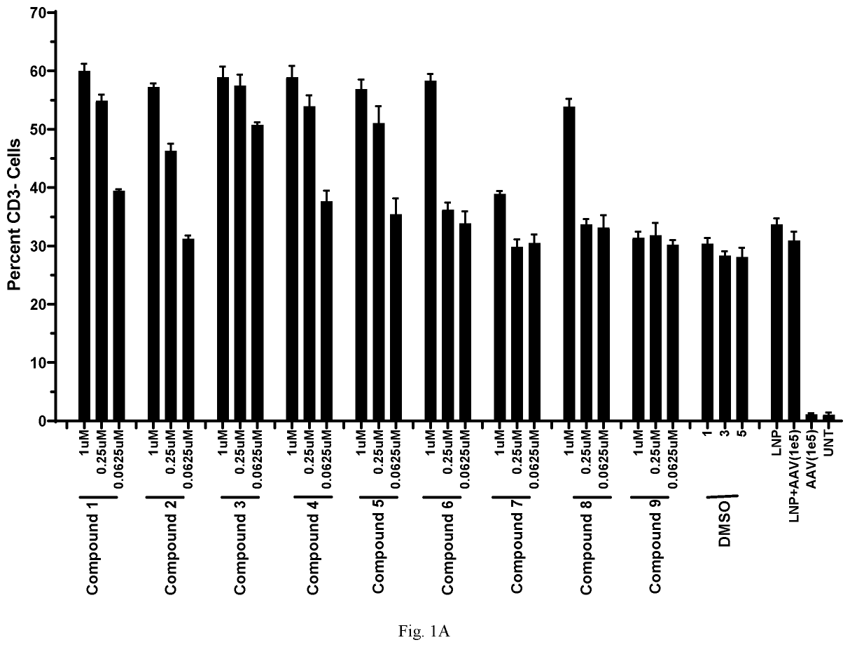

On day 5 post-edit, T cells were phenotyped by flow cytometry to determine endogenous TCR knockout and GFP insertion. The T cell receptor alpha chain encoded by TRAC is required for T cell receptor/CD3 complex assembly and translocation to the cell surface. Accordingly, disruption of the TRAC gene by genome editing leads to a loss of CD3 protein on the cell surface of T cells. Briefly, edited T cells were stained with FACS buffer (PBS pH 7.4, 2% FBS, 1 mM EDTA) containing antibody targeting CD3 (1:200) and incubated on ice, protected from light for 30 minutes. Cells were subsequently washed and resuspended in FACS buffer containing DAPI (1:5,000) and incubated on ice, protected from light for 10 min. Post-staining, T cells were washed, resuspended in FACS buffer, and analyzed using a CytoFLEX LX cytometer. T cells were gated on size, DAPI staining, and GFP and CD3 expression. Results are shown in Table 2 and FIG. 1A. GFP positive cells were gated within the CD3 negative population as in Table 3 and FIG. 1B.

TABLE 2
Percent CD3 negative T cells following editing
in the presence of indicated DNAPK inhibitors

| | Compound | | Mean % CD3⁻ | SD % CD3⁻ |
|---|---|---|---|---|
| Compound | Concentration | | Cells(n = 3) | Cells |
| Compound 1 | 1 | uM | 60.00 | 1.23 |
| | 0.25 | uM | 54.87 | 1.08 |
| | 0.0625 | uM | 39.43 | 0.25 |
| Compound 2 | 1 | uM | 57.23 | 0.64 |
| | 0.25 | uM | 46.30 | 1.21 |
| | 0.0625 | uM | 31.20 | 0.56 |
| Compound 3 | 1 | uM | 58.90 | 1.82 |
| | 0.25 | uM | 57.47 | 1.88 |
| | 0.0625 | uM | 50.73 | 0.42 |
| Compound 4 | 1 | uM | 58.93 | 1.94 |
| | 0.25 | uM | 53.93 | 1.85 |
| | 0.0625 | uM | 37.60 | 1.85 |
| Compound 5 | 1 | uM | 56.83 | 1.70 |
| | 0.25 | uM | 51.03 | 2.90 |
| | 0.0625 | uM | 35.40 | 2.75 |
| Compound 6 | 1 | uM | 58.30 | 1.15 |
| | 0.25 | uM | 36.20 | 1.22 |
| | 0.0625 | uM | 33.87 | 2.07 |
| Compound 7 | 1 | uM | 38.90 | 0.50 |
| | 0.25 | uM | 29.83 | 1.29 |
| | 0.0625 | uM | 30.47 | 1.46 |
| Compound 8 | 1 | uM | 53.87 | 1.36 |
| | 0.25 | uM | 33.67 | 0.91 |
| | 0.0625 | uM | 33.10 | 2.17 |
| Compound 9 | 1 | uM | 31.37 | 1.07 |
| | 0.25 | uM | 31.77 | 2.15 |
| | 0.0625 | uM | 30.20 | 0.79 |

| | | | |
|---|---|---|---|
| DMSO | 1 | 30.37 | 1.01 |
| | 3 | 28.30 | 0.79 |
| | 5 | 28.07 | 1.63 |
| No inhibitor | LNP | 33.63 | 1.07 |
| No inhibitor | LNP + AAV(1e5) | 30.93 | 1.53 |
| No inhibitor | AAV(1e5) | 1.09 | 0.25 |
| No inhibitor | Untreated (UNT) | 1.07 | 0.37 |

TABLE 3
Percent GFP positive of the CD3 negative T cells following
editing in the presence of indicated DNAPK inhibitors

| | | | Mean % CD3⁻/ | SD % CD3⁻/ |
|---|---|---|---|---|
| Compound | Concentration | | GFP+ Cells (n = 3) | GFP+ Cells |
| Compound 1 | 1 | uM | 81.37 | 1.56 |
| | 0.25 | uM | 78.00 | 1.37 |
| | 0.0625 | uM | 65.63 | 0.83 |
| Compound 2 | 1 | uM | 79.40 | 0.82 |
| | 0.25 | uM | 70.07 | 2.31 |
| | 0.0625 | uM | 47.93 | 1.66 |
| Compound 3 | 1 | uM | 79.60 | 1.65 |
| | 0.25 | uM | 79.97 | 0.95 |
| | 0.0625 | uM | 75.50 | 0.70 |
| Compound 4 | 1 | uM | 80.07 | 1.72 |
| | 0.25 | uM | 78.73 | 0.85 |
| | 0.0625 | uM | 58.13 | 3.57 |
| Compound 5 | 1 | uM | 78.97 | 0.15 |
| | 0.25 | uM | 73.30 | 0.96 |
| | 0.0625 | uM | 53.00 | 3.64 |
| Compound 6 | 1 | uM | 74.40 | 1.32 |
| | 0.25 | uM | 53.50 | 1.61 |
| | 0.0625 | uM | 35.10 | 0.69 |
| Compound 7 | 1 | uM | 43.43 | 0.60 |
| | 0.25 | uM | 37.43 | 1.61 |
| | 0.0625 | uM | 29.90 | 1.87 |
| Compound 8 | 1 | uM | 70.07 | 0.85 |
| | 0.25 | uM | 44.43 | 2.72 |
| | 0.0625 | uM | 32.23 | 1.46 |
| Compound 9 | 1 | uM | 27.83 | 1.27 |
| | 0.25 | uM | 28.50 | 2.03 |
| | 0.0625 | uM | 28.27 | 1.90 |

| | | | |
|---|---|---|---|
| DMSO | 1 | 27.10 | 0.85 |
| | 3 | 26.20 | 0.66 |
| | 5 | 25.43 | 1.16 |
| No inhibitor | LNP | 0.13 | 0.07 |
| No inhibitor | LNP + AAV(1e5) | 27.83 | 0.96 |
| No inhibitor | AAV(1e5) | 0.81 | 0.72 |
| No inhibitor | Untreated (UNT) | 0.76 | 0.13 |

### Example 12—Engineering Functionally Active TCR T Cells with CRISPR/Cas9 and DNA-PK Inhibitors

The use of DNA-PK inhibitors to boost transgenic TCR (tgTCR) insertion in T cells without perturbing T cell expansion, cytotoxicity or cytokine release was evaluated.

### 12.1. T Cell Isolation

Healthy human donor apheresis was obtained commercially (HemaCare) from three donors (referred to as 007HD, 008HD, and 009HD). Cells were washed and re-suspended in CliniMACS PBS/EDTA buffer (Miltenyi cat. 130-070-525) on the LOVO device. T cells were isolated via positive selection using CD4 and CD8 magnetic beads (Miltenyi BioTec cat. 130-030-401/130-030-801) using the CliniMACS Plus and CliniMACS LS disposable kit. T cells were aliquoted into vials and cryopreserved in a 1:1 formulation of Cryostor CS10 (StemCell Technologies cat. 07930) and Plasmalyte A (Baxter cat. 2B2522X) for future use.

### 12.2. T Cell Media and Thaw

T cells were thawed from liquid N2 storage and rested overnight in 2.5% human serum T cell activation media (TCAM: CTS OpTmizer (Thermofisher #A3705001) with 2.5% heat inactivated human AB serum (Gemini #100-512), 1× GlutaMAX (Thermofisher #35050061), 1% Penicillin/Streptomycin (Thermofisher #15140-122), 10 mM HEPES pH 7.4 (Thermofisher #15630080), IL-2 (200 U/mL, Peptrotech #200-02), IL-7 (5 ng/mL, Peptrotech #200-07), and IL-15 (5 ng/mL, Peptrotech #200-15)).

### 12.3. T cell Engineering

Rested T cells were counted and resuspended in TCGM at a density of 2×10⁶ cells/mL with TransAct reagent added at a 1:50 dilution. Meanwhile, 5 μg/mL of TRBC-LNP formulated with mRNA encoding Cas9 (SEQ ID NO: 8) and an sgRNA targeting TRBC (G016239) (SEQ ID NO: 2) was incubated in TCAM with 1 μg/mL recombinant human ApoE3 before being mixed 1:1 by volume with T cells and incubated at 37° C. for 48 hours. After 48h of activation, T cells were harvested, washed, and resuspended in TCAM to a concentration of 1×10⁶ cells/mL. TRAC-LNP-ApoE solution was prepared at 5 μg/mL in TCAM with 5 μg/mL ApoE3. The TRAC-LNP was formulated with mRNA encoding Cas9 (SEQ ID NO: 8) and an sgRNA targeting TRAC (G013006) (SEQ ID NO: 1). The LNP-ApoE mix and T cells were mixed 1:1 by volume. An AAV encoding a homology directed repair template for insertion into the TRAC locus of HD1, a WT1-specific TCR, (SEQ ID NO: 13) was added at a MOI of 3×10⁵ viral genomes/cell. DNA-PK inhibitors were added as indicated in Table 4 at a concentration of 0.25 uM. The next day T cells were washed, resuspended in T cell expansion media (TCEM: as described for TCAM with exception of 5% human AB serum instead of 2.5%) before being transferred to GREX plates (Wilson Wolf #80240M), and expanded for 6 additional days with cytokine replenishment every 2-3 days. Control samples were processed as described above with the omission of any DNA-PK inhibitor treatment. Post expansion cells were harvested, counted using Vi-CELL XR Cell Counter, and characterized by flow cytometry. Fold expansion was determined by dividing the total cell count yield at endpoint by the number of cells in each group at Day 0 (i.e. starting material). There was no impact of T cell expansion observed by DNA-PK treatment with compounds 6, 7, and 8 (Table 4). T cells were cryopreserved in Cryostor CS10 freezing media for future analysis.

TABLE 4
Fold T cell expansion following editing in
the presence of indicated DNAPK inhibitors

| Donor | No Inhibitor | Compound 1 | Compound 3 | Compound 4 |
|---|---|---|---|---|
| 007HD | 130.64 | 110.18 | 110.47 | 118.46 |
| 008HD | 104.43 | 114.56 | 111.44 | 108.81 |
| 009HD | 142.91 | 159.08 | 143.01 | 140.77 |
| Mean | 125.99 | 127.94 | 121.64 | 122.68 |
| SD | 19.66 | 27.06 | 18.51 | 16.39 |
| P-value relative | | 0.88 | 0.65 | 0.56 |
| to No Inhibitor | | | | |

### 12.4. Flow Cytometry

After engineering and expansion, T cells were characterized for editing efficiency and memory phenotype using flow cytometry. Briefly, T cells were stained with a cocktail of antibodies targeting CD4, CD8, CD3R, V08, CD45RA, CD45RO, CD62L, and CCR7 diluted in FACS buffer (PBS pH 7.4, 2% FBS, 1 mM EDTA) for 30 minutes at room temperature. Vβ8 antibody recognizes the specific VO-chain used by the WT1-TCR. Post-staining T cells were washed, resuspended in FACS buffer, and analyzed using a CytoFLEX LX cytometer. There was no impact of the inhibitor on the percent of CD8+ T cells within each group (FIG. 2A). We observed a statistically significant increase (p<0.05, Students T test) in the percent of CD8+ cells with WT1-TCR insertion (CD3ε+, Vβ8⁺) in all DNA-PK inhibitor treated groups relative to the non-treated groups (Table 5, FIG. 2C), as well as a trend towards increased endogenous TCR KO (FIG. 2B).

TABLE 5
Percent CD3ε+, Vβ8+ cells among CD8+ cells following
editing in the presence of indicated DNAPK inhibitors.

| Donor | No Inhibitor | Compound 1 | Compound 3 | Compound 4 |
|---|---|---|---|---|
| 007HD | 59.3 | 87.7 | 88.9 | 87.7 |
| 008HD | 59.7 | 85 | 84.6 | 83.6 |
| 009HD | 47.2 | 81.3 | 82 | 80.5 |
| Mean | 55.4 | 84.7 | 85.2 | 83.9 |
| SD | 7.1 | 3.2 | 3.5 | 3.6 |

| | | | |
|---|---|---|---|
| P-value relative | 0.008 | 0.009 | 0.009 |
| to No Inhibitor | | | |

### 12.5. WT1 TCR T Cell Mediated Cytotoxicity and Cytokine Release in Response to 697 ALL and K562-HLA-A*02:01 CML Cell Lines

The ability of WT1-TCR T cells engineered from donors 007HD and 008HD to kill hematological cancer cells expressing natural levels of WT1 and release cytokines was evaluated. TCR KO cells generated as described above but without TRAC/AAV addition were used as a negative, non-killing control. Briefly, T cells were co-cultured with luciferase expressing 697 ALL cells (697-luc2), or K562-luc2 cells transduced to express HLA-A*02:01 (K5692-HLA-A*02:01-luc2) at various effector to target (E:T) ratios (2:1, 1:1, 0.5:1) in TCEM without the addition of IL-2, IL-7, or IL-15. Notably effector to target ratios were normalized for relative WT1 TCR insertion in each group leading to the same amount of absolute WT1 TCR expressing cells across inhibitor and no treatment groups. After 24h of co-culture, supernatants from the 2:1 E:T ratio group were harvested and used in an MSD U/R-PLEX assay to quantify IL-2, TNFα, IFNγ, and Granzyme B following manufacturers protocol (Mesoscale Discovery). After 48h of co-culture luciferase activity was quantified using the Bright-GLO Luciferase Assay System (Promega) in relative luminescence units (RLU). Percent specific lysis was determined using the formula:

% Specific Lysis=100−((RLU[Experimental Well]/RLU[Target Only Well])*100)

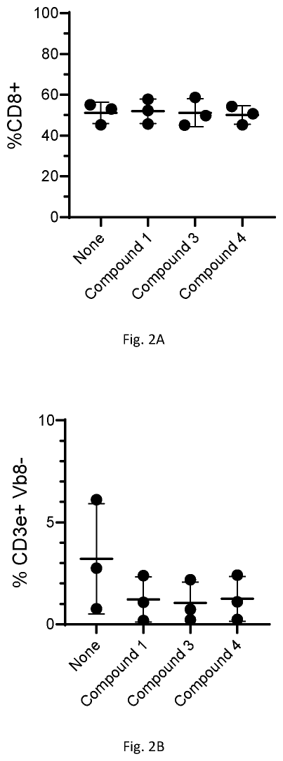

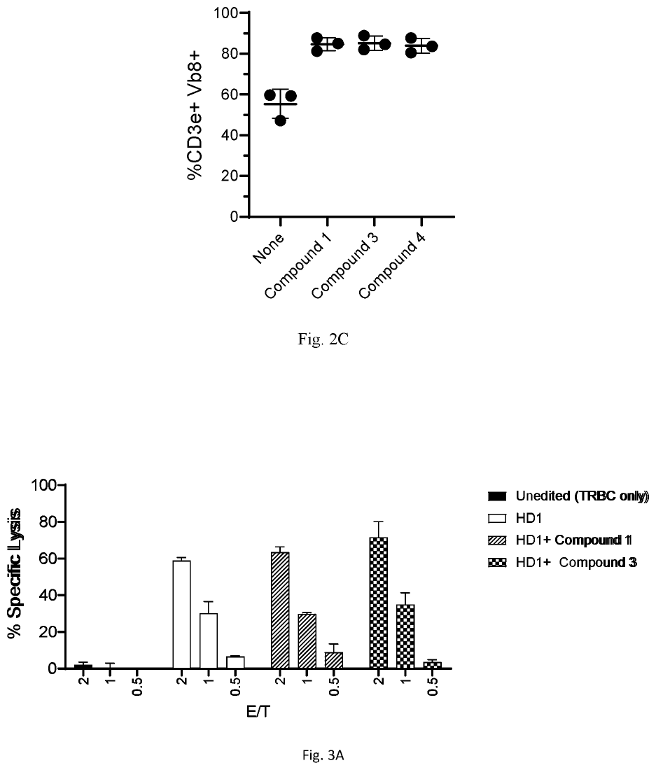

Results from the cytotoxicity assay are shown in FIGS. 3A-3D and Table 6, while cytokine release is reported in Table 7 and FIGS. 4A-4H. No notable differences on T cell functionality in groups treated with the DNA-PK inhibitors were observed.

TABLE 6
Percent specific lysis by WT1-T cells of hematological cancer lines
697 ALL and K562-HLA-A*2:1 CML with DNApk inhibitor treatment

| | | | Target cells | | | | | | HD1 + | | HD1 + | |
|---|---|---|---|---|---|---|---|---|---|---|---|---|
| E:T | Donor | Target | only | | TCR KO | | HD1 | | Compound 1 | | Compound 3 | |
| 2 | 008HD | 697-ALL | −1.23 | 0.82 | −5.13 | 1.05 | 98.47 | 97.79 | 98.25 | 98.09 | 98.98 | 98.97 |
| 1 | 008HD | 697-ALL | 6.36 | 5.22 | 4.69 | −1.55 | 89.95 | 92.88 | 81.24 | 77.92 | 95.29 | 94.31 |
| 0.5 | 008HD | 697-ALL | −7.58 | −3.07 | −0.16 | 0.43 | 66.16 | 61.16 | 44.29 | 43.47 | 63.10 | 72.62 |
| 2 | 007HD | 697-ALL | 11.92 | 4.41 | 3.20 | 0.99 | 57.73 | 60.09 | 61.43 | 65.54 | 77.64 | 65.49 |
| 1 | 007HD | 697-ALL | −0.13 | 1.13 | 1.39 | −6.18 | 25.72 | 34.75 | 30.44 | 29.28 | 39.43 | 30.39 |
| 0.5 | 007HD | 697-ALL | −2.63 | −10.18 | −13.43 | −23.72 | 6.87 | 6.48 | 5.54 | 12.14 | 4.60 | 2.72 |
| 2 | 008HD | K562-HLA-A*02:01 | −5.39 | 6.94 | 27.43 | 28.91 | 99.91 | 99.91 | 99.66 | 99.29 | 99.96 | 99.84 |
| 1 | 008HD | K562-HLA-A*02:01 | 4.16 | 9.14 | 16.05 | 23.82 | 96.39 | 97.08 | 79.54 | 83.56 | 98.67 | 99.08 |
| 0.5 | 008HD | K562-HLA-A*02:01 | 0.45 | 1.64 | 12.12 | 15.91 | 66.18 | 77.06 | 58.18 | 56.76 | 72.45 | 71.77 |
| 2 | 007HD | K562-HLA-A*02:01 | −3.90 | 7.88 | 11.29 | 12.66 | 81.43 | 83.60 | 92.50 | 95.40 | 98.79 | 96.99 |
| 1 | 007HD | K562-HLA-A*02:01 | −8.53 | 3.90 | 10.94 | 9.09 | 50.55 | 54.44 | 70.95 | 69.32 | 78.53 | 75.83 |
| 0.5 | 007HD | K562-HLA-A*02:01 | −8.18 | 1.78 | 2.32 | 5.70 | 31.39 | 29.30 | 36.80 | 37.10 | 47.03 | 41.52 |

TABLE 7
Quantification of Granzyme B, IFNg, IL-2, and TNF-a cytokines in WT1-T
cells in hematological cancer lines 697 ALL and K562-HLA-A*2:1 CML

| Donor | Target | Analyte | TCR KO | | HD1 | | | HD1 + Compound 3 | | |
|---|---|---|---|---|---|---|---|---|---|---|
| 008HD | 697-ALL | Granzyme B | 155 | 174.52 | 1430.80 | 1713.88 | 1277.93 | 1625.49 | 1609.68 | 1777.28 |
| 007HD | 697-ALL | Granzyme B | 621.7 | 514.38 | 1879.36 | 1500.30 | 1786.77 | 1972.02 | 1664.25 | 1441.06 |
| 008HD | 697-ALL | IFN-gamma | 69.82 | 77.42 | 5495.13 | 5187.14 | 5076.15 | 5420.40 | 6733.05 | 7169.43 |
| 007HD | 697-ALL | IFN-gamma | 254.3 | 173.8 | 905.84 | 586.24 | 1031.41 | 1051.85 | 977.71 | 923.97 |
| 008HD | 697-ALL | IL-2 | 1.1 | 15.41 | 5.44 | 7.14 | 5.91 | 9.12 | 7.14 | 8.1 |
| 007HD | 697-ALL | IL-2 | 2.34 | 1.43 | 1.13 | 1.74 | 2.17 | 2.85 | 4.32 | 2.23 |
| 008HD | 697-ALL | TNF-alpha | <LLOD | 1.61 | 11.87 | 17.08 | 13.97 | 15.53 | 16.3 | 18.57 |
| 007HD | 697-ALL | TNF-alpha | 0.47 | 1.37 | 1.49 | 3.06 | 1.76 | 4.16 | 3.46 | 5.29 |
| 008HD | K562-HLA-A2.1 | Granzyme B | 286.55 | 341.39 | 18412.65 | 17768.81 | 10683.96 | 10971.57 | 18197.02 | 17134.42 |
| 007HD | K562-HLA-A2.1 | Granzyme B | 300.93 | 332.79 | 15390.31 | 18751.76 | 20618.69 | 22750.44 | 20713.6 | 17178.54 |
| 008HD | K562-HLA-A2.1 | IFN-gamma | 276.26 | 363.41 | 182918.7 | 187385.1 | 155269.4 | 141164.2 | 216197.4 | 200584.6 |
| 007HD | K562-HLA-A2.1 | IFN-gamma | 105.58 | 109.31 | 35852.74 | 44189.57 | 55131.36 | 54976.52 | 59686.42 | 41527.06 |
| 008HD | K562-HLA-A2.1 | IL-2 | <LLOD | 3.59 | 315.05 | 344.58 | 431.46 | 378.49 | 524.89 | 484.52 |
| 007HD | K562-HLA-A2.1 | IL-2 | 1.6 | 1.23 | 128.34 | 301.06 | 295.95 | 308.81 | 415.72 | 176.34 |
| 008HD | K562-HLA-A2.1 | TNF-alpha | <LLOD | 2.64 | 242.11 | 314.86 | 166.01 | 155.87 | 324.18 | 302.06 |
| 007HD | K562-HLA-A2.1 | TNF-alpha | <LLOD | 0.16 | 28.07 | 46.98 | 51.48 | 60.7 | 73.76 | 66.56 |

### Example 13—Editing in B Cells Using DNA Protein Kinase Inhibitors

The effect of DNA protein kinase inhibitors (DNA-PKI) on editing efficiency in B cells was assessed.

B cells were isolated from a healthy human donor leukopak by CD19 positive selection using the StraightFrom Leukopak CD19 MicroBead kit (Miltenyi, 130-117-021) on a MultiMACS Cell24 Separator Plus instrument. Following MACS isolation, CD19+ B cells were activated in EVIDM media or Stemspan media and frozen until needed. Base media are EVIDM (Corning, 10-016-CV) or StemSpan SFEM (StemCell Technologies, 9650) supplemented with 1% Penicillin/Streptomycin (Corning, 30-002-CI), 50 ng/ml hIL-2 (Peprotech, 200-02), 50 ng/ml hIL-10 (Peprotech, 200-10), 10 ng/ml hIL-15 (Peprotech, 200-15), 100 ng/ml MEGACD40L, 1 ug/ml CpG ODN 2006 (Invivogen, TLR-2006) and 10% fetal bovine serum (FBS). B cells were thawed and cultured in Stemspan media supplemented with 1% Penicillin/Streptomycin (Corning, 30-002-CI), 50 ng/ml hIL-2 (Peprotech, 200-02), 50 ng/ml hIL-10 (Peprotech, 200-10), 10 ng/ml hIL-15 (Peprotech, 200-15), 1 ng/ml MEGACD40L, 1 ug/ml CpG ODN 2006 (Invivogen, TLR-2006) and 5% human AB Serum (Gemini Bio-Products, 100-512). Following two days of culture, cells were harvested and resuspended at 100,000 cells/100 μl in StemSpan media with 1% Penicillin/Streptomycin, supplemented with 2× the final concentration of the cytokine, 2 ug/ml CpG ODN 2006 (Invivogen, TLR-2006) and 2 ng/ml MEGACD40L prior to treatment with LNP compositions delivering mRNA encoding Cas9 (SEQ ID NO: 8) and gRNA G000529 targeting B2M.

LNPs were generally prepared as described in Example 1 with the lipid composition of 50/38.5/10/1.5, expressed as the molar ratio of ionizable lipid/cholesterol/DSPC/PEG, respectively. LNPs were preincubated at a concentration of 5 μg/ml total RNA cargo with 1.25 μg/ml ApoE4 (Peprotech, 350-04) at 37° C. for about 15 minutes in StemSpan media supplemented with 1% Penicillin/Streptomycin and 5% human AB serum (Gemini Bio-Products, 100-512). The pre-incubated LNPs were added to B cells at a final concentration 2.5 ug/ml total RNA cargo followed by addition of 0.25 ug/ml DNAPK inhibitor Compound 1, Compound 3, or Compound 4.

B cells were phenotyped for the presence of B2M surface protein on day 7 post LNP composition treatment. For this, B cells were incubated with antibodies targeting CD86 (Biolegend, 374216) and B2M (Biolegend, 316312). Cells were subsequently stained with a viability dye (Biolegend, 422801), washed, processed on a Cytoflex instrument (Beckman Coulter) and analyzed using the FlowJo software package. B cells were gated on size and viability status, followed by B2M expression on the total live population. Percent B2M negative cells is shown in Table 8. Increased percentage of B2M negative B cells were observed in the presence of DNA-PKI compared to no DNA-PKI, indicating increased gene editing.

TABLE 8
Percentage of B2M negative cells following editing
with DNA-PKI and LNP composition targeting B2M.

% B2M−

| Sample | Mean | SD |
|---|---|---|
| No Inhibitor | 11.2 | 1.5 |
| Compound 1 | 19.2 | 2.3 |
| Compound 3 | 27.4 | 2.6 |
| Compound 4 | 24.1 | 1.0 |
| No edit | 2.5 | 0.5 |

### 13.2. Editing in B Cells from Multiple Donors Using DNAPK Inhibitors

B cells were isolated from PBMC derived from 3 donors as described in Example 23.1. Following MACS isolation, CD19+ B cells were activated in Stemspan media with 1 ug/ml CpG ODN 2006 (Invivogen, TLR-2006), 2.5% human AB serum (Gemini Bio-Products, 100-512), 1% penicillin-streptomycin (ThermoFisher, 15140122), 50 ng/ml IL-2 (Peprotech, 200-02), 50 ng/ml IL-10 (Peprotech, 200-10), and 10 ng/ml IL-15 (Peprotech, 200-15) and 1 ng/ml CD40L (Enzo Life Sciences, ALX-522-110-C010). Two days following activation, B cells were treated with LNP compositions delivering mRNA encoding Cas9 (SEQ ID NO: 8) and gRNA G000529 targeting B2M. B cells were plated at 50,000 cells per well in triplicate as indicated in Table 9 in complete Stemspan media as described above.

LNPs were generally prepared as in Example 1 with the lipid composition of 50/38.5/10/1.5, expressed as the molar ratio of ionizable lipid/cholesterol/DSPC/PEG, respectively. LNPs were preincubated at 37° C. for 15 minutes with Stemspan media containing 1 μg/mL CpG ODN 2006, 2.5% human AB serum, 1% penicillin-streptomycin, 50 ng/mL IL-2, 50 ng/mL IL-10, and 10 ng/ml IL-15, 1 ng/ml CD40L, and 1.25 μg/mL ApoE4. The pre-incubated LNP compositions were added to B cells at a final concentration 2.5 μg/mL total RNA cargo followed by addition of 0.25 μg/mL DNAPK inhibitor Compound 1 or Compound 4. Seventy-two hours post-LNP composition addition, cells were washed, resuspended in Stemspan media containing 1 μg/mL CpG ODN 2006, 2.5% human AB serum, 1% penicillin-streptomycin, 50 ng/mL IL-2, 50 ng/mL IL-10, and 10 ng/mL IL-15, and 100 ng/mL CD40L and transferred to a 48-well plate.

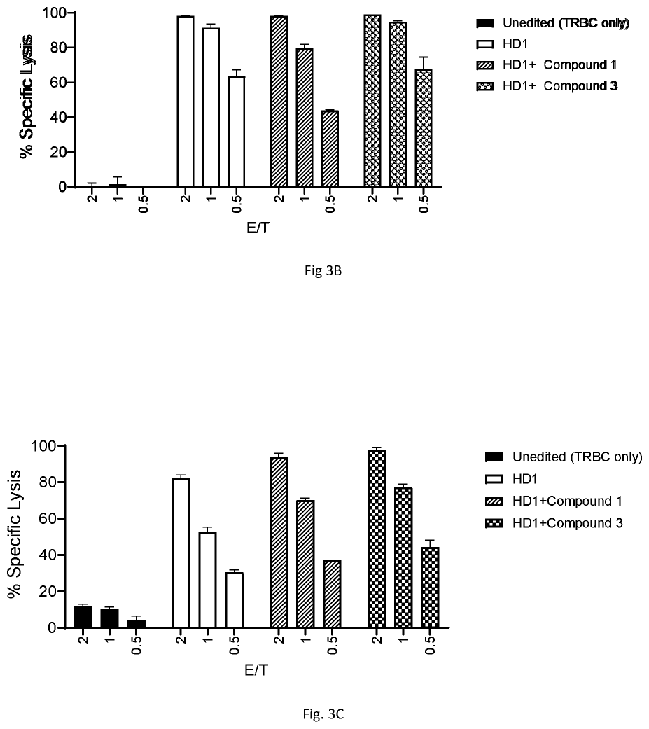

Seven days post-LNP composition treatment, cells were phenotyped by flow cytometry. Briefly, B cells were incubated with antibodies targeting CD19 (Biolegend, 363010A), CD20 (Biolegend, 302322), CD86 (Biolegend, 374216) and B2M (Biolegend, 395806) followed by viability dye DAPI (Biolegend, 422801). Cells were subsequently washed and processed on a Cytoflex instrument (Beckman Coulter) and analyzed using the FlowJo software package. B cells were gated on size and viability status, followed by B2M expression on the total live population. Table 9 and FIG. 5 show mean percent of B2M negative cells following editing with DNAPK inhibitors. Addition of DNAPK inhibitors moderately improved editing efficiency.

TABLE 9
Mean percent B2M negative cells following editing with DNAPK inhibitors

| | Unedited, | | | | | | | | | | | |
|---|---|---|---|---|---|---|---|---|---|---|---|---|
| | No inhibitor | | | No inhibitor | | | Compound 1 | | | Compound 4 | | |
| Donor | Mean | SD | N | Mean | SD | N | Mean | SD | N | Mean | SD | N |
| Donor 150 | 3.74 | 1.62 | 3 | 50.57 | 1.54 | 3 | 56.41 | 4.39 | 3 | 59.42 | 4.16 | 3 |
| Donor 200 | 5.11 | 0.06 | 2 | 27.60 | 4.16 | 3 | 36.77 | 1.79 | 3 | 34.88 | 8.44 | 3 |
| Donor 340 | 0.70 | 0.43 | 3 | 45.61 | 3.23 | 3 | 56.28 | 3.01 | 3 | 57.59 | 3.52 | 3 |

### Example 14—Insertion into NK Cells Using DNAPK Inhibitors

NK cells were assessed for the impact of DNA protein kinase inhibitors (DNA-PKI) on indel and insertion rates. NK cells were treated with LNP compositions delivering mRNA encoding Cas9 (SEQ ID NO: 8) and gRNA G000562 targeting AAVS1 in the presence of DNA protein kinase inhibitors. A subset of samples was also treated with AAV encoding a GFP coding sequence flanked by regions of homology to the AAVS1 edit site (SEQ ID NO: 16).

NK cells were isolated from a commercially obtained leukopak using the EasySep Human NK Cell Isolation Kit (STEMCELL, Cat. No. 17955) according to the manufacturer's protocol. Human primary NK cells were activated and expanded using K562-41BBL cells as feeder cells in OpTmizer media with 5% human AB serum, 500 U/mL IL-2, and 5 ng/ml IL-15 for 3 days. NK cells were plated at 50,000 cells per well in triplicate in OpTmizer supplemented as described above with DNA-PKI at concentrations indicated in Tables 10 and 11. LNPs were preincubated with 10 ug/ml APOE3 at 37° C. for about 15 minutes in OpTmizer media with 2.5% human AB serum, 500 U/mL IL-2 and 5 ng/ml IL-15. The pre-incubated LNP compositions were added to NK cells suspended in the same media at a final concentration of 10 ug/ml of total RNA cargo in triplicate. For a subset of samples, AAV encoding GFP flanked by regions homologous to the AAVS1 edit site were added at a multiplicity of infection (MOI) of 600,000 genome copies following editing. At seven days post LNP composition treatment, cells were phenotyped by flow cytometry to measure GFP insertion rates. Briefly, NK cells were incubated with antibodies targeting CD3 (Biolegend, Cat. No. 317336) and CD56 (Biolegend, Cat. No. 318310). Cells were subsequently washed, processed on a Cytoflex instrument (Beckman Coulter) and analyzed using the FlowJo software package. NK cells were gated on size, CD3/CD56 status, and GFP expression. High GFP-expressing cells were gated as targeted GFP insertion in AAVS1 locus and low GFP-expressing cells were gated as episomal retention. Cells were then collected for NGS analysis as described in Example 1.4.

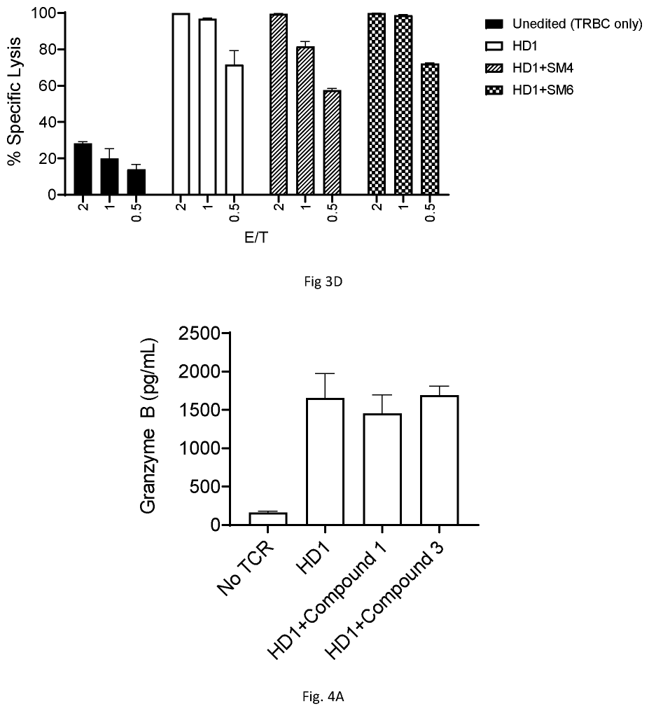

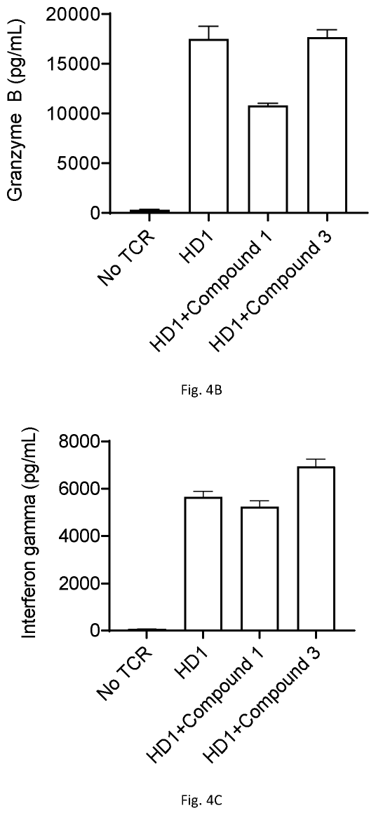

Tables 10 and 11 and FIGS. 6A and 6B show percent editing following treatment with LNP compositions, AAV, and varying concentrations of the DNAPK inhibitors Compound 1 and Compound 4. Both indel formation and insertion increased in the presence of DNAPK inhibitors.

TABLE 10
Mean percent editing at AAVS1 with varying doses of DNA-PKI

| | 0 uM | | 0.125 uM | | 0.25 uM | | 0.5 uM | |
|---|---|---|---|---|---|---|---|---|
| Sample | Mean | SD | Mean | SD | Mean | SD | Mean | SD |
| Unedited | 0.67 | 0.60 | | | | | | |
| No DNA-PKI | 93.17 | 0.12 | | | | | | |
| Compound 1 | | | 96.10 | 1.01 | 96.97 | 0.21 | 98.13 | 0.65 |
| Compound 4 | | | 96.77 | 0.67 | 97.37 | 0.06 | 97.67 | 1.55 |

TABLE 11
Percent of NK cells with high GFP expression seven days
following editing with LNP compositions, AAV and DNA-PKI.

| | 0 uM | | 0.125 uM | | 0.25 uM | | 0.5 uM | |
|---|---|---|---|---|---|---|---|---|
| Sample | Mean | SD | Mean | SD | Mean | SD | Mean | SD |
| AAV only | 2.68 | 0.37 | | | | | | |
| No DNA- | 74.93 | 4.09 | | | | | | |
| PKI | | | | | | | | |
| Compound 1 | | | 85.47 | 2.48 | 90.23 | 1.66 | 92.30 | 1.66 |
| Compound 4 | | | 88.10 | 1.00 | 90.63 | 1.01 | 90.27 | 3.09 |

### Example 15—Multiediting with Two Insertions in T Cells

To demonstrate engineering of T cells with five distinct Cas9 edits, healthy donor cells were treated sequentially with five LNP compositions co-formulated with an mRNA encoding Cas9 (SEQ ID NO: 8) and a sgRNA targeting either TRAC (G013006), TRBC (G016239), CIITA (G013676), HLA-A (G018995), or AAVS1(G000562). A transgenic WT1 targeting TCR was site-specifically integrated into the TRAC cut site by delivering a homology directed repair template (SEQ ID NO: 14) using AAV. As a proof-of-concept we also site-specifically integrated GFP into the AAVS1 target site using a second homology repair template (SEQ ID NO: 15).

T cells were isolated from the leukapheresis products of two healthy HLA-A*02:01+ donors (STEMCELL Technologies). T cells were isolated using EasySep Human T cell Isolation kit (STEMCELL Technologies, 17951) following manufacturer's protocol and cryopreserved using Cryostor CS10 (STEMCELL Technologies, 07930). The day before initiating T cell editing, cells were thawed and rested overnight in T cell activation media (TCAM: CTS OpTmizer (Thermofisher, A3705001) supplemented with 2.5% human AB serum (Gemini, 100-512), 1× GlutaMAX (Thermofisher, 35050061), 10 mM HEPES (Thermofisher, 15630080), 200 U/mL IL-2 (Peprotech, 200-02), 5 ng/mL IL-7 (Peprotech, 200-07), and 5 ng/mL IL-15 (Peprotech, 200-15).

### LNP Composition Treatment and Expansion of T Cells

LNPs were generally prepared as described in Example 1 with the lipid composition of 50/38.5/10/1.5, expressed as the molar ratio of ionizable lipid/cholesterol/DSPC/PEG, respectively. Immediately prior to exposure to T cells, LNP compositions were preincubated in ApoE containing media. Experimental design of the sequential editing steps and control groups is found in Table 12.

TABLE 12
Experimental Design

| Group | Day 1 | Day 2 | Day 3 | Day 4 | Day 5 | Purpose |
|---|---|---|---|---|---|---|
| Unedited | None | None | None | None | None | Negative control |
| GFP-AAV | None | None | None | AAV-GFP | None | Control for GFP |
| only | | | | | | episomal expression |
| TCR edits | None | None | TRAC LNP + | None | TRBC | Control for TCR |
| only | | | WT1 AAV | | | replacement |
| Quintuple | CIITA | HLA-A | TRAC LNP + | AAVS1 + | TRBC | Experimental |
| Edit | | | WT1 AAV | AAV-GFP | | |

Day 1: LNP compositions targeting CIITA as indicated in Table 12 were incubated at a concentration of 5 ug/mL in TCAM containing 5 ug/mL rhApoE3 (Peprotech 350-02). T cells were harvested, washed, and resuspended at a density of 2×10\hat{ }6 cells/mL in TCAM with a 1:50 dilution of T Cell TransAct, human reagent (Miltenyi, 130-111-160). T cells and LNP-ApoE solutions were then mixed at a 1:1 ratio by volume and T cells plated in culture flasks overnight.

Day 2: LNP compositions targeting HLA-A as indicated in Table 12 were incubated at a concentration of 25 ug/mL in TCAM containing 20 ug/mL rhApoE3 (Peprotech 350-02). LNP-ApoE solution was then added to the appropriate culture at a 1:10 ratio by volume.

Day 3: LNP compositions targeting TRAC were incubated at a concentration of 5 ug/mL in TCAM containing 5 ug/mL rhApoE3 (Peprotech 350-02). T cells were harvested, washed, and resuspended at a density of 1×10\hat{ }6 cells/mL in TCAM. T cells and LNP-ApoE media were mixed at a 1:1 ratio by volume and T cells plated in culture flasks. WT1 AAV was then added to each group at a MOI of 3×10\hat{ }5 genome copies/cell. The DNA-PK inhibitor Compound 4 was added to each group at a concentration of 0.25 μM Day 4: LNP compositions targeting AAVS1 were incubated at a concentration of 5 ug/mL in TCAM containing 5 ug/mL rhApoE3 (Peprotech 350-02). Meanwhile, T cells were harvested, washed, and resuspended at a density of 1×10\hat{ }6 cells/mL in TCAM. T cells and LNP-ApoE media were mixed at a 1:1 ratio by volume and T cells plated in culture flasks. GFP-AAV was then added to each group at a MOI of 3×10\hat{ }5 genome copies/cell. The DNA-PK inhibitor Compound 4 was added to each group at a concentration of 0.25 μM.

Day 5: LNP compositions targeting TRBC as indicated in Table 12 were incubated at a concentration of 5 ug/mL in TCAM containing 5 ug/mL rhApoE3 (Peprotech 350-02). T cells were harvested, washed, and resuspended at a density of 1×10\hat{ }6 cells/mL in TCAM. LNP-ApoE solution was then added to the appropriate culture at a 1:1 ratio by volume.

Day 6-11: T cells were transferred to a 24-well GREX plate (Wilson Wolf, 80192) in T cell expansion media (TCEM: CTS OpTmizer (Thermofisher, A3705001) supplemented with 5% CTS Immune Cell Serum Replacement (Thermofisher, A2596101), 1× GlutaMAX (Thermofisher, 35050061), 10 mM HEPES (Thermofisher, 15630080), 200 U/mL IL-2 (Peprotech, 200-02), 5 ng/ml IL-7 (Peprotech, 200-07), 5 ng/ml IL-15 (Peprotech, 200-15)) and expanded per manufacturer's protocols. Briefly, T cells were expanded for 6 days, with media exchanges every other day.

### Quantification of T Cell Editing by Flow Cytometry and NGS

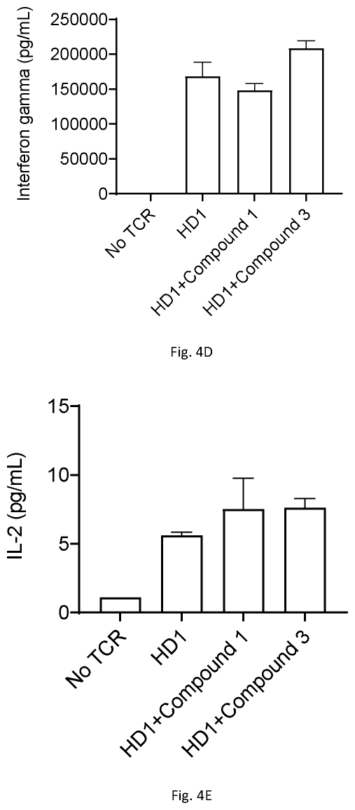

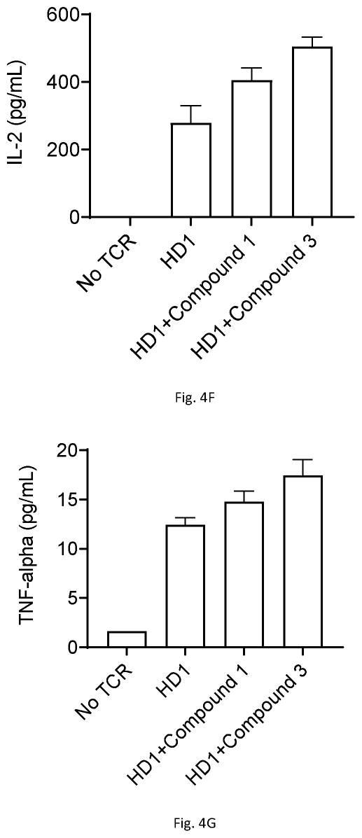

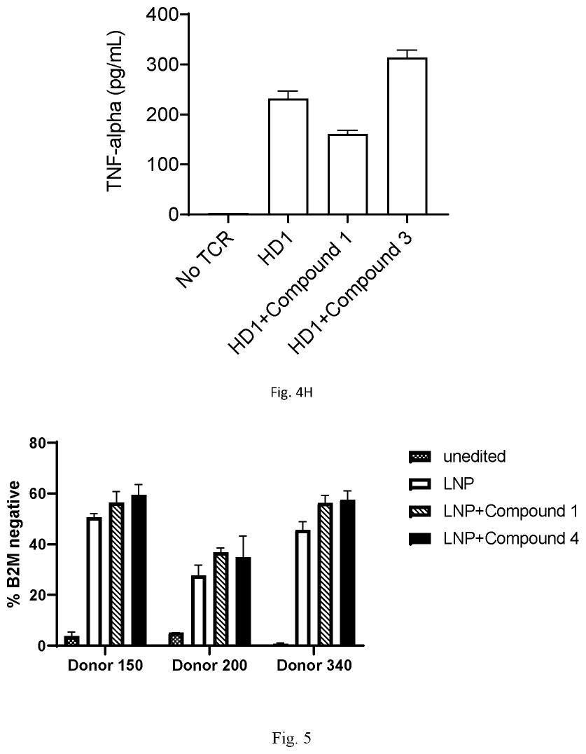

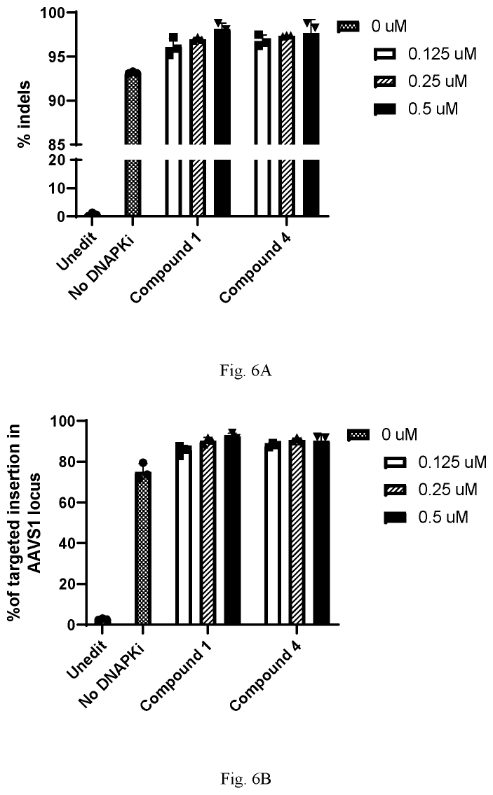

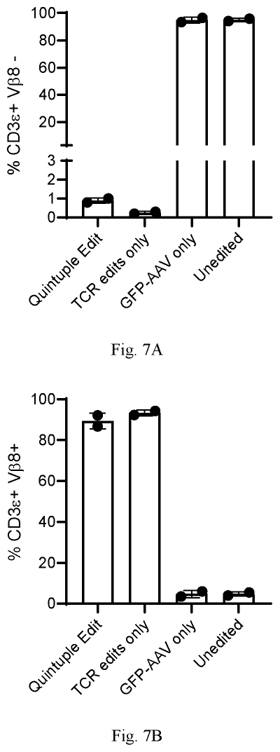

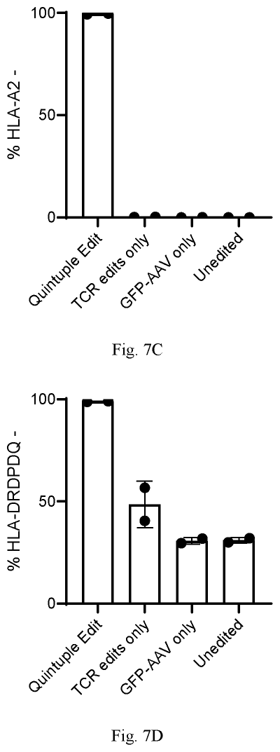

Post expansion, edited T cells were stained with antibodies targeting HLA-A*02:01 (Biolegend 343307), HLA-DR-DP-DQ (Biolegend 361712), WT1-TCR (Vb8⁺, Biolegend 348104), CD3e (Biolegend 300328), CD4 (Biolegend 317434), CD8 (Biolegend 301046), and Viakrome 808 Live/Dead (Cat. C36628). This cocktail was used to determine HLA-A*02:01 knockout (HLA-A2-), HLA-DR-DP-DQ knockdown via CIITA knockout (HLA-DRDPDQ-), WT1-TCR insertion (CD3⁺Vb8⁺), and the percentage of cells expressing residual endogenous TCR (CD3⁺Vb8⁻). Insertion into the AAVS1 site was tracked by monitoring GFP expression. Following antibody incubation, cells were washed, processed on a Cytoflex LX instrument (Beckman Coulter) and analyzed using the FlowJo software package. T cells were gated on size and CD4/CD8 status prior to examining editing and insertion markers. Editing and insertion rates can be found in Table 13 and Table 14 for CD8+ and CD4+ T cells, respectively. FIGS. 7A-7F show graphs of the editing rates of all targets in CD8+ T cells. The percent of T cells with all intended edits (i.e., insertion of the WT1-TCR and GFP, combined with knockout of HLA-A and CIITA) was gated as % CD3⁺Vb8⁺GFP⁺HLA-A⁻ HLA-DRDPDQ⁻. High levels of HLA-A and CIITA knockout, as well as GFP and WT1-TCR insertion were observed in quintuple edited samples from both donors, yielding >75% of fully edited CD8+ T cells and >85% of fully edited CD4+ T cells.

TABLE 13
Editing rates in CD8+ T cells in Donors A and B

| | | | GFP-AAV | | TCR edits | | Quintuple | |
|---|---|---|---|---|---|---|---|---|
| | Unedited | | only | | only | | Edit | |
| Marker | A | B | A | B | A | B | A | B |
| GFP+ | 0.0 | 0.0 | 0.3 | 0.7 | 0.0 | 0.0 | 88.4 | 93.1 |
| HLA-A2− | 0.1 | 0.0 | 0.0 | 0.0 | 0.3 | 0.2 | 99.7 | 99.4 |
| HLA-DRDPDQ− | 31.9 | 29.6 | 30.0 | 32.0 | 40.5 | 56.7 | 99.0 | 98.7 |
| CD3+ Vb8− | 93.3 | 96.4 | 94.1 | 95.8 | 0.2 | 0.3 | 1.0 | 0.8 |
| CD3+ Vb8+ | 6.0 | 3.5 | 5.5 | 4.0 | 92.3 | 94.3 | 86.7 | 92.1 |
| CD3+ Vb8+ | 0.0 | 0.0 | 0.0 | 0.0 | 0.0 | 0.0 | 76.7 | 84.8 |
| GFP+ HLA-A−, | | | | | | | | |
| HLA-DRDPDQ− | | | | | | | | |

TABLE 14
Editing rates in CD4+ T cells in Donors A and B

| | | | GFP-AAV | | TCR edits | | Quintuple | |
|---|---|---|---|---|---|---|---|---|
| | Unedited | | only | | only | | Edit | |
| Marker | A | B | A | B | A | B | A | B |
| GFP+ | 0.0 | 0.0 | 0.9 | 1.3 | 0.0 | 0.0 | 86.4 | 91.3 |
| HLA-A2− | 0.1 | 0.0 | 0.0 | 0.1 | 0.2 | 0.2 | 99.5 | 99.2 |
| HLA-DRDPDQ− | 77.0 | 73.6 | 71.3 | 75.0 | 93.1 | 96.1 | 99.2 | 99.1 |
| CD3+ Vb8− | 94.6 | 94.6 | 94.8 | 94.2 | 0.3 | 0.4 | 1.3 | 1.3 |
| CD3+ Vb8+ | 5.1 | 5.1 | 4.9 | 5.4 | 86.3 | 90.9 | 80.7 | 91.0 |
| Vb8+ GFP+ | 0.6 | 0.0 | 0.1 | 0.0 | 0.0 | 0.0 | 86.1 | 90.6 |
| HLA-A− | | | | | | | | |
| HLA-DRDPDQ− | | | | | | | | |

### Example 16—LNP Composition Activity Evaluated in Serum Media Conditions

To evaluate LNP composition editing efficacy, LNP compositions were tested in vitro to evaluate the effect of alternative media conditions on insertion efficiency in CD3 positive T cells. T cells were treated with LNP compositions with varied molar ratios of lipid components encapsulating Cas9 mRNA and a sgRNA targeting the TRAC gene. An AAV6 viral construct delivered a homology directed repair template (HDRT) that encoded a GFP reporter flanked by homology arms for site-specific integration into the TRAC locus (Vigene; SEQ ID NO: 17). TRAC gene disruption was assessed by flow cytometry for loss of T cell receptor surface proteins. Insertion was assessed by flow cytometry for GFP luminescence.

LNP compositions were preincubated with ApoE3. Equal volume of ApoE3 media was added to each well. Subsequently 100 μL of the LNP-ApoE mix was added to each T cell plate. The final concentration of LNPs at the top dose was set to be 5 μg/mL. Final concentrations of ApoE3 at 5 μg/mL and T cells were at a final density of 0.5e6 cells/mL. Plates were incubated at 37° C. with 5% CO₂ for 7 days and then harvested for flow cytometry analysis.

LNPs were generally prepared as described in Example 1 with the lipid composition as indicated in Table 15, expressed as the molar ratio of ionizable lipid A/cholesterol/DSPC/PEG, respectively. LNP compositions delivered mRNA encoding Cas9 (SEQ ID NO: 8) and sgRNA targeting and sgRNA (SEQ ID NO. 1) targeting human TRAC. The cargo ratio of sgRNA to Cas9 mRNA was 1:2 by weight.

TABLE 15
LNP formulation analysis results

| | Composition | | Z-Ave | | Num Ave | |
|---|---|---|---|---|---|---|
| | Molar | Encapsulation | Size | | Size | N/P |
| LNP ID | Ratio | (%) | (nm) | PDI | (nm) | ratio |
| COMPOSITION 1 | 50/38.5/10/1.5 | 98% | 113 | 0.03 | 102 | 6 |
| (Comparative) | | | | | | |
| COMPOSITION 16 | 35/47.5/15/2.5 | 98% | 78 | 0.03 | 72 | 6 |

T cells from a single donor (Lot #W0106) were prepared as described in Example 1 with the following media modifications. T cells were plated with media supplemented with either 2.5% human AB serum (HABS), 2.5% CTS Immune Cell SR (Gibco, Cat #A25961-01) serum replacement (SR), 5% serum replacement (SR), or the combination of 2.5% human AB serum and 2.5% serum replacement. T cells were activated 24 hours post thaw as described in Example 1.2. Two days post activation, T cells were transfected with LNP compositions as described in Example 16.1 at LNP concentrations of 0.31 μg/mL, 0.63 μg/mL, 1.25 μg/mL, and 2.5 μg/mL. AAV6 encoding homology directed repair template (HDRT) that encoded a GFP reporter (Vigene; SEQ ID NO: 13) flanked by homology arms for site-specific integration into the TRAC locus and was added to each well at a multiplicity of infection (MOI) of 3×105 viral particles/cell. The small molecule inhibitor of DNA-dependent protein kinase, Compound 4, was added at 0.25 μM.

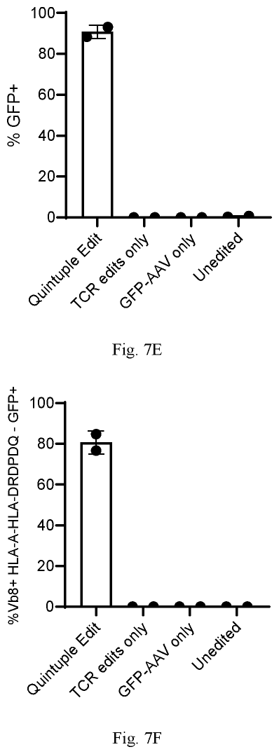

Five days post transfection, T cells were phenotyped by flow cytometry analysis as described in Example 14 to evaluate the insertion efficiency of the LNP compositions. Table 16 shows the percent of CD3 negative cells. The T cell receptor alpha chain encoded by TRAC is required for T cell receptor/CD3 complex assembly and translocation to the cell surface. Accordingly, disruption of the TRAC gene by genome editing leads to a loss of CD3 protein on the cell surface of T cells. The mean percentage of GFP positive T cells for each media condition is shown in Table 17 and FIGS. 8A-8B. Cells expressing GFP protein indicate successful insertion into genome.

TABLE 16
Percent CD3 negative T cells following treatment of activated
T cells with AAV and indicated LNP formulation.

| | | | | | | | | 2.5% HABS | |
|---|---|---|---|---|---|---|---|---|---|
| | LNP | 2.5% HABS | | 2.5% SR | | 5% SR | | & 2.5% SR | |
| Composition | (ug/ml) | Mean | SD | Mean | SD | Mean | SD | Mean | SD |
| 50/38.5/10/1.5 | 2.5 | 94.55 | 0.07 | 99.00 | 0.14 | 97.95 | 0.07 | 99.25 | 0.07 |
| | 1.25 | 92.25 | 0.35 | 96.05 | 1.20 | 92.10 | 2.55 | 95.95 | 0.21 |
| | 0.63 | 76.55 | 0.21 | 76.60 | 3.11 | 63.55 | 2.47 | 74.70 | 1.27 |
| | 0.31 | 48.35 | 1.34 | 25.95 | 1.20 | 16.55 | 2.47 | 41.55 | 1.20 |
| 35/47.5/15/2.5 | 2.5 | 99.40 | 0.00 | 98.80 | 0.42 | 98.65 | 0.07 | 98.70 | 0.14 |
| | 1.25 | 98.85 | 0.07 | 98.95 | 0.64 | 98.50 | 0.14 | 97.25 | 0.35 |
| | 0.63 | 94.10 | 0.28 | 96.80 | 0.42 | 95.25 | 0.35 | 84.60 | 1.41 |
| | 0.31 | 75.20 | 0.14 | 68.75 | 9.97 | 64.20 | 0.57 | 50.30 | 1.56 |

TABLE 17
Percent GFP+ cells following treatment of activated
T cells with AAV and indicated LNP formulations.

| | | | | | | | | 2.5% HABS | |
|---|---|---|---|---|---|---|---|---|---|
| | | 2.5% HABS | | 2.5% SR | | 5% SR | | and 2.5% SR | |
| | Dose | Insertion % | | Insertion % | | Insertion % | | Insertion % | |
| LNP | (ug/mL) | (mean) | SD | (mean) | SD | (mean) | SD | (mean) | SD |
| 50/38.5/10/1.5 | 2.5 | 94.6 | 0.0 | 95.4 | 0.4 | 93.7 | 0.6 | 90.0 | 0.1 |
| | 1.25 | 92.3 | 0.3 | 92.3 | 0.5 | 87.7 | 1.8 | 75.1 | 1.1 |
| | 0.63 | 76.6 | 0.1 | 76.3 | 1.3 | 63.1 | 1.8 | 31.2 | 2.6 |
| | 0.31 | 48.4 | 0.9 | 30.5 | 1.0 | 19.5 | 2.5 | 5.0 | 0.7 |
| 35/47.5/15/2.5 | 2.5 | 95.6 | 0.0 | 96.3 | 0.5 | 95.6 | 0.1 | 94.7 | 0.4 |
| | 1.25 | 94.8 | 0.5 | 96.0 | 1.4 | 95.6 | 0.0 | 91.1 | 0.2 |
| | 0.63 | 89.6 | 0.6 | 93.3 | 0.5 | 91.1 | 0.2 | 78.8 | 1.0 |
| | 0.31 | 74.7 | 0.1 | 75.0 | 0.0 | 64.4 | 0.4 | 48.8 | 1.2 |

### 16.1. LNP Transfection of T Cells

The LNP dose response curves (DRCs) transfection was performed on the Hamilton Microlab STAR liquid handling system. The liquid handler was provided with the following: (a) 4× the desired highest LNP dose in the top row of a deep well 96-deep well plate, (b) ApoE3 diluted in media at 20 μg/mL, (c) complete T cell growth media composed of CTS OpTmizer Base Media as previously described in Example 1 and (d) T cells plated at 10⁶/ml density in 100 uL in 96-well flat bottom tissue culture plates. The liquid handler first performed an 8-point two-fold serial dilution of the LNPs starting from the 4×LNP dose in the deep well plate. Equal volume of ApoE3 media was then added to each well resulting in a 1:1 dilution of both LNP and ApoE3. Subsequently, 100 uL of the LNP-ApoE mix was added to each T cell plate. The final concentration of LNPs at the top dose was set to be 5 μg/mL. Final concentrations of ApoE3 at 5 pg/mL and T cells were at a final density of 0.5×10⁶ cells/mL. Plates were incubated at 37° C. with 5% CO₂ for 24 or 48 hours for activated or on-activated T cells, respectively. Post-incubation, T cells treated with LNPs were harvested and analyzed for on-target editing or Cas9 protein expression detection. Remaining cells were cultured for 7-10 days post LNP composition treatment and protein surface expression assessed by flow cytometry.

### Example 17—Off-Target Structural Variation Comparison of DNA-PKI by UnIT

In this experiment, sgRNA targeting TRAC (G013006) treated with Compound 3 or Compound 4 were assayed for off-target structural variation translocations compared to unedited or untreated TRAC sgRNA.

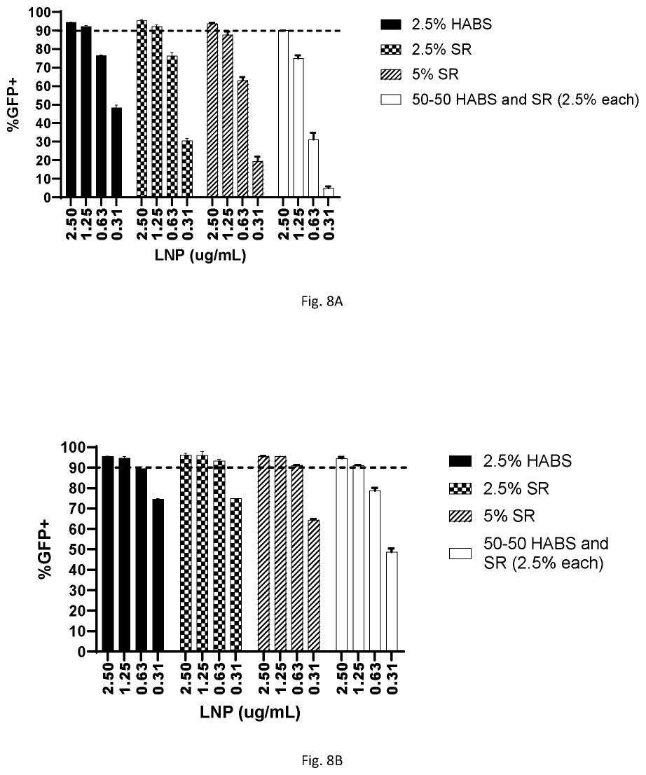

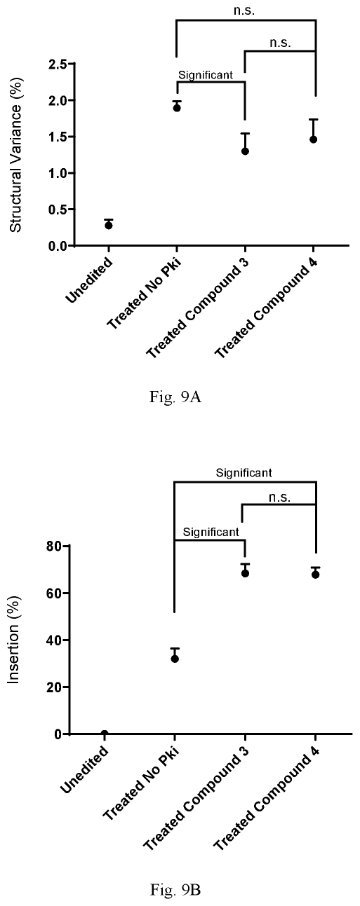

On day 8 post-editing, T cells from Example 12 from the untreated, unedited, Compound 3 and Compound 4 guides samples were collected, spun down and the pellets were directly used as input material for gDNA isolation using “Zymo Quick DNA/RNA Mag Beads Kit” (Zymo Cat. R2131). The UnIT structural variant characterization assay was applied to these gDNA samples. High molecular weight genomic DNA is simultaneously fragmented and sequence-tagged (‘tagmented’) with the Tn5 transposase and an adapter with a partial Illumina P5 sequence and a 12 bp unique molecular identifier (UMI). Two sequential PCRs using a primer to P5 and hemi-nested gene specific primers (GSP) imparting the Illumina the P7 sequence to create two Illumina compatible NGS libraries per sample (Illumina, Ref 15033624). Sequencing across both directions of the CRISPR/Cas9 targeted cut site with the two libraries allows the inference and quantification of structural variants in DNA repair outcomes after genome editing. If the two fragments were aligned to different chromosomes, the SV was classified as an “inter-chromosomal translocation.” The magnitude of structural variation was shown in FIG. 9A and Table 18. The insertion percent was shown in FIG. 9B and Table 19.

TABLE 18
Unintended Structural Variance

| Sample | Average | SD |
|---|---|---|
| Unedited | 0.28 | 0.08 |
| Treated No Pki | 1.89 | 0.09 |
| Treated Compound 3 | 1.30 | 0.24 |
| Treated Compound 4 | 1.46 | 0.28 |

TABLE 19
Insertion Percent

| Sample | Average | SD |
|---|---|---|
| Unedited | 0.11 | 0.19 |
| Treated No Pki | 32.02 | 4.41 |
| Treated Compound 3 | 68.35 | 3.99 |
| Treated Compound 4 | 67.82 | 2.98 |

### Example 18—Off-Target Analysis of TRAC and TRBC Guides with DNA-PKI

T Cells from Example 12 were screened for validation of off-target genomic sites targeting TRAC and TRBC and was performed according to the Integrated DNA Technologies, IDT rhAmpSeq rhPCR Protocol. In this experiment, 2 sgRNA targeting TRAC and TRBC in combination with DNA-PKI Compound 3 and Compound 4 were screened for validation of off-target profiles. The number of validated off-target sites for sgRNAs targeting TRAC (G013006) and TRBC (G016239) were shown in Table 20. Off-target sites were validated if the p value was less than 0.05 percent indel. Of the 173 off-target sites identified for the sgRNA targeting TRAC, 0 sites were validated. Of the 92 off-target sites identified for the sgRNA targeting TRBC, 0 sites were validated.

TABLE 20
Off-Target Site Validation of TRAC and
TRBC Guides with DNA-PKI

| | | | | Off- | |
|---|---|---|---|---|---|
| gRNA | | | Guide Sequence | Target | Sites |
| ID | DNA-PKI | Target | (SEQ ID NO:) | Sites | Validated |
| G013006 | N/A | TRAC | CUCUCAGCUGGUACACGGCA | 173 | 0 |
| | | | (SEQ ID NO: 19) | | |
| G013006 | Compound 3 | TRAC | CUCUCAGCUGGUACACGGCA | 173 | 0 |
| | | | (SEQ ID NO: 19) | | |
| G013006 | Compound 4 | TRAC | CUCUCAGCUGGUACACGGCA | 173 | 0 |
| | | | (SEQ ID NO: 19) | | |
| G016239 | N/A | TRBC | GGCCUCGGCGCUGACGAUCU | 92 | 0 |
| | | | (SEQ ID NO: 20) | | |
| G016239 | Compound 3 | TRBC | GGCCUCGGCGCUGACGAUCU | 92 | 0 |
| | | | (SEQ ID NO: 20) | | |
| G016239 | Compound 4 | TRBC | GGCCUCGGCGCUGACGAUCU | 92 | 0 |
| | | | (SEQ ID NO: 20) | | |

### Example 19—SpyCas9-Mediated Insertion of an Immunological Receptor within TRAC with or without DNA-PK Inhibitors

### 19.1. T Cell Preparation

Healthy human donor apheresis was obtained commercially (Hemacare, Cat. PB001F-2), and cells were washed, re-suspended in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930).

Upon thaw, T cells were plated at a density of 1.0×10\hat{ }6 cells/mL in T cell growth media (TCGM) composed of CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/ml recombinant human interleukin 7 (Peprotech, Cat. 200-07), and 5 ng/mL recombinant human interleukin 15 (Peprotech, Cat. 200-15). T cells were rested in this media for 24 hours, at which time they were activated with T Cell TransAct™, human reagent (Miltenyi, Cat. 130-111-160) added at a 1:100 ratio by volume. T cells were activated for 48 hours prior to LNP treatments.

### Example 19.2. T Cell Treatment and Expansion

48 hours post-activation, T cells were harvested, centrifuged at 500 g for 5 min, and resuspended at a concentration of 6.41×10\hat{ }5 T cells/mL in T cell plating media (TCPM): a serum-free version of TCGM containing 400 U/mL recombinant human interleukin-2 (Peprotech, Cat. 200-02), 10 ng/mL recombinant human interleukin 7 (Peprotech, Cat. 200-07), and 10 ng/mL recombinant human interleukin 15 (Peprotech, Cat. 200-15). 50 μL of T cells in TCPM (3.2×10⁴ T cells) were added per well to be treated in flat-bottom 96-well plates.

LNPs were generated as described in Example 1 at a ratio of 50/38.5/10/1.5 (Lipid A/cholesterol/DSPC/PEG2k-DMG). Prior to T cell treatment, two separate LNP mixes (referred below as mixes “A” and “B”) were prepared in T cell treatment media (TC™): a version of TCGM containing 20 μg/mL rhApoE3 in the absence of interleukins 2, 15, or 7.

Mix “A” consisted of an LNP with G013006 (SEQ ID NO: 1) diluted to 13.36 μg/mL, while mix “B” consisted of an LNP with Cas9 mRNA 60 (SEQ ID NO: 11) diluted to 13.36 μg/mL. LNP mixes “A” and “B” were incubated at 37° C. for 15 minutes. Mix “A” was serially diluted 1:2 in TCTM, and mixed 1:1 by volume with mix “B”. 25 μL of the resulting solution was added to 3.2×10\hat{ }4 T cells in 96-well plates.

Next, a repair template in the form of an adeno-associated virus (AAV) encoding the HD3 TCR (SEQ ID NO: 18 was diluted in TCTM to 3.84×10\hat{ }11 genome copies/mL in the presence or absence of Compound 4 diluted to 2 μM. 25 μL of the resulting solution was added to T cells that were treated with LNPs in the prior step. To enable editing assessments by NGS without the interference of repair templates, an arm of this experiment received 25 μL of TCTM with or without Compound 4 diluted to 2 μM in the absence of AAVs.

Following the addition of LNPs, repair templates and Compound 4, T cells were incubated at 37° C. for 48 hours, at which time T cells were centrifuged at 500 g for 5 min, resuspended in 200 μL of TCGM and returned to the incubator.

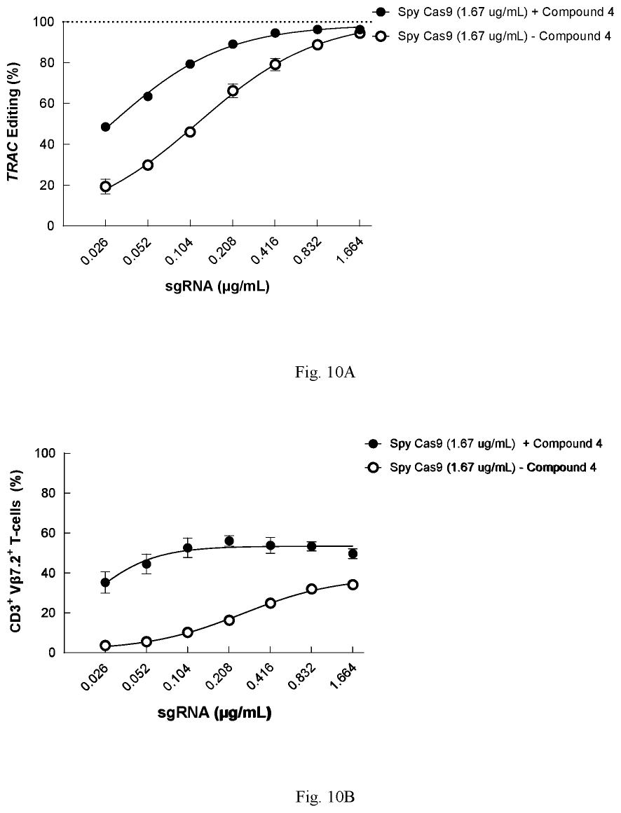

On day 4 post-treatment, cells that did not receive AAV templates were centrifuged at 500 g for 5 min, subjected to lysis, PCR amplification of each targeted locus and subsequent NGS analysis, as described in Example 1. Results for indel percent are shown in Table 21 and FIG. 10A.

Also on day 4 post-treatment, cells that received AAV templates were mixed and sub-cultured at a 1:4 ratio (v/v) in TCGM. On day 7 post-treatment, cells that received AAV templates were evaluated by flow cytometry.

### Example 19.3. Flow Cytometry

On day 7 post-LNP treatment, 50 μL of cells were transferred to U-bottom 96-well plates and spun down for 5 min at 500 g. The supernatant was discarded, cells were resuspended in 100 μL of FACS buffer containing Viakrome 808 (Beckman C., Cat. C36628) (1:100), PC5.5 anti-CD3 (Biolegend, Cat. 317336) (1:200), BV421 anti-CD4 (Biolegend, Cat. 317434) (1:100), BV785 anti-CD8 (Biolegend, Cat. 301046) (1:100), and anti-V07.2 (Beckman C., IM3604) (1:50) and stained for 30 min at 4° C. in the dark. Cells were washed once with 200 μL of FACS buffer, resuspended in 100 μL of FACS buffer and processed on a Cytoflex LX flow cytometer. Results for percent HD3 TCR insertion are shown in Table 22 and FIG. 10B.

TABLE 21
Percent indel with and without the presence of Compound 4.

| | Spy Cas9 + Compound 4 | | Spy Cas9 − Compound 4 | |
|---|---|---|---|---|
| sgRNA | Mean TRAC | St. dev. TRAC | Mean TRAC | St. dev. TRAC |
| (μg/mL) | Editing % | Editing % | Editing % | Editing % |
| 1.67 | 96.10 | 0.85 | 94.30 | 0.14 |
| 0.835 | 96.15 | 0.49 | 88.70 | 1.70 |
| 0.418 | 94.45 | 0.92 | 79.05 | 3.04 |
| 0.209 | 89.00 | 0.42 | 66.20 | 3.39 |
| 0.104 | 79.25 | 0.64 | 46.00 | 2.12 |
| 0.052 | 63.45 | 0.07 | 29.80 | 0.71 |
| 0.026 | 48.55 | 0.49 | 19.35 | 3.61 |
| 0 | 0.20 | 0.00 | 0.10 | 0.00 |

TABLE 22
Percent HD3 TCR insertion with and
without the presence of Compound 4.

| | Spy Cas9 + Compound 4 | | Spy Cas9 − Compound 4 | |
|---|---|---|---|---|
| | Mean CD3⁺ | St. dev. CD3⁺ | Mean CD3⁺ | St. dev. CD3⁺ |
| sgRNA | Vβ7.2⁺ | Vβ7.2⁺ | Vβ7.2⁺ | Vβ7.2⁺ |
| (μg/mL) | T Cells % | T Cells % | T Cells % | T Cells % |
| 1.67 | 49.65 | 2.47 | 34.10 | 0.85 |
| 0.835 | 53.30 | 2.26 | 31.95 | 1.20 |
| 0.418 | 53.80 | 3.96 | 24.85 | 0.35 |
| 0.209 | 56.10 | 2.69 | 16.30 | 0.28 |
| 0.104 | 52.60 | 4.95 | 10.20 | 0.00 |
| 0.052 | 44.50 | 4.95 | 5.54 | 0.43 |
| 0.026 | 35.20 | 5.37 | 3.67 | 0.50 |
| 0 | 0.79 | 0.08 | 0.65 | 0.14 |

### Additional Sequence Table

In the following table and throughout, the terms “mA,” “mC,” “mU,” or “mG” are used to denote a nucleotide that has been modified with 2′-O-Me.

In the following table, a “*k” is used to depict a PS modification. In this application, the terms A*, C*, U*, or G* may be used to denote a nucleotide that is linked to the next (e.g., 3′) nucleotide with a PS bond.

It is understood that if a DNA sequence (comprising Ts) is referenced with respect to an RNA, then Ts should be replaced with Us (which may be modified or unmodified depending on the context), and vice versa.

In the following table, single amino acid letter code is used to provide peptide sequences.

| | SEQ | |
|---|---|---|
| Descrip- | ID | |
| tion | NO | Sequence |
| G013006 | 1 | mC*mU*mC*UCAGCUGGUACACGGCAGUUUUAGAm |
| | | GmCmUmAmGmAmAmAmUmAmGmCAAGUUAAAAUAA |
| | | GGCUAGUCCGUUAUCAmAmCmUmUmGmAmAmAmAm |
| | | AmGmUmGmGmCmAmCmCmGmAmGmUmCmGmGmUmG |
| | | mCmU*mU*mU*mU |
| G016239 | 2 | mG*mG*mC*CUCGGCGCUGACGAUCUGUUUUAGAm |
| | | GmCmUmAmGmAmAmAmUmAmGmCAAGUUAAAAUAA |
| | | GGCUAGUCCGUUAUCAmAmCmUmUmGmAmAmAmAm |
| | | AmGmUmGmGmCmAmCmCmGmAmGmUmCmGmGmUmG |
| | | mCmU*mU*mU*mU |
| G000529 | 3 | mG*mG*mC*CACGGAGCGAGACAUCUGUUUUAGAm |
| | | GmCmUmAmGmAmAmAmUmAmGmCAAGUUAAAAUAA |
| | | GGCUAGUCCGUUAUCAmAmCmUmUmGmAmAmAmAm |
| | | AmGmUmGmGmCmAmCmCmGmAmGmUmCmGmGmUmG |
| | | mCmU*mU*mU*mU |
| G013676 | 4 | mU*mG*mG*UCAGGGCAAGAGCUAUUGUUUUAGAm |
| | | GmCmUmAmGmAmAmAmUmAmGmCAAGUUAAAAUAA |
| | | GGCUAGUCCGUUAUCAmAmCmUmUmGmAmAmAmAm |
| | | AmGmUmGmGmCmAmCmCmGmAmGmUmCmGmGmUmG |
| | | mCmU*mU*mU*mU |
| G018995 | 5 | mA*mC*mA*GCGACGCCGCGAGCCAGGUUUUAGAm |
| | | GmCmUmAmGmAmAmAmUmAmGmCAAGUUAAAAUAA |
| | | GGCUAGUCCGUUAUCAmAmCmUmUmGmAmAmAmAm |
| | | AmGmUmGmGmCmAmCmCmGmAmGmUmCmGmGmUmG |
| | | mCmU*mU*mU*mU |
| G000562 | 6 | mC*mC*mA*AUAUCAGGAGACUAGGAGUUUUAGAm |
| | | GmCmUmAmGmAmAmAmUmAmGmCAAGUUAAAAUAA |
| | | GGCUAGUCCGUUAUCAmAmCmUmUmGmAmAmAmAm |
| | | AmGmUmGmGmCmAmCmCmGmAmGmUmCmGmGmUmG |
| | | mCmU*mU*mU*mU |
| tracrRNA | 7 | AACAGCAUAGCAAGUUAAAAUAAGGCUAGUCCGUU |
| | | AUCAACUUGAAAAAGUGGCACCGAGUCGGUGCUUU |
| | | UUUU |
| Recom- | 8 | MDKKYSIGLDIGTNSVGWAVITDEYKVPSKKFKVL |
| binant | | GNTDRHSIKKNLIGALLFDSGETAEATRLKRTARR |
| Cas9-NLS | | RYTRRKNRICYLQEIFSNEMAKVDDSFFHRLEESF |
| amino | | LVEEDKKHERHPIFGNIVDEVAYHEKYPTIYHLRK |
| acid | | KLVDSTDKADLRLIYLALAHMIKFRGHFLIEGDLN |
| sequence | | PDNSDVDKLFIQLVQTYNQLFEENPINASGVDAKA |
| | | ILSARLSKSRRLENLIAQLPGEKKNGLFGNLIALS |
| | | LGLTPNFKSNFDLAEDAKLQLSKDTYDDDLDNLLA |
| | | QIGDQYADLFLAAKNLSDAILLSDILRVNTEITKA |
| | | PLSASMIKRYDEHHQDLTLLKALVRQQLPEKYKEI |
| | | FFDQSKNGYAGYIDGGASQEEFYKFIKPILEKMDG |
| | | TEELLVKLNREDLLRKQRTFDNGSIPHQIHLGELH |
| | | AILRRQEDFYPFLKDNREKIEKILTFRIPYYVGPL |
| | | ARGNSRFAWMTRKSEETITPWNFEEVVDKGASAQS |
| | | FIERMTNFDKNLPNEKVLPKHSLLYEYFTVYNELT |
| | | KVKYVTEGMRKPAFLSGEQKKAIVDLLFKTNRKVT |
| | | VKQLKEDYFKKIECFDSVEISGVEDRFNASLGTYH |
| | | DLLKIIKDKDFLDNEENEDILEDIVLTLTLFEDRE |
| | | MIEERLKTYAHLFDDKVMKQLKRRRYTGWGRLSRK |
| | | LINGIRDKQSGKTILDFLKSDGFANRNFMQLIHDD |
| | | SLTFKEDIQKAQVSGQGDSLHEHIANLAGSPAIKK |
| | | GILQTVKVVDELVKVMGRHKPENIVIEMARENQTT |
| | | QKGQKNSRERMKRIEEGIKELGSQILKEHPVENTQ |
| | | LQNEKLYLYYLQNGRDMYVDQELDINRLSDYDVDH |
| | | IVPQSFLKDDSIDNKVLTRSDKNRGKSDNVPSEEV |
| | | VKKMKNYWRQLLNAKLITQRKFDNLTKAERGGLSE |
| | | LDKAGFIKRQLVETRQITKHVAQILDSRMNTKYDE |
| | | NDKLIREVKVITLKSKLVSDFRKDFQFYKVREINN |
| | | YHHAHDAYLNAVVGTALIKKYPKLESEFVYGDYKV |
| | | YDVRKMIAKSEQEIGKATAKYFFYSNIMNFFKTEI |
| | | TLANGEIRKRPLIETNGETGEIVWDKGRDFATVRK |
| | | VLSMPQVNIVKKTEVQTGGFSKESILPKRNSDKLI |
| | | ARKKDWDPKKYGGFDSPTVAYSVLVVAKVEKGKSK |
| | | KLKSVKELLGITIMERSSFEKNPIDFLEAKGYKEV |
| | | KKDLIIKLPKYSLFELENGRKRMLASAGELQKGNE |
| | | LALPSKYVNFLYLASHYEKLKGSPEDNEQKQLFVE |
| | | QHKHYLDEIIEQISEFSKRVILADANLDKVLSAYN |
| | | KHRDKPIREQAENIIHLFTLTNLGAPAAFKYFDTT |
| | | IDRKRYTSTKEVLDATLIHQSITGLYETRIDLSQL |
| | | GGDGGGSPKKKRKV |
| ORF | 9 | ATGGACAAGAAGTACAGCATCGGACTGGACATCGG |
| encoding | | AACAAACAGCGTCGGATGGGCAGTCATCACAGACG |
| Sp. Cas9 | | AATACAAGGTCCCGAGCAAGAAGTTCAAGGTCCTG |
| | | GGAAACACAGACAGACACAGCATCAAGAAGAACCT |
| | | GATCGGAGCACTGCTGTTCGACAGCGGAGAAACAG |
| | | CAGAAGCAACAAGACTGAAGAGAACAGCAAGAAGA |
| | | AGATACACAAGAAGAAAGAACAGAATCTGCTACCT |
| | | GCAGGAAATCTTCAGCAACGAAATGGCAAAGGTCG |
| | | ACGACAGCTTCTTCCACAGACTGGAAGAAAGCTTC |
| | | CTGGTCGAAGAAGACAAGAAGCACGAAAGACACCC |
| | | GATCTTCGGAAACATCGTCGACGAAGTCGCATACC |
| | | ACGAAAAGTACCCGACAATCTACCACCTGAGAAAG |
| | | AAGCTGGTCGACAGCACAGACAAGGCAGACCTGAG |
| | | ACTGATCTACCTGGCACTGGCACACATGATCAAGT |
| | | TCAGAGGACACTTCCTGATCGAAGGAGACCTGAAC |
| | | CCGGACAACAGCGACGTCGACAAGCTGTTCATCCA |
| | | GCTGGTCCAGACATACAACCAGCTGTTCGAAGAAA |
| | | ACCCGATCAACGCAAGCGGAGTCGACGCAAAGGCA |
| | | ATCCTGAGCGCAAGACTGAGCAAGAGCAGAAGACT |
| | | GGAAAACCTGATCGCACAGCTGCCGGGAGAAAAGA |
| | | AGAACGGACTGTTCGGAAACCTGATCGCACTGAGC |
| | | CTGGGACTGACACCGAACTTCAAGAGCAACTTCGA |
| | | CCTGGCAGAAGACGCAAAGCTGCAGCTGAGCAAGG |
| | | ACACATACGACGACGACCTGGACAACCTGCTGGCA |
| | | CAGATCGGAGACCAGTACGCAGACCTGTTCCTGGC |
| | | AGCAAAGAACCTGAGCGACGCAATCCTGCTGAGCG |
| | | ACATCCTGAGAGTCAACACAGAAATCACAAAGGCA |
| | | CCGCTGAGCGCAAGCATGATCAAGAGATACGACGA |
| | | ACACCACCAGGACCTGACACTGCTGAAGGCACTGG |
| | | TCAGACAGCAGCTGCCGGAAAAGTACAAGGAAATC |
| | | TTCTTCGACCAGAGCAAGAACGGATACGCAGGATA |
| | | CATCGACGGAGGAGCAAGCCAGGAAGAATTCTACA |
| | | AGTTCATCAAGCCGATCCTGGAAAAGATGGACGGA |
| | | ACAGAAGAACTGCTGGTCAAGCTGAACAGAGAAGA |
| | | CCTGCTGAGAAAGCAGAGAACATTCGACAACGGAA |
| | | GCATCCCGCACCAGATCCACCTGGGAGAACTGCAC |
| | | GCAATCCTGAGAAGACAGGAAGACTTCTACCCGTT |
| | | CCTGAAGGACAACAGAGAAAAGATCGAAAAGATCC |
| | | TGACATTCAGAATCCCGTACTACGTCGGACCGCTG |
| | | GCAAGAGGAAACAGCAGATTCGCATGGATGACAAG |
| | | AAAGAGCGAAGAAACAATCACACCGTGGAACTTCG |
| | | AAGAAGTCGTCGACAAGGGAGCAAGCGCACAGAGC |
| | | TTCATCGAAAGAATGACAAACTTCGACAAGAACCT |
| | | GCCGAACGAAAAGGTCCTGCCGAAGCACAGCCTGC |
| | | TGTACGAATACTTCACAGTCTACAACGAACTGACA |
| | | AAGGTCAAGTACGTCACAGAAGGAATGAGAAAGCC |
| | | GGCATTCCTGAGCGGAGAACAGAAGAAGGCAATCG |
| | | TCGACCTGCTGTTCAAGACAAACAGAAAGGTCACA |
| | | GTCAAGCAGCTGAAGGAAGACTACTTCAAGAAGAT |
| | | CGAATGCTTCGACAGCGTCGAAATCAGCGGAGTCG |
| | | AAGACAGATTCAACGCAAGCCTGGGAACATACCAC |
| | | GACCTGCTGAAGATCATCAAGGACAAGGACTTCCT |
| | | GGACAACGAAGAAAACGAAGACATCCTGGAAGACA |
| | | TCGTCCTGACACTGACACTGTTCGAAGACAGAGAA |
| | | ATGATCGAAGAAAGACTGAAGACATACGCACACCT |
| | | GTTCGACGACAAGGTCATGAAGCAGCTGAAGAGAA |
| | | GAAGATACACAGGATGGGGAAGACTGAGCAGAAAG |
| | | CTGATCAACGGAATCAGAGACAAGCAGAGCGGAAA |
| | | GACAATCCTGGACTTCCTGAAGAGCGACGGATTCG |
| | | CAAACAGAAACTTCATGCAGCTGATCCACGACGAC |
| | | AGCCTGACATTCAAGGAAGACATCCAGAAGGCACA |
| | | GGTCAGCGGACAGGGAGACAGCCTGCACGAACACA |
| | | TCGCAAACCTGGCAGGAAGCCCGGCAATCAAGAAG |
| | | GGAATCCTGCAGACAGTCAAGGTCGTCGACGAACT |
| | | GGTCAAGGTCATGGGAAGACACAAGCCGGAAAACA |
| | | TCGTCATCGAAATGGCAAGAGAAAACCAGACAACA |
| | | CAGAAGGGACAGAAGAACAGCAGAGAAAGAATGAA |
| | | GAGAATCGAAGAAGGAATCAAGGAACTGGGAAGCC |
| | | AGATCCTGAAGGAACACCCGGTCGAAAACACACAG |
| | | CTGCAGAACGAAAAGCTGTACCTGTACTACCTGCA |
| | | GAACGGAAGAGACATGTACGTCGACCAGGAACTGG |
| | | ACATCAACAGACTGAGCGACTACGACGTCGACCAC |
| | | ATCGTCCCGCAGAGCTTCCTGAAGGACGACAGCAT |
| | | CGACAACAAGGTCCTGACAAGAAGCGACAAGAACA |
| | | GAGGAAAGAGCGACAACGTCCCGAGCGAAGAAGTC |
| | | GTCAAGAAGATGAAGAACTACTGGAGACAGCTGCT |
| | | GAACGCAAAGCTGATCACACAGAGAAAGTTCGACA |
| | | ACCTGACAAAGGCAGAGAGAGGAGGACTGAGCGAA |
| | | CTGGACAAGGCAGGATTCATCAAGAGACAGCTGGT |
| | | CGAAACAAGACAGATCACAAAGCACGTCGCACAGA |
| | | TCCTGGACAGCAGAATGAACACAAAGTACGACGAA |
| | | AACGACAAGCTGATCAGAGAAGTCAAGGTCATCAC |
| | | ACTGAAGAGCAAGCTGGTCAGCGACTTCAGAAAGG |
| | | ACTTCCAGTTCTACAAGGTCAGAGAAATCAACAAC |
| | | TACCACCACGCACACGACGCATACCTGAACGCAGT |
| | | CGTCGGAACAGCACTGATCAAGAAGTACCCGAAGC |
| | | TGGAAAGCGAATTCGTCTACGGAGACTACAAGGTC |
| | | TACGACGTCAGAAAGATGATCGCAAAGAGCGAACA |
| | | GGAAATCGGAAAGGCAACAGCAAAGTACTTCTTCT |
| | | ACAGCAACATCATGAACTTCTTCAAGACAGAAATC |
| | | ACACTGGCAAACGGAGAAATCAGAAAGAGACCGCT |
| | | GATCGAAACAAACGGAGAAACAGGAGAAATCGTCT |
| | | GGGACAAGGGAAGAGACTTCGCAACAGTCAGAAAG |
| | | GTCCTGAGCATGCCGCAGGTCAACATCGTCAAGAA |
| | | GACAGAAGTCCAGACAGGAGGATTCAGCAAGGAAA |
| | | GCATCCTGCCGAAGAGAAACAGCGACAAGCTGATC |
| | | GCAAGAAAGAAGGACTGGGACCCGAAGAAGTACGG |
| | | AGGATTCGACAGCCCGACAGTCGCATACAGCGTCC |
| | | TGGTCGTCGCAAAGGTCGAAAAGGGAAAGAGCAAG |
| | | AAGCTGAAGAGCGTCAAGGAACTGCTGGGAATCAC |
| | | AATCATGGAAAGAAGCAGCTTCGAAAAGAACCCGA |
| | | TCGACTTCCTGGAAGCAAAGGGATACAAGGAAGTC |
| | | AAGAAGGACCTGATCATCAAGCTGCCGAAGTACAG |
| | | CCTGTTCGAACTGGAAAACGGAAGAAAGAGAATGC |
| | | TGGCAAGCGCAGGAGAACTGCAGAAGGGAAACGAA |
| | | CTGGCACTGCCGAGCAAGTACGTCAACTTCCTGTA |
| | | CCTGGCAAGCCACTACGAAAAGCTGAAGGGAAGCC |
| | | CGGAAGACAACGAACAGAAGCAGCTGTTCGTCGAA |
| | | CAGCACAAGCACTACCTGGACGAAATCATCGAACA |
| | | GATCAGCGAATTCAGCAAGAGAGTCATCCTGGCAG |
| | | ACGCAAACCTGGACAAGGTCCTGAGCGCATACAAC |
| | | AAGCACAGAGACAAGCCGATCAGAGAACAGGCAGA |
| | | AAACATCATCCACCTGTTCACACTGACAAACCTGG |
| | | GAGCACCGGCAGCATTCAAGTACTTCGACACAACA |
| | | ATCGACAGAAAGAGATACACAAGCACAAAGGAAGT |
| | | CCTGGACGCAACACTGATCCACCAGAGCATCACAG |
| | | GACTGTACGAAACAAGAATCGACCTGAGCCAGCTG |
| | | GGAGGAGACGGAGGAGGAAGCCCGAAGAAGAAGAG |
| | | AAAGGTCTAG |
| ORF | 10 | ATGGACAAGAAGTACTCCATCGGCCTGGACATCGG |
| encoding | | CACCAACTCCGTGGGCTGGGCCGTGATCACCGACG |
| Sp. Cas9 | | AGTACAAGGTGCCCTCCAAGAAGTTCAAGGTGCTG |
| | | GGCAACACCGACCGGCACTCCATCAAGAAGAACCT |
| | | GATCGGCGCCCTGCTGTTCGACTCCGGCGAGACCG |
| | | CCGAGGCCACCCGGCTGAAGCGGACCGCCCGGCGG |
| | | CGGTACACCCGGCGGAAGAACCGGATCTGCTACCT |
| | | GCAGGAGATCTTCTCCAACGAGATGGCCAAGGTGG |
| | | ACGACTCCTTCTTCCACCGGCTGGAGGAGTCCTTC |
| | | CTGGTGGAGGAGGACAAGAAGCACGAGCGGCACCC |
| | | CATCTTCGGCAACATCGTGGACGAGGTGGCCTACC |
| | | ACGAGAAGTACCCCACCATCTACCACCTGCGGAAG |
| | | AAGCTGGTGGACTCCACCGACAAGGCCGACCTGCG |
| | | GCTGATCTACCTGGCCCTGGCCCACATGATCAAGT |
| | | TCCGGGGCCACTTCCTGATCGAGGGCGACCTGAAC |
| | | CCCGACAACTCCGACGTGGACAAGCTGTTCATCCA |
| | | GCTGGTGCAGACCTACAACCAGCTGTTCGAGGAGA |
| | | ACCCCATCAACGCCTCCGGCGTGGACGCCAAGGCC |
| | | ATCCTGTCCGCCCGGCTGTCCAAGTCCCGGCGGCT |
| | | GGAGAACCTGATCGCCCAGCTGCCCGGCGAGAAGA |
| | | AGAACGGCCTGTTCGGCAACCTGATCGCCCTGTCC |
| | | CTGGGCCTGACCCCCAACTTCAAGTCCAACTTCGA |
| | | CCTGGCCGAGGACGCCAAGCTGCAGCTGTCCAAGG |
| | | ACACCTACGACGACGACCTGGACAACCTGCTGGCC |
| | | CAGATCGGCGACCAGTACGCCGACCTGTTCCTGGC |
| | | CGCCAAGAACCTGTCCGACGCCATCCTGCTGTCCG |
| | | ACATCCTGCGGGTGAACACCGAGATCACCAAGGCC |
| | | CCCCTGTCCGCCTCCATGATCAAGCGGTACGACGA |
| | | GCACCACCAGGACCTGACCCTGCTGAAGGCCCTGG |
| | | TGCGGCAGCAGCTGCCCGAGAAGTACAAGGAGATC |
| | | TTCTTCGACCAGTCCAAGAACGGCTACGCCGGCTA |
| | | CATCGACGGCGGCGCCTCCCAGGAGGAGTTCTACA |
| | | AGTTCATCAAGCCCATCCTGGAGAAGATGGACGGC |
| | | ACCGAGGAGCTGCTGGTGAAGCTGAACCGGGAGGA |
| | | CCTGCTGCGGAAGCAGCGGACCTTCGACAACGGCT |
| | | CCATCCCCCACCAGATCCACCTGGGCGAGCTGCAC |
| | | GCCATCCTGCGGCGGCAGGAGGACTTCTACCCCTT |
| | | CCTGAAGGACAACCGGGAGAAGATCGAGAAGATCC |
| | | TGACCTTCCGGATCCCCTACTACGTGGGCCCCCTG |
| | | GCCCGGGGCAACTCCCGGTTCGCCTGGATGACCCG |
| | | GAAGTCCGAGGAGACCATCACCCCCTGGAACTTCG |
| | | AGGAGGTGGTGGACAAGGGCGCCTCCGCCCAGTCC |
| | | TTCATCGAGCGGATGACCAACTTCGACAAGAACCT |
| | | GCCCAACGAGAAGGTGCTGCCCAAGCACTCCCTGC |
| | | TGTACGAGTACTTCACCGTGTACAACGAGCTGACC |
| | | AAGGTGAAGTACGTGACCGAGGGCATGCGGAAGCC |
| | | CGCCTTCCTGTCCGGCGAGCAGAAGAAGGCCATCG |
| | | TGGACCTGCTGTTCAAGACCAACCGGAAGGTGACC |
| | | GTGAAGCAGCTGAAGGAGGACTACTTCAAGAAGAT |
| | | CGAGTGCTTCGACTCCGTGGAGATCTCCGGCGTGG |
| | | AGGACCGGTTCAACGCCTCCCTGGGCACCTACCAC |
| | | GACCTGCTGAAGATCATCAAGGACAAGGACTTCCT |
| | | GGACAACGAGGAGAACGAGGACATCCTGGAGGACA |
| | | TCGTGCTGACCCTGACCCTGTTCGAGGACCGGGAG |
| | | ATGATCGAGGAGCGGCTGAAGACCTACGCCCACCT |
| | | GTTCGACGACAAGGTGATGAAGCAGCTGAAGCGGC |
| | | GGCGGTACACCGGCTGGGGCCGGCTGTCCCGGAAG |
| | | CTGATCAACGGCATCCGGGACAAGCAGTCCGGCAA |
| | | GACCATCCTGGACTTCCTGAAGTCCGACGGCTTCG |
| | | CCAACCGGAACTTCATGCAGCTGATCCACGACGAC |
| | | TCCCTGACCTTCAAGGAGGACATCCAGAAGGCCCA |
| | | GGTGTCCGGCCAGGGCGACTCCCTGCACGAGCACA |
| | | TCGCCAACCTGGCCGGCTCCCCCGCCATCAAGAAG |
| | | GGCATCCTGCAGACCGTGAAGGTGGTGGACGAGCT |
| | | GGTGAAGGTGATGGGCCGGCACAAGCCCGAGAACA |
| | | TCGTGATCGAGATGGCCCGGGAGAACCAGACCACC |
| | | CAGAAGGGCCAGAAGAACTCCCGGGAGCGGATGAA |
| | | GCGGATCGAGGAGGGCATCAAGGAGCTGGGCTCCC |
| | | AGATCCTGAAGGAGCACCCCGTGGAGAACACCCAG |
| | | CTGCAGAACGAGAAGCTGTACCTGTACTACCTGCA |
| | | GAACGGCCGGGACATGTACGTGGACCAGGAGCTGG |
| | | ACATCAACCGGCTGTCCGACTACGACGTGGACCAC |
| | | ATCGTGCCCCAGTCCTTCCTGAAGGACGACTCCAT |
| | | CGACAACAAGGTGCTGACCCGGTCCGACAAGAACC |
| | | GGGGCAAGTCCGACAACGTGCCCTCCGAGGAGGTG |
| | | GTGAAGAAGATGAAGAACTACTGGCGGCAGCTGCT |
| | | GAACGCCAAGCTGATCACCCAGCGGAAGTTCGACA |
| | | ACCTGACCAAGGCCGAGCGGGGCGGCCTGTCCGAG |
| | | CTGGACAAGGCCGGCTTCATCAAGCGGCAGCTGGT |
| | | GGAGACCCGGCAGATCACCAAGCACGTGGCCCAGA |
| | | TCCTGGACTCCCGGATGAACACCAAGTACGACGAG |
| | | AACGACAAGCTGATCCGGGAGGTGAAGGTGATCAC |
| | | CCTGAAGTCCAAGCTGGTGTCCGACTTCCGGAAGG |
| | | ACTTCCAGTTCTACAAGGTGCGGGAGATCAACAAC |
| | | TACCACCACGCCCACGACGCCTACCTGAACGCCGT |
| | | GGTGGGCACCGCCCTGATCAAGAAGTACCCCAAGC |
| | | TGGAGTCCGAGTTCGTGTACGGCGACTACAAGGTG |
| | | TACGACGTGCGGAAGATGATCGCCAAGTCCGAGCA |
| | | GGAGATCGGCAAGGCCACCGCCAAGTACTTCTTCT |
| | | ACTCCAACATCATGAACTTCTTCAAGACCGAGATC |
| | | ACCCTGGCCAACGGCGAGATCCGGAAGCGGCCCCT |
| | | GATCGAGACCAACGGCGAGACCGGCGAGATCGTGT |
| | | GGGACAAGGGCCGGGACTTCGCCACCGTGCGGAAG |
| | | GTGCTGTCCATGCCCCAGGTGAACATCGTGAAGAA |
| | | GACCGAGGTGCAGACCGGCGGCTTCTCCAAGGAGT |
| | | CCATCCTGCCCAAGCGGAACTCCGACAAGCTGATC |
| | | GCCCGGAAGAAGGACTGGGACCCCAAGAAGTACGG |
| | | CGGCTTCGACTCCCCCACCGTGGCCTACTCCGTGC |
| | | TGGTGGTGGCCAAGGTGGAGAAGGGCAAGTCCAAG |
| | | AAGCTGAAGTCCGTGAAGGAGCTGCTGGGCATCAC |
| | | CATCATGGAGCGGTCCTCCTTCGAGAAGAACCCCA |
| | | TCGACTTCCTGGAGGCCAAGGGCTACAAGGAGGTG |
| | | AAGAAGGACCTGATCATCAAGCTGCCCAAGTACTC |
| | | CCTGTTCGAGCTGGAGAACGGCCGGAAGCGGATGC |
| | | TGGCCTCCGCCGGCGAGCTGCAGAAGGGCAACGAG |
| | | CTGGCCCTGCCCTCCAAGTACGTGAACTTCCTGTA |
| | | CCTGGCCTCCCACTACGAGAAGCTGAAGGGCTCCC |
| | | CCGAGGACAACGAGCAGAAGCAGCTGTTCGTGGAG |
| | | CAGCACAAGCACTACCTGGACGAGATCATCGAGCA |
| | | GATCTCCGAGTTCTCCAAGCGGGTGATCCTGGCCG |
| | | ACGCCAACCTGGACAAGGTGCTGTCCGCCTACAAC |
| | | AAGCACCGGGACAAGCCCATCCGGGAGCAGGCCGA |
| | | GAACATCATCCACCTGTTCACCCTGACCAACCTGG |
| | | GCGCCCCCGCCGCCTTCAAGTACTTCGACACCACC |
| | | ATCGACCGGAAGCGGTACACCTCCACCAAGGAGGT |
| | | GCTGGACGCCACCCTGATCCACCAGTCCATCACCG |
| | | GCCTGTACGAGACCCGGATCGACCTGTCCCAGCTG |
| | | GGCGGCGACGGCGGCGGCTCCCCCAAGAAGAAGCG |
| | | GAAGGTGTGA |
| Open | 11 | AUGGACAAGAAGUACUCCAUCGGCCUGGACAUCGG |
| reading | | CACCAACUCCGUGGGCUGGGCCGUGAUCACCGACG |
| frame for | | AGUACAAGGUGCCCUCCAAGAAGUUCAAGGUGCUG |
| Cas9 with | | GGCAACACCGACCGGCACUCCAUCAAGAAGAACCU |
| Hibit tag | | GAUCGGCGCCCUGCUGUUCGACUCCGGCGAGACCG |
| | | CCGAGGCCACCCGGCUGAAGCGGACCGCCCGGCGG |
| | | CGGUACACCCGGCGGAAGAACCGGAUCUGCUACCU |
| | | GCAGGAGAUCUUCUCCAACGAGAUGGCCAAGGUGG |
| | | ACGACUCCUUCUUCCACCGGCUGGAGGAGUCCUUC |
| | | CUGGUGGAGGAGGACAAGAAGCACGAGCGGCACCC |
| | | CAUCUUCGGCAACAUCGUGGACGAGGUGGCCUACC |
| | | ACGAGAAGUACCCCACCAUCUACCACCUGCGGAAG |
| | | AAGCUGGUGGACUCCACCGACAAGGCCGACCUGCG |
| | | GCUGAUCUACCUGGCCCUGGCCCACAUGAUCAAGU |
| | | UCCGGGGCCACUUCCUGAUCGAGGGCGACCUGAAC |
| | | CCCGACAACUCCGACGUGGACAAGCUGUUCAUCCA |
| | | GCUGGUGCAGACCUACAACCAGCUGUUCGAGGAGA |
| | | ACCCCAUCAACGCCUCCGGCGUGGACGCCAAGGCC |
| | | AUCCUGUCCGCCCGGCUGUCCAAGUCCCGGCGGCU |
| | | GGAGAACCUGAUCGCCCAGCUGCCCGGCGAGAAGA |
| | | AGAACGGCCUGUUCGGCAACCUGAUCGCCCUGUCC |
| | | CUGGGCCUGACCCCCAACUUCAAGUCCAACUUCGA |
| | | CCUGGCCGAGGACGCCAAGCUGCAGCUGUCCAAGG |
| | | ACACCUACGACGACGACCUGGACAACCUGCUGGCC |
| | | CAGAUCGGCGACCAGUACGCCGACCUGUUCCUGGC |
| | | CGCCAAGAACCUGUCCGACGCCAUCCUGCUGUCCG |
| | | ACAUCCUGCGGGUGAACACCGAGAUCACCAAGGCC |
| | | CCCCUGUCCGCCUCCAUGAUCAAGCGGUACGACGA |
| | | GCACCACCAGGACCUGACCCUGCUGAAGGCCCUGG |
| | | UGCGGCAGCAGCUGCCCGAGAAGUACAAGGAGAUC |
| | | UUCUUCGACCAGUCCAAGAACGGCUACGCCGGCUA |
| | | CAUCGACGGCGGCGCCUCCCAGGAGGAGUUCUACA |
| | | AGUUCAUCAAGCCCAUCCUGGAGAAGAUGGACGGC |
| | | ACCGAGGAGCUGCUGGUGAAGCUGAACCGGGAGGA |
| | | CCUGCUGCGGAAGCAGCGGACCUUCGACAACGGCU |
| | | CCAUCCCCCACCAGAUCCACCUGGGCGAGCUGCAC |
| | | GCCAUCCUGCGGCGGCAGGAGGACUUCUACCCCUU |
| | | CCUGAAGGACAACCGGGAGAAGAUCGAGAAGAUCC |
| | | UGACCUUCCGGAUCCCCUACUACGUGGGCCCCCUG |
| | | GCCCGGGGCAACUCCCGGUUCGCCUGGAUGACCCG |
| | | GAAGUCCGAGGAGACCAUCACCCCCUGGAACUUCG |
| | | AGGAGGUGGUGGACAAGGGCGCCUCCGCCCAGUCC |
| | | UUCAUCGAGCGGAUGACCAACUUCGACAAGAACCU |
| | | GCCCAACGAGAAGGUGCUGCCCAAGCACUCCCUGC |
| | | UGUACGAGUACUUCACCGUGUACAACGAGCUGACC |
| | | AAGGUGAAGUACGUGACCGAGGGCAUGCGGAAGCC |
| | | CGCCUUCCUGUCCGGCGAGCAGAAGAAGGCCAUCG |
| | | UGGACCUGCUGUUCAAGACCAACCGGAAGGUGACC |
| | | GUGAAGCAGCUGAAGGAGGACUACUUCAAGAAGAU |
| | | CGAGUGCUUCGACUCCGUGGAGAUCUCCGGCGUGG |
| | | AGGACCGGUUCAACGCCUCCCUGGGCACCUACCAC |
| | | GACCUGCUGAAGAUCAUCAAGGACAAGGACUUCCU |
| | | GGACAACGAGGAGAACGAGGACAUCCUGGAGGACA |
| | | UCGUGCUGACCCUGACCCUGUUCGAGGACCGGGAG |
| | | AUGAUCGAGGAGCGGCUGAAGACCUACGCCCACCU |
| | | GUUCGACGACAAGGUGAUGAAGCAGCUGAAGCGGC |
| | | GGCGGUACACCGGCUGGGGCCGGCUGUCCCGGAAG |
| | | CUGAUCAACGGCAUCCGGGACAAGCAGUCCGGCAA |
| | | GACCAUCCUGGACUUCCUGAAGUCCGACGGCUUCG |
| | | CCAACCGGAACUUCAUGCAGCUGAUCCACGACGAC |
| | | UCCCUGACCUUCAAGGAGGACAUCCAGAAGGCCCA |
| | | GGUGUCCGGCCAGGGCGACUCCCUGCACGAGCACA |
| | | UCGCCAACCUGGCCGGCUCCCCCGCCAUCAAGAAG |
| | | GGCAUCCUGCAGACCGUGAAGGUGGUGGACGAGCU |
| | | GGUGAAGGUGAUGGGCCGGCACAAGCCCGAGAACA |
| | | UCGUGAUCGAGAUGGCCCGGGAGAACCAGACCACC |
| | | CAGAAGGGCCAGAAGAACUCCCGGGAGCGGAUGAA |
| | | GCGGAUCGAGGAGGGCAUCAAGGAGCUGGGCUCCC |
| | | AGAUCCUGAAGGAGCACCCCGUGGAGAACACCCAG |
| | | CUGCAGAACGAGAAGCUGUACCUGUACUACCUGCA |
| | | GAACGGCCGGGACAUGUACGUGGACCAGGAGCUGG |
| | | ACAUCAACCGGCUGUCCGACUACGACGUGGACCAC |
| | | AUCGUGCCCCAGUCCUUCCUGAAGGACGACUCCAU |
| | | CGACAACAAGGUGCUGACCCGGUCCGACAAGAACC |
| | | GGGGCAAGUCCGACAACGUGCCCUCCGAGGAGGUG |
| | | GUGAAGAAGAUGAAGAACUACUGGCGGCAGCUGCU |
| | | GAACGCCAAGCUGAUCACCCAGCGGAAGUUCGACA |
| | | ACCUGACCAAGGCCGAGCGGGGCGGCCUGUCCGAG |
| | | CUGGACAAGGCCGGCUUCAUCAAGCGGCAGCUGGU |
| | | GGAGACCCGGCAGAUCACCAAGCACGUGGCCCAGA |
| | | UCCUGGACUCCCGGAUGAACACCAAGUACGACGAG |
| | | AACGACAAGCUGAUCCGGGAGGUGAAGGUGAUCAC |
| | | CCUGAAGUCCAAGCUGGUGUCCGACUUCCGGAAGG |
| | | ACUUCCAGUUCUACAAGGUGCGGGAGAUCAACAAC |
| | | UACCACCACGCCCACGACGCCUACCUGAACGCCGU |
| | | GGUGGGCACCGCCCUGAUCAAGAAGUACCCCAAGC |
| | | UGGAGUCCGAGUUCGUGUACGGCGACUACAAGGUG |
| | | UACGACGUGCGGAAGAUGAUCGCCAAGUCCGAGCA |
| | | GGAGAUCGGCAAGGCCACCGCCAAGUACUUCUUCU |
| | | ACUCCAACAUCAUGAACUUCUUCAAGACCGAGAUC |
| | | ACCCUGGCCAACGGCGAGAUCCGGAAGCGGCCCCU |
| | | GAUCGAGACCAACGGCGAGACCGGCGAGAUCGUGU |
| | | GGGACAAGGGCCGGGACUUCGCCACCGUGCGGAAG |
| | | GUGCUGUCCAUGCCCCAGGUGAACAUCGUGAAGAA |
| | | GACCGAGGUGCAGACCGGCGGCUUCUCCAAGGAGU |
| | | CCAUCCUGCCCAAGCGGAACUCCGACAAGCUGAUC |
| | | GCCCGGAAGAAGGACUGGGACCCCAAGAAGUACGG |
| | | CGGCUUCGACUCCCCCACCGUGGCCUACUCCGUGC |
| | | UGGUGGUGGCCAAGGUGGAGAAGGGCAAGUCCAAG |
| | | AAGCUGAAGUCCGUGAAGGAGCUGCUGGGCAUCAC |
| | | CAUCAUGGAGCGGUCCUCCUUCGAGAAGAACCCCA |
| | | UCGACUUCCUGGAGGCCAAGGGCUACAAGGAGGUG |
| | | AAGAAGGACCUGAUCAUCAAGCUGCCCAAGUACUC |
| | | CCUGUUCGAGCUGGAGAACGGCCGGAAGCGGAUGC |
| | | UGGCCUCCGCCGGCGAGCUGCAGAAGGGCAACGAG |
| | | CUGGCCCUGCCCUCCAAGUACGUGAACUUCCUGUA |
| | | CCUGGCCUCCCACUACGAGAAGCUGAAGGGCUCCC |
| | | CCGAGGACAACGAGCAGAAGCAGCUGUUCGUGGAG |
| | | CAGCACAAGCACUACCUGGACGAGAUCAUCGAGCA |
| | | GAUCUCCGAGUUCUCCAAGCGGGUGAUCCUGGCCG |
| | | ACGCCAACCUGGACAAGGUGCUGUCCGCCUACAAC |
| | | AAGCACCGGGACAAGCCCAUCCGGGAGCAGGCCGA |
| | | GAACAUCAUCCACCUGUUCACCCUGACCAACCUGG |
| | | GCGCCCCCGCCGCCUUCAAGUACUUCGACACCACC |
| | | AUCGACCGGAAGCGGUACACCUCCACCAAGGAGGU |
| | | GCUGGACGCCACCCUGAUCCACCAGUCCAUCACCG |
| | | GCCUGUACGAGACCCGGAUCGACCUGUCCCAGCUG |
| | | GGCGGCGACGGCGGCGGCUCCCCCAAGAAGAAGCG |
| | | GAAGGUGUCCGAGUCCGCCACCCCCGAGUCCGUGU |
| | | CCGGCUGGCGGCUGUUCAAGAAGAUCUCCUGA |
| HD1 TCR | 12 | ttggccactccctctctgcgcgctcgctcgctcac |
| insertion | | tgaggccgggcgaccaaaggtegcccgacgcccgg |
| including | | gctttgcccgggcggcctcagtgagcgagcgagcg |
| ITRs | | cgcagagagggagtggccaactccatcactagggg |
| | | ttcctagatcttgccaacataccataaacctccca |
| | | ttctgctaatgcccagcctaagttggggagaccac |
| | | tccagattccaagatgtacagtttgctttgctggg |
| | | cctttttcccatgcctgcctttactctgccagagt |
| | | tatattgctggggttttgaagaagatcctattaaa |
| | | taaaagaataagcagtattattaagtagccctgca |
| | | tttcaggtttccttgagtggcaggccaggcctggc |
| | | cgtgaacgttcactgaaatcatggcctcttggcca |
| | | agattgatagcttgtgcctgtccctgagtcccagt |
| | | ccatcacgagcagctggtttctaagatgctatttc |
| | | ccgtataaagcatgagaccgtgacttgccagcccc |
| | | acagagccccgcccttgtccatcactggcatctgg |
| | | actccagcctgggttggggcaaagagggaaatgag |
| | | atcatgtcctaaccctgatcctcttgtcccacaga |
| | | tatccagaaccctgaccctgcggctccggtgcccg |
| | | tcagtgggcagagcgcacatcgcccacagtccccg |
| | | agaagttggggggaggggtcggcaattgaaccggt |
| | | gcctagagaaggtggcgcggggtaaactgggaaag |
| | | tgatgtcgtgtactggctccgcctttttcccgagg |
| | | ggggggagaaccgtatataagtgcagtagtcgccg |
| | | tgaacgttctttttcgcaacgggtttgccgccaga |
| | | acacaggtaagtgccgtgtgtggttcccgcgggcc |
| | | tggcctctttacgggttatggcccttgcgtgcctt |
| | | gaattacttccacgcccctggctgcagtacgtgat |
| | | tcttgatcccgagcttcgggttggaagtgggtggg |
| | | agagttcgaggccttgcgcttaaggagccccttcg |
| | | cctcgtgcttgagttgaggcctggcttgggcgctg |
| | | gggccgccgcgtgcgaatctggtggcaccttcgcg |
| | | cctgtctcgctgctttcgataagtctctagccatt |
| | | taaaatttttgatgacctgctgcgacgcttttttt |
| | | ctggcaagatagtcttgtaaatgcgggccaagatg |
| | | tgcacactggtatttcggtttttggggccgcgggg |
| | | gcgacggggcccgtgcgtcccagcgcacatgttcg |
| | | gcgaggcggggcctgcgagcgcggccaccgagaat |
| | | cggacgggggtagtctcaagctggccggcctgctc |
| | | tggtgcctggcctcgcgccgccgtgtatcgccccg |
| | | ccctgggcggcaaggctggcccggtcggcaccagt |
| | | tgcgtgagcggaaagatggccgcttcccggccctg |
| | | ctgcagggagctcaaaatggaggacgcggcgctcg |
| | | ggagagcggggggtgagtcacccacacaaaggaaa |
| | | agggcctttccgtcctcagccgtcgcttcatgtga |
| | | ctccacggagtaccgggcgccgtccaggcacctcg |
| | | attagttctcgagcttttggagtacgtcgtcttta |
| | | ggttggggggaggggttttatgcgatggagtttcc |
| | | ccacactgagtgggtggagactgaagttaggccag |
| | | cttggcacttgatgtaattctccttggaatttgcc |
| | | ctttttgagtttggatcttggttcattctcaagcc |
| | | tcagacagtggttcaaagtttttttcttccatttc |
| | | aggtgtcgtgatgcggccgccaccatgggatcttg |
| | | gacactgtgttgcgtgtccctgtgcatcctggtgg |
| | | ccaagcacacagatgccggcgtgatccagtctcct |
| | | agacacgaagtgaccgagatgggccaagaagtgac |
| | | cctgcgctgcaagcctatcagcggccacgattacc |
| | | tgttctggtacagacagaccatgatgagaggcctg |
| | | gaactgctgatctacttcaacaacaacgtgcccat |
| | | cgacgacagcggcatgcccgaggatagattcagcg |
| | | ccaagatgcccaacgccagcttcagcaccctgaag |
| | | atccagcctagcgagcccagagatagcgccgtgta |
| | | cttctgcgccagcagaaagacaggcggctacagca |
| | | atcagccccagcactttggagatggcacccggctg |
| | | agcatcctggaagatctgaagaacgtgttcccacc |
| | | tgaggtggccgtgttcgagccttctgaggccgaga |
| | | tcagccacacacagaaagccacactcgtgtgtctg |
| | | gccaccggcttctatcccgatcacgtggaactgtc |
| | | ttggtgggtcaacggcaaagaggtgcacagcggcg |
| | | tcagcaccgatcctcagcctctgaaagagcagccc |
| | | gctctgaacgacagcagatactgcctgagcagcag |
| | | actgagagtgtccgccaccttctggcagaacccca |
| | | gaaaccacttcagatgccaggtgcagttctacggc |
| | | ctgagcgagaacgatgagtggacccaggatagagc |
| | | caagcctgtgacacagatcgtgtctgccgaagcct |
| | | ggggcagagccgattgtggctttaccagcgagagc |
| | | taccagcagggcgtgctgtctgccacaatcctgta |
| | | cgagatcctgctgggcaaagccactctgtacgccg |
| | | tgctggtgtctgccctggtgctgatggccatggtc |
| | | aagcggaaggatagcaggggcggctccggtgccac |
| | | aaacttctccctgctcaagcaggccggagatgtgg |
| | | aagagaaccctggccctatggaaaccctgctgaag |
| | | gtgctgagcggcacactgctgtggcagctgacatg |
| | | ggtccgatctcagcagcctgtgcagtctcctcagg |
| | | ccgtgattctgagagaaggcgaggacgccgtgatc |
| | | aactgcagcagctctaaggccctgtacagcgtgca |
| | | ctggtacagacagaagcacggcgaggcccctgtgt |
| | | tcctgatgatcctgctgaaaggcggcgagcagaag |
| | | ggccacgagaagatcagcgccagcttcaacgagaa |
| | | gaagcagcagtccagcctgtacctgacagccagcc |
| | | agctgagctacagcggcacctacttttgtggcacc |
| | | gcctggatcaacgactacaagctgtctttcggagc |
| | | cggcaccacagtgacagtgcgggccaatattcaga |
| | | accccgatcctgccgtgtaccagctgagagacagc |
| | | aagagcagcgacaagagcgtgtgcctgttcaccga |
| | | cttcgacagccagaccaacgtgtcccagagcaagg |
| | | acagcgacgtgtacatcaccgataagactgtgctg |
| | | gacatgcggagcatggacttcaagagcaacagcgc |
| | | cgtggcctggtccaacaagagcgatttcgcctgcg |
| | | ccaacgccttcaacaacagcattatccccgaggac |
| | | acattcttcccaagtcctgagagcagctgcgacgt |
| | | gaagctggtggaaaagagcttcgagacagacacca |
| | | acctgaacttccagaacctgagcgtgatcggcttc |
| | | agaatcctgctgctcaaggtggccggcttcaacct |
| | | gctgatgaccctgagactgtggtccagctaacctC |
| | | GACTGTGCCTTCTAGTTGCCAGCCATCTGTTGTTT |
| | | GCCCCTCCCCCGTGCCTTCCTTGACCCTGGAAGGT |
| | | GCCACTCCCACTGTCCTTTCCTAATAAAATGAGGA |
| | | AATTGCATCGCATTGTCTGAGTAGGTGTCATTCTA |
| | | TTCTGGGGGGGGGGTGGGGCAGGACAGCAAGGGGG |
| | | AGGATTGGGAAGACAATAGCAGGCATGCTGGGGAT |
| | | GCGGTGGGCTCTATGGcttctgaggcggaaagaac |
| | | cagctggggctctagggggtatccccactagtcgt |
| | | gtaccagctgagagactctaaatccagtgacaagt |
| | | ctgtctgcctattcaccgattttgattctcaaaca |
| | | aatgtgtcacaaagtaaggattctgatgtgtatat |
| | | cacagacaaaactgtgctagacatgaggtctatgg |
| | | acttcaagagcaacagtgctgtggcctggagcaac |
| | | aaatctgactttgcatgtgcaaacgccttcaacaa |
| | | cagcattattccagaagacaccttcttccccagcc |
| | | caggtaagggcagctttggtgccttcgcaggctgt |
| | | ttccttgcttcaggaatggccaggttctgcccaga |
| | | gctctggtcaatgatgtctaaaactcctctgattg |
| | | gtggtctcggccttatccattgccaccaaaaccct |
| | | ctttttactaagaaacagtgagccttgttctggca |
| | | gtccagagaatgacacgggaaaaaagcagatgaag |
| | | agaaggtggcaggagagggcacgtggcccagcctc |
| | | agtctctagatctaggaacccctagtgatggagtt |
| | | ggccactccctctctgcgcgctcgctcgctcactg |
| | | aggccgcccgggcaaagcccgggcgtcgggcgacc |
| | | tttggtcgcccggcctcagtgagcgagcgagcgcg |
| | | cagagagggagtggccaa |
| pINT- | 13 | taatcagaattggttaattggttgtaacattattc |
| 2v547 | | agattgggcttgatttaaaacttcatttttaattt |
| | | aaaaggatctaggtgaagatcctttttgataatct |
| | | catgaccaaaatcccttaacgtgagttttcgttcc |
| | | actgagcgtcagaccccgtagaaaagatcaaagga |
| | | tcttcttgagatcctttttttctgcgcgtaatctg |
| | | ctgcttgcaaacaaaaaaaccaccgctaccagcgg |
| | | tggtttgtttgccggatcaagagctaccaactctt |
| | | tttccgaaggtaactggcttcagcagagcgcagat |
| | | accaaatactgttcttctagtgtagccgtagttag |
| | | gccaccacttcaagaactctgtagcaccgcctaca |
| | | tacctcgctctgctaatcctgttaccagtggctgc |
| | | tgccagtggcgataagtcgtgtcttaccgggttgg |
| | | actcaagacgatagttaccggataaggcgcagcgg |
| | | tcgggctgaacggggggttcgtgcacacagcccag |
| | | cttggagcgaacgacctacaccgaactgagatacc |
| | | tacagcgtgagctatgagaaagcgccacgcttccc |
| | | gaagggagaaaggcggacaggtatccggtaagcgg |
| | | cagggtcggaacaggagagcgcacgagggagcttc |
| | | cagggggaaacgcctggtatctttatagtcctgtc |
| | | gggtttcgccacctctgacttgagcgtcgattttt |
| | | gtgatgctcgtcaggggggcggagcctatggaaaa |
| | | acgccagcaacgcggcctttttacggttcctggcc |
| | | ttttgctggccttttgctcacatgttctttcctgc |
| | | gttatcccctgattctgtggataaccgtattaccg |
| | | cctttgagtgagctgataccgctcgccgcagccga |
| | | acgaccgagcgcagcgagtcagtgagcgaggaagc |
| | | ggaagagcgcccaatacgcaaaccgcctctccccg |
| | | cgcgttggccgattcattaatgcagctggcacgac |
| | | aggtttcccgactggaaagcgggcagtgagcgcaa |
| | | cgcaattaatgtgagttagctcactcattaggcac |
| | | cccaggctttacactttatgcttccggctcgtatg |
| | | ttgtgtggaattgtgagcggataacaatttcacac |
| | | aggaaacagctatgaccatgattacaccacgcgtT |
| | | TGGCCACTCCCTCTCTGCGCGCTCGCTCGCTCACT |
| | | GAGGCCGGGCGACCAAAGGTCGCCCGACGCCCGGG |
| | | CTTTGCCCGGGCGGCCTCAGTGAGCGAGCGAGCGC |
| | | GCAGAGAGGGAGTGGCCAACTCCATCACTAGGGGT |
| | | TCCTagatcttgccaacataccataaacctcccat |
| | | tctgctaatgcccagcctaagttggggagaccact |
| | | ccagattccaagatgtacagtttgctttgctgggc |
| | | ctttttcccatgcctgcctttactctgccagagtt |
| | | atattgctggggttttgaagaagatcctattaaat |
| | | aaaagaataagcagtattattaagtagccctgcat |
| | | ttcaggtttccttgagtggcaggccaggcctggcc |
| | | gtgaacgttcactgaaatcatggcctcttggccaa |
| | | gattgatagcttgtgcctgtccctgagtcccagtc |
| | | catcacgagcagctggtttctaagatgctatttcc |
| | | cgtataaagcatgagaccgtgacttgccagcccca |
| | | cagagccccgcccttgtccatcactggcatctgga |
| | | ctccagcctgggttggggcaaagagggaaatgaga |
| | | tcatgtcctaaccctgatcctcttgtcccacagAT |
| | | ATCCAGAACCCTGACCCTGCcGAGGGCCGCGGCAG |
| | | CCTGCTGACCTGCGGCGACGTGGAGGAGAAtCCCG |
| | | GCCCCATGgtgAGCAAGGGCGAGGAGCTGTTCACC |
| | | GGGGTGGTGCCCATCCTGGTCGAGCTGGACGGCGA |
| | | CGTAAACGGCCACAAGTTCAGCGTGTCCGGCGAGG |
| | | GCGAGGGCGATGCCACCTACGGCAAGCTGACCCTG |
| | | AAGTTCATCTGCACCACCGGCAAGCTGCCCGTGCC |
| | | CTGGCCCACCCTCGTGACCACCCTGACCTACGGCG |
| | | TGCAGTGCTTCAGCCGCTACCCCGACCACATGAAG |
| | | CAGCACGACTTCTTCAAGTCCGCCATGCCCGAAGG |
| | | CTACGTCCAGGAGCGCACCATCTTCTTCAAGGACG |
| | | ACGGCAACTACAAGACCCGCGCCGAGGTGAAGTTC |
| | | GAGGGCGACACCCTGGTGAACCGCATCGAGCTGAA |
| | | GGGCATCGACTTCAAGGAGGACGGCAACATCCTGG |
| | | GGCACAAGCTGGAGTACAACTACAACAGCCACAAC |
| | | GTCTATATCATGGCCGACAAGCAGAAGAACGGCAT |
| | | CAAGGTGAACTTCAAGATCCGCCACAACATCGAGG |
| | | ACGGCAGCGTGCAGCTCGCCGACCACTACCAGCAG |
| | | AACACCCCCATCGGCGACGGCCCCGTGCTGCTGCC |
| | | CGACAACCACTACCTGAGCACCCAGTCCGCCCTGA |
| | | GCAAAGACCCCAACGAGAAGCGCGATCACATGGTC |
| | | CTGCTGGAGTTCGTGACCGCCGCCGGGATCACTCT |
| | | CGGCATGGACGAGCTGTACAAGTAAcctCGACTGT |
| | | GCCTTCTAGTTGCCAGCCATCTGTTGTTTGCCCCT |
| | | CCCCCGTGCCTTCCTTGACCCTGGAAGGTGCCACT |
| | | CCCACTGTCCTTTCCTAATAAAATGAGGAAATTGC |
| | | ATCGCATTGTCTGAGTAGGTGTCATTCTATTCTGG |
| | | GGGGGGGGTGGGGCAGGACAGCAAGGGGGAGGATT |
| | | GGGAAGACAATAGCAGGCATGCTGGGGATGCGGTG |
| | | GGCTCTATGGcttctgaggcggaaagaaccagctg |
| | | gggctctagggggtatccccACTAGTCGTGTACCA |
| | | GCTGAGAGACTCTAAATCCAGTGACAAGTCTGTCT |
| | | GCCTATTCACCGATTTTGATTCTCAAACAAATGTG |
| | | TCACAAAGTAAGGATTCTGATGTGTATATCACAGA |
| | | CAAAACTGTGCTAGACATGAGGTCTATGGACTTCA |
| | | AGAGCAACAGTGCTGTGGCCTGGAGCAACAAATCT |
| | | GACTTTGCATGTGCAAACGCCTTCAACAACAGCAT |
| | | TATTCCAGAAGACACCTTCTTCCCCAGCCCAGgta |
| | | agggcagctttggtgccttcgcaggctgtttcctt |
| | | gcttcaggaatggccaggttctgcccagagctctg |
| | | gtcaatgatgtctaaaactcctctgattggtggtc |
| | | tcggccttatccattgccaccaaaaccctcttttt |
| | | actaagaaacagtgagccttgttctggcagtccag |
| | | agaatgacacgggaaaaaagcagatgaagagaagg |
| | | tggcaggagagggcacgtggcccagcctcagtctc |
| | | tAGATCTAGGAACCCCTAGTGATGGAGTTGGCCAC |
| | | TCCCTCTCTGCGCGCTCGCTCGCTCACTGAGGCCG |
| | | CCCGGGCAAAGCCCGGGCGTCGGGCGACCTTTGGT |
| | | CGCCCGGCCTCAGTGAGCGAGCGAGCGCGCAGAGA |
| | | GGGAGTGGCCAAgaattctctggccgtcgttttac |
| | | aacgtcgtgactgggaaaaccctggcgttacccaa |
| | | cttaatcgccttgcagcacatccccctttcgccag |
| | | ctggcgtaatagcgaagaggcccgcaccgatcgcc |
| | | cttcccaacagttgcgcagcctgaatggcgaatgg |
| | | cgcctgatgcggtattttctccttacgcatctgtg |
| | | cggtatttcacaccgcatatggtgcactctcagta |
| | | caatctgctctgatgccgcatagttaagccagccc |
| | | cgacacccgccaacacccgctgacgcgccctgacg |
| | | ggcttgtctgctcccggcatccgcttacagacaag |
| | | ctgtgaccgtctccgggagctgcatgtgtcagagg |
| | | ttttcaccgtcatcaccgaaacgcgcgatgcagct |
| | | ctggcccgtgtctcaaaatctctgatgttacattg |
| | | cacaagataaaaatatatcatcatgaacaataaaa |
| | | ctgtctgcttacataaacagtaatacaaggggtgt |
| | | tatgagccatattcaacgggaaacgtcgaggccgc |
| | | gattaaattccaacatggatgctgatttatatggg |
| | | tataaatgggctcgcgataatgtcgggcaatcagg |
| | | tgcgacaatctatcgcttgtatgggaagcccgatg |
| | | cgccagagttgtttctgaaacatggcaaaggtagc |
| | | gttgccaatgatgttacagatgagatggtcagact |
| | | aaactggctgacggaatttatgcctcttccgacca |
| | | tcaagcattttatccgtactcctgatgatgcatgg |
| | | ttactcaccactgcgatccccggaaaaacagcatt |
| | | ccaggtattagaagaatatcctgattcaggtgaaa |
| | | atattgttgatgcgctggcagtgttcctgcgccgg |
| | | ttgcattcgattcctgtttgtaattgtccttttaa |
| | | cagcgatcgcgtatttcgtctcgctcaggcgcaat |
| | | cacgaatgaataacggtttggttgatgcgagtgat |
| | | tttgatgacgagcgtaatggctggcctgttgaaca |
| | | agtctggaaagaaatgcataaacttttgccattct |
| | | caccggattcagtcgtcactcatggtgatttctca |
| | | cttgataaccttatttttgacgaggggaaattaat |
| | | aggttgtattgatgttggacgagtcggaatcgcag |
| | | accgataccaggatcttgccatcctatggaactgc |
| | | ctcggtgagttttctccttcattacagaaacggct |
| | | ttttcaaaaatatggtattgataatcctgatatga |
| | | ataaattgcagtttcatttgatgctcgatgagttt |
| | | ttc |
| pINT | 14 | ttggccactccctctctgcgcgctcgctcgctcac |
| 1405, | | tgaggccgggcgaccaaaggtcgcccgacgcccgg |
| HD1 TCR | | gctttgcccgggcggcctcagtgagcgagcgagcg |
| insertion | | cgcagagagggagtggccaactccatcactagggg |
| including | | ttcctagatcttgccaacataccataaacctccca |
| ITRs | | ttctgctaatgcccagcctaagttggggagaccac |
| | | tccagattccaagatgtacagtttgctttgctggg |
| | | cctttttcccatgcctgcctttactctgccagagt |
| | | tatattgctggggttttgaagaagatcctattaaa |
| | | taaaagaataagcagtattattaagtagccctgca |
| | | tttcaggtttccttgagtggcaggccaggcctggc |
| | | cgtgaacgttcactgaaatcatggcctcttggcca |
| | | agattgatagcttgtgcctgtccctgagtcccagt |
| | | ccatcacgagcagctggtttctaagatgctatttc |
| | | ccgtataaagcatgagaccgtgacttgccagcccc |
| | | acagagccccgcccttgtccatcactggcatctgg |
| | | actccagcctgggttggggcaaagagggaaatgag |
| | | atcatgtcctaaccctgatcctcttgtcccacaga |
| | | tatccagaaccctgaccctgcggctccggtgcccg |
| | | tcagtgggcagagcgcacatcgcccacagtccccg |
| | | agaagttggggggaggggtcggcaattgaaccggt |
| | | gcctagagaaggtggcgcggggtaaactgggaaag |
| | | tgatgtcgtgtactggctccgcctttttcccgagg |
| | | ggggggagaaccgtatataagtgcagtagtcgccg |
| | | tgaacgttctttttcgcaacgggtttgccgccaga |
| | | acacaggtaagtgccgtgtgtggttcccgcgggcc |
| | | tggcctctttacgggttatggcccttgcgtgcctt |
| | | gaattacttccacgcccctggctgcagtacgtgat |
| | | tcttgatcccgagcttcgggttggaagtgggtggg |
| | | agagttcgaggccttgcgcttaaggagccccttcg |
| | | cctcgtgcttgagttgaggcctggcttgggcgctg |
| | | gggccgccgcgtgcgaatctggtggcaccttcgcg |
| | | cctgtctcgctgctttcgataagtctctagccatt |
| | | taaaatttttgatgacctgctgcgacgcttttttt |
| | | ctggcaagatagtcttgtaaatgcgggccaagatg |
| | | tgcacactggtatttcggtttttggggccgcgggc |
| | | ggcgacggggcccgtgcgtcccagcgcacatgttc |
| | | ggcgaggcggggcctgcgagcgcggccaccgagaa |
| | | tcggacgggggtagtctcaagctggccggcctgct |
| | | ctggtgcctggcctcgcgccgccgtgtatcgcccc |
| | | gccctgggcggcaaggctggcccggtcggcaccag |
| | | ttgcgtgagcggaaagatggccgcttcccggccct |
| | | gctgcagggagctcaaaatggaggacgcggcgctc |
| | | gggagagcgggcgggtgagtcacccacacaaagga |
| | | aaagggcctttccgtcctcagccgtcgcttcatgt |
| | | gactccacggagtaccgggcgccgtccaggcacct |
| | | cgattagttctcgagcttttggagtacgtcgtctt |
| | | taggttggggggaggggttttatgcgatggagttt |
| | | ccccacactgagtgggtggagactgaagttaggcc |
| | | agcttggcacttgatgtaattctccttggaatttg |
| | | ccctttttgagtttggatcttggttcattctcaag |
| | | cctcagacagtggttcaaagtttttttcttccatt |
| | | tcaggtgtcgtgatgcggccgccaccatgggatct |
| | | tggacactgtgttgcgtgtccctgtgcatcctggt |
| | | ggccaagcacacagatgccggcgtgatccagtctc |
| | | ctagacacgaagtgaccgagatgggccaagaagtg |
| | | accctgcgctgcaagcctatcagcggccacgatta |
| | | cctgttctggtacagacagaccatgatgagaggcc |
| | | tggaactgctgatctacttcaacaacaacgtgccc |
| | | atcgacgacagcggcatgcccgaggatagattcag |
| | | cgccaagatgcccaacgccagcttcagcaccctga |
| | | agatccagcctagcgagcccagagatagcgccgtg |
| | | tacttctgcgccagcagaaagacaggcggctacag |
| | | caatcagccccagcactttggagatggcacccggc |
| | | tgagcatcctggaagatctgaagaacgtgttccca |
| | | cctgaggtggccgtgttcgagccttctgaggccga |
| | | gatcagccacacacagaaagccacactcgtgtgtc |
| | | tggccaccggcttctatcccgatcacgtggaactg |
| | | tcttggtgggtcaacggcaaagaggtgcacagcgg |
| | | cgtcagcaccgatcctcagcctctgaaagagcagc |
| | | ccgctctgaacgacagcagatactgcctgagcagc |
| | | agactgagagtgtccgccaccttctggcagaaccc |
| | | cagaaaccacttcagatgccaggtgcagttctacg |
| | | gcctgagcgagaacgatgagtggacccaggataga |
| | | gccaagcctgtgacacagatcgtgtctgccgaagc |
| | | ctggggcagagccgattgtggctttaccagcgaga |
| | | gctaccagcagggcgtgctgtctgccacaatcctg |
| | | tacgagatcctgctgggcaaagccactctgtacgc |
| | | cgtgctggtgtctgccctggtgctgatggccatgg |
| | | tcaagcggaaggatagcaggggcggctccggtgcc |
| | | acaaacttctccctgctcaagcaggccggagatgt |
| | | ggaagagaaccctggccctatggaaaccctgctga |
| | | aggtgctgagcggcacactgctgtggcagctgaca |
| | | tgggtccgatctcagcagcctgtgcagtctcctca |
| | | ggccgtgattctgagagaaggcgaggacgccgtga |
| | | tcaactgcagcagctctaaggccctgtacagcgtg |
| | | cactggtacagacagaagcacggcgaggcccctgt |
| | | gttcctgatgatcctgctgaaaggcggcgagcaga |
| | | agggccacgagaagatcagcgccagcttcaacgag |
| | | aagaagcagcagtccagcctgtacctgacagccag |
| | | ccagctgagctacagcggcacctacttttgtggca |
| | | ccgcctggatcaacgactacaagctgtctttcgga |
| | | gccggcaccacagtgacagtgcgggccaatattca |
| | | gaaccccgatcctgccgtgtaccagctgagagaca |
| | | gcaagagcagcgacaagagcgtgtgcctgttcacc |
| | | gacttcgacagccagaccaacgtgtcccagagcaa |
| | | ggacagcgacgtgtacatcaccgataagactgtgc |
| | | tggacatgcggagcatggacttcaagagcaacagc |
| | | gccgtggcctggtccaacaagagcgatttcgcctg |
| | | cgccaacgccttcaacaacagcattatccccgagg |
| | | acacattcttcccaagtcctgagagcagctgcgac |
| | | gtgaagctggtggaaaagagcttcgagacagacac |
| | | caacctgaacttccagaacctgagcgtgatcggct |
| | | tcagaatcctgctgctcaaggtggccggcttcaac |
| | | ctgctgatgaccctgagactgtggtccagctaacc |
| | | tCGACTGTGCCTTCTAGTTGCCAGCCATCTGTTGT |
| | | TTGCCCCTCCCCCGTGCCTTCCTTGACCCTGGAAG |
| | | GTGCCACTCCCACTGTCCTTTCCTAATAAAATGAG |
| | | GAAATTGCATCGCATTGTCTGAGTAGGTGTCATTC |
| | | TATTCTGGGGGGTGGGGTGGGGCAGGACAGCAAGG |
| | | GGGAGGATTGGGAAGACAATAGCAGGCATGCTGGG |
| | | GATGCGGTGGGCTCTATGGcttctgaggcggaaag |
| | | aaccagctggggctctagggggtatccccactagt |
| | | cgtgtaccagctgagagactctaaatccagtgaca |
| | | agtctgtctgcctattcaccgattttgattctcaa |
| | | acaaatgtgtcacaaagtaaggattctgatgtgta |
| | | tatcacagacaaaactgtgctagacatgaggtcta |
| | | tggacttcaagagcaacagtgctgtggcctggagc |
| | | aacaaatctgactttgcatgtgcaaacgccttcaa |
| | | caacagcattattccagaagacaccttcttcccca |
| | | gcccaggtaagggcagctttggtgccttcgcaggc |
| | | tgtttccttgcttcaggaatggccaggttctgccc |
| | | agagctctggtcaatgatgtctaaaactcctctga |
| | | ttggtggtctcggccttatccattgccaccaaaac |
| | | cctctttttactaagaaacagtgagccttgttctg |
| | | gcagtccagagaatgacacgggaaaaaagcagatg |
| | | aagagaaggtggcaggagagggcacgtggcccagc |
| | | ctcagtctctagatctaggaacccctagtgatgga |
| | | gttggccactccctctctgcgcgctcgctcgctca |
| | | ctgaggccgcccgggcaaagcccgggcgtcgggcg |
| | | acctttggtcgcccggcctcagtgagcgagcgagc |
| | | gcgcagagagggagtggccaa |
| AAV6-1008 | 15 | tgcatcatcaccgtttttctggacaaccccaaagt |
| GFP | | accccgtctccctggctttagccacctctccatcc |
| insert | | tcttgctttctttgcctggacaccccgttctcctg |
| for AAVS1 | | tggattcgggtcacctctcactcctttcatttggg |
| | | cagctcccctaccccccttacctctctagtctgtg |
| | | ctagctcttccagccccctgtcatggcatcttcca |
| | | ggggtccgagagctcagctagtcttcttcctccaa |
| | | cccgggcccctatgtccacttcaggacagcatgtt |
| | | tgctgcctccagggatcctgtgtccccgagctggg |
| | | accaccttatattcccagggccggttaatgtggct |
| | | ctggttctgggtacttttatctgtcccctccaccc |
| | | cacagtggggccactagggacaggattggtgacag |
| | | aaaagccccatccttaggcctcctccttccgagta |
| | | attcatacaaaaggactcgcccctgccttggggaa |
| | | tcccagggaccgtcgttaaactcccactaacgtag |
| | | aacccagagatcgctgcgttcccgccccctcaccc |
| | | gcccgctctcgtcatcactgaggtggagaagagca |
| | | tgcgtgaggctccggtgcccgtcagtgggcagagc |
| | | gcacatcgcccacagtccccgagaagttgggggga |
| | | ggggtcggcaattgaaccggtgcctagagaaggtg |
| | | gcgcggggtaaactgggaaagtgatgtcgtgtact |
| | | ggctccgcctttttcccgagggtgggggagaaccg |
| | | tatataagtgcagtagtcgccgtgaacgttctttt |
| | | tcgcaacgggtttgccgccagaacacaggtaagtg |
| | | ccgtgtgtggttcccgcgggcctggcctctttacg |
| | | ggttatggcccttgcgtgccttgaattacttccac |
| | | gcccctggctgcagtacgtgattcttgatcccgag |
| | | cttcgggttggaagtgggtgggagagttcgaggcc |
| | | ttgcgcttaaggagccccttcgcctcgtgcttgag |
| | | ttgaggcctggcttgggcgctggggccgccgcgtg |
| | | cgaatctggtggcaccttcgcgcctgtctcgctgc |
| | | tttcgataagtctctagccatttaaaatttttgat |
| | | gacctgctgcgacgctttttttctggcaagatagt |
| | | cttgtaaatgcgggccaacatctgcacactggtat |
| | | ttcggtttttggggccgcgggcggcgacggggccc |
| | | gtgcgtcccagcgcacatgttcggcgaggcggggc |
| | | ctgcgagcgcggccaccgagaatcggacgggggta |
| | | gtctcaagctggccggcctgctctggtgcctggcc |
| | | tcgcgccgccgtgtatcgccccgccctgggcggca |
| | | aggctggcccggtcggcaccagttgcgtgagcgga |
| | | aagatggccgcttcccggccctgctgcagggagct |
| | | caaaatggaggacgcggcgctcgggagagcggggg |
| | | gtgagtcacccacacaaaggaaaagggcctttccg |
| | | tcctcagccgtcgcttcatgtgactccacggagta |
| | | ccgggcgccgtccaggcacctcgattagttctcga |
| | | gcttttggagtacgtcgtctttaggttggggggag |
| | | gggttttatgcgatggagtttccccacactgagtg |
| | | ggtggagactgaagttaggccagcttggcacttga |
| | | tgtaattctccttggaatttgccctttttgagttt |
| | | ggatcttggttcattctcaagcctcagacagtggt |
| | | tcaaagtttttttcttccatttcaggtgtcgtgac |
| | | gctagcgctaccggactcaatctcgagctcaagct |
| | | tcgaattctgcagtcgacggtaccgcgggcccggg |
| | | atccaccggtcgccaccatggtgAGCAAGGGCGAG |
| | | GAGCTGTTCACCGGGGTGGTGCCCATCCTGGTCGA |
| | | GCTGGACGGCGACGTAAACGGCCACAAGTTCAGCG |
| | | TGTCCGGCGAGGGCGAGGGCGATGCCACCTACGGC |
| | | AAGCTGACCCTGAAGTTCATCTGCACCACCGGCAA |
| | | GCTGCCCGTGCCCTGGCCCACCCTCGTGACCACCC |
| | | TGACCTACGGCGTGCAGTGCTTCAGCCGCTACCCC |
| | | GACCACATGAAGCAGCACGACTTCTTCAAGTCCGC |
| | | CATGCCCGAAGGCTACGTCCAGGAGCGCACCATCT |
| | | TCTTCAAGGACGACGGCAACTACAAGACCCGCGCC |
| | | GAGGTGAAGTTCGAGGGCGACACCCTGGTGAACCG |
| | | CATCGAGCTGAAGGGCATCGACTTCAAGGAGGACG |
| | | GCAACATCCTGGGGCACAAGCTGGAGTACAACTAC |
| | | AACAGCCACAACGTCTATATCATGGCCGACAAGCA |
| | | GAAGAACGGCATCAAGGTGAACTTCAAGATCCGCC |
| | | ACAACATCGAGGACGGCAGCGTGCAGCTCGCCGAC |
| | | CACTACCAGCAGAACACCCCCATCGGCGACGGCCC |
| | | CGTGCTGCTGCCCGACAACCACTACCTGAGCACCC |
| | | AGTCCGCCCTGAGCAAAGACCCCAACGAGAAGCGC |
| | | GATCACATGGTCCTGCTGGAGTTCGTGACCGCCGC |
| | | CGGGATCACTCTCGGCATGGACGAGCTGTACAAGt |
| | | aatagcggccgcgactctagatcataatcagccat |
| | | accacatttgtagaggttttacttgctttaaaaaa |
| | | cctcccacacctccccctgaacctgaaacataaaa |
| | | tgaatgcaattgttgttgttaacttgtttattgca |
| | | gcttataatggttacaaataaagcaatagcatcac |
| | | aaatttcacaaataaagcatttttttcactgcatt |
| | | ctagttgtggtttgtccaaactcatcaatgtatct |
| | | taaggcgttagtctcctgatattgggtctaacccc |
| | | cacctcctgttaggcagattccttatctggtgaca |
| | | cacccccatttcctggagccatctctctccttgcc |
| | | agaacctctaaggtttgcttacgatggagccagag |
| | | aggatcctgggagggagagcttggcagggggggga |
| | | gggaagggggggatgcgtgacctgcccggttctca |
| | | gtggccaccctgcgctaccctctcccagaacctga |
| | | gctgctctgacgcggccgtctggtgcgtttcactg |
| | | atcctggtgctgcagcttccttacacttcccaaga |
| | | ggagaagcagtttggaaaaacaaaatcagaataag |
| | | ttggtcctgagttctaactttggctcttcaccttt |
| | | ctagtccccaatttatattgttcctccgtgcgtca |
| | | gttttacctgtgagataaggccagtagccagcccc |
| | | gt |
| AAV6-231 | 16 | gaccactttgagctctactggcttctgcgccgcct |
| GFP | | ctggcccactgtttccccttcccaggcaggtcctg |
| insert | | ctttctctgacctgcattctctcccctgggcctgt |
| for AAVS1 | | gccgctttctgtctgcagcttgtggcctgggtcac |
| | | ctctacggctggcccagatccttccctgccgcctc |
| | | cttcaggttccgtcttcctccactccctcttcccc |
| | | ttgctctctgctgtgttgctgcccaaggatgctct |
| | | ttccggagcacttccttctcggcgctgcaccacgt |
| | | gatgtcctctgagcggatcctccccgtgtctgggt |
| | | cctctccgggcatctctcctccctcacccaacccc |
| | | atgccgtcttcactcgctgggttcccttttccttc |
| | | tccttctggggcctgtgccatctctcgtttcttag |
| | | gatggccttctccgacggatgtctcccttgcgtcc |
| | | cgcctccccttcttgtaggcctgcatcatcaccgt |
| | | ttttctggacaaccccaaagtaccccgtctccctg |
| | | gctttagccacctctccatcctcttgctttctttg |
| | | cctggacaccccgttctcctgtggattcgggtcac |
| | | ctctcactcctttcatttgggcagctcccctaccc |
| | | cccttacctctctagtctgtgctagctcttccagc |
| | | cccctgtcatggcatcttccaggggtccgagagct |
| | | cagctagtcttcttcctccaacccgggcccctatg |
| | | tccacttcaggacagcatgtttgctgcctccaggg |
| | | atcctgtgtccccgagctgggaccaccttatattc |
| | | ccagggccggttaatgtggctctggttctgggtac |
| | | ttttatctgtcccctccaccccacagtggggccac |
| | | tagggacaggattggtgacagaaaagccccatcct |
| | | taggcctcctccttagttattaatgagtaattcat |
| | | acaaaaggactcgcccctgccttggggaatcccag |
| | | ggaccgtcgttaaactcccactaacgtagaaccca |
| | | gagatcgctgcgttcccgccccctcacccgcccgc |
| | | tctcgtcatcactgaggtggagaagagcatgcgtg |
| | | aggctccggtgcccgtcagtgggcagagcgcacat |
| | | cgcccacagtccccgagaagttggggggaggggtc |
| | | ggcaattgaaccggtgcctagagaaggtggcgcgg |
| | | ggtaaactgggaaagtgatgtcgtgtactggctcc |
| | | gcctttttcccgagggtgggggagaaccgtatata |
| | | agtgcagtagtcgccgtgaacgttctttttcgcaa |
| | | cgggtttgccgccagaacacaggtaagtgccgtgt |
| | | gtggttcccgcgggcctggcctctttacgggttat |
| | | ggcccttgcgtgccttgaattacttccacgcccct |
| | | ggctgcagtacgtgattcttgatcccgagcttcgg |
| | | gttggaagtgggtgggagagttcgaggccttgcgc |
| | | ttaaggagccccttcgcctcgtgcttgagttgagg |
| | | cctggcttgggcgctggggccgccgcgtgcgaatc |
| | | tggtggcaccttcgcgcctgtctcgctgctttcga |
| | | taagtctctagccatttaaaatttttgatgacctg |
| | | ctgcgacgctttttttctggcaagatagtcttgta |
| | | aatgcgggccaaCatctgcacactggtatttcggt |
| | | ttttggggccgcgggcggcgacggggcccgtgcgt |
| | | cccagcgcacatgttcggcgaggcggggcctgcga |
| | | gcgcggccaccgagaatcggacgggggtagtctca |
| | | agctggccggcctgctctggtgcctggcctcgcgc |
| | | cgccgtgtatcgccccgccctgggcggcaaggctg |
| | | gcccggtcggcaccagttgcgtgagcggaaagatg |
| | | gccgcttcccggccctgctgcagggagctcaaaat |
| | | ggaggacgcggcgctcgggagagcgggcgggtgag |
| | | tcacccacacaaaggaaaagggcctttccgtcctc |
| | | agccgtcgcttcatgtgactccacggagtaccggg |
| | | cgccgtccaggcacctcgattagttctcgagcttt |
| | | tggagtacgtcgtctttaggttggggggaggggtt |
| | | ttatgcgatggagtttccccacactgagtgggtgg |
| | | agactgaagttaggccagcttggcacttgatgtaa |
| | | ttctccttggaatttgccctttttgagtttggatc |
| | | ttggttcattctcaagcctcagacagtggttcaaa |
| | | gtttttttcttccatttcaggtgtcgtgacgctag |
| | | cgctaccggactcaatctcgagctcaagcttcgaa |
| | | ttctgcagtcgacggtaccgcgggcccgggatcca |
| | | ccggtcgccaccATGgtgAGCAAGGGCGAGGAGCT |
| | | GTTCACCGGGGTGGTGCCCATCCTGGTCGAGCTGG |
| | | ACGGCGACGTAAACGGCCACAAGTTCAGCGTGTCC |
| | | GGCGAGGGCGAGGGCGATGCCACCTACGGCAAGCT |
| | | GACCCTGAAGTTCATCTGCACCACCGGCAAGCTGC |
| | | CCGTGCCCTGGCCCACCCTCGTGACCACCCTGACC |
| | | TACGGCGTGCAGTGCTTCAGCCGCTACCCCGACCA |
| | | CATGAAGCAGCACGACTTCTTCAAGTCCGCCATGC |
| | | CCGAAGGCTACGTCCAGGAGCGCACCATCTTCTTC |
| | | AAGGACGACGGCAACTACAAGACCCGCGCCGAGGT |
| | | GAAGTTCGAGGGCGACACCCTGGTGAACCGCATCG |
| | | AGCTGAAGGGCATCGACTTCAAGGAGGACGGCAAC |
| | | ATCCTGGGGCACAAGCTGGAGTACAACTACAACAG |
| | | CCACAACGTCTATATCATGGCCGACAAGCAGAAGA |
| | | ACGGCATCAAGGTGAACTTCAAGATCCGCCACAAC |
| | | ATCGAGGACGGCAGCGTGCAGCTCGCCGACCACTA |
| | | CCAGCAGAACACCCCCATCGGCGACGGCCCCGTGC |
| | | TGCTGCCCGACAACCACTACCTGAGCACCCAGTCC |
| | | GCCCTGAGCAAAGACCCCAACGAGAAGCGCGATCA |
| | | CATGGTCCTGCTGGAGTTCGTGACCGCCGCCGGGA |
| | | TCACTCTCGGCATGGACGAGCTGTACAAGTAAtag |
| | | cggccgcgactctagatcataatcagccataccac |
| | | atttgtagaggttttacttgctttaaaaaacctcc |
| | | cacacctccccctgaacctgaaacataaaatgaat |
| | | gcaattgttgttgttaacttgtttattgcagctta |
| | | taatggttacaaataaagcaatagcatcacaaatt |
| | | tcacaaataaagcatttttttcactgcattctagt |
| | | tgtggtttgtccaaactcatcaatgtatcttaagg |
| | | cgtgtctaacccccacctcctgttaggcagattcc |
| | | ttatctggtgacacacccccatttcctggagccat |
| | | ctctctccttgccagaacctctaaggtttgcttac |
| | | gatggagccagagaggatcctgggagggagagctt |
| | | ggcagggggtgggagggaagggggggatgcgtgac |
| | | ctgcccggttctcagtggccaccctgcgctaccct |
| | | ctcccagaacctgagctgctctgacgcggccgtct |
| | | ggtgcgtttcactgatcctggtgctgcagcttcct |
| | | tacacttcccaagaggagaagcagtttggaaaaac |
| | | aaaatcagaataagttggtcctgagttctaacttt |
| | | ggctcttcacctttctagtccccaatttatattgt |
| | | tcctccgtgcgtcagttttacctgtgagataaggc |
| | | cagtagccagccccgtcctggcagggctgtggtga |
| | | ggaggggggtgtccgtgtggaaaactccctttgtg |
| | | agaatggtgcgtcctaggtgttcaccaggtcgtgg |
| | | ccgcctctactccctttctctttctccatccttct |
| | | ttccttaaagagtccccagtgctatctgggacata |
| | | ttcctccgcccagagcagggtcccgcttccctaag |
| | | gccctgctctgggcttctgggtttgagtccttggc |
| | | aagcccaggagaggcgctcaggcttccctgtcccc |
| | | cttcctcgtccaccatctcatgcccctggctctcc |
| | | tgccccttccctacaggggttcctggctctgctct |
| | | tcagactgagccccgttcccctgcatccccgttcc |
| | | cctgcatcccccttcccctgcatcccccagaggcc |
| | | ccaggccacctacttggcctggaccccacgagagg |
| | | ccaccccagccctgtctaccaggctgccttttggg |
| | | tggattctcctccaa |
| Full HDRT | 17 | GAGGGCCGCGGCAGCCTGCTGACCTGCGGCGACGT |
| template- | | GGAGGAGAAtCCCGGCCCCATGgtgAGCAAGGGCG |
| GFP T2A | | AGGAGCTGTTCACCGGGGTGGTGCCCATCCTGGTC |
| insert | | GAGCTGGACGGCGACGTAAACGGCCACAAGTTCAG |
| GFP: | | CGTGTCCGGCGAGGGCGAGGGCGATGCCACCTACG |
| P00894 | | GCAAGCTGACCCTGAAGTTCATCTGCACCACCGGC |
| | | AAGCTGCCCGTGCCCTGGCCCACCCTCGTGACCAC |
| | | CCTGACCTACGGCGTGCAGTGCTTCAGCCGCTACC |
| | | CCGACCACATGAAGCAGCACGACTTCTTCAAGTCC |
| | | GCCATGCCCGAAGGCTACGTCCAGGAGCGCACCAT |
| | | CTTCTTCAAGGACGACGGCAACTACAAGACCCGCG |
| | | CCGAGGTGAAGTTCGAGGGCGACACCCTGGTGAAC |
| | | CGCATCGAGCTGAAGGGCATCGACTTCAAGGAGGA |
| | | CGGCAACATCCTGGGGCACAAGCTGGAGTACAACT |
| | | ACAACAGCCACAACGTCTATATCATGGCCGACAAG |
| | | CAGAAGAACGGCATCAAGGTGAACTTCAAGATCCG |
| | | CCACAACATCGAGGACGGCAGCGTGCAGCTCGCCG |
| | | ACCACTACCAGCAGAACACCCCCATCGGCGACGGC |
| | | CCCGTGCTGCTGCCCGACAACCACTACCTGAGCAC |
| | | CCAGTCCGCCCTGAGCAAAGACCCCAACGAGAAGC |
| | | GCGATCACATGGTCCTGCTGGAGTTCGTGACCGCC |
| | | GCCGGGATCACTCTCGGCATGGACGAGCTGTACAA |
| | | GTAAcctCGACTGTGCCTTCTAGTTGCCAGCCATC |
| | | TGTTGTTTGCCCCTCCCCCGTGCCTTCCTTGACCC |
| | | TGGAAGGTGCCACTCCCACTGTCCTTTCCTAATAA |
| | | AATGAGGAAATTGCATCGCATTGTCTGAGTAGGTG |
| | | TCATTCTATTCTGGGGGGTGGGGTGGGGCAGGACA |
| | | GCAAGGGGGAGGATTGGGAAGACAATAGCAGGCAT |
| | | GCTGGGGATGCGGTGGGCTCTATGGcttctgaggc |
| | | ggaaagaaccagctggggctctagggggtatcccc |
| | | ACTAGTCGTGTACCAGCTGAGAGACTCTAAATCCA |
| | | GTGACAAGTCTGTCTGCCTATTCACCGATTTTGAT |
| | | TCTCAAACAAATGTGTCACAAAGTAAGGATTCTGA |
| | | TGTGTATATCACAGACAAAACTGTGCTAGACATGA |
| | | GGTCTATGGACTTCAAGAGCAACAGTGCTGTGGCC |
| | | TGGAGCAACAAATCTGACTTTGCATGTGCAAACGC |
| | | CTTCAACAACAGCATTATTCCAGAAGACACCTTCT |
| | | TCCCCAGCCCAGgtaagggcagctttggtgccttc |
| | | gcaggctgtttccttgcttcaggaatggccaggtt |
| | | ctgcccagagctctggtcaatgatgtctaaaactc |
| | | ctctgattggtggtctcggccttatccattgccac |
| | | caaaaccctctttttactaagaaacagtgagcctt |
| | | gttctggcagtccagagaatgacacgggaaaaaag |
| | | cagatgaagagaaggtggcaggagagggcacgtgg |
| | | cccagcctcagtctct |
| pINT | 18 | TTGGCCACTCCCTCTCTGCGCGCTCGCTCGCTCAC |
| 1280, | | TGAGGCCGGGCGACCAAAGGTCGCCCGACGCCCGG |
| HD3 TCR | | GCTTTGCCCGGGCGGCCTCAGTGAGCGAGCGAGCG |
| insertion | | CGCAGAGAGGGAGTGGCCAACTCCATCACTAGGGG |
| including | | TTCCTagatcttgccaacataccataaacctccca |
| ITRs | | ttctgctaatgcccagcctaagttggggagaccac |
| | | tccagattccaagatgtacagtttgctttgctggg |
| | | cctttttcccatgcctgcctttactctgccagagt |
| | | tatattgctggggttttgaagaagatcctattaaa |
| | | taaaagaataagcagtattattaagtagccctgca |
| | | tttcaggtttccttgagtggcaggccaggcctggc |
| | | cgtgaacgttcactgaaatcatggcctcttggcca |
| | | agattgatagcttgtgcctgtccctgagtcccagt |
| | | ccatcacgagcagctggtttctaagatgctatttc |
| | | ccgtataaagcatgagaccgtgacttgccagcccc |
| | | acagagccccgcccttgtccatcactggcatctgg |
| | | actccagcctgggttggggcaaagagggaaatgag |
| | | atcatgtcctaaccctgatcctcttgtcccacagA |
| | | TATCCAGAACCCTGACCCTGCGGCTCCGGTGCCCG |
| | | TCAGTGGGCAGAGCGCACATCGCCCACAGTCCCCG |
| | | AGAAGTTGGGGGGAGGGGTCGGCAATTGAACCGGT |
| | | GCCTAGAGAAGGTGGCGCGGGGTAAACTGGGAAAG |
| | | TGATGTCGTGTACTGGCTCCGCCTTTTTCCCGAGG |
| | | GTGGGGGAGAACCGTATATAAGTGCAGTAGTCGCC |
| | | GTGAACGTTCTTTTTCGCAACGGGTTTGCCGCCAG |
| | | AACACAGGTAAGTGCCGTGTGTGGTTCCCGCGGGC |
| | | CTGGCCTCTTTACGGGTTATGGCCCTTGCGTGCCT |
| | | TGAATTACTTCCACGCCCCTGGCTGCAGTACGTGA |
| | | TTCTTGATCCCGAGCTTCGGGTTGGAAGTGGGTGG |
| | | GAGAGTTCGAGGCCTTGCGCTTAAGGAGCCCCTTC |
| | | GCCTCGTGCTTGAGTTGAGGCCTGGCTTGGGCGCT |
| | | GGGGCCGCCGCGTGCGAATCTGGTGGCACCTTCGC |
| | | GCCTGTCTCGCTGCTTTCGATAAGTCTCTAGCCAT |
| | | TTAAAATTTTTGATGACCTGCTGCGACGCTTTTTT |
| | | TCTGGCAAGATAGTCTTGTAAATGCGGGCCAAGAT |
| | | gTGCACACTGGTATTTCGGTTTTTGGGGCCGCGGG |
| | | CGGCGACGGGGCCCGTGCGTCCCAGCGCACATGTT |
| | | CGGCGAGGCGGGGCCTGCGAGCGCGGCCACCGAGA |
| | | ATCGGACGGGGGTAGTCTCAAGCTGGCCGGCCTGC |
| | | TCTGGTGCCTGGCCTCGCGCCGCCGTGTATCGCCC |
| | | CGCCCTGGGCGGCAAGGCTGGCCCGGTCGGCACCA |
| | | GTTGCGTGAGCGGAAAGATGGCCGCTTCCCGGCCC |
| | | TGCTGCAGGGAGCTCAAAATGGAGGACGCGGCGCT |
| | | CGGGAGAGCGGGCGGGTGAGTCACCCACACAAAGG |
| | | AAAAGGGCCTTTCCGTCCTCAGCCGTCGCTTCATG |
| | | TGACTCCACGGAGTACCGGGCGCCGTCCAGGCACC |
| | | TCGATTAGTTCTCGAGCTTTTGGAGTACGTCGTCT |
| | | TTAGGTTGGGGGGAGGGGTTTTATGCGATGGAGTT |
| | | TCCCCACACTGAGTGGGTGGAGACTGAAGTTAGGC |
| | | CAGCTTGGCACTTGATGTAATTCTCCTTGGAATTT |
| | | GCCCTTTTTGAGTTTGGATCTTGGTTCATTCTCAA |
| | | GCCTCAGACAGTGGTTCAAAGTTTTTTTCTTCCAT |
| | | TTCAGGTGTCGTGAtgcggCCGCCACCATGGGATG |
| | | TAGACTTCTGTGTTGCGCCGTGCTGTGTCTGCTTG |
| | | GAGCTGGCGAACTGGTGCCTATGGAAACCGGCGTG |
| | | ACCCAGACACCTAGACACCTGGTCATGGGCATGAC |
| | | AAACAAGAAAAGCCTGAAGTGCGAGCAGCACCTGG |
| | | GCCACAATGCCATGTACTGGTACAAGCAGAGCGCC |
| | | AAGAAACCCCTGGAACTGATGTTCGTGTACAGCCT |
| | | GGAAGAGAGGGTCGAGAACAACAGCGTGCCCAGCA |
| | | GATTCAGCCCTGAGTGCCCTAATAGCAGCCACCTG |
| | | TTTCTGCATCTGCACACCCTGCAGCCTGAGGACTC |
| | | TGCCCTGTATCTGTGTGCCAGCAGCCAGGACTACC |
| | | TGGTGTCCAACGAGAAGCTGTTCTTCGGCAGCGGC |
| | | ACACAGCTGAGCGTGCTGGAAGATCTGAAGAACGT |
| | | GTTCCCACCTGAGGTGGCCGTGTTCGAGCCTTCTG |
| | | AGGCCGAGATCAGCCACACACAGAAAGCCACACTC |
| | | GTGTGTCTGGCCACCGGCTTCTATCCCGATCACGT |
| | | GGAACTGTCTTGGTGGGTCAACGGCAAAGAGGTGC |
| | | ACAGCGGCGTCAGCACCGATCCTCAGCCTCTGAAA |
| | | GAGCAGCCCGCTCTGAACGACAGCAGATACTGCCT |
| | | GAGCAGCAGACTGAGAGTGTCCGCCACCTTCTGGC |
| | | AGAACCCCAGAAACCACTTCAGATGCCAGGTGCAG |
| | | TTCTACGGCCTGAGCGAGAACGATGAGTGGACCCA |
| | | GGATAGAGCCAAGCCTGTGACACAGATCGTGTCTG |
| | | CCGAAGCCTGGGGCAGAGCCGATTGTGGCTTTACC |
| | | AGCGAGAGCTACCAGCAGGGCGTGCTGTCTGCCAC |
| | | AATCCTGTACGAGATCCTGCTGGGAAAAGCCACTC |
| | | TGTACGCTGTGCTGGTGTCCGCTCTGGTGCTGATG |
| | | GCCATGGTCAAGCGGAAGGATAGCAGGGGCGGCTC |
| | | CGGTGCCACAAACTTCTCCCTGCTCAAGCAGGCCG |
| | | GAGATGTGGAAGAGAACCCTGGCCCTATGATCAGC |
| | | CTGAGAGTGCTGCTGGTCATCCTGTGGCTGCAGCT |
| | | GTCTTGGGTCTGGTCCCAGCGGAAAGAGGTGGAAC |
| | | AGGACCCCGGACCTTTCAATGTGCCTGAAGGCGCC |
| | | ACCGTGGCCTTCAACTGCACCTACAGCAATAGCGC |
| | | CAGCCAGAGCTTCTTCTGGTACAGACAGGACTGCC |
| | | GGAAAGAACCCAAGCTGCTGATGAGCGTGTACAGC |
| | | AGCGGCAACGAGGACGGCAGATTCACAGCCCAGCT |
| | | GAACAGAGCCAGCCAGTACATCAGCCTGCTGATCC |
| | | GGGATAGCAAGCTGAGCGATAGCGCCACCTACCTG |
| | | TGCGTGGTCAACCTGCTGTCTAATCAAGGCGGCAA |
| | | GCTGATCTTCGGCCAGGGCACAGAGCTGAGCGTGA |
| | | AGCCCAACATTCAGAACCCCGATCCTGCCGTGTAC |
| | | CAGCTGAGAGACAGCAAGAGCAGCGACAAGAGCGT |
| | | GTGCCTGTTCACCGACTTCGACAGCCAGACCAACG |
| | | TGTCCCAGAGCAAGGACAGCGACGTGTACATCACC |
| | | GATAAGACcGTGCTGGACATGCGGAGCATGGACTT |
| | | CAAGAGCAACAGCGCCGTGGCCTGGTCCAACAAGA |
| | | GCGATTTCGCCTGCGCCAACGCCTTCAACAACAGC |
| | | ATTATCCCCGAGGACACATTCTTCCCAAGTCCTGA |
| | | GAGCAGCTGCGACGTGAAGCTGGTGGAAAAGAGCT |
| | | TCGAGACAGACACCAACCTGAACTTCCAGAACCTG |
| | | TCCGTGATCGGCTTCCGGATCCTGCTGCTGAAAGT |
| | | GGCCGGCTTCAACCTCCTGATGACCCTGAGACTGT |
| | | GGTCCAGCTAAcctCGACTGTGCCTTCTAGTTGCC |
| | | AGCCATCTGTTGTTTGCCCCTCCCCCGTGCCTTCC |
| | | TTGACCCTGGAAGGTGCCACTCCCACTGTCCTTTC |
| | | CTAATAAAATGAGGAAATTGCATCGCATTGTCTGA |
| | | GTAGGTGTCATTCTATTCTGGGGGGTGGGGTGGGG |
| | | CAGGACAGCAAGGGGGAGGATTGGGAAGACAATAG |
| | | CAGGCATGCTGGGGATGCGGTGGGCTCTATGGctt |
| | | ctgaggcggaaagaaccagctggggctctaggggg |
| | | tatccccACTAGTCGTGTACCAGCTGAGAGACTCT |
| | | AAATCCAGTGACAAGTCTGTCTGCCTATTCACCGA |
| | | TTTTGATTCTCAAACAAATGTGTCACAAAGTAAGG |
| | | ATTCTGATGTGTATATCACAGACAAAACTGTGCTA |
| | | GACATGAGGTCTATGGACTTCAAGAGCAACAGTGC |
| | | TGTGGCCTGGAGCAACAAATCTGACTTTGCATGTG |
| | | CAAACGCCTTCAACAACAGCATTATTCCAGAAGAC |
| | | ACCTTCTTCCCCAGCCCAGgtaagggcagctttgg |
| | | tgccttcgcaggctgtttccttgcttcaggaatgg |
| | | ccaggttctgcccagagctctggtcaatgatgtct |
| | | aaaactcctctgattggtggtctcggccttatcca |
| | | ttgccaccaaaaccctctttttactaagaaacagt |
| | | gagccttgttctggcagtccagagaatgacacggg |
| | | aaaaaagcagatgaagagaaggtggcaggagaggg |
| | | cacgtggcccagcctcagtctctAGATCTAGGAAC |
| | | CCCTAGTGATGGAGTTGGCCACTCCCTCTCTGCGC |
| | | GCTCGCTCG |

1. A compound having the structure of Formula I: 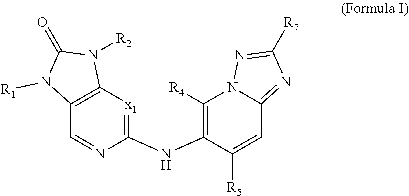
or a salt thereof,
wherein:
x₁ is C—R₃ or N;
R₁ is C₁-C₃ alkyl;
R₂ is cycloalkyl or heterocyclyl, and cycloalkyl and heterocyclyl are optionally substituted with one or more R₆;
R₃ is H or C₁-C₃ alkyl;
R₄ is H or C₁-C₃ alkyl;
R₅ is C₁-C₃ alkyl;
each R₆ is independently selected from hydroxy, halo, alkyl, alkoxy, cycloalkyl, amino, and cyano, or two R₆, taken together with the atom or atoms to which they are bonded, form a spirocyclic or fused ring; and
R₇ is H or C₁-C₃ alkyl,
provided that at least one of the following applies:
(a) x₁ is C—R₃;
(b) R₁ is C₂-C₃ alkyl;
(c) R₄ is C₁-C₃ alkyl;
(d) R₂ is substituted with one R₆, and R₆ is halo;
(e) R₂ is substituted with two R₆ that, taken together with the atom or atoms to which they are bonded, form a spirocyclic or fused ring; and
(f) R₂ is C₃-C₅ cycloalkyl optionally substituted with one or more R₆.

2. The compound of claim 1, wherein x₁ is C—R₃.

3. The compound of claim 2, wherein R₃ is H or methyl.

4. The compound of claim 1, wherein x₁ is N.

5. The compound of claim 1, wherein R₁ is C₂-C₃ alkyl.

6. The compound of claim 1, wherein R₁ is selected from methyl and ethyl.

7. (canceled)

8. The compound of claim 1, wherein R₄ is C₁-C₃ alkyl.

9. The compound of claim 1, wherein R₄ is H or methyl.

10. (canceled)

11. The compound of claim 1, wherein R₂ is cycloalkyl.

12-14. (canceled)

15. The compound of claim 1, wherein R₂ is heterocyclyl.

16-18. (canceled)

19. The compound of claim 1, wherein R₂ is optionally substituted with one or more R₆ independently selected from hydroxy, halo, methoxy, methyl, and cycloalkyl, or two R₆, taken together with the atom or atoms to which they are bonded, form a spirocyclic or fused ring.

20-24. (canceled)

25. The compound of claim 1, wherein R₅ is methyl.

26. The compound of claim 1, wherein R₇ is H or methyl.

27. A compound selected from: 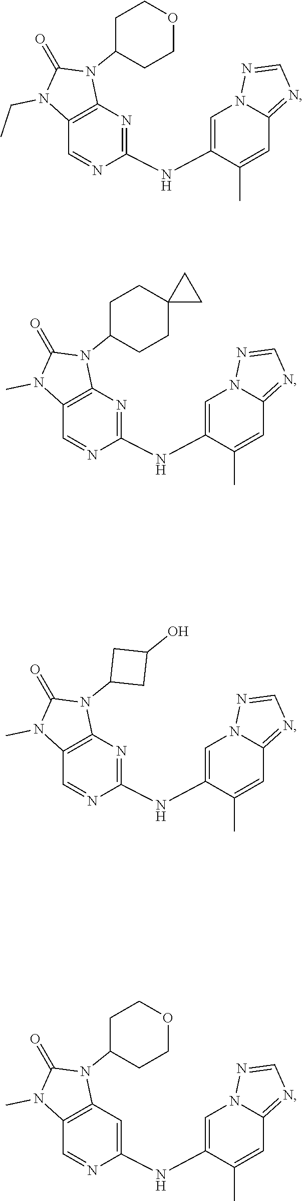 [SMILES: CCn1c(=O)n(C2CCOCC2)c2nc(Nc3cn4ncnc4cc3C)ncc21 ; Cc1cc2ncnn2cc1Nc1ncc2c(n1)n(C1CCC3(CC1)CC3)c(=O)n2C ; Cc1cc2ncnn2cc1Nc1ncc2c(n1)n(C1CC(O)C1)c(=O)n2C ; Cc1cc2ncnn2cc1Nc1cc2c(cn1)n(C)c(=O)n2C1CCOCC1] 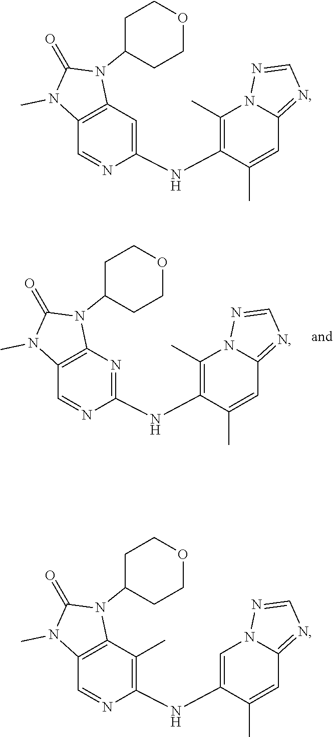 [SMILES: Cc1cc2ncnn2c(C)c1Nc1cc2c(cn1)n(C)c(=O)n2C1CCOCC1 ; Cc1cc2ncnn2c(C)c1Nc1ncc2c(n1)n(C1CCOCC1)c(=O)n2C ; Cc1cc2ncnn2cc1Nc1ncc2c(c1C)n(C1CCOCC1)c(=O)n2C]
or a salt thereof.

28-37. (canceled)

38. A composition comprising
a) a DNA protein kinase inhibitor (DNA-PKI);
b) a DNA cutting agent;
c) optionally, a cell; and
d) optionally, a donor DNA;
wherein the DNA-PKI is a compound of Formula I 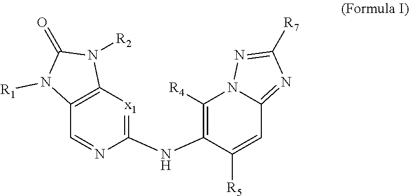
or a salt thereof,
wherein:
x₁ is C—R₃ or N;
R₁ is C₁-C₃ alkyl;
R₂ is cycloalkyl or heterocyclyl, and cycloalkyl and heterocyclyl are optionally substituted with one or more R₆;
R₃ is H or C₁-C₃ alkyl;
R₄ is H or C₁-C₃ alkyl;
R₅ is C₁-C₃ alkyl;
each R₆ is independently selected from hydroxy, halo, alkyl, alkoxy, cycloalkyl, amino, and cyano, or two R₆, taken together with the atom or atoms to which they are bonded, form a spirocyclic or fused ring; and
R₇ is H or C₁-C₃ alkyl.

39. The composition of claim 38, wherein x₁ is N.

40. The composition of claim 38, wherein R₁ is methyl.

41. The composition of claim 38, wherein R₄ is H.

42. The composition of claim 38, wherein R₂ is selected from cyclohexyl, tetrahydropyranyl, and tetrahydrofuranyl.

43-44. (canceled)

45. The composition of claim 38, wherein R₂ is optionally substituted with one or more R₆ independently selected from hydroxy, methoxy, and methyl.

46. The composition of claim 38, wherein R₅ is methyl.

47. The composition of claim 38, wherein R₇ is H or methyl.

48. (canceled)

49. A composition comprising
a) a DNA protein kinase inhibitor (DNA-PKI);
b) a DNA cutting agent;
c) optionally, a cell; and
d) optionally, a donor DNA;
wherein the DNA-PKI is a compound of claim 27.

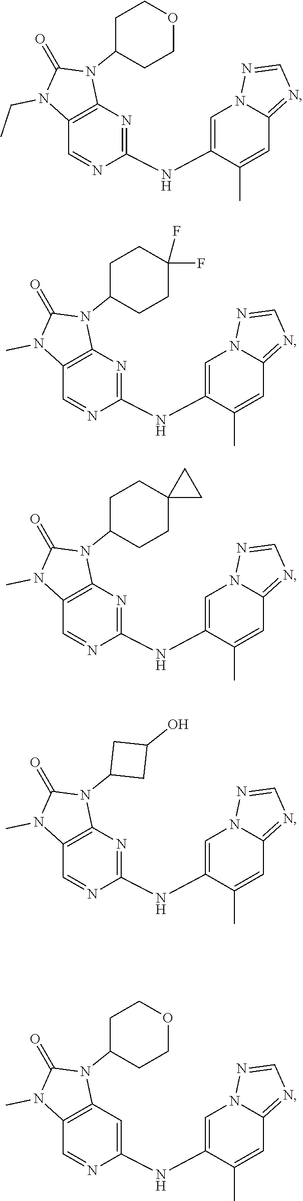 [SMILES: CCn1c(=O)n(C2CCOCC2)c2nc(Nc3cn4ncnc4cc3C)ncc21 ; Cc1cc2ncnn2cc1Nc1ncc2c(n1)n(C1CCC(F)(F)CC1)c(=O)n2C ; Cc1cc2ncnn2cc1Nc1ncc2c(n1)n(C1CCC3(CC1)CC3)c(=O)n2C ; Cc1cc2ncnn2cc1Nc1ncc2c(n1)n(C1CC(O)C1)c(=O)n2C ; Cc1cc2ncnn2cc1Nc1cc2c(cn1)n(C)c(=O)n2C1CCOCC1]

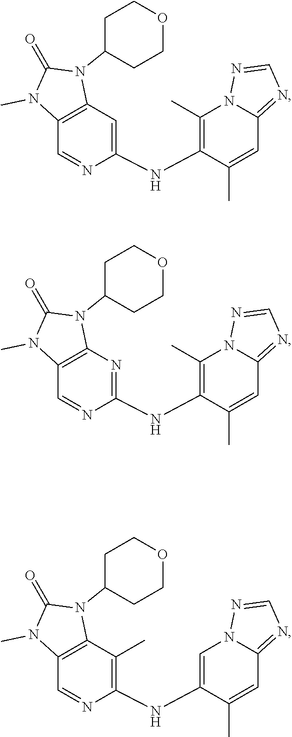 [SMILES: Cc1cc2ncnn2c(C)c1Nc1cc2c(cn1)n(C)c(=O)n2C1CCOCC1 ; Cc1cc2ncnn2c(C)c1Nc1ncc2c(n1)n(C1CCOCC1)c(=O)n2C ; Cc1cc2ncnn2cc1Nc1ncc2c(c1C)n(C1CCOCC1)c(=O)n2C]

50-61. (canceled)

62. The composition of claim 38, comprising a cell.

63. The composition of claim 62, wherein the cell is a eukaryotic cell.

64-66. (canceled)

67. The composition of claim 65, wherein the cell is a stem cell or an immune cell.

68-78. (canceled)

79. The composition of claim 38, wherein the DNA cutting agent comprises a CRISPR/Cas nuclease component and optionally a guide RNA component.

80-81. (canceled)

82. The composition of claim 79, wherein the CRISPR/Cas nuclease component comprises a Cas nuclease or an mRNA encoding the Cas nuclease.

83-84. (canceled)

85. The composition of claim 82, wherein the Cas nuclease is a Cas9 nuclease.

86. The composition of claim 82, wherein the Cas nuclease is a S. pyogenes Cas9 nuclease or a N. meningitidis Cas9 nuclease.

87-89. (canceled)

90. The composition of claim 38, comprising a modified RNA.

91. The composition of claim 79, wherein the guide RNA component is a guide RNA nucleic acid such as a modified or unmodified guide RNA.

92. (canceled)

93. The composition of claim 91, wherein the guide RNA nucleic acid is or encodes a dual-guide RNA (dgRNA) or is or encodes a single-guide (sgRNA).

94-95. (canceled)

96. The composition of claim 91, wherein the guide RNA nucleic acid is a modified gRNA; and the modified gRNA comprises a modification at one or more of the first five nucleotides at the 5′ end or at the 3′ end.

97-98. (canceled)

99. The composition of claim 38, comprising the donor DNA.

100. The composition of claim 99, wherein the donor DNA comprises a template comprising a sequence encoding a protein, a regulatory sequence, or a sequence encoding structural RNA.

101. The composition of claim 38, wherein the DNA cutting agent is present in a lipid nanoparticle (LNP) composition.

102-123. (canceled)

124. A method for targeted genome editing in a cell, a method of repairing a double stranded DNA break in a cell, a method of inhibiting or suppressing repair of a DNA break in a cell via a non-homologous end joining (NHEJ) pathway, or a method of targeted insertion of a donor DNA into the genome of a cell, comprising contacting the cell with a DNA cutting agent and a DNA-PKI, wherein the DNA-PKI is a compound of Formula I 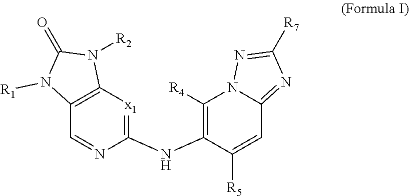
or a salt thereof,
wherein:
x₁ is C—R₃ or N;
R₁ is C₁-C₃ alkyl;
R₂ is cycloalkyl or heterocyclyl, and cycloalkyl and heterocyclyl are optionally substituted with one or more R₆;
R₃ is H or C₁-C₃ alkyl;
R₄ is H or C₁-C₃ alkyl;
R₅ is C₁-C₃ alkyl;
each R₆ is independently selected from hydroxy, halo, alkyl, alkoxy, cycloalkyl, amino, and cyano, or two R₆, taken together with the atom or atoms to which they are bonded, form a spirocyclic or fused ring; and
R₇ is H or C₁-C₃ alkyl.

125-148. (canceled)

149. A method for targeted genome editing in a cell, a method of repairing a double stranded DNA break in a cell, a method of inhibiting or suppressing repair of a DNA break in a cell via a non-homologous end joining (NHEJ) pathway, or a method of targeted insertion of a donor DNA into the genome of a cell, comprising contacting the cell with a DNA cutting agent and a DNA-PKI, wherein the DNA-PKI is a compound of claim 27

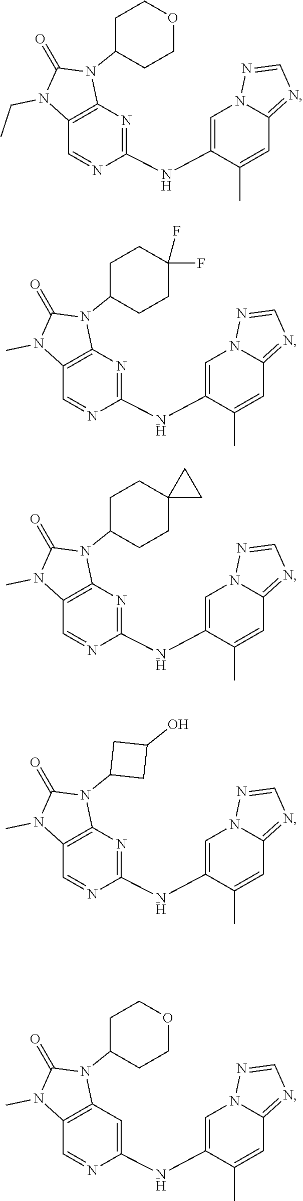 [SMILES: CCn1c(=O)n(C2CCOCC2)c2nc(Nc3cn4ncnc4cc3C)ncc21 ; Cc1cc2ncnn2cc1Nc1ncc2c(n1)n(C1CCC(F)(F)CC1)c(=O)n2C ; Cc1cc2ncnn2cc1Nc1ncc2c(n1)n(C1CCC3(CC1)CC3)c(=O)n2C ; Cc1cc2ncnn2cc1Nc1ncc2c(n1)n(C1CC(O)C1)c(=O)n2C ; Cc1cc2ncnn2cc1Nc1cc2c(cn1)n(C)c(=O)n2C1CCOCC1]

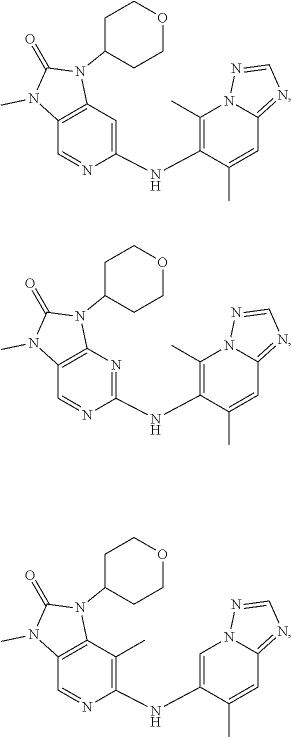 [SMILES: Cc1cc2ncnn2c(C)c1Nc1cc2c(cn1)n(C)c(=O)n2C1CCOCC1 ; Cc1cc2ncnn2c(C)c1Nc1ncc2c(n1)n(C1CCOCC1)c(=O)n2C ; Cc1cc2ncnn2cc1Nc1ncc2c(c1C)n(C1CCOCC1)c(=O)n2C]

150-181. (canceled)

182. The method of claim 124, wherein the DNA cutting agent is selected from a zine finger nuclease, a TALE effector domain nuclease (TALEN), a CRISPR/Cas nuclease component, and combinations thereof.

183-191. (canceled)

192. The method of claim 124, further comprising contacting the cell with a modified RNA.

193. The method of claim 124, further comprising contacting the cell with a guide RNA nucleic acid.

194-197. (canceled)

198. The method of claim 193, wherein the guide RNA nucleic acid is a modified gRNA; and the modified gRNA comprises a modification at one or more of the first five nucleotides at the 5′ end or at the 3′ end.

199-200. (canceled)

201. The method of claim 124, further comprising contacting the cell with a donor DNA.

202. (canceled)

203. The method claim 201, wherein the donor DNA comprises a template comprising a sequence encoding a protein, a regulatory sequence, a sequence encoding structural RNA.

204-217. (canceled)

218. The method of claim 124, further comprising contacting the cell with a vector.

219-240. (canceled)
